(19) United States
(12) Patent Application Publication
(10) Pub. No.: US 2019/0142810 A1
(43) Pub. Date: May 16, 2019

(54) MITOCHONDRIAL INHIBITORS FOR THE TREATMENT OF PROLIFERATION DISORDERS

(71) Applicant: Basilea Pharmaceutica International AG, Basel (CH)

(72) Inventors: Heidi LANE, Basel (CH); Florian RICHALET, Basel (CH); Mahmoud EL SHEMERLY, Basel (CH)

(21) Appl. No.: 16/310,918

(22) PCT Filed: Jun. 29, 2017
(86) PCT No.: PCT/EP2017/066129

§ 371 Date: Dec. 18, 2018

(30) Foreign Application Priority Data
Jun. 30, 2016 (EP) 16177193.6

(51) Int. Cl. A61K 31/433; A61P 35/00

(52) U.S. Cl. CPC A61K 31/433; A61P 35/00

(57) ABSTRACT

The invention provides compounds of formula I or pharmaceutically acceptable salt, solvate or hydrate thereof (I) wherein ring A represents group A-I or A-II (A-I, A-II) A1, A2, A3, A4 represent independently C(R4aa) or N, wherein no more than one of A1, A2, A3, and A4 represents N; A5 represents C(R4b) or N; B1, B2, B3 and B4 represent independently C(R3) or N, wherein no more than two of B1, B2, B3 and B4 represent N; n is 1 or 2; and R1, R2, R3, R4a and R4aa and R4b are as defined in the claims, as well as methods of using the compounds to treat proliferation diseases, in particular cancer.

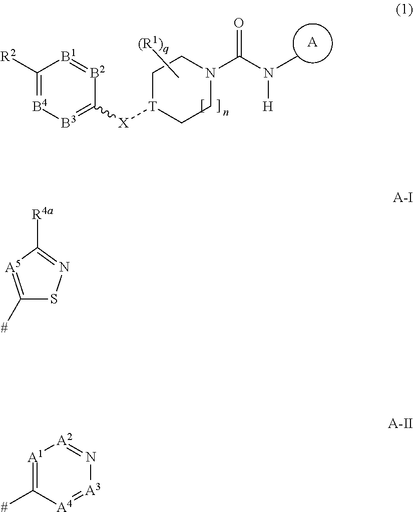

The present invention relates to mitochondrial inhibitors and their use in the treatment of proliferation disorders, in particular cancer.

Mitochondria are the power house of the cell because they generate most of the adenosine triphosphate (ATP), used as a source of chemical energy (Campbell, Neil A.; Brad Williamson; Robin J. Heyden. Biology: Exploring Life 2006^{th} Edition, Publisher: Pearson Prentice Hall, 2006). In addition, mitochondria are involved in other functions, such as cellular signaling, differentiation and death, as well as maintaining control of the cell cycle and cell growth (McBride H. M. et. al., Curr. Biol., vol. 16, no. 14, R551-60, 2006).

Cancer cells reprogram their metabolism in favour of glycolysis, regardless of oxygen presence, according to a phenomenon known as anaerobic glycolysis. This so-called “Warburg phenotype” involves high glucose uptake and a high glycolytic activity (O. Warburg, Science, vol. 123, no. 3191, pages 309-314, 1956). Nevertheless, cancer cells are also dependent on mitochondria for ATP production through oxidative phosphorylation (OXPHOS) (Marchetti P. et al., International Journal of Cell Biology, vol. 2015, pages 1-17, 2015 and Solaini G. et al., Biochim. Biophys. Acta, vol. 2, page: 314-323, 2010). Mitochondrial metabolism is now recognized as a potential target for anticancer agents due to the metabolic characteristic of cancer cells. Indeed, human cancer is associated with mitochondrial dysregulation, which promotes cancer cell survival, tumor progression and metastases as well as resistance to current anticancer drugs (Marchetti P. et al., International Journal of Cell Biology, Volume 2015, pages 1-17, 2015, Boland M. L. et al., Frontieres in Oncology, vol. 3, Article 292, pages 1-28, 2013 and Solaini G. et al., Biochim. Biophys. Acta, Volume 1797, page: 1171-1177, 2010). Metabolic reprogramming in cancer cells results in the maintenance of energy (ATP) production even under stressed conditions, contributing to tumor growth and survival through (for example) mitochondrial utilization of alternative carbon sources such as glutamine and fatty acids to generate ATP (Solaini G. et al., Biochim. Biophys. Acta, Volume 1797, page: 1171-1177, 2010). Indeed, as a result of the separation of glycolytic flux from mitochondria, mitochondrial glutaminolysis is preferentially used to produce ATP and, therefore, contribute to cancer cell survival (DeBerardinis R. J. et al., Proceedings of the National Academy of Sciences of the United States of America, vol. 104, no. 49, pages 19345-19350, 2007) being crucial for the development (Strohecker A. M. et al., Cancer Discovery, vol. 3, no. 11, page 1272-1285, 2013) and anchorage-independent growth (Weinberg F. et al., Proceedings of the National Academy of Sciences of the United States of America, vol. 107, no. 19, pages 8788-8793, 2010) of certain tumor types.

Moreover, mitochondrial activity has also been associated with the development of drug resistance. For example, chemotherapeutic and targeted drugs (e.g. BRAF inhibitors) have been shown to induce a shift in cancer metabolism leading to mitochondrial dependency (addiction) characterized for example by upregulation of OXPHOS and mitochondrial biogenesis in surviving cells (Marchetti P. et al., International Journal of Cell Biology, Volume 2015, pages 1-17, 2015; and Vellinga T. T. et al., Clinical Cancer Research, vol. 21, no. 12, pages 2870-2879, 2015). In the case of BRAF inhibitors, acquired resistance was associated with maintenance of an OXPHOS phenotype regardless of the underlying resistance mechanism (Corazao Rozas P. et al., Oncotarget, vol. 4, no. 11, pages 1986-1998, 2013), suggesting a potential metabolic arena that could be exploited on a therapeutic level. Hence, taken together, accumulating data provide convincing evidence supporting the involvement of mitochondria in cancer development and a strong rationale for developing mitochondrial targeted agents to fight cancer. Based on growing interest in mitochondria as therapeutic targets for cancer, in recent years a number of mitochondrial-targeting investigational agents have entered clinical development. For example, the anti-diabetic medication metformin, which inhibits OXPHOS through inhibition of complex I of the mitochondrial respiratory chain (El-Mir et al., J. Biol. Chem. Vol. 275, pages 223-228, 2000, and Wheaton W. W. et al., eLife vol. 3, 2014) is currently being investigated in a number of clinical trials in cancer patients (Chae Y. K. et al., Oncotarget, Mar. 19, 2016). These trials were stimulated by preclinical data in tumor models (Chae Y. K. et al., Oncotarget, Mar. 19, 2016) and the observation that type 2 diabetics treated with metformin had a decreased risk of developing various types of cancer (Quinn B. J., Kitagawa H., Memmott R. M., et al. Trends Endocrinol. Metab. vol. 24 pages 469-80, 2000 and Chae Y. K. et al., Oncotarget, Mar. 19, 2016). Subsequently, increased interest in this therapeutic approach has led to other complex 1 inhibitor classes being investigated (WO2014/031928, WO2014/031936, Ziegelbauer et al., Cancer Medicine, vol. 2 no. 5, pages 611-624, 2013 and WO2010/054763). Hence, targeting mitochondrial metabolism is of great interest for the development of novel therapeutic approaches for cancer treatment.

Accordingly in a first aspect the present invention provides compound of formula I or pharmaceutically acceptable salt, solvate or hydrate thereof for use for the treatment of proliferation diseases, in particular cancer, in a subject selected from a mammal, in particular in a human, wherein the compound of formula I is

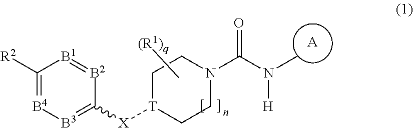

wherein

ring A represents group A-I or A-II

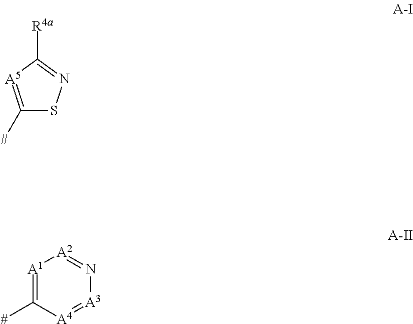

A1, A2, A3, A4 represent independently C(R4aa) or N, wherein no more than one of A1, A2, A3, and A4 represents N;

A5 represents C(R4b) or N;

B1, B2, B3 and B4 represent independently C(R3) or N, wherein no more than two of B1, B2, B3 and B4 represent N;

T represents >N—, >C═ or >CH—;

X represents —C(R6a)(R6b)-, —C(R6a)=, —O—, —S— or —C(O)—, providing that X is not —C(O)—, —O— or —S— when T is >N—;

R1 represents independently at each occurrence halogen, cyano, hydroxyl, —N(R5a)(R5b), C1-C6alkyl, C1-C6haloalkyl or C1-C6alkyl wherein one or two carbon atoms are independently replaced by —O— or —N(R5a)- and wherein the alkyl moiety is optionally substituted by one or more halogen;

R2 represents halogen, cyano, hydroxyl, mercapto, C1-C6alkyl optionally substituted by one to five R14, C2-C6alkenyl optionally substituted by one to five R14, C2-C6alkynyl optionally substituted by one to five R14, C1-C6alkoxy optionally substituted by one to five R14, —N(R9a)(R9b), —C1-C6alkylene-N(R9a)(R9b), —CHO, —C1-C6alkylene-CHO, —C(O)OR10, —C1-C6alkylene-C(O)OR10, —C(O)N(R11a)(R11b), —C1-C6alkylene-C(O)N(R11a)(R11b), —N(R12)C(O)R13, —C1-C6alkylene-N(R12)C(O)R13, C1-C6alkylthio, C1-C6alkylsulfinyl, C1-C6alkylsulfonyl, Cycle-P, —C1-C6alkylene-Cycle-P, Cycle-Q or —C1-C6alkylene-Cycle-Q;

R3 represents independently at each occurrence hydrogen, halogen, cyano, C1-C4alkyl, C1-C4haloalkyl, C1-C4alkoxy, C1-C4haloalkoxy or —N(R8a)(R8b);

R4a and R4b represent independently hydrogen, amino, —NH(C1-C4alkyl), —N(C1-C4alkyl)₂ or —C1-C4alkylene-R4c;

R4aa represents independently at each occurrence hydrogen, amino, —NH(C1-C4alkyl), —N(C1-C4alkyl)₂, —C1-C4alkylene-R4c or C3-C4cycloalkyl;

R4c represents independently at each occurrence hydrogen, cyano, hydroxyl, amino, C1-C4alkoxy, —CONH₂, —NH(C1-C4alkyl), —N(C1-C4alkyl)₂, Cycle-P or Cycle-Q;

R5a and R5b represent independently at each occurrence hydrogen or C1-C6alkyl;

R6a and R6b represent independently hydrogen or C1-C4alkyl;

each R8a and R8b represents independently at each occurrence hydrogen or C1-C4alkyl;

R9a represents hydrogen, C1-C6alkyl optionally substituted by one to five R14, —C1-C6alkylene-Cycle-P, —C1-C6alkylene-Cycle-Q, Cycle-P or Cycle-Q;

R9b, R11a, R11b and R12 represent independently hydrogen or C1-C6alkyl;

R10 and R13 represent independently at each occurrence C1-C6alkyl;

R14 represents independently at each occurrence halogen, cyano, hydroxyl, C1-C6alkoxy, amino, —NH(C1-C4alkyl), —N(C1-C4alkyl)₂ or —N(R12)C(O)R13;

Cycle-P represents independently at each occurrence a saturated or partially unsaturated 3- to 8-membered carbocyclic ring optionally substituted by 1 to 3 R16, or a saturated or partially unsaturated 3- to 8-membered heterocyclic ring optionally substituted by 1 to 3 R16 containing carbon atoms as ring members and one or two ring members independently selected from N and O, wherein N optionally may bear R15;

Cycle-Q represents independently at each occurrence phenyl optionally substituted by 1 to 3 R17 or a 5- to 6-membered heteroaryl ring containing one to four heteroatoms selected from O, S and N, optionally substituted by 1 to 3 R17;

R15 represents independently at each occurrence hydrogen or C1-C4alkyl;

R16 and R17 represent independently at each occurrence cyano, C1-C4alkyl, C1-C4haloalkyl, C1-C4alkoxy or C1-C4haloalkoxy;

n is 1 or 2; and

q is 0, 1, 2, 3 or 4.

The dotted bond between X and T represents a single bond or a double bond.

In a further aspect the invention provides use of a compound of formula I or pharmaceutically acceptable salt, solvate or hydrate thereof in the manufacture of a medicament for use for the treatment of proliferation diseases, in particular cancer, in a subject selected from a mammal, in particular in a human. In a further aspect the invention provides a method of treating proliferation diseases, in particular cancer, in a subject selected from a mammal, in particular in a human comprising administering the compound of formula I or pharmaceutically acceptable salt, solvate or hydrate thereof to said subject.

In a further aspect the invention provides a pharmaceutical composition comprising a compound of formula I or pharmaceutically acceptable salt, solvate or hydrate thereof and a pharmaceutically acceptable excipient.

Some compounds of formula I are known for uses other than as for the treatment of proliferation diseases and in a further aspect the invention provides compounds of formula I, pharmaceutically acceptable salt, solvate or hydrate thereof as described above wherein the compound of formula I, pharmaceutically acceptable salt, solvate or hydrate thereof is not:
1-Piperidinecarboxamide, 4-[(4-fluorophenyl)methyl]-N-(3-methyl-1,2,4-thiadiazol-5-yl)- (CAS 1244911-24-7);
1-Piperidinecarboxamide, 4-(4-fluorobenzoyl)-N-(2-methyl-4-pyridinyl)- (CAS 1808697-60-0);
1-Piperazinecarboxamide, 4-[(3,4-dibromophenyl)methyl]-N-4-pyridinyl- (CAS 898236-64-1, WO 2006/074025);
1-Piperazinecarboxamide, 4-[(3,4-dibromophenyl)methyl]-N-4-pyrimidinyl- (CAS 898237-02-0, WO 2006/074025);
1-Piperazinecarboxamide, 4-[(4-chlorophenyl)methyl]-N-4-pyrimidinyl- (CAS 1935818-02-2);

and preferably wherein T represents >C═ or >CH— when ring A represents group A-II.

Optionally the compound of formula I, pharmaceutically acceptable salt, solvate or hydrate thereof is not:
1-Piperidinecarboxamide, 4-(4-chlorobenzoyl)-N-4-pyridinyl- (CAS 2093733-82-3);
1H-1,4-Diazepine-1-carboxamide, hexahydro-4-[(4-methoxyphenyl)methyl]-N-(2-methyl-4-pyrimidinyl)- (CAS 1957980-10-7).

The above compounds may optionally be excluded from any aspect of the invention and/or the proviso that T represents >C═ or >CH— when ring A represents group A-II may likewise optionally apply to any aspect of the invention.

Each alkyl moiety either alone or as part of a larger group such as alkoxy is a straight or branched chain and is preferably C1-C6alkyl, more preferably C1-C4alkyl. Examples include methyl, ethyl, n-propyl, prop-2-yl, n-butyl, but-2-yl, 2-methyl-prop-1-yl or 2-methyl-prop-2-yl.

Each alkylene moiety is a straight or branched chain and is, for example, —CH₂—, —CH₂—CH₂—, —CH(CH₃)—, —CH₂—CH₂—CH₂—, —CH(CH₃)—CH₂—, or —CH(CH₂CH₃)—.

Each alkenyl moiety either alone or as part of a larger group such as alkenyloxy is a straight or branched chain and is preferably C2-C6alkenyl, more preferably C2-C4alkenyl. Each moiety can be of either the (E)- or (Z)-configuration. Examples include vinyl and allyl.

Each alkynyl moiety either alone or as part of a larger group such as alkynyloxy is a straight or branched chain and is preferably C2-C6alkynyl, more preferably C2-C4alkynyl. Examples are ethynyl and propargyl.

Each haloalkyl moiety either alone or as part of a larger group such as haloalkoxy is an alkyl group substituted by one or more of the same or different halogen atoms. Examples include difluoromethyl, trifluoromethyl, chlorodifluoromethyl and 2,2,2-trifluoro-ethyl. Haloalkyl moieties include for example 1 to 5 halo substituents, or 1 to 3 halo substituents.

Each haloalkenyl moiety either alone or as part of a larger group such as haloalkenyloxy is an alkenyl group substituted by one or more of the same or different halogen atoms. Examples include 2-difluoro-vinyl and 1,2-dichloro-2-fluoro-vinyl. Haloalkenyl moieties include for example 1 to 5 halo substituents, or 1 to 3 halo substituents.

Each cycloalkyl moiety can be in mono- or bi-cyclic form and preferably contains 3 to 8 carbon atoms, more preferably 3 to 6 carbon atoms. Examples of monocyclic cycloalkyl groups include cyclopropyl, cyclobutyl and cyclohexyl. An example of a bicyclic cycloalkyl group is bicyclo[2.2.1]heptan-2-yl. Halogen is fluorine, chlorine, bromine, or iodine, preferably fluorine, chlorine or bromine.

The term “amino” refers to —NH₂.

The term “mercapto” refers to SH.

The term “heteroaryl” refers to an aromatic ring system containing at least one heteroatom, and preferably up to four, more preferably three, heteroatoms selected from nitrogen, oxygen and sulfur as ring members. Heteroaryl rings do not contain adjacent oxygen atoms, adjacent sulfur atoms, or adjacent oxygen and sulfur atoms within the ring. Examples include pyridinyl, pyrimidinyl, pyrazinyl, pyridazinyl, pyrrolyl, pyrazolyl, imidazolyl, triazolyl, isoxazolyl, oxazolyl, oxadiazolyl, isothiazolyl, thiazolyl, thiadiazolyl, tetrazolyl, furanyl, and thiophenyl.

The term “heterocyclic ring” refers to a saturated or partially unsaturated carbocyclic ring containing one to four heteroatoms selected from nitrogen, oxygen and sulfur as ring members. Such rings do not contain adjacent oxygen atoms, adjacent sulfur atoms, or adjacent oxygen and sulfur atoms within the ring.

Examples include tetrahydrofuranyl, azetidinyl, pyrrolidinyl, piperidinyl, piperazinyl, dioxanyl and morpholinyl.

Where a group is said to be optionally substituted, it may be substituted or unsubstituted, preferably there are optionally 1-5 substituents, more preferably optionally 1-3 substituents.

The bond between T and X may be a single bond or a double bond depending on the identity of T and X. Certain compounds of formula I may contain one or two or more centers of chirality and such compounds may be provided as pure enantiomers or pure diastereoisomers as well as mixtures thereof in any ratio. For example, where T is CH and n is 2, or n is 1 and at least one R1 is different than H, the H on T may be in the axial or equatorial configuration and the invention includes both isomers in any ratio. The compounds of the invention also include all cis/trans-isomers (for example where the bond between T and X is the —C═C— moiety) as well as mixtures thereof in any ratio.

The compounds of the invention also include all tautomeric forms of the compounds of formula I.

The compounds of formula I may also be solvated, especially hydrated, which are also included in the compounds of formula I. Solvation and hydration may take place during the preparation process. Reference to compounds of the invention includes pharmaceutically acceptable salts of said compounds. Such salts may also exist as hydrates and solvates. Examples of pharmacologically acceptable salts of the compounds of formula (I) are salts of physiologically acceptable mineral acids, such as hydrochloric acid, sulfuric acid and phosphoric acid, or salts of organic acids, such as methane-sulfonic acid, p-toluenesulfonic acid, lactic acid, acetic acid, trifluoroacetic acid, citric acid, succinic acid, fumaric acid, maleic acid and salicylic acid. Further examples of pharmacologically acceptable salts of the compounds of formula (I) are alkali metal and alkaline earth metal salts such as, for example, sodium, potassium, lithium, calcium or magnesium salts, ammonium salts or salts of organic bases such as, for example, methylamine, dimethylamine, triethylamine, piperidine, ethylenediamine, lysine, choline hydroxide, meglumine, morpholine or arginine salts.

The following preferred substituent definitions may be combined in any combination.

Examples of group A-I are group A-Ia and group A-Ib:

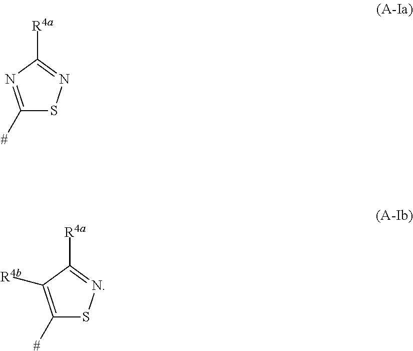

Preferably group A-I is group A-Ia or group A-Ib-1:

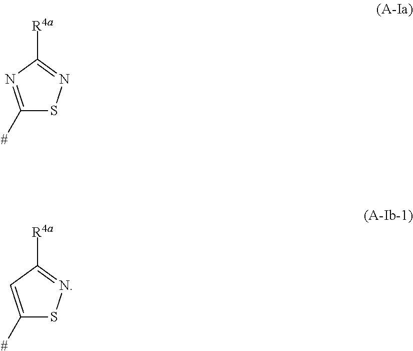

When R4a is R4a*, wherein R4a* is as defined for R4a but is other than hydrogen, group A-Ia and group A-Ib-1 may be group A-Ia-a, group A-Ia-b, group A-Ib-1a or group A-Ib-1b:

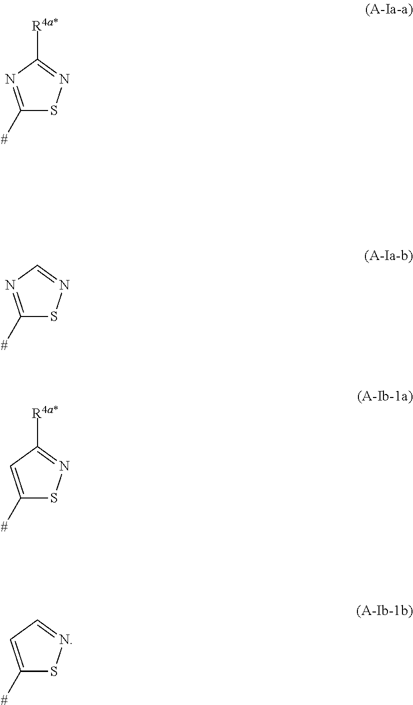

Preferred specific examples when ring A is group A-I include the following:

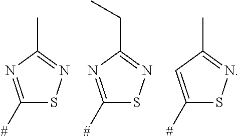

Group A-II maybe group A-IIa, group A-IIb or Group A-IIc, preferably Group A-IIa:

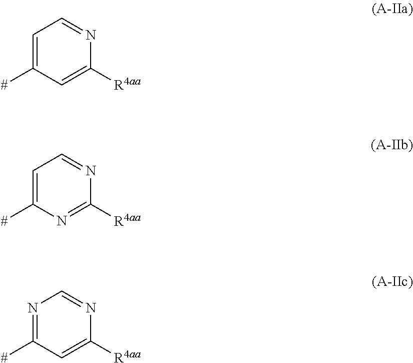

When R4aa is R4aa* and wherein R4aa* is as defined for R4aa but is other than hydrogen group A-IIa and group A-IIb may be group A-IIa-1, group A-IIa-2, group A-IIb-1, group A-IIb-2 or group A-IIc-1:

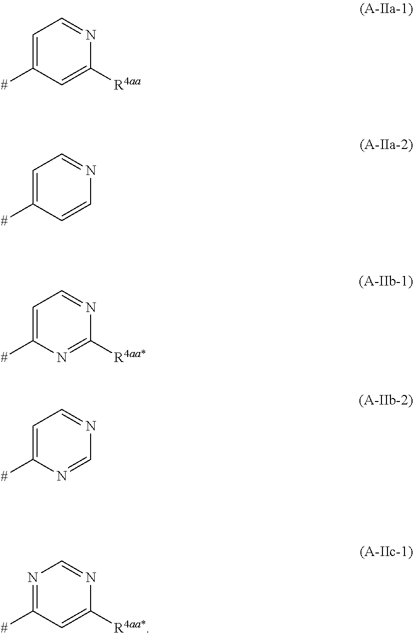

Examples of preferred group A-II are group A-IIa-1a, group A-IIa-2, group A-IIb-1a, group A-IIb-2 and group A-IIc-1a

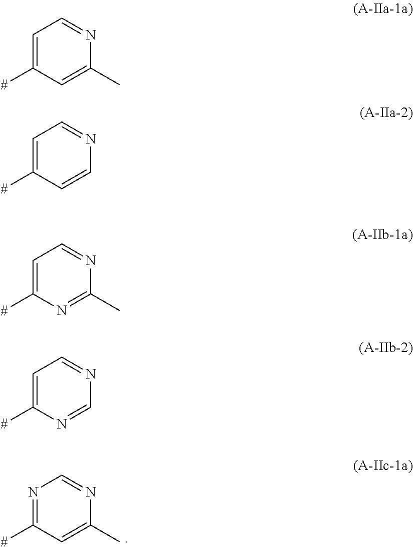

When ring A is group A-II, preferably one of A2 and A3 represent C(R4aa) and the other represents CH and A1 and A4 represent CH. Preferred specific examples include the following groups:

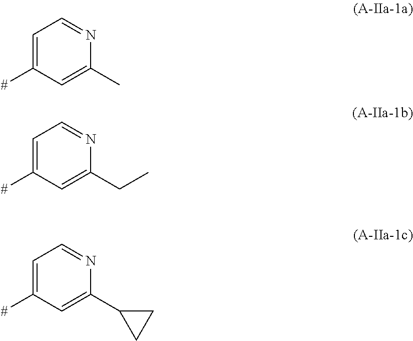

A further example of group A-II is when one of A2 and A3 represents N and the other represents C(R4aa) and A1 and A4 both represent CH.

B1, B2, B3 and B4 preferably represent independently C(R3) or N, wherein no more than one of B1, B2, B3 and B4 represents N. Structural examples of the ring comprising B1, B2, B3 and B4 as ring members are represented by group B-I, group B-II and group B-III:

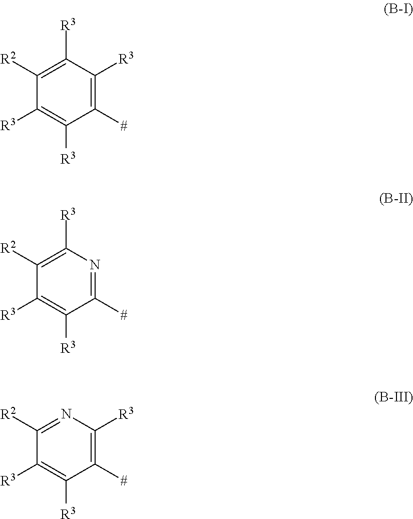

Preferably B1, B2, B3 and B4 represent independently C(R3a), C(R3b) or N wherein no more than two of B1, B2, B3 and B4 represent C(R3a), wherein no more than one of B1, B2, B3 and B4 represents N, wherein each R3a is independently R3 and each R3b represents hydrogen.

Preferred structural examples of the ring comprising B1, B2, B3 and B4 as ring members are represented by group B-Ia, group B-Ib, group B-IIa and group BIIIa:

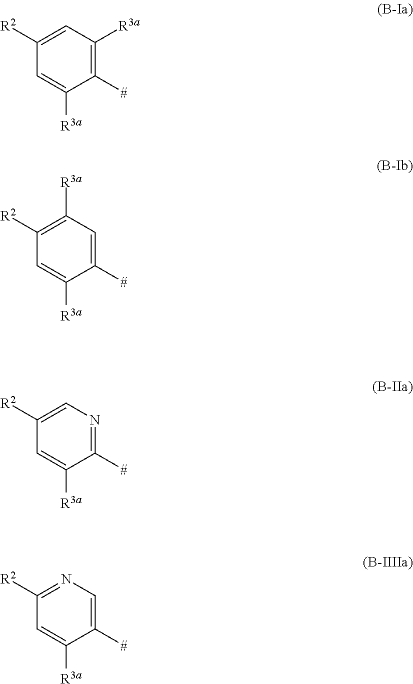

When R3a is R3a*, wherein R3a* is as defined for R3a but is other than hydrogen, preferred structural examples of the ring comprising B1, B2, B3 and B4 as ring members include group B-Ia-1, group B-Ia-2, group B-Ia-3, group B-Ib-1, group B-Ib-2, group B-IIa-1, group B-IIa-2, group B-IIIa-1 and group B-IIIa-2:

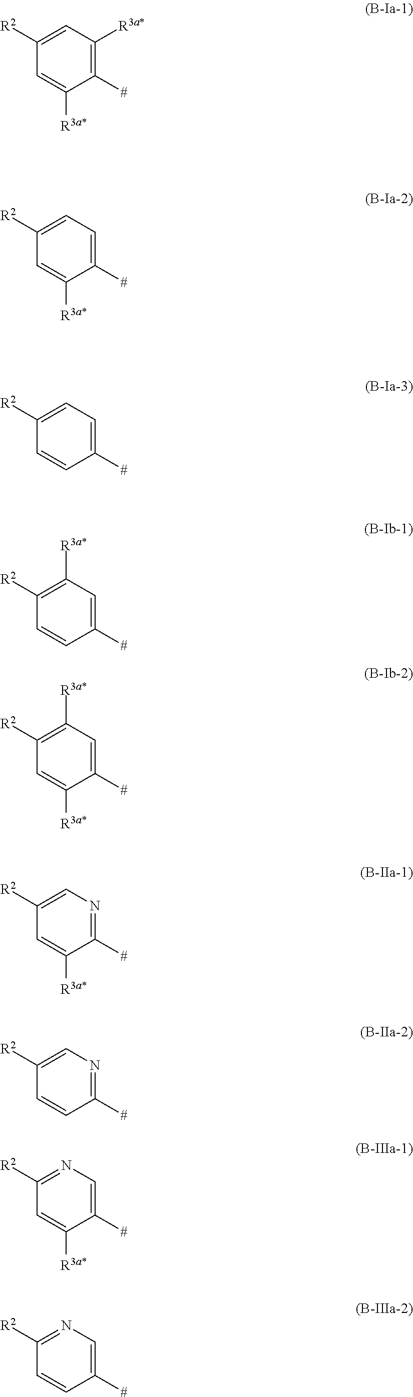

Of these B-Ia-1, B-Ia-2 and B-Ia-3 are particularly preferred.

Preferred examples of the ring comprising B1, B2, B3 and B4 as ring members include include the following groups:

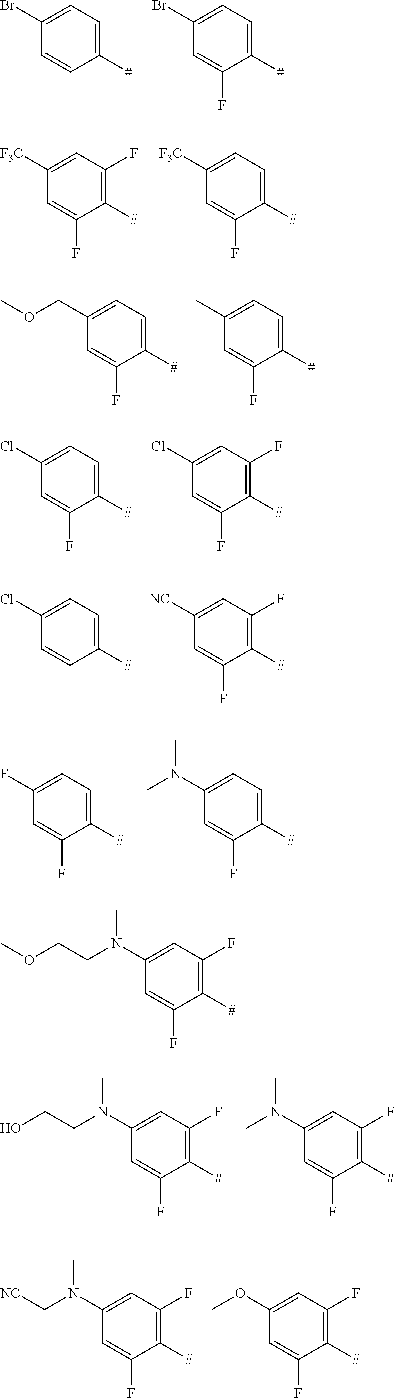

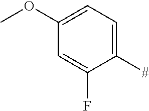

X preferably represents —C(R6a)(R6b)-, —C(R6a)= or —C(O)—, more preferably —CH₂—, —CH═ or C(O). Preferably T represents >C═ and X represents —CH═, or T represents >CH— and X represents —C(O)— or T represents >CH— and X represents —CH₂—, more preferably T represents >C═ and X represents —CH═, or T represents >CH— and X represents —CH₂—.

It will be clear that when T represents >C═ then X represents —C(R6a)= in view of the double bond. R1 preferably represents independently at each occurrence halogen, cyano, hydroxyl, amino, —NH(C1-C4alkyl), —N(C1-C4alkyl)₂, C1-C6alkyl, C1-C6haloalkyl or C1-C6alkyl wherein one carbon atom is replaced by —O—, more preferably halogen, hydroxyl, C1-C4alkyl, C1-C4alkoxy or C1-C3alkoxy-C1-C3alkyl, even more preferably fluoro, hydroxyl, methyl, ethyl, propyl, methoxy, ethoxy, methoxymethyl or methoxyethyl, and in particular fluoro, methyl, ethyl, propyl or methoxy.

Preferably R2 represents halogen, cyano, hydroxyl, C1-C6alkyl optionally substituted by one to five R14, C1-C6alkoxy optionally substituted by one to five R14, —N(R9a)(R9b) or —C1-C6alkylene-N(R9a)(R9b), more preferably fluoro, chloro, bromo, cyano, hydroxyl, C1-C6alkyl, C1-C6haloalkyl, C1-C6alkyl wherein one or two non-adjacent carbon atoms in the alkyl other than the connecting carbon atom are replaced independently by —O—, —OH, —NH—, —NH₂, —N(CH₃)—, —NH(CH₃), —N(CH₃)₂ or —CN, or C1-C6haloalkyl wherein one or two non-adjacent carbon atoms in the haloalkyl other than the connecting carbon atom are replaced independently by —O—, —OH, —NH—, —NH₂, —N(CH₃)—, —NH(CH₃), —N(CH₃)₂ or —CN, or C1-C6alkoxy, C1-C6alkoxy wherein one carbon atom in the alkoxy other than the carbon atom connected to the oxygen is replaced by —O—, —OH, —NH—, —NH₂, —N(CH₃)— or —CN, or —N(R9a)(R9b) or —C1-C6alkylene-N(R9a)(R9b) and wherein R9a represents hydrogen, C1-C6alkyl wherein one or two non-adjacent carbon atoms in the alkyl, preferably other than the carbon atom connected to the nitrogen atom, are replaced independently by —O—, —OH, —NH—, —NH₂, —N(CH₃)—, —NH(CH₃), —N(CH₃)₂ or —CN, or R9a represents —C1-C6-alkylene-Cycle-P or Cycle-P, wherein Cycle-P preferably represents a saturated 4- to 6-membered heterocyclic ring containing one or two heteroatoms selected from O and N(R15), wherein the heterocyclic ring is optionally substituted by one to three substituents selected from methyl. R9b represents hydrogen, methyl or ethyl, preferably hydrogen or methyl, and R15 represents independently at each occurrence hydrogen or methyl, even more preferably R2 represents fluoro, chloro, bromo, cyano, hydroxyl, C1-C6alkyl, C1-C6haloalkyl, C1-C6alkoxy, C1-C6haloalkoxy, —C1-C4alkylene-methoxy, —N(R9b)-C1-C4alkylene-R18, —N(R9b)-C1-C4alkylene-Cycle-P or —N(R9b)-Cycle-P, wherein Cycle-P represents tetrahydrofuranyl, azetidinyl, pyrrolidinyl, piperidinyl, piperazinyl, dioxanyl or morpholinyl wherein N is substituted by R15 in each case, R9b represents hydrogen, methyl or ethyl, R15 represents independently at each occurrence hydrogen or methyl, and R18 represents —OH, —OCH₃, —CN, —NH₂, —NH(CH₃), or —N(CH₃)₂.

Specific examples of R2 include fluoro, chloro, bromo, cyano, amino, hydroxyl, methyl, ethyl, propyl, butyl, methoxy, ethoxy, propoxy, butoxy, methoxymethyl, trifluromethyl, trifluoromethoxy, —C(O)OCH₃, —C(O)NH₂, —CHO, —CH₂OH, —N(CH₃)₂, —NH(CH₃), —NHCH₂CH₂NH₂, —NHCH₂CH₂CH₂NH₂, —N(CH₃)CH₂CH₂OH, —OCH₂CH₂CH₂NH₂, —OCH₂CH₂CH₂OH, —CH₂N(CH₃)CH₂CH₂OH, —CH₂NHCH₂CH₂CH₂-morpholinyl (e.g. —CH₂NHCH₂CH₂CH₂-morpholin-4-yl), —CH₂-morpholinyl (e.g. —CH₂-morpholin-4-yl), methyloxadiazolyl (e.g. 3-methyl-oxadiazolyl), -pryrolidinyl (e.g. -pryrolidin-1-yl), SO₂CH₃, —N(CH₃)CH₂CH₂OCH₃, —N(CH₃)CH₂CN, —N(CH₃)CH₂(1-methylazetidinyl) (e.g. —N(CH₃)CH₂(1-methylazetidin-3-yl)), —N(CH₃)-tetrahydrofuran (e.g. N(CH₃)-3-tetrahydrofuran), —N(CH₃)(CH₂)₃NH₂, —NHCH₂CH₃, —NH-tetrahydrofuran (e.g. NH-3-tetrahydrofuran), —N(CH₃)CH₂CH₂NH₂, —N(CH₂CH₃)₂, —N(CH₃)CH₂CH₂NHC(O)CH₃, —N(CH₃)(CH₂)₄NH₂, and —C≡C—CH₂OH. Preferred specific examples are fluoro, chloro, bromo, cyano, methyl, trifluromethyl, N(CH₃)₂, methoxy, methoxymethyl, —N(CH₃)CH₂CH₂OH, —N(CH₃)CH₂CH₂OCH₃, and —N(CH₃)CH₂CN.

R3 preferably represents independently at each occurrence hydrogen, halogen, cyano, methyl, halomethyl, methoxy, amino, —NH(CH₃) or —N(CH₃)₂, more preferably hydrogen, fluoro, chloro, bromo, cyano, methyl, halomethyl, methoxy or amino, even more preferably hydrogen, fluoro, chloro, methyl or methoxy, and in particular hydrogen or fluoro. Preferably no more than two R3 are other than hydrogen. Particularly preferably each R3 on B1, B2, B3 and B4 is hydrogen, or each R3 on B1, B2 and B4 is hydrogen and R3 on B3 is halogen, in particular fluoro, or each R3 on B1 and B4 is hydrogen and each R3 on B2 and B3 is independently halogen, preferably fluoro.

R4a may represent hydrogen, amino, —NH(C1-C4alkyl), —N(C1-C4alkyl)₂ or —C1-C4alkylene-R4c. Preferably R4a represents hydrogen, amino, C1-C4alkyl, C1-C4alkyl wherein one CH₂ is replaced by —NH— or —N(CH₃)—, —C1-C4alkylene-cyano, —C1-C4alkylene-hydroxyl, —C1-C4alkylene-amino, —C1-C4alkylene-Cycle-P, wherein Cycle-P is a 5- to 6-membered heterocyclic ring, more preferably hydrogen, methyl, ethyl, amino, —CH₂CH₂CN, —CH₂CH₂-morpholinyl (e.g. —CH₂CH₂-morpholin-4-yl) or —CH₂CH₂OH, even more preferably methy or ethyl.

R4aa may represent independently at each occurrence hydrogen, amino, —NH(C1-C4alkyl), —N(C1-C4alkyl)₂ or —C1-C4alkylene-R4c or C3-C4cycloalkyl.

Preferably R4aa represents independently at each occurrence hydrogen, amino, C1-C4alkyl, C1-C4alkyl wherein one CH₂ is replaced by —NH— or —N(CH₃)—, C3-C4cycloalkyl, —C1-C4alkylene-cyano, —C1-C4alkylene-hydroxyl, —C1-C4alkylene-amino, —C1-C4alkylene-methoxy, —C1-C4alkylene-C3-C4cycloalkyl or —C1-C4alkylene-Cycle-P, wherein Cycle-P is a 5- to 6-membered heterocyclic ring, more preferably hydrogen, methyl, ethyl, amino, —CH₂CH₂CN, —CH₂CH₂-morpholinyl (e.g. —CH₂CH₂-morpholin-4-yl), —CH₂OH, —CH₂CH₂OH, —CH₂OCH₃ or cyclopropyl, even more preferably hydrogen methyl, ethyl or cyclopropyl, in particular methyl, ethyl or cyclopropyl. In one embodiment R4aa is C3-C4cycloalkyl, preferably cyclopropyl.

R4b may represent hydrogen, amino, —NH(C1-C4alkyl), —N(C1-C4alkyl)₂ or —C1-C4alkylene-R4c. Preferably R4b represents hydrogen, amino, C1-C4alkyl, C1-C4alkyl wherein one CH₂ is replaced by —NH— or —N(CH₃)—, —C1-C4alkylene-cyano, —C1-C4alkylene-hydroxyl, —C1-C4alkylene-amino or —C1-C4alkylene-Cycle-P, wherein Cycle-P is a 5- to 6-membered heterocyclic ring, more preferably hydrogen, methyl, ethyl, amino, —CH₂CH₂CN, —CH₂CH₂-morpholinyl (e.g. —CH₂CH₂-morpholin-4-yl) or —CH₂CH₂OH, even more preferably R4b represents hydrogen.

R4c preferably represents independently at each occurrence hydrogen, cyano, hydroxyl, amino, C1-C4alkoxy, —NH(C1-C4alkyl), —N(C1-C4alkyl)₂, C3-C4cycloalkyl or Cycle-P, more preferably hydrogen, cyano, hydroxyl, amino, methoxy, —NH(CH₃), —N(CH₃)₂, C3-C4cycloalkyl or Cycle-P, wherein Cycle-P is a 5- to 6-membered heterocyclic ring, even more preferably hydrogen, cyano, hydroxyl, amino, methoxy, cyclopropyl or morpholinyl.

R5a represents independently at each occurrence hydrogen or C1-C6alkyl, more preferably hydrogen or methyl, more preferably hydrogen.

R5b represents independently at each occurrence hydrogen or C1-C6alkyl, more preferably hydrogen or methyl, more preferably hydrogen.

R6a represents hydrogen or C1-C4alkyl, preferably hydrogen or methyl, more preferably hydrogen.

R6b represents hydrogen or C1-C4alkyl, preferably hydrogen or methyl, more preferably hydrogen.

R8a represents independently at each occurrence hydrogen or C1-C4alkyl, preferably hydrogen or methyl, more preferably hydrogen.

R8b represents independently at each occurrence hydrogen or C1-C4alkyl, preferably hydrogen or methyl, more preferably hydrogen.

R9a preferably represents hydrogen or C1-C6alkyl optionally substituted by one to five R14, more preferably hydrogen or C1-C6alkyl wherein one or two non-adjacent carbon atoms in the alkyl are replaced independently by —O—, —OH, —NH—, —NH₂, —N(CH₃)—, —NH(CH₃) or —N(CH₃)₂, or —CN, or R9a represents —C1-C6-alkylene-Cycle-P or Cycle-P, wherein Cycle-P preferably represents a saturated 4- to 6-membered heterocyclic ring containing one or two heteroatoms selected from O and N(R15), wherein the heterocyclic ring is optionally substituted by one to three substituents selected from methyl, and R15 represents independently at each occurrence hydrogen or methyl, more preferably R9a represents-C1-C4alkylene-R18, —C1-C4alkylene-Cycle-P or Cycle-P, wherein Cycle-P represents tetrahydrofuranyl, azetidinyl, pyrrolidinyl, piperidinyl, piperazinyl, dioxanyl or morpholinyl wherein N is substituted by R15 in each case and R15 represents independently at each occurrence hydrogen or methyl, and wherein R18 represents —OH, —OCH₃, —CN, —NH₂, —NH(CH₃), or —N(CH₃)₂.

R9b represents hydrogen or C1-C6alkyl, preferably hydrogen or methyl or ethyl, more preferably hydrogen or methyl.

R11a represents hydrogen or C1-C6alkyl, preferably hydrogen or methyl.

R11b represents hydrogen or C1-C6alkyl, preferably hydrogen or methyl.

R12 represents hydrogen or C1-C6alkyl, preferably hydrogen or methyl.

R10 preferably represents methyl or ethyl.

R13 preferably represents methyl or ethyl.

R14 preferably represents independently at each occurrence halogen, cyano, hydroxyl, C1-C6alkoxy, amino, —NH(C1-C4alkyl) or —N(C1-C4alkyl)₂.

Cycle-P preferably represents independently at each occurrence a saturated 4-membered ring or a saturated or partially unsaturated 5- to -6-membered heterocyclic ring optionally substituted by 1 to 3 R16 containing carbon atoms as ring members and one or two ring members independently selected from N and O, wherein N optionally may bear R15. More preferably Cycle-P represents a saturated 4- to 6-membered heterocyclic ring containing one or two heteroatoms selected from O and N(R15), wherein the heterocyclic ring is optionally substituted by one to three substituents selected from methyl, and R15 represents independently at each occurrence hydrogen or methyl, even more preferably Cycle-P represents tetrahydrofuranyl, azetidinyl, pyrrolidinyl, piperidinyl, piperazinyl, dioxanyl or morpholinyl wherein N is substituted by R15 in each case and wherein R15 represents independently at each occurrence hydrogen or methyl. Specific examples include morpholinyl and pyrrolidinyl, tetrahydrofuranyl, 1-methylazetidinyl (e.g. 1-methylazetidin-3-yl).

Cycle-Q represents independently at each occurrence a 5- to 6-membered heteroaryl ring containing one to four heteroatoms selected from O, S and N, optionally substituted by 1 to 3 R17. Specific examples include oxadiazolyl, in particular 3-methyl-oxadiazolyl.

R15 represents independently at each occurrence hydrogen or C1-C4alkyl, preferably hydrogen or methyl, more preferably hydrogen.

R16 represents independently at each occurrence cyano, C1-C4alkyl, C1-C4haloalkyl, C1-C4alkoxy or C1-C4haloalkoxy, preferably cyano, methyl, halomethyl, methoxy or halomethoxy, even more preferably cyano, methyl, trifluoromethyl or methoxy.

R17 represents independently at each occurrence cyano, C1-C4alkyl, C1-C4haloalkyl, C1-C4alkoxy or C1-C4haloalkoxy, preferably cyano, methyl, halomethyl, methoxy or halomethoxy, even more preferably cyano, methyl, trifluoromethyl or methoxy.

q is preferably 0, 1 or 2, and preferably when q is 2 the R1 substituents are on the same carbon atom, more preferably 0 or 1, even more preferably 0.

Any embodiment relating to the chemical structure of the compounds of the invention may be combined with any other embodiment where possible, including with any of the substituent definitions or preferred substituent definitions given above.

In one embodiment ring A represents group A-I, preferably wherein

A5 represents C(R4b) or N;

R4a represents hydrogen, amino, C1-C4alkyl, C1-C4alkyl wherein one CH₂ is replaced by —NH— or —N(CH₃)—, —C1-C4alkylene-cyano, —C1-C4alkylene-hydroxyl, —C1-C4alkylene-amino or —C1-C4alkylene-Cycle-P, wherein Cycle-P is a 5- to 6-membered heterocyclic ring, preferably hydrogen, methyl, ethyl, amino, —CH₂CH₂CN, —CH₂CH₂-morpholin-4-yl or —CH₂CH₂OH; and R4b represents hydrogen.

In one embodiment ring A represents group A-II, preferably wherein one of A2 and A3 represent C(R4aa) and the other represents CH;

A1 and A4 represent CH; and

R4aa represents independently at each occurrence hydrogen, amino, C1-C4alkyl, C1-C4alkyl wherein one CH₂ is replaced by —NH— or —N(CH₃)—, C3-C4cycloalkyl, —C1-C4alkylene-cyano, —C1-C4alkylene-hydroxyl, —C1-C4alkylene-amino, —C1-C4alkylene-methoxy, —C1-C4alkylene-C3-C4cycloalkyl or —C1-C4alkylene-Cycle-P, wherein Cycle-P is a 5- to 6-membered heterocyclic ring, preferably hydrogen, methyl, ethyl, amino, —CH₂CH₂CN, —CH₂CH₂-morpholinyl (e.g. —CH₂CH₂-morpholin-4-yl), CH₂OH, —CH₂CH₂OH, —CH₂OCH₃ or cyclopropyl, even more preferably hydrogen methyl, ethyl or cyclopropyl, in particular methyl, ethyl or cyclopropyl.

In one embodiment ring A represents group A-II, preferably wherein

one of A2 and A3 represent C(R4aa) and the other represents CH;

A1 and A4 represent CH; and

R4aa represents hydrogen, amino, C1-C4alkyl, C1-C4alkyl wherein one CH₂ is replaced by —NH— or —N(CH₃)—, —C1-C4alkylene-cyano, —C1-C4alkylene-hydroxyl, —C1-C4alkylene-amino or —C1-C4alkylene-Cycle-P, wherein Cycle-P is a 5- to 6-membered heterocyclic ring, preferably hydrogen, methyl, ethyl, amino, —CH₂CH₂CN, —CH₂CH₂-morpholin-4-yl or —CH₂CH₂OH.

In one embodiment ring A represents group A-II preferably wherein one of A2 and A3 represent C(R4aa) and the other represents CH;

A1 and A4 represent CH; and

R4aa represents C3-C4cylcloalkyl, preferably cyclopropyl.

In one embodiment n is 1.

In one embodiment n is 2.

In one embodiment T represents >C═ and X represents —C(R6a)=.

In one embodiment T represents >CH— and X represents —C(R6a)(R6b)-.

In one embodiment T represents >N— and X represents —C(R6a)(R6b)-.

In one embodiment T represents >CH— and X represents —C(O)—.

In one embodiment T represents >CH— and X represents —O—.

In one embodiment T represents >CH— and X represents —S—.

In one embodiment T represents >C═ and X represents —C(CH₃)═.

In one embodiment T represents >C═ and X represents —CH═.

In one embodiment T represents >CH— and X represents —CH₂—.

In one embodiment T represents >N— and X represents —CH₂—.

In one embodiment T represents >CH— and X represents —CH(CH₃)—.

In one embodiment T represents >CH— and X represents —C(O)—.

In one embodiment T represents >CH— and X represents —O—.

In one embodiment T represents >CH— and X represents —S—.

In one embodiment T represents >C═ or >CH—.

In one embodiment ring A represents group A-I, T represents >C═ and X represents —CH═.

In one embodiment ring A represents group A-I, A5 represents N, T represents >C═ and X represents —CH═.

In one embodiment ring A represents group A-II, T represents >C═ and X represents —CH═.

In one embodiment ring A represents group A-II, one of A2 and A3 represent C(R4aa) and the other represents CH, A1 and A4 represent CH, T represents >C═ and X represents —CH═.

In one embodiment ring A represents group A-I, T represents >CH— and X represents —CH₂—.

In one embodiment ring A represents group A-I, A5 represents N, T represents >CH— and X represents —CH₂—.

In one embodiment ring A represents group A-II, group T represents >CH— and X represents —CH₂—.

In one embodiment ring A represents group A-II, one of A2 and A3 represent C(R4aa) and the other represents CH, A1 and A4 represent CH, T represents >CH— and X represents —CH₂—.

In one embodiment ring A represents group A-I, T represents >C═ or >CH— and X represents —CH₂— or —CH═.

In one embodiment ring A represents group A-I, A5 represents N, T represents >C═ or >CH— and X represents —CH₂— or —CH═.

In one embodiment ring A represents group A-II, T represents >C═ or >CH— and X represents —CH₂— or —CH═.

In one embodiment ring A represents group A-II, one of A2 and A3 represent C(R4aa) and the other represents CH, A1 and A4 represent CH, T represents >C═ or >CH— and X represents —CH₂— or —CH═.

In one embodiment:

R2 represents halogen, cyano, hydroxyl, C1-C6alkyl optionally substituted by one to five R14, C1-C6alkoxy optionally substituted by one to five R14, —N(R9a)(R9b) or —C1-C6alkylene-N(R9a)(R9b); R9a represents hydrogen, C1-C6alkyl optionally substituted by one to five R14, —C1-C6alkylene-Cycle-P or Cycle-P;

R9b represents hydrogen or methyl;

R14 represents independently at each occurrence halogen, cyano, hydroxyl, C1-C6alkoxy, amino, —NH(C1-C4alkyl) or —N(C1-C4 alkyl)₂.

In one embodiment:

R2 represents halogen, cyano, hydroxyl, C1-C6alkyl optionally substituted by one to five R14, C1-C6alkoxy optionally substituted by one to five R14, —N(R9a)(R9b) or —C1-C6alkylene-N(R9a)(R9b);

R9a represents hydrogen or C1-C6alkyl optionally substituted by one to five R14;

R9b represents hydrogen or methyl;

R14 represents independently at each occurrence halogen, cyano, hydroxyl, C1-C6alkoxy, amino, —NH(C1-C4alkyl) or —N(C1-C4alkyl)₂.

In one embodiment when T is N and ring A is group A-II then at least one R4aa is not hydrogen.

In one embodiment (Embodiment 1a):

A1 and A4 represent CH;

one of A2 and A3 represent C(R4aa) and the other represents CH;

A5 represents CH or N;

B1, B2, B3 and B4 represent independently C(R3a), C(R3b) or N, wherein no more than one of B1, B2, B3 and B4 represents N, no more than two of B1, B2, B3 and B4 represents C(R3a);

T represents >N—, >C═ or >CH—;

X represents —CH₂—, —CH═ or —C(O)— providing that X is not —C(O)— when T is >N—;

preferably T represents >C═ and X represents —CH═, or T represents >CH— and X represents —C(O)— or T represents >CH— and X represents —CH₂—;

R1 represents independently at each occurrence halogen, C1-C4alkyl or C1-C4alkoxy;

R2 represents halogen, cyano, hydroxyl, C1-C6alkyl optionally substituted by one to five R14, C1-C6alkoxy optionally substituted by one to five R14, —N(R9a)(R9b) or —C1-C6alkylene-N(R9a)(R9b);

R3a represents independently at each occurrence hydrogen, halogen, cyano, methyl, halomethyl, methoxy, amino, —NH(CH₃) or —N(CH₃)₂;

R3b represents hydrogen;

R4a represents hydrogen, amino, C1-C4alkyl, C1-C4alkyl wherein one CH₂ is replaced by —NH— or —N(CH₃)—, —C1-C4alkylene-cyano, —C1-C4alkylene-hydroxyl, —C1-C4alkylene-amino or —C1-C4alkylene-Cycle-P, preferably hydrogen, methyl, ethyl, amino, —CH₂CH₂CN, —CH₂CH₂-morpholinyl or —CH₂CH₂OH;

R4aa represents independently at each occurrence hydrogen, amino, C1-C4alkyl, C1-C4alkyl wherein one CH₂ is replaced by —NH— or —N(CH₃)—, C3-C4cycloalkyl, —C1-C4alkylene-cyano, —C1-C4alkylene-hydroxyl, —C1-C4alkylene-amino, —C1-C4alkylene-methoxy, —C1-C4alkylene-C3-C4cycloalkyl or —C1-C4alkylene-Cycle-P, wherein Cycle-P is a 5- to 6-membered heterocyclic ring, preferably hydrogen, methyl, ethyl, amino, —CH₂CH₂CN, —CH₂CH₂-morpholinyl, CH₂OH, —CH₂CH₂OH, —CH₂OCH₃ or cyclopropyl;

R9a represents hydrogen, C1-C6alkyl optionally substituted by one to five R14, —C1-C6-alkylene-Cycle-P or Cycle-P;

R9b represents hydrogen or methyl;

R14 represents independently at each occurrence halogen, cyano, hydroxyl, C1-C6alkoxy, amino, —NH(C1-C4alkyl) or —N(C1-C4alkyl)₂;

Cycle-P is a 5- to 6-membered heterocyclic ring;

n is 1 or 2; and

q is 0, 1 or 2.

In one embodiment (Embodiment 1b):

A1 and A4 represent CH;

one of A2 and A3 represent C(R4aa) and the other represents CH;

A5 represents CH or N;

B1, B2, B3 and B4 represent independently C(R3a), C(R3b) or N, wherein no more than one of B1, B2, B3 and B4 represents N, no more than one of B1, B2, B3 and B4 represents C(R3a);

T represents >N—, >C═ or >CH—;

X represents —CH₂—, —CH═ or —C(O)— providing that X is not —C(O)— when T is >N—;

preferably T represents >C═ and X represents —CH═, or T represents >CH— and X represents —C(O)— or T represents >CH— and X represents —CH₂—;

R1 represents independently at each occurrence halogen or C1-C4alkyl;

R2 represents halogen, cyano, hydroxyl, C1-C6alkyl optionally substituted by one to five R14, C1-C6alkoxy optionally substituted by one to five R14, —N(R9a)(R9b) or —C1-C6alkylene-N(R9a)(R9b);

R3a represents independently at each occurrence hydrogen, halogen, cyano, methyl, halomethyl, methoxy, amino, —NH(CH₃) or —N(CH₃)₂;

R3b represents hydrogen;

R4a and R4aa represent hydrogen, amino, C1-C4alkyl, C1-C4alkyl wherein one CH₂ is replaced by NH or N(CH₃), —C1-C4alkylene-cyano, —C1-C4alkylene-hydroxyl, —C1-C4alkylene-amino or —C1-C4alkylene-Cycle-P, wherein Cycle-P is a 5- to 6-membered heterocyclic ring, preferably hydrogen, methyl, ethyl, amino, —CH₂CH₂CN, —CH₂CH₂-morpholin-4-yl or —CH₂CH₂OH;

R9a represents hydrogen or C1-C6alkyl optionally substituted by one to five R14;

R9b represents hydrogen or methyl;

R14 represents independently at each occurrence halogen, cyano, hydroxyl, C1-C6alkoxy, amino, —NH(C1-C4alkyl) or —N(C1-C4alkyl)₂;

n is 1 or 2; and

q is 0, 1 or 2.

In one embodiment (Embodiment 2):

Ring A represents Group A-I;

A5 represents N;

T represents >C═ and X represents —CH═, or T represents >CH— and X represents —C(O)— or T represents >CH— and X represents —CH₂—;

B1, B2, B3 and B4 represent independently C(R3a), C(R3b) or N, wherein no more than one of B1, B2, B3 and B4 represents N, no more than two of B1, B2, B3 and B4 represents C(R3a);

R2 represents fluoro, chloro, bromo, cyano, hydroxyl, C1-C6alkyl, C1-C6haloalkyl, C1-C6alkyl wherein one or two non-adjacent carbon atoms in the alkyl other than the connecting carbon atom are replaced independently by —O—, —OH, —NH—, —NH₂, —N(CH₃)—, —NH(CH₃) or —N(CH₃)₂, or C1-C6haloalkyl wherein one or two non-adjacent carbon atoms in the haloalkyl other than the connecting carbon atom are replaced independently by —O—, —OH, —NH—, —NH₂, —N(CH₃)—, —NH(CH₃) or —N(CH₃)₂, or C1-C6alkoxy, C1-C6alkoxy wherein one carbon atom in the alkoxy other than the carbon atom connected to the oxygen is replaced by —O—, —OH, —NH—, —NH₂ or —N(CH₃)—, or —N(R9a)(R9b) or —C1-C6alkylene-N(R9a)(R9b) and wherein R9a represents hydrogen or C1-C6alkyl wherein one or two non-adjacent carbon atoms in the alkyl are replaced independently by —O—, —OH, —NH—, —NH₂, —N(CH₃)—, —NH(CH₃) or —N(CH₃)₂ and R9b represents hydrogen or methyl;

R3a represents independently at each occurrence hydrogen fluoro, chloro, methyl or methoxy;

R3b represents hydrogen;

R4a represents hydrogen, amino, C1-C4alkyl, C1-C4alkyl wherein one CH₂ is replaced by —NH— or —N(CH₃)—, —C1-C4alkylene-cyano, —C1-C4alkylene-hydroxyl, —C1-C4alkylene-amino or —C1-C4alkylene-Cycle-P, wherein Cycle-P is a 5- to 6-membered heterocyclic ring, preferably hydrogen, methyl, ethyl, amino, —CH₂CH₂CN, —CH₂CH₂-morpholin-4-yl or —CH2CH2OH;

n is 1 or 2; and

q is 0.

In one embodiment (Embodiment 3)

Ring A represents group A-I;

B1, B2, B3 and B4 represent independently C(R3a) or C(R3b);

T represents >C═ or >CH—;

X represents —CH₂—, —CH═ or —C(O)—;

R4a represents methyl;

n is 1;

q is 0;

and wherein R2, R3a and R3b are as defined in embodiment E2.

In one embodiment (Embodiment 4a):

Ring A represents group A-II;

A1 and A4 represent CH;

one of A2 and A3 represent C(R4aa) and the other represents CH;

B1, B2, B3 and B4 represent independently C(R3a), C(R3b) or N, wherein no more than one of B1, B2, B3 and B4 represents N, no more than two of B1, B2, B3 and B4 represents C(R3a);

T represents >C═ and X represents —CH═, or T represents >CH— and X represents —C(O)— or T represents >CH— and X represents —CH₂—;

R2 represents fluoro, chloro, bromo, cyano, hydroxyl, C1-C6alkyl, C1-C6haloalkyl, C1-C6alkyl wherein one or two non-adjacent carbon atoms in the alkyl other than the connecting carbon atom are replaced independently by —O—, —OH, —NH—, —NH₂, —N(CH₃)—, —NH(CH₃), —N(CH₃)₂ or —CN, or C1-C6haloalkyl wherein one or two non-adjacent carbon atoms in the haloalkyl other than the connecting carbon atom are replaced independently by —O—, —OH, —NH—, —NH₂, —N(CH₃)—, —NH(CH₃), —N(CH₃)₂ or —CN, or C1-C6alkoxy, C1-C6alkoxy wherein one carbon atom in the alkoxy other than the carbon atom connected to the oxygen is replaced by —O—, —OH, —NH—, —NH₂, —N(CH₃)—, —NH(CH₃), —N(CH₃)₂ or —CN, or —C1-C6alkylene-N(R9a)(R9b) and wherein R9a represents hydrogen, C1-C6alkyl wherein one or two non-adjacent carbon atoms in the alkyl and preferably other than the carbon atom connected to the nitrogen atom are replaced independently by —O—, —OH, —NH—, —NH₂, —N(CH₃)—, —NH(CH₃), —N(CH₃)₂ or —CN, or R9a represents —C1-C6-alkylene-Cycle-P or Cycle-P, wherein Cycle-P preferably represents a saturated 4- to 6-membered heterocyclic ring containing one or two heteroatoms selected from O and N(R15), wherein the heterocyclic ring is optionally substituted by one to three substituents selected from methyl, R9b represents hydrogen, methyl or ethyl, preferably hydrogen or methyl, and R15 represents hydrogen or methyl;

R3a represents independently at each occurrence hydrogen fluoro, chloro, methyl or methoxy;

R3b represents independently at each occurrence hydrogen fluoro, chloro, methyl or methoxy;

R4aa represents hydrogen, amino, C1-C4alkyl, C1-C4alkyl wherein one CH₂ is replaced by —NH— or —N(CH₃)—, C3-C4cycloalkyl, —C1-C4alkylene-cyano, —C1-C4alkylene-hydroxyl, —C1-C4alkylene-amino, —C1-C4alkylene-methoxy, —C1-C4alkylene-C3-C4cycloalkyl or —C1-C4alkylene-Cycle-P, wherein Cycle-P is a 5- to 6-membered heterocyclic ring, more preferably hydrogen, methyl, ethyl, amino, —CH₂CH₂CN, —CH₂CH₂-morpholinyl (e.g. —CH₂CH₂-morpholin-4-yl), CH₂OH, —CH₂CH₂OH, —CH₂OCH₃ or cyclopropyl, even more preferably hydrogen methyl, ethyl or cyclopropyl;

n is 1 or 2; and

q is 0.

In one embodiment (Embodiment 4b):

Ring A represents group A-II;

A1 and A4 represent CH;

one of A2 and A3 represent C(R4aa) and the other represents CH;

B1, B2, B3 and B4 represent independently C(R3a), C(R3b) or N, wherein no more than one of B1, B2, B3 and B4 represents N, no more than two of B1, B2, B3 and B4 represents C(R3a);

T represents >C═ and X represents —CH═, or T represents >CH— and X represents —C(O)— or T represents >CH— and X represents —CH₂—;

R2 represents fluoro, chloro, bromo, cyano, hydroxyl, C1-C6alkyl, C1-C6haloalkyl, C1-C6alkyl wherein one or two non-adjacent carbon atoms in the alkyl other than the connecting carbon atom are replaced independently by —O—, —OH, —NH—, —NH₂, —N(CH₃)—, —NH(CH₃) or —N(CH₃)₂, or C1-C6haloalkyl wherein one or two non-adjacent carbon atoms in the haloalkyl other than the connecting carbon atom are replaced independently by —O—, —OH, —NH—, —NH₂, —N(CH₃)—, —NH(CH₃) or —N(CH₃)₂, or C1-C6alkoxy, C1-C6alkoxy wherein one carbon atom in the alkoxy other than the carbon atom connected to the oxygen is replaced by —O—, —OH, —NH—, —NH₂, —N(CH₃)—, —NH(CH₃) or —N(CH₃)₂, or —C1-C6alkylene-N(R9a)(R9b) and wherein R9a represents hydrogen or C1-C6alkyl wherein one or two non-adjacent carbon atoms in the alkyl are replaced independently by —O—, —OH, —NH—, —NH₂, —N(CH₃)—, —NH(CH₃) or —N(CH₃)₂ and R9b represents hydrogen or methyl;

R3a represents independently at each occurrence hydrogen fluoro, chloro, methyl or methoxy;

R3b represents hydrogen;

R4aa represents hydrogen, amino, C1-C4alkyl, C1-C4alkyl wherein one CH₂ is replaced by —NH— or —N(CH₃)—, —C1-C4alkylene-cyano, —C1-C4alkylene-hydroxyl, —C1-C4alkylene-amino or —C1-C4alkylene-Cycle-P, wherein Cycle-P is a 5- to 6-membered heterocyclic ring, preferably hydrogen, methyl, ethyl, amino, —CH₂CH₂CN, —CH₂CH₂-morpholin-4-yl or —CH₂CH₂OH, more preferably hydrogen or methyl;

n is 1 or 2; and

q is 0.

In one embodiment (Embodiment 5a):

Ring A represents group A-II;

B1, B2, B3 and B4 represent independently C(R3a) or C(R3b);

T represents >C═ or >CH—;

X represents —CH₂—, —CH═ or —C(O)—;

R4aa represents methyl, ethyl or cyclopropyl;

n is 1;

q is 0;

and wherein R2, R3a and R3b are as defined in embodiment E4a.

In one embodiment (Embodiment 5b):

Ring A represents group A-II;

B1, B2, B3 and B4 represent independently C(R3a) or C(R3b);

T represents >C═ or >CH—;

X represents —CH₂—, —CH═ or —C(O)—;

R4aa represents methyl;

n is 1;

q is 0;

and wherein R2, R3a and R3b are as defined in embodiment E4b.

In one embodiment (Embodiment 6):

Ring A represents Group A-I;

A5 represents N;

B1 and B4 represent CH, and one of B2 and B3 represents C(R3a) and the other represents C(R3b);

T represents >C═ and X represents —CH═, or T represents >CH— and X represents —C(O)— or T represents >CH— and X represents —CH₂—;

R2 represents fluoro, chloro, bromo, cyano, hydroxyl, C1-C6alkyl, C1-C6haloalkyl, C1-C6alkoxy, C1-C6haloalkoxy, —C1-C4alkylene-methoxy, —N(R9b)-C1-C4alkylene-R18, —N(R9b)-C1-C4alkylene-Cycle-P or —N(R9b)-Cycle-P, wherein Cycle-P represents tetrahydrofuranyl, azetidinyl, pyrrolidinyl, piperidinyl, piperazinyl, dioxanyl or morpholinyl wherein each N is substituted by R15;

R3a represents independently at each occurrence hydrogen fluoro, chloro, methyl or methoxy;

R3b represents independently at each occurrence hydrogen or fluoro;

R4a represents more preferably hydrogen, methyl, ethyl, amino, —CH₂CH₂CN, —CH₂CH₂-morpholinyl or —CH₂CH₂OH;

R9b represents hydrogen, methyl or ethyl;

R15 represents independently at each occurrence hydrogen or methyl;

R18 represents —OH, —OCH₃, —CN, —NH₂, —NH(CH₃), or —N(CH₃)₂;

n is 1 or 2; and

q is 0.

In one embodiment (Embodiment 7):

Ring A represents group A-II;

A1 and A4 represent CH;

one of A2 and A3 represent C(R4aa) and the other represents CH;

B1 and B4 represent CH, and one of B2 and B3 represents C(R3a) and the other represents C(R3b);

T represents >C═ and X represents —CH═, or T represents >CH— and X represents —C(O)— or T represents >CH— and X represents —CH₂—;

R2 represents fluoro, chloro, bromo, cyano, hydroxyl, C1-C6alkyl, C1-C6haloalkyl, C1-C6alkoxy, C1-C6haloalkoxy, —C1-C4alkylene-methoxy, —N(R9b)-C1-C4alkylene-R18, —N(R9b)-C1-C4alkylene-Cycle-P or —N(R9b)-Cycle-P, wherein Cycle-P represents tetrahydrofuranyl, azetidinyl, pyrrolidinyl, piperidinyl, piperazinyl, dioxanyl or morpholinyl wherein each N is substituted by R15;

R3a represents independently at each occurrence hydrogen fluoro, chloro, methyl or methoxy;

R3b represents independently at each occurrence hydrogen or fluoro;

R4aa represents hydrogen, methyl, ethyl, amino, —CH₂CH₂CN, —CH₂CH₂-morpholinyl (e.g. —CH₂CH₂-morpholin-4-yl), CH₂OH, —CH₂CH₂OH, —CH₂OCH₃ or cyclopropyl;

R9b represents hydrogen, methyl or ethyl;

R15 represents hydrogen or methyl;

R18 represents —OH, —OCH₃, —CN, —NH₂, —NH(CH₃), or —N(CH₃)₂;

n is 1 or 2; and

q is 0.

In one embodiment (Embodiment 8):

Ring A represents Group A-I;

A5 represents N;

B1 and B4 represent CH, and one of B2 and B3 represents C(R3a) and the other represents C(R3b);

T represents >C═ and X represents —CH═, or T represents >CH— and X represents —C(O)— or T represents >CH— and X represents —CH₂—.

R2 represents fluoro, chloro, cyano, methyl, trifluromethyl, N(CH₃)₂ or methoxy;

R3a hydrogen or fluoro;

R3b represents fluoro;

R4a methyl;

n is 1; and

q is 0.

In one embodiment (Embodiment 9):

Ring A represents group A-II;

A1 and A4 represent CH;

one of A2 and A3 represent C(R4aa) and the other represents CH;

B1 and B4 represent CH, and one of B2 and B3 represents C(R3a) and the other represents C(R3b);

T represents >C═ and X represents —CH═, or T represents >CH— and X represents —C(O)— or T represents >CH— and X represents —CH₂—;

R2 represents fluoro, chloro, cyano, methyl, trifluromethyl, N(CH₃)₂ or methoxy;

R3a hydrogen or fluoro;

R3b represents fluoro;

R4aa represents methyl, ethyl or cyclopropyl;

n is 1; and

q is 0.

In one embodiment (Embodiment 10):

T represents >C═ and X represents —CH═, or T represents >CH— and X represents —C(O)— or T represents >CH— and X represents —CH₂—;

R4a, R4aa and R4b represent independently at each occurrence hydrogen, amino, —NH(C1-C4alkyl), —N(C1-C4 alkyl)₂ or —C1-C4alkylene-R4c; and

R9a represents hydrogen, C1-C6alkyl optionally substituted by one to five R14, —C1-C6alkylene-Cycle-P or —C1-C6alkylene-Cycle-Q.

In one embodiment the compound of formula I is a compound of formula Ia

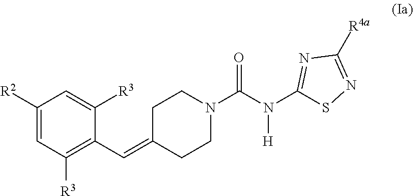

wherein R2, R3 and R4a are as defined for the compound of formula I, including preferred definitions thereof, and preferably wherein R2, R3 and R4a are as defined in any one of embodiments 1a, 1b, 2, 3, 6 or 8, in which case R3 is R3a.

In one embodiment the compound of formula I is a compound of formula Ib

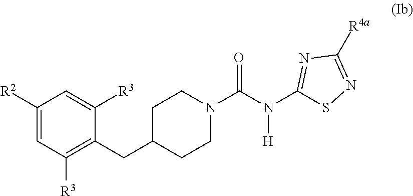

wherein R2, R3 and R4a are as defined for the compound of formula I, including preferred definitions thereof, and preferably wherein R2, R3 and R4a are as defined in any one of embodiments 1a, 1b, 2, 3, 6 or 8, in which case R3 is R3a.

In one embodiment the compound of formula I is a compound of formula Ic

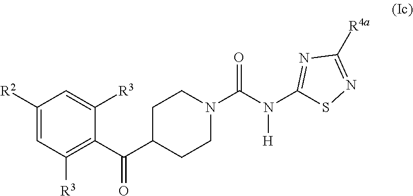

wherein R2, R3 and R4a are as defined for the compound of formula I, including preferred definitions thereof, and preferably wherein R2, R3 and R4a are as defined in any one of embodiments 1a, 1b, 2, 3, 6 or 8, in which case R3 is R3a.

In one embodiment the compound of formula I is a compound of formula Id

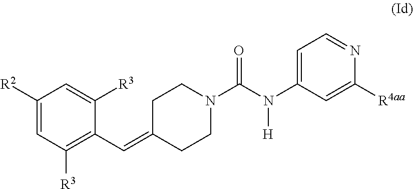

wherein R2, R3 and R4aa are as defined for the compound of formula I, including preferred definitions thereof, and preferably wherein R2, R3 and R4aa are as defined in any one of embodiments 1a, 1b, 4a, 4b, 5a, 5b, 7 or 9, in which case R3 is R3a.

In one embodiment the compound of formula I is a compound of formula Ie

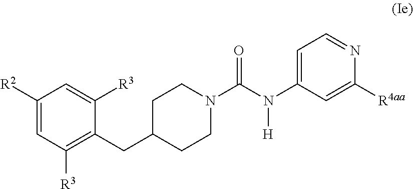

wherein R2, R3 and R4aa are as defined for the compound of formula I, including preferred definitions thereof and preferably wherein R2, R3 and R4a are as defined in any one of embodiments 1a, 1b, 4a, 4b, 5a, 5b, 7 or 9, in which case R3 is R3a.

In one embodiment the compound of formula I is a compound of formula If

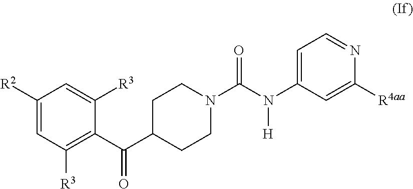

wherein R2, R3 and R4aa are as defined for the compound of formula I, including preferred definitions thereof, and preferably wherein R2, R3 and R4aa are as defined in any one of embodiments 1a, 1b, 4a, 4b, 5a, 5b, 7 or 9, in which case R3 is R3a.

In further embodiments the invention also provides the compounds or pharmaceutically acceptable salts, solvates or hydrates thereof of each of the compounds depicted in Table 1 below. A depiction in Table 1 of a compound as a salt does not limit the compound to that salt for the purposes of these embodiments of the invention. Embodiments of particular interest include the following:
4-[(4-chloro-2,6-difluoro-phenyl)methyl]-N-(3-methyl-1,2,4-thiadiazol-5-yl)piperidine-1-carboxamide (e.g. Example 25);
4-[(4-chloro-2,6-difluoro-phenyl)methylene]-N-(3-methyl-1,2,4-thiadiazol-5-yl)piperidine-1-carboxamide (e.g. Example 26);
4-[(2-fluoro-4-methyl-phenyl)methyl]-N-(3-methyl-1,2,4-thiadiazol-5-yl)piperidine-1-carboxamide (e.g. Example 27);
4-[(4-chloro-2-fluoro-phenyl)methylene]-N-(3-methyl-1,2,4-thiadiazol-5-yl)piperidine-1-carboxamide (e.g. Example 39);
4-[[2,6-difluoro-4-(trifluoromethyl)phenyl]methyl]-N-(3-methyl-1,2,4-thiadiazol-5-yl)piperidine-1-carboxamide (e.g. Example 40);
4-[[2,6-difluoro-4-(trifluoromethyl)phenyl]methylene]-N-(3-methyl-1,2,4-thiadiazol-5-yl)piperidine-1-carboxamide (e.g. Example 41);
4-[[4-(dimethylamino)-2-fluoro-phenyl]methylene]-N-(3-methyl-1,2,4-thiadiazol-5-yl)piperidine-1-carboxamide (e.g. Example 47);
4-[(4-chloro-2-fluoro-phenyl)methyl]-N-(3-methyl-1,2,4-thiadiazol-5-yl)azepane-1-carboxamide (e.g. Example 49);
4-[(2,4-difluorophenyl)methylene]-N-(3-methyl-1,2,4-thiadiazol-5-yl)piperidine-1-carboxamide (e.g. Example 59);
4-[(2,4-difluorophenyl)methyl]-N-(3-methyl-1,2,4-thiadiazol-5-yl)piperidine-1-carboxamide (e.g. Example 60);
4-[(4-chloro-2-fluoro-phenyl)methyl]-N-(3-methylisothiazol-5-yl)piperidine-1-carboxamide (e.g. Example 63);
4-[(4-chloro-2-fluoro-phenyl)methyl]-N-(3-ethyl-1,2,4-thiadiazol-5-yl)piperidine-1-carboxamide (e.g. Example 70);
4-[(4-chloro-2-fluoro-phenyl)methyl]-N-(2-methyl-4-pyridyl)piperidine-1-carboxamide (e.g. Example 72);
4-(4-chlorobenzoyl)-N-(3-methyl-1,2,4-thiadiazol-5-yl)piperidine-1-carboxamide (e.g. Example 74);
4-(2,4-difluorobenzoyl)-N-(3-methyl-1,2,4-thiadiazol-5-yl)piperidine-1-carboxamide (e.g. Example 75);
4-(4-bromobenzoyl)-N-(3-methyl-1,2,4-thiadiazol-5-yl)piperidine-1-carboxamide (e.g. Example 77);
(4E)-4-[(4-chloro-2-fluoro-phenyl)methylene]-3-methyl-N-(3-methyl-1,2,4-thiadiazol-5-yl)piperidine-1-carboxamide (e.g. Example 82);
4-[(4-chloro-2-fluoro-phenyl)methyl]-3-methyl-N-(3-methyl-1,2,4-thiadiazol-5-yl)piperidine-1-carboxamide (e.g. Example 84a);
(4E)-4-[(4-chloro-2-fluoro-phenyl)methylene]-3,3-dimethyl-N-(3-methyl-1,2,4-thiadiazol-5-yl)piperidine-1-carboxamide (e.g. Example 85);
(4E)-4-[(4-chloro-2-fluoro-phenyl)methylene]-2-methyl-N-(3-methyl-1,2,4-thiadiazol-5-yl)piperidine-1-carboxamide (e.g. Example 86);
4-(4-chloro-2-fluoro-benzoyl)-N-(3-methyl-1,2,4-thiadiazol-5-yl)piperidine-1-carboxamide (e.g. Example 92);
4-[[2-fluoro-4-(methoxymethyl)phenyl]methylene]-N-(3-methyl-1,2,4-thiadiazol-5-yl)piperidine-1-carboxamide (e.g. Example 93);
4-[(4-cyano-2,6-difluoro-phenyl)methylene]-N-(3-methyl-1,2,4-thiadiazol-5-yl)piperidine-1-carboxamide (e.g. Example 95);
4-[(2,6-difluoro-4-methoxy-phenyl)methylene]-N-(3-methyl-1,2,4-thiadiazol-5-yl)piperidine-1-carboxamide (e.g. Example 97);
4-[(2,6-difluoro-4-methoxy-phenyl)methyl]-N-(3-methyl-1,2,4-thiadiazol-5-yl)piperidine-1-carboxamide (e.g. Example 98);
4-[[4-(dimethylamino)-2,6-difluoro-phenyl]methylene]-N-(3-methyl-1,2,4-thiadiazol-5-yl)piperidine-1-carboxamide (e.g. Example 112);
4-[[4-(dimethylamino)-2,6-difluoro-phenyl]methyl]-N-(3-methyl-1,2,4-thiadiazol-5-yl)piperidine-1-carboxamide (e.g. Example 113);
4-[(4-chloro-2,6-difluoro-phenyl)methyl]-N-(3-methyl-1,2,4-thiadiazol-5-yl)azepane-1-carboxamide (e.g. Example 114);
(4Z)-4-[[4-(dimethylamino)-2,6-difluoro-phenyl]methylene]-N-(3-methyl-1,2,4-thiadiazol-5-yl)azepane-1-carboxamide (e.g. Example 115);
(4Z)-4-[(4-chloro-2-fluoro-phenyl)methylene]-N-(3-methyl-1,2,4-thiadiazol-5-yl)azepane-1-carboxamide (e.g. Example 118);
4-[[2-fluoro-4-(trifluoromethyl)phenyl]methyl]-N-(3-methyl-1,2,4-thiadiazol-5-yl)azepane-1-carboxamide (e.g. Example 120);
4-[[4-(dimethylamino)-2,6-difluoro-phenyl]methyl]-N-(3-methyl-1,2,4-thiadiazol-5-yl)azepane-1-carboxamide (e.g. Example 121);
4-[(4-chloro-2,6-difluoro-phenyl)methyl]-N-(3-methyl-1,2,4-thiadiazol-5-yl)azepane-1-carboxamide (e.g. Example 127);
4-[(4-cyano-2,6-difluoro-phenyl)methyl]-N-(3-methyl-1,2,4-thiadiazol-5-yl)piperidine-1-carboxamide (e.g. Example 129);
4-[(4-chloro-2,6-difluoro-phenyl)methylene]-N-(2-methyl-4-pyridyl)piperidine-1-carboxamide (e.g. Example 137);
4-(2-fluoro-4-methoxy-benzoyl)-N-(3-methyl-1,2,4-thiadiazol-5-yl)piperidine-1-carboxamide (e.g. Example 140);
4-(4-bromo-2-fluoro-benzoyl)-N-(3-methyl-1,2,4-thiadiazol-5-yl)piperidine-1-carboxamide (e.g. Example 141);
4-[(4-chloro-2-fluoro-phenyl)methylene]-N-(2-ethyl-4-pyridyl)piperidine-1-carboxamide (e.g. Example 144);
4-[[2,6-difluoro-4-[2-methoxyethyl(methyl)amino]phenyl]methylene]-N-(3-methyl-1,2,4-thiadiazol-5-yl)piperidine-1-carboxamide (e.g. Example 146);
4-[[4-(dimethylamino)-2,6-difluoro-phenyl]methylene]-N-(3-methylisothiazol-5-yl)piperidine-1-carboxamide (e.g. Example 148);
4-[[4-(dimethylamino)-2,6-difluoro-phenyl]methylene]-N-(3-ethyl-1,2,4-thiadiazol-5-yl)piperidine-1-carboxamide (e.g. Example 149);
4-[[4-(dimethylamino)-2,6-difluoro-phenyl]methyl]-N-(3-methylisothiazol-5-yl)piperidine-1-carboxamide (e.g. Example 151);
4-[[4-(dimethylamino)-2,6-difluoro-phenyl]methyl]-N-(3-ethyl-1,2,4-thiadiazol-5-yl)piperidine-1-carboxamide (e.g. Example 152);
4-[[2,6-difluoro-4-[2-hydroxyethyl(methyl)amino]phenyl]methylene]-N-(3-methyl-1,2,4-thiadiazol-5-yl)piperidine-1-carboxamide (e.g. Example 158);
4-[(4-chloro-2-fluoro-phenyl)methyl]-6-methoxy-N-(3-methyl-1,2,4-thiadiazol-5-yl)-1,4-diazepane-1-carboxamide (e.g. Example 160);
4-[[4-[cyanomethyl(methyl)amino]-2,6-difluoro-phenyl]methylene]-N-(3-methyl-1,2,4-thiadiazol-5-yl)piperidine-1-carboxamide (e.g. Example 164);
4-[(4-cyano-2,6-difluoro-phenyl)methylene]-N-(2-ethyl-4-pyridyl)piperidine-1-carboxamide (e.g. Example 179);
4-[(4-cyano-2,6-difluoro-phenyl)methylene]-N-(2-cyclopropyl-4-pyridyl)piperidine-1-carboxamide (e.g. Example 182); and
4-[(4-chloro-2-fluoro-phenyl)methylene]-N-(2-cyclopropyl-4-pyridyl)piperidine-1-carboxamide (e.g. Example 183).

The present invention relates also to pharmaceutical compositions that comprise a compound of formula (I) as active ingredient or or pharmaceutically acceptable salt, solvate or hydrate thereof, e.g. present in a therapeutically-effective amount, which can be used especially in the treatment of the proliferation disorders, in particular cancer, as described herein. Compositions may be formulated for non-parenteral administration, such as nasal, buccal, rectal, pulmonary, vaginal, sublingual, topical, transdermal, ophthalmic, otic or, especially, for oral administration, e.g. in the form of oral solid dosage forms, e.g. granules, pellets, powders, tablets, coated tablets (e.g. film or sugar coated), effervescent tablets, hard and soft gelatin or HPMC capsules, coated as applicable, orally disintegrating tablets, solutions, emulsions (e.g. lipid emulsions) or suspensions, or for parenteral administration, such as intravenous, intramuscular or subcutaneous, intrathecal, intradermal or epidural administration, to mammals, especially humans, e.g. in the form of solutions, lipid emulsions or suspensions containing microparticles or nanoparticles. The compositions may comprise the active ingredient alone or, preferably, together with a pharmaceutically acceptable carrier.

The compounds of formula I or pharmaceutically acceptable salt, solvate or hydrate thereof can be processed with pharmaceutically inert, inorganic or organic excipients for the production of oral solid dosage forms, e.g. granules, pellets, powders, tablets, coated tablets (e.g. film or sugar coated), effervescent tablets and hard gelatin or HPMC capsules or orally disintegrating tablets. Fillers e.g. lactose, cellulose, mannitol, sorbitol, calcium phosphate, starch (e.g. corn starch) or derivatives thereof, binders e.g. cellulose, starch, polyvinylpyrrolidone, or derivatives thereof, glidants e.g. talcum, stearic acid or its salts, flowing agents e.g. fumed silica, can be used as such excipients e.g. for formulating and manufacturing of oral solid dosage forms, such as granules, pellets, powders, tablets, film or sugar coated tablets, effervescent tablets, hard gelatine or HPMC capsules, or orally disintegrating tablets. Suitable excipients for soft gelatin capsules are e.g. vegetable oils, waxes, fats, semisolid and liquid polyols etc.

Suitable excipients for the manufacture of solutions (e.g. oral solutions), lipid emulsions or suspensions are e.g. water, alcohols, polyols, saccharose, invert sugar, glucose etc.

Suitable excipients for parenteral formulations (e.g. injection solutions) are e.g. water, alcohols, polyols, glycerol, vegetable oils, lecithin, surfactants etc. . . . .

Moreover, the pharmaceutical preparations can contain preservatives, solubilizers, stabilizers, wetting agents, emulsifiers, sweeteners, colorants, flavorants, salts for varying the osmotic pressure, buffers, masking agents or antioxidants. They can also contain still other therapeutically valuable substances. The dosage can vary within wide limits and will, of course, be fitted to the individual requirements in each particular case. In general, in the case of oral administration a daily dosage of about 1 to 1000 mg, e.g. 10 to 1000 mg per person of a compound of general formula I should be appropriate, although the above upper limit (and likewise the lower limit) can also be exceeded when necessary.

The compounds of formula (I) can also be used in combination with one or more other pharmaceutically active compounds, which are either effective against the same disease, preferably using a different mode of action, or which reduce or prevent possible undesired side effects of the compounds of formula (I). The combination partners can be administered in such a treatment either simultaneously, e.g. by incorporating them into a single pharmaceutical formulation, or consecutively by administration of two or more different dosage forms, each containing one or more than one of the combination partners.

Compounds of formula I according to the invention as described above or pharmaceutically acceptable salts, hydrates or solvates thereof are particularly useful for the treatment of proliferation disorders and/or diseases such as cancer, in particular carcinoma, sarcoma, leukemia, myeloma and lymphoma and cancers of the brain and spinal cord. Examples of such proliferation disorders and diseases include, but are not limited to, epithelial neoplasms, squamous cell neoplasms, basal cell neoplasms, transitional cell papillomas and carcinomas, adenomas and adenocarcinomas, adnexal and skin appendage neoplasms, mucoepidermoid neoplasms, cystic neoplasms, mucinous and serous neoplasms, ducal-, lobular and medullary neoplasms, acinar cell neoplasms, complex epithelial neoplasms, specialized gonadal neoplasms, paragangliomas and glomus tumours, naevi and melanomas, soft tissue tumours and sarcomas, fibromatous neoplasms, myxomatous neoplasms, lipomatous neoplasms, myomatous neoplasms, complex mixed and stromal neoplasms, fibroepithelial neoplasms, synovial like neoplasms, mesothelial neoplasms, germ cell neoplasms, trophoblastic neoplasms, mesonephromas, blood vessel tumours, lymphatic vessel tumours, osseous and chondromatous neoplasms, giant cell tumours, miscellaneous bone tumours, odontogenic tumours, gliomas, neuroepitheliomatous and neuroendocrine neoplasms, meningiomas, nerve sheath tumours, granular cell tumours and alveolar soft part sarcomas, Hodgkin's and non-Hodgkin's lymphomas, B-cell lymphoma, T-cell lymphoma, hairy cell lymphoma, Burkitts lymphoma and other lymphoreticular neoplasms, plasma cell tumours, mast cell tumours, immunoproliferative diseases, leukemias, miscellaneous myeloproliferative disorders, lymphoproliferative disorders and myelodysplastic syndromes.

Examples of cancers in terms of the organs and parts of the body affected include, but are not limited to, the breast, cervix, ovaries, colon, rectum (including colon and rectum i.e. colorectal cancer), lung (including small cell lung cancer, non-small cell lung cancer, large cell lung cancer and mesothelioma), endocrine system, bone, adrenal gland, thymus, liver, stomach (gastric cancer), intestine, pancreas, bone marrow, hematological malignancies (such as lymphoma, leukemia, myeloma or lymphoid malignancies), bladder, urinary tract, kidneys, skin, thyroid, brain, head, neck, prostate and testis. Preferably the cancer is selected from the group consisting of breast cancer, prostate cancer, cervical cancer, ovarian cancer, gastric cancer, colorectal cancer, pancreatic cancer, liver cancer, brain cancer, neuroendocrine cancer, lung cancer, kidney cancer, hematological malignancies, melanoma and sarcomas.

The term “treatment” or “treating” as used herein in the context of treating a disease or disorder, pertains generally to treatment and therapy, whether of a human or an animal (e.g., in veterinary applications), in which some desired therapeutic effect is achieved, for example, the inhibition of the progress of the disease or disorder, and includes a reduction in the rate of progress, a halt in the rate of progress, alleviation of symptoms of the disease or disorder, amelioration of the disease or disorder, and cure of the disease or disorder. Treatment as a prophylactic measure (i.e., prophylaxis) is also included. For example, use with patients who have not yet developed the disease or disorder, but who are at risk of developing the disease or disorder, is encompassed by the term “treatment.” For example, treatment includes the prophylaxis of cancer, reducing the incidence of cancer, alleviating the symptoms of cancer, etc.

The term “therapeutically-effective amount,” as used herein, pertains to that amount of a compound, or a material, composition or dosage form comprising a compound, which is effective for producing some desired therapeutic effect, commensurate with a reasonable benefit/risk ratio, when administered in accordance with a desired treatment regimen.

The compounds according to the present invention, pharmaceutically acceptable salts, solvates, hydrates thereof can be prepared e.g. by one of the processes (a), (b), (c) and (d) described below; followed, if necessary, by:

removing any protecting groups;

forming a pharmaceutically acceptable salt; or

forming a pharmaceutically acceptable solvate or hydrate.

The schemes and processes described herein are not intended to present an exhaustive list of methods for preparing the compounds of formula I; rather, additional techniques of which the skilled chemist is aware may be also used for the compound synthesis.

Process (a):

This process variant can be used for the manufacture of compounds of formula I as defined above, wherein T is >C═ or >CH— and X is —C(R6a)= (double bond Z, E or Z/E) or —C(R6a)(R6b), in which formulae R6a is hydrogen or a C1-C4alkyl and R6b is hydrogen.

In this process a compound of formula II-1

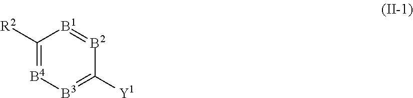

is reacted with a compound of formula III-1

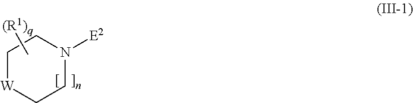

to generate a compound of formula IV-1

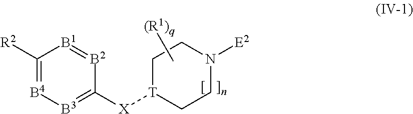

in which formulae,

B1, B2, B3, B4, R1, q and n are as in formula I,

R2 is as in formula 1, or R2 is —NO2,

E2 is a hydrogen or an amino protecting group,

T is >C═,

X is —C(R6a)= (double bond Z, E or Z/E).

When Y1 is —CH(R6a)-Y2,

wherein Y2 is a phosphonium salt or a phosphonate,

then W is >C═O.

When Y1 is a halogen or a leaving group such as mesylate, tosylate, triflate,

then W is >C═C—Y3,

wherein Y3 is a boronic acid or a boronic ester.

When E2 is an amino protecting group, the amino protection of compounds of formula IV-1 can be first removed to generate compounds of formula IV-1 wherein E2 is a hydrogen.

When E2 is a hydrogen, the compound of formula IV-1 is further reacted with a compound of formula V-1

A-NH-E3 (V-1)

wherein ring A is as in formula I,

E3 is —C(O)—Y4,

wherein Y4 is a halogen, or a leaving group such as imidazole, 4-nitrophenol, phenol or 1-hydroxypyrrolidine-2,5-dione,

to generate a compound of formula I-1

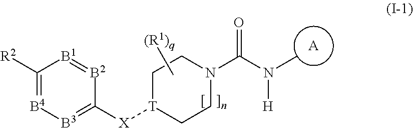

Similarly, when E2 is a hydrogen, compounds of formula IV-1 can first be converted to a compound of formula IV-1 for which E2 is —C(O)—Y4, wherein Y4 is a halogen, or a leaving group such as imidazole, 4-nitrophenol, phenol or 1-hydroxypyrrolidine-2,5-dione. The resulting compound of formula IV-1 is then reacted with a compound of formula V-1, wherein E3 is a hydrogen to generate a compound of formula I-1.

Alternatively, the compound of formula III-1 wherein E2 is a hydrogen can react with compound of formula V-1, wherein E3 is —C(O)—Y4 and Y4 is a halogen, or a leaving group such as imidazole, 4-nitrophenol, phenol or 1-hydroxypyrrolidine-2,5-dione to generate a compound of formula III-1 wherein E2 is —C(O)—NH-A and ring A is as in formula I. The obtained compounds can further react with a compound of formula II-1 to generate a compound of formula I-1 in a similar manner.

The compounds of formulae IV-1 and I-1 for which T is >C═, X is —C(R6a)= (double bond Z, E or Z/E), can further be reduced to generate compounds of formulae IV-2 and I-2, respectively, wherein T is >CH— and X is —C(R6a)(R6b), in which formulae R6a is a C1-C4alkyl and R6b is a hydrogen.

In addition, compounds of formula IV-2 can react further with a compound of formula V-1 to generate compounds of formula I-2 following similar procedures described for the preparation of a compound of formula I-1 from a compound of formula IV-1.

When R2 is a nitro group, compounds of formulae IV-1, IV-2, I-1 or I-2 can be converted to a compound of formulae IV-1, IV-2, I-1 or I-2, respectively, wherein R2 is an amino group.

When R2 is —OCH3, compounds of formulae IV-1, IV-2, I-1 or I-2 can be converted to a compound of formulae IV-1, IV-2, I-1 or I-2, respectively, wherein R2 is a hydroxyl group.

When R2 is —COOH or —C1-C6alkylene-COOH, compounds of formulae IV-1, IV-2, I-1 or I-2 is further reacted with a compound of formula VI

R′2-Y5 (VI)

wherein Y5 is —NH₂, >NH or —NHE, E being an amino protecting group to generate compounds of formulae IV-1, IV-2, I-1 or I-2, respectively, wherein R2 is —C(O)N(R11a)(R11b) or —C1-C6alkylene-C(O)N(R11a)(R11b), R11a and R11b are as in Formula I.

When R2 is —NH₂ or —C1-C6alkylene-NH₂, compounds of formulae IV-1, IV-2, I-1 or I-2 is further reacted with a compound of formula VI, wherein Y5 is —CHO or Y5 is a halogen or a leaving group (such as mesylate, tosylate or triflate) to generate compounds of formulae IV-1, IV-2, I-1 or I-2, respectively, wherein R2 is —N(R9a)(R9b) or —C1-C6alkylene-N(R9a)(R9b), R9a and R9b are as in formula I.

When R2 is —NH₂ or —C1-C6alkylene-NH₂, compounds of formulae IV-1, IV-2, I-1 or I-2 is further reacted with a compound of formula VI, wherein Y5 is —COOH to generate compounds of formulae IV-1, IV-2, I-1 or I-2, respectively, wherein R2 is —N(R12)C(O)(R13) or —C1-C6alkylene-N(R12)C(O)(R13), R12 and R13 are as in formula I.

When R2 is —CHO or —C1-C6alkylene-CHO, compounds of formulae IV-1, IV-2, I-1 or I-2 is further reacted with a compound of formula VI, wherein Y5 is —NH₂ or >NH to generate compounds of formulae IV-1, IV-2, I-1 or I-2, respectively, wherein R2 is —C1-C6alkylene-N(R9a)(R9b), R9a and R9b are as in formula I.

When R2 is —C1-C6alkylene-E4, wherein E4 is a halogen or a leaving group such as mesylate, tosylate or triflate, compounds of formulae IV-1, IV-2, I-1 or I-2 is further reacted with a compound of formula VI, wherein Y5 is —OH, —NH2, >NH or —NHE, E being an amino protecting group, to generate compounds of formulae IV-1, IV-2, I-1 or I-2, respectively, wherein R2 is —C1-C6akyl substituted with one to five R14, at least one R14 is C1-C6alkoxy, or —C1-C6alkylene —N(R9a)(R9b), R9a and R9b are as in formula I.

When R2 is —OH, compounds of formulae IV-1, IV-2, I-1 or I-2 is further reacted with a compound of formula VI wherein Y5 is halogen or leaving group such as mesylate, toylsate or triflate to generate compounds of formulae IV-1, IV-2, I-1 or I-2, respectively, wherein R2 is C1-C6-alkyloxy optionally substituted by one to five R14, R14 is as defined for formula I.

When R2 is a halogen atom or a triflate, compounds of formulae IV-1, IV-2, I-1 or I-2 is further reacted with a compound of formula VI, wherein Y5 is —OH, —NH2 or >NH to generate compounds of formulae IV-1, IV-2, I-1 or I-2, respectively, wherein R2 is C1-C6alkoxy optionally substituted by one to five R14 or —N(R9a)(R9b), R14, R9a and R9b are as in formula I.

When R4c is a halogen or a leaving group such as mesylate, tosylate or triflate, then compounds of formulae I-1 or I-2 can further react with a compound of formula VI, wherein Y5 is —CN, —OH or >NH to generate compounds of formulae I-1 or I-2, respectively, wherein R4c is a nitril, C1-C4alkoxy, Cycle-P or Cycle-Q is as in formula I.

It will be clear to the person skilled in the art that in the above explanations, R′2 represents any additional substituents present in the given description of R2 after the chemical reaction with the functional group represented by Y5 has taken place.

Process (b):

This process variant can be used for the manufacture of compounds of formula I as defined above, wherein T is >CH— and X is —O— or —S—.

In this process a compound of formula II-2

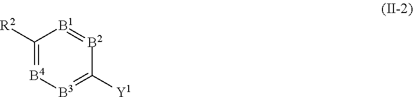

is reacted with a compound of formula III-2

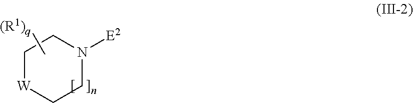

to generate a compound of formula IV-3

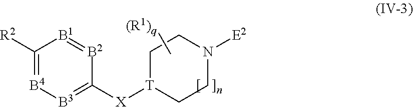

in which formulae,

B1, B2, B3, B4, R1, q and n are as in formula I,

R2 is as in formula 1,

E2 is a hydrogen or an amino protecting group,

T is >CH—,

X is —O— or —S—,

Y1 is —OH or —SH,

W is >CH—Z,

wherein Z is —OH, a halogen or a leaving group such mesylate, tosylate or triflate.

Following procedures already described in process (a), the compound of formula IV-3 can react with a compound of formula V-1 to generate a compound of formula I-3.

Process (c):

This process variant can be used for the manufacture of compounds of formula I as defined above, wherein T is >N— and X is —C(R6a)(R6b), wherein R6a and R6b are as defined for formula I.

In this process a compound of formula II-3

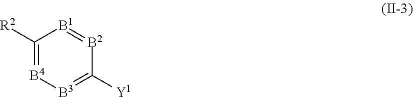

is reacted with a compound of formula III-3

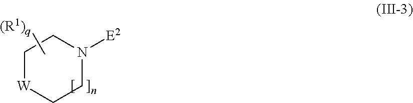

to generate a compound of formula IV-4

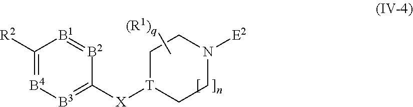

in which formulae,

B1, B2, B3, B4, R1, q and n are as in formula I,

R2 is as in formula 1,

E2 is a hydrogen or an amino protecting group,

T is >N—,

X is —C(R6a)(R6b), wherein R6a and R6b are as defined for formula I,

Y1 is —C(R6a)(R6b)Z or —C(O)(R6a),

wherein Z is a halogen or a leaving group such as mesylate, tosylate or triflate,

W is >NH,

Following procedures already described in process (a), the compounds of formula IV-4 can react with a compound of formula V-1 to generate a compound of formula I-4.

Process (d):

This process variant can be used for the manufacture of compounds of formula I as defined above, wherein T is >CH— and X is —C(O)—.

In this process a compound of formula II-4

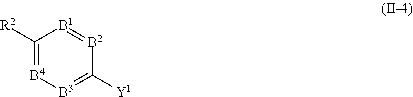

is reacted with a compound of formula III-4

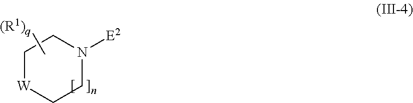

to generate a compound of formula IV-5

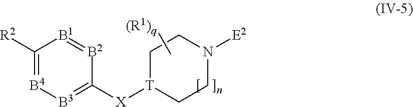

in which formulae,

B1, B2, B3, B4, R1, q and n are as in formula I,

R2 is as in formula 1,

E2 is a hydrogen or an amino protecting group (such as N-acetyl),

T is >CH—,

X is —C(O)—,

Y1 is a hydrogen atom or a halogen atom.

W is >CH—Z,

wherein Z is —C(O)C1 or —C(O)E5, E5 being a leaving group.

Alternatively, a compound of formula IV-1, wherein X is >CH═ and T is >C═, can be oxidized to generate a compound of formula VII

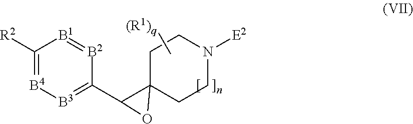

Compounds of formula VII can be then converted to compounds for formula IV-5.

Following procedures already described in process (a), the compound of formula IV-5 can react with a compound of formula V-1 to generate a compound of formula I-5.

The compounds of formula I can be prepared by methods given below, by methods given in the experimental part below or by analogous methods. Optimum reaction conditions may vary with the particular reactants or solvents used, but such conditions can be determined by a person skilled in the art by routine optimization procedures. It is understood by one skilled in the art of organic synthesis that the functionality present on various portions of the molecule must be compatible with the reagents and reactions proposed. Such restrictions to the substituents, which are compatible with the reaction conditions, will be readily apparent to one skilled in the art and alternate methods must then be used.

The necessary starting materials for the synthetic methods as described herein, if not commercially available, may be made by procedures which are described in the scientific literature, or may be made from commercially available compounds using adaptations of processes reported in the scientific literature. The reader is further referred to Advanced Organic Chemistry, 7^{th} Edition, by J. March and M. Smith, published by John Wiley & Sons, 20013 for general guidance on reaction conditions and reagents.

In some cases, the final product may be further modified, for example, by manipulations of substituents to give a new final product. These manipulations may include, but are not limited to, reduction, oxidation, alkylation, acylation, and hydrolysis reactions which are commonly known by those skilled in the art. The compounds obtained may also be converted into salts, especially pharmaceutically acceptable salts in a manner known per se.

Furthermore in some of the reactions mentioned herein it may be necessary or desirable to protect any sensitive groups in compounds. For the purpose of this discussion, it will be assumed that such protecting groups as necessary are in place. Conventional protecting groups may be used in accordance with standard practice and the use of protecting groups is well known in the art (for illustration see Protective Groups in Organic Synthesis, 5^{th} Edition, by T. W. Greene and P. G. M. Wuts, published by John Wiley & Sons, 2014).

The protecting groups may be removed at any convenient stage in the synthesis using conventional techniques well known in the art, or they may be removed during a later reaction step or work-up.

The compounds of formula I wherein T is >C═ or >CH— and X is —C(R6a)= (double bond Z, E or Z/E) or —C(R6a)(R6b), in which formulae R6a is hydrogen or a C1-C4alkyl and R6b is hydrogen, can be obtained as summarized in Scheme 1.

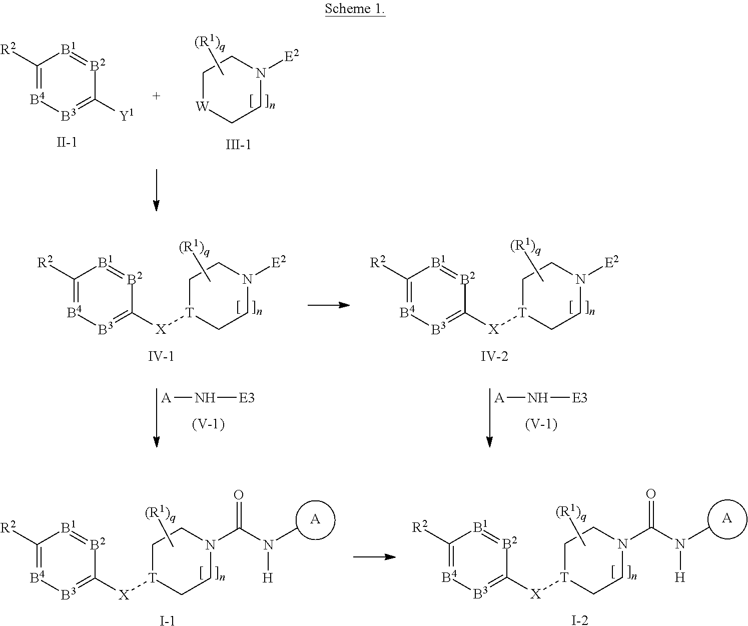

In Scheme 1, all the symbols have the same meanings as previously described in process (a).

When W is >C═O, compounds of formula III-1 can react with compounds of formula II-1 for which Y1 is —CH(R6a)-Y2 and Y2 is a phosphonium salt or a phosphonate via a Wittig or Horner-Wadsworth-Emmons reaction, respectively, to generate compounds of formula IV-1 for which X is —C(R6a)= (double bond Z, E or Z/E).

The Wittig reaction is the reaction of an aldehyde or ketone with a triphenyl phosphonium ylide to afford an alkene and triphenylphosphine oxide. The Wittig reagent is usually prepared from a phosphonium salt, which is, in turn, prepared by alkylation of triphenylphosphine with a benzyl halide. To form the Wittig reagent (benzyl ylide), the phosphonium salt is suspended in a solvent such as diethyl ether or tetrahydrofuran and a strong base such as n-butyl lithium or lithium bis(trimethylsilyl)amide is added. With simple ylides, the product is usually mainly the Z-isomer, although a lesser amount of the E-isomer also is often formed. If the reaction is performed in N,N-dimethylformamide in the presence of lithium or sodium iodide, the product is almost exclusively the Z-isomer. If the E-isomer is the desired product, the Schlosser modification may be used.

Alternatively the Horner-Wadsworth-Emmons reaction produces predominantly E-alkenes. The Horner-Wadsworth-Emmons reaction is the condensation of stabilized phosphonate carbanions with aldehydes or ketones in presence of a base such as sodium hydride or lithium bis(trimethylsilyl)amide in a solvent such as tetrahydrofuran or N,N-dimethylformamide, between 0° C. and 80° C. In contrast to phosphonium ylides used in the Wittig reaction, phosphonate-stabilized carbanions are more nucleophilic and more basic. Diethyl benzylphosphonates can be easily prepared from readily available benzyl halides.

An alternative route to prepare compounds IV-1 can be used. When W is >C═C—Y3, (Y3 is a boronic acid or a boronic ester), compounds of formula III-1 can react with compounds of formula II-1 for which Y1 is a halogen or a leaving group such as triflate via Suzuki cross-coupling reaction, to generate compounds of formula IV-1 for which X is —C(R6a)= (double bond Z, E or Z/E).

The Suzuki reaction is a palladium-catalyzed cross-coupling reaction between organoboronic acids and aryl or vinyl halides or triflates. Typical catalysts include palladium(II) acetate, tetrakis(triphenylphosphine)palladium(0), bis(triphenylphosphine)palladium(II) dichloride and [1,1′bis(diphenylphosphino)ferrocene]dichloropalladium(II). The reaction can be carried out in a variety of organic solvents including toluene, tetrahydrofuran, dioxane, 1,2-dichloroethane, N,N-dimethylformamide, dimethylsulfoxide and acetonitrile, aqueous solvents and under biphasic conditions. Reactions are typically run from room temperature to 150° C. Additives such as cesium fluoride, potassium fluoride, potassium hydroxide, potassium carbonate, potassium acetate, potassium phosphate or sodium ethylate frequently accelerate the coupling. Potassium trifluoroborates and organoboranes or boronate esters may be used in place of boronic acids. Although there are numerous components in the Suzuki reaction such as the particular palladium catalyst, the ligand, additives, solvent, temperature, numerous protocols have been identified. One skilled in the art will be able to identify a satisfactory protocol without undue experimentation.

Organoboronic acids or esters III-1 are generally obtained from diboron reagents (such as bis(pinacolato)diboron or bis-boronic acid) and vinyl halides via Miyaura borylation (J. Org. Chem., 1995, 60, 7508) in presence of a palladium catalyst such as tris(dibenzylideneacetone)dipalladium-chloroform complex or chloro(2-dicyclohexylphosphino-2′,4′,6′-triisopropyl-1,1′-biphenyl)[2-(2′-amino-1,1′-biphenyl)]palladium(II) and a ligand such as triphenylphosphine or 2-(dicyclohexylphosphino)-2′,4′, 6′-tri-isopropyl-1,1′-biphenyl. The reaction can be carried out in a variety of organic solvents including toluene, tetrahydrofuran, dioxane, 1,2-dichloroethane, N,N-dimethylformamide, dimethylsulfoxide and acetonitrile, aqueous solvents and under biphasic conditions. Reactions are typically run from room temperature to 150° C. (more frequently 100° C.). Crucial for the success of the borylation reaction is the choice of an appropriate base, as strong activation of the product enables the competing Suzuki coupling. The use of potassium acetate (J. Org. Chem., 1995, 60, 7508) and potassium phenolate (J. Am. Chem. Soc., 2002, 124, 8001) is actually the result of a screening of different reaction conditions by the Miyaura group. Other bases such as potassium hydroxide, potassium carbonate, potassium phosphate or sodium ethylate are frequently used as well. As for the Suzuki reaction, there are numerous components in the Miyaura borylation reaction such as the particular palladium catalyst, the ligand, additives, solvent, temperature and numerous protocols have been identified. One skilled in the art will be able to identify a satisfactory protocol without undue experimentation.

Vinyl halides used for the preparation of organoboronic acids or esters III-1 can be prepared via a Wittig reaction between compounds III-1 (wherein W is >C═O) and a halide-methyl triphenylphosphonium salt following procedures previously described.

The amino protecting group E2 is introduced by reacting the corresponding free amine with allyl, fluorenylmethyl or benzyl chloroformate, or with di-tert-butyl dicarbonate in presence of a base such as sodium hydroxide, sodium hydrogen carbonate, triethylamine, 4-dimethylaminopyridine or imidazole. The free amine can also be protected as N-benzyl derivatives by reaction with benzyl bromide or chloride in presence of a base such as sodium carbonate or triethylamine. Alternatively, N-benzyl derivatives can be obtained through reductive amination in presence of benzaldehyde. The free amide can also be protected as N-acetyl derivatives by reaction with acetyl chloride or acetic anhydride in presence of a base such as sodium carbonate or trimethylamine. Further strategies to introduce other amino protecting groups have been described in Protective Groups in Organic Synthesis, 5^{th} Edition, by T. W. Greene and P. G. M. Wuts, published by John Wiley & Sons, 2014.

The amino protecting group E2 can further be removed under standard conditions. For example the benzyl carbamates are deprotected by hydrogenolysis over a noble metal catalyst (e.g. palladium or palladium hydroxide on activated carbon or other suitable catalyst e.g. Raney-Ni). The Boc group is removed under acidic conditions such as hydrochloric acid in an organic solvent such as methanol, dioxane or ethyl acetate, or trifluoroacetic acid neat or diluted in a solvent such as dichloromethane. The Alloc group is removed in presence of a palladium salt such as palladium acetate or tetrakis(triphenylphosphine)palladium(0) and an allyl cation scavenger such as morpholine, pyrrolidine, dimedone or tributylstannane between 0° C. and 70° C. in a solvent such as tetrahydrofuran. The N-benzyl protected amines are deprotected by hydrogenolysis over a noble metal catalyst (e.g. palladium hydroxide on activated carbon or other suitable catalyst e.g. Raney-Ni). The Fmoc protecting group is removed under mild basic conditions such as diluted morpholine or piperidine in N,N-dimethylformamide or acetonitrile. The N-acetyl protected amines are deprotected by hydrolysis using either acidic or basic aqueous solution at a temperature ranging between 0 and 100° C. Further general methods to remove amine protecting groups have been described in Protective Groups in Organic Synthesis, 5th Edition, by T. W. Greene and P. G. M. Wuts, published by John Wiley & Sons, 2014.

Compounds of formula I-1 are generated by the coupling reaction between a compound of formula IV-1 (for which E2 is hydrogen) and a compound of formula V-1 (for which E3 is —C(O)—Y4 and Y4 is a halogen, or a leaving group such as imidazole, 4-nitrophenol, phenol or 1-hydroxypyrrolidine-2,5-dione).

The reaction can be performed in a variety of organic solvents such as tetrahydofuran, dichloromethane, 1,2-dichloroethane, diethylether, ethyl acetate, dimethylsulfoxide, N,N-dimethylformamide, and acetonitrile, aqueous solvents and a mixture of theses solvents under biphasic conditions (more frequently in N,N-dimethylformamide) in a presence of an inorganic base such as sodium hydride, sodium carbonate or sodium hydrogen carbonate or in the presence of an organic base such as triethylamine, pyridine or a like (more frequently triethylamine). Reactions are typically run from −20° C. to 80° C.

The compounds of formula V-1, for which E3 is —C(O)—Y4 and Y4 is a leaving group such as imidazole (which can be activated by methylation prior to the reaction), 4-nitrophenol, phenol or 1-hydroxypyrrolidine-2,5-dione are typically obtained by the coupling reaction of a compound of formula V-1, for which E3 is a hydrogen and 1,1′-carbonyldiimidazole, 4-nitrophenyl chloroformate, phenyl chloroformate or N,N′-Disuccinimidyl carbonate, respectively, in presence of a base, such as sodium hydride, triethylamine, pyridine (diluted or neat), 4-(dimethylamino)pyridine in aprotic solvents such as dichloromethane, chloroform, acetonitrile, tetrahydrofuran, ethyl acetate. Reactions are typically run from −10° C. to 50° C.

The compounds of formula V-1, for which E3 is —C(O)—Y4 and Y4 is a halogen, are generally prepared in situ by the reaction of a compound of formula V-1, for which E3 is a hydrogen and phosgene or a phosgene precursor (such as bis(trichloromethyl) carbonate or trichloromethyl chloroformate). The reaction is typically performed in aprotic solvents such as dichloromethane, chloroform, acetonitrile, tetrahydrofuran, ethyl acetate in presence of a base such as triethylamine, 4-(dimethylamino)pyridine or N,N-diisopropylethylamine. Reactions are typically run from −40° C. to 50° C. The low stability of such intermediate does not often allow isolation and are generally prepared in situ. The coupling with a compound of formula IV-1 is then performed subsequently following procedures described above.

Alternatively, compounds of formula I-1 can be prepared from the reaction between any compounds of formula IV-1 for which E2 is —C(O)—Y4 and Y4 is a halogen, or a leaving group such as imidazole, 4-nitrophenol or 1-hydroxypyrrolidine-2,5-dione and a compound of formula V-1, for which E3 is a hydrogen using the same procedures previously described above. Compounds of formula IV-1 for which E2 is —C(O)—Y4 and Y4 is a halogen, or a leaving group such as imidazole, 4-nitrophenol or 1-hydroxypyrrolidine-2,5-dione can be prepared from the corresponding compounds of formula IV-1 wherein E2 is hydrogen using methods described above for the preparation of compounds of formula V-1.

In certain cases, compounds of formula I-1 can also be obtained using similar procedures to obtain compounds of formula IV-1 starting from compounds of formula II-1 and compounds of formula III-1 for which E2 is already —C(O)—NH-A instead of E2 is an amino protecting group. Compounds of formula III-1 wherein E2 is a hydrogen, can be converted to compounds of formula III-1 wherein E2 is —C(O)—NH-A by reaction with a compound of formula V-1 wherein Y4 is Y4 is a halogen, or a leaving group such as imidazole, 4-nitrophenol or 1-hydroxypyrrolidine-2,5-dione using similar conditions described above.

Finally, the compounds of formula IV-1 and I-1 for which T is >C═, X is —C(R6a)= (double bond Z, E or Z/E), can further be reduced to generate compounds of formulae IV-2 and I-2, respectively, wherein T is >CH— and X is —C(R6a)(R6b), in which formulae R6a is a C1-C4alkyl and R6b is a hydrogen. The reduction reaction is usually performed by hydrogenation over a noble metal catalyst (e.g. palladium, palladium hydroxide on activated carbon (Chem. Eur. J., 1999, 5, 1055), platinum dioxide) or other suitable catalyst. This hydrogenation step can be performed at any convenient stage during the synthesis.

Using the procedures described above for the preparation of compounds of formula I-1 from compounds of formula IV-1, compounds of formula I-2 can be prepared from compounds of formula IV-2 in similar manner.

In addition, when R2 is a nitro group, compounds of formulae IV-1, IV-2, I-1 or I-2 can be converted to a compound of formulae IV-1, IV-2, I-1 or I-2, respectively, wherein R2 is an amino group, via selective reduction of the aryl-nitro group (Bechamp reduction) using iron powder in the presence of aqueous acidic solution. The nitro group can also be reduced via catalytic hydrogenolysis over a noble metal catalyst (such as palladium on activated carbon) but the reaction leads to compounds of formulae IV-2 or I-2 only.

When R2 is —OCH3, compounds of formulae IV-1, IV-2, I-1 or I-2 can be converted to a compound of formulae IV-1, IV-2, I-1 or I-2, respectively, wherein R2 is a hydroxyl group, via dealkylation of aromatic ether using boron tribromide in an organic solvent such as dichloromethane (J. Am. Chem. Soc., 2002, 12946). The reaction can also be performed using trimethylsilyl bromide or iodide in an organic solvent such as acetonitrile and at a temperature ranging 0° C. to 90° C. Optionally, sodium iodide can be used to help for a good outcome of the reaction.

When R2 is —COOH, or —C1-C6alkylene-COOH, compounds of formulae IV-1, IV-2, I-1 or I-2, can further react with a compound of formula VI, wherein Y5 is —NH₂, >—NH or —NHE, E being an amino protecting group via a peptidic coupling reaction, to generate a compound of formulae IV-1, IV-2, I-1 or I-2, respectively, wherein R2 is —C(O)N(R11a)(R11b), or —C1-C6alkylene-C(O)N(R11a)(R11b). The reaction takes place in the presence of an activating agent such as N,N′-dicyclohexylcarbodiimide or N— (3-dimethylaminopropyl)-N′-ethylcarbodiimide hydrochloride, with the optional addition of 1-hydroxybenzotriazole. Other suitable coupling agents may be utilized such as, O-(7-azabenzotriazol-1-yl)-N,N,N′,N′-tetramethyluronium hexafluorophosphate, 2-ethoxy-1-ethoxycarbonyl-1,2-dihydroquinoline, carbonyldiimidazole or diethylphosphorylcyanide. Optionally, a base like triethylamine, N,N-diisopropylethylamine or pyridine can be added to perform the coupling. The peptidic coupling is conducted at a temperature comprised between −20° C. and 80° C., in an inert solvent, preferably a dry aprotic solvent like dichloromethane, acetonitrile or N,N-dimethylformamide and chloroform. Alternatively, the carboxylic acid can be activated by conversion into its corresponding acid chloride or its corresponding activated ester, such as the N-hydroxysuccinimidyl ester (Org. Process Res. &Dev., 2002, 863) or the benzothiazolyl thioester (J. Antibiotics, 2000, 1071). The generated activated entity can react at a temperature comprised between −20° C. and 80° C. with a compound of formula VI in an aprotic solvent like dichloromethane, chloroform, acetonitrile, N,N-dimethylformamide and tetrahydrofuran to generate a compound of formulae IV-1, IV-2, I-1 or I-2. Optionally, a base like triethylamine, N,N-diisopropylethylamine, pyridine, sodium hydroxide, sodium carbonate, potassium carbonate can be added to perform the coupling.

When R2 is —NH₂ or —C1-C6alkylene-NH₂, compounds of formulae IV-1, IV-2, I-1 or I-2, can further react with a compound of formula VI, wherein Y5 is —CHO via reductive amination reaction to generate compounds of formulae IV-1, IV-2, I-1 or I-2, respectively, wherein R2 is —N(R9a)(R9b) or —C1-C6alkylene-N(R9a)(R9b), R9a and R9b are as defined in formula I. The reductive amination reaction between the amine and the aldehyde to form an intermediate imine is conducted in a solvent system allowing the removal of the formed water through physical or chemical means (e.g. distillation of the solvent-water azeotrope or presence of drying agents such as molecular sieves, magnesium sulfate or sodium sulfate). Such solvent is typically toluene, n-hexane, tetrahydrofuran, dichloromethane N,N-dimethylformamide, N,N-dimethylacetamide, acetonitrile, 1,2-dichloroethane or mixture of solvents such as methanol or 1,2-dichloroethane. The reaction can be catalyzed by traces of acid (usually acetic acid). The intermediate imine is reduced subsequently or simultaneously with a suitable reducing agent (e.g. sodium borohydride, sodium cyanoborohydride, sodium triacetoxyborohydride; R. O. and M. K. Hutchins, Comprehensive Organic Synthesis, B. M. Trost, I. Fleming, Eds; Pergamon Press: New York (1991), vol. 8, p. 25-78) or through hydrogenation over a suitable catalyst such as palladium on activated carbon. The reaction is usually carried out between −10° C. and 110° C., preferably between 0° C. and 60° C. The reaction can also be carried out in one pot. It can also be performed in protic solvents such as methanol or water in presence of a picoline-borane complex (Tetrahedron, 2004, 60, 7899).

In addition, when R2 is —NH₂ or —C1-C6alkylene-NH₂, compounds of formulae IV-1, IV-2, I-1 or I-2, can further react with a compound of formula VI, wherein Y5 is a halogen or a leaving group such as mesylate, tosylate, trifalte, via substitution reaction to generate compounds of formulae IV-1, IV-2, I-1 or I-2, respectively, wherein R2 is —N(R9a)(R9b) or —C1-C6alkylene-N(R9a)(R9b), R9a and R9b are as defined in formula I. The substitution reaction can be performed in presence of an inorganic base such as sodium hydride, potassium carbonate, cesium carbonate or the like or an organic base such as triethylamine or the like in a solvent such as acetonitrile, tetrahydrofuran or N,N-dimethylformamide at a temperature ranging between −20° C. and 100° C.

Finally, when R2 is —NH₂ or —C1-C6alkylene-NH₂, compounds of formulae IV-1, IV-2, I-1 or I-2, can further react with a compound of formula VI, wherein Y5 is —COOH, via peptidic coupling reaction as previously described above to generate compounds of formulae IV-1, IV-2, I-1 or I-2, respectively, wherein R2 is —N(R12)C(O)R13 or —C1-C6alkylene-N(R12)C(O)R13, R12 and R13 are as defined in formula I.

When R2 is —CHO or —C1-C6alkylene-CHO, compounds of formulae IV-1, IV-2, I-1 or I-2, can further react with a compound of formula VI, wherein Y5 is —NH₂ or >NH, via reductive amination reaction as previously described above to generate compounds of formulae IV-1, IV-2, I-1 or I-2, respectively, wherein R2 is —C1-C6alkylene-N(R9a)(R9b), R9a and R9b are as defined in formula I.

In certain cases, compounds of formulae IV-1, IV-2, I-1 or I-2, wherein R2 is —CHO or —C1-C6alkylene-CHO can be generated from the corresponding compounds for which R2 is an ester group or a carboxylic acid function. The ester derivatives are further reduced into their corresponding alcohols. This reduction is performed with a reducing agent like boron or aluminium hydride reducing agent such as lithium aluminium hydride, lithium borohydride, sodium borohydride in a solvent such as tetrahydrofuran, methanol or ethanol between −20° C. and 80° C. Alternatively, the ester function is hydrolyzed into its corresponding carboxylic acid using an alkali hydroxide such as sodium hydroxide, potassium hydroxide or lithium hydroxide in water or in a mixture of water with polar protic or aprotic organic solvents such as dioxane, tetrahydrofuran or methanol between −10° C. and 80° C. The resulting carboxylic acid is further reduced into the corresponding alcohol using a borane derivative such as borane-tetrahydrofuran complex in a solvent such as tetrahydrofuran between −10° C. and 80° C. The generated alcohol is then transformed into its corresponding aldehyde through oxidation under Swern, Dess Martin, Sarett or Corey-Kim conditions respectively, or via NaOCl oxidation. Further methods are described in Comprehensive Organic Transformations. A guide to functional Group Preparations; 2^{nd} Edition, R. C. Larock, Wiley-VC; New York, Chichester, Weinheim, Brisbane, Singapore, Toronto, 1999. Section aldehydes and ketones, p. 1235-1236 and 1238-1246.

When R2 is —C1-C6alkylene-E4, wherein E4 is an hydroxyl group, which needs to be activated prior to the reaction as described below, or a halogen, compounds of formulae IV-1, IV-2, I-1 or I-2, can further react with a compound of formula VI, wherein Y5 is —OH, —NH₂, >NH or —NHE, E being an amino protecting group, via substitution reaction as previously described above to generate compounds of formulae IV-1, IV-2, I-1 or I-2, respectively, wherein R2 is —C1-C6akyl substituted with one to five R14 and at least one R14 is C1-C6alkoxy, or —C1-C6alkylene-N(R9a)(R9b), R9a and R9b are as defined in formula I. Activation of the hydroxyl group of compounds of formulae IV-1, IV-2, I-1 or I-2 wherein R2 is —C1-C6alkylene-OH as for example a mesylate, a tosylate or a triflate can be achieved by reacting the corresponding alcohol with methanesulfonyl chloride or methanesulfonic anhydride, p-toluenesulfonyl chloride, trifluoromethanesulfonyl chloride or trifluoromethanesulfonic anhydride, respectively, in presence of a base such as triethylamine or the like in a dry aprotic solvent such as pyridine, acetonitrile, tetrahydrofuran or dichloromethane between −30° C. and 80° C.

When R2 is —OH, compounds of formulae IV-1, IV-2, I-1 or I-2, can further react with a compound of formula VI, wherein Y5 is a halogen or a leaving group such as mesylate, tosylate or triflate, via substitution reaction as previously described above to generate compounds of formulae IV-1, IV-2, I-1 or I-2, respectively, wherein R2 is C1-C6alkoxy optionally substituted by one to five R14, R14 is as defined for formula I.

When R2 is a halogen or a triflate, compounds of formulae IV-1, IV-2, I-1 or I-2, can further react with a compound of formula VI, wherein Y5 is —NH₂ or >NH, via Buchwald-Hartwig amination reaction to generate compounds of formulae IV-1, IV-2, I-1 or I-2, respectively, wherein R2 is —N(R9a)(R9b), R9a and R9b are as defined in formula I. The Buchwald-Hartwig amination reaction (Chem. Sci., 2011, 2, 27) is a palladium-catalyzed cross-coupling reaction of amines and aryl halides or triflates. Typical catalysts include palladium(II) acetate, or tris(dibenzylideneacetone)dipalladium chloroform complex. The reaction is typically run at a temperature comprised between 0° C. to 150° C. Usually the reaction is performed in the presence of a ligand such as di-tert-butyl-[3,6-dimethoxy-2-(2,4,6-triisopropylphenyl)phenyl]-phosphane, 2-(dicyclohexylphosphino)biphenyl or the like and a base such as sodium tert-butylate, cesium carbonate, potassium carbonate in a large variety of inert solvents such as toluene, tetrahydrofuran, dioxane, 1,2-dichloroethane, N,N-dimethylformamide, dimethylsulfoxide and acetonitrile, aqueous solvents and under biphasic conditions.

Several versions of the reaction employing complexes of copper and nickel rather than palladium have also been developed (Angew. Chem. Int. Ed., 1998, 37, 2046). The reaction can be performed using microwave irradiation.

In addition, when R2 is a halogen or a triflate, compounds of formulae IV-1, IV-2, I-1 or I-2, can further react with a compound of formula VI, wherein Y5 is —OH, via Buchwald-Hartwig type reaction as previously described above to generate compounds of formulae IV-1, IV-2, I-1 or I-2, respectively, wherein R2 is C1-C6alkoxy optionally substituted with one to five R14, R14 is as defined in formula I.

When R4c is an hydroxyl group, which needs to be activated prior to the reaction as described above with a mesylate, tosylate or trifate, or a halogen, compounds of formulae I-1 or I-2, can further react with a compound of formula VI, wherein Y5 is —OH, —CN or >NH, via substitution reaction as previously described above to generate compounds of formulae I-1 or I-2, respectively, wherein R4c is cyano, C1-C4alkoxy or Cycle-P, Cycle-P is as in formula I.

Compounds for which R4c is —OH might be obtained starting from the corresponding esters and using classical conditions of ester reduction as previously described. The hydroxyl group can also be substituted by a halogen atom using classical conditions of halogenation. These reactions can be carried out using halogenated reagents such as carbon tetrabromide, phosphorus tribromide or N-bromosuccinimide, in the presence or not of triphenylphosphine and in an appropriate organic solvent such as tetrahydrofuran, dichloromethane at a temperature ranging between 0° C. and 90° C.

In Scheme 1, the amino protecting groups can be removed at any convenient step of the process.

The compounds of formula I wherein T is >CH— and X is —O— or —S—, can be obtained as summarized in Scheme 2.

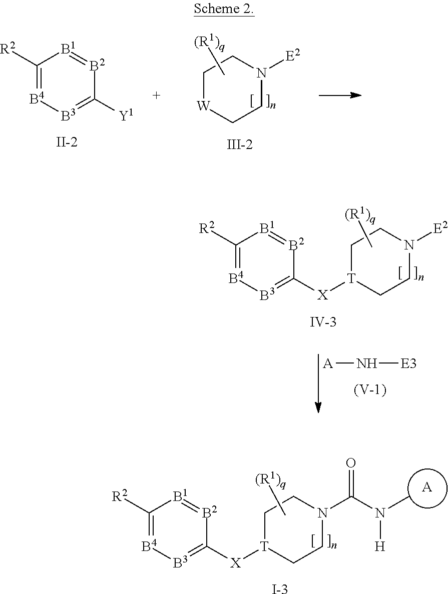

In Scheme 2, all the symbols have the same meanings as previously described in process (b).

Compounds of formula IV-3, wherein X is —O— can be obtained from compounds of formula II-2 wherein Y1 is —OH via a Mitsunobu coupling (as reviewed in O. Mitsunobu, Synthesis, 1981, 1) with compounds of formula III-2 for which Z is a hydroxyl group. The reaction is for example performed in the presence of diethyl or diisopropyl azodicarboxylate and triphenylphosphine, in a wide range of solvents such as N,N-dimethylformamide, tetrahydrofuran, 1,2-dimethoxyethane or dichloromethane and within a wide range of temperatures (between −20° C. and 60° C.). The reaction might also be performed using polymer-supported triphenylphosphine.

An alternative route to form compounds of formula IV-3 wherein X is —O— consists of reacting compounds of formula II-2 wherein Y1 is a hydroxyl group with compounds of formula III-2 for which Z is a hydroxyl group, which needs to be activated prior to the reaction as described above, or a halogen atom by substitution reaction as previously described above.

The same procedure can also be applied to generate compounds of formula IV-3 wherein X is —S— starting from compounds of formula II-2 wherein Y1 is —SH and compounds of formula III-2 wherein Z is a halogen atom or a leaving group such as mesylate, tosylate or triflate.

Further conversion of compounds of formula IV-3 into compounds of formula I-3 is performed following methods described above in Scheme 1 for the preparation of compounds of formula I-1 and I-2.

In Scheme 2, the amino protecting groups can be removed at any convenient step of the process.

The compounds of formula I wherein T is >N— and X is —C(R6a)(R6b), wherein R6a and R6b are as defined for formula I, can be obtained as summarized in Scheme 3.

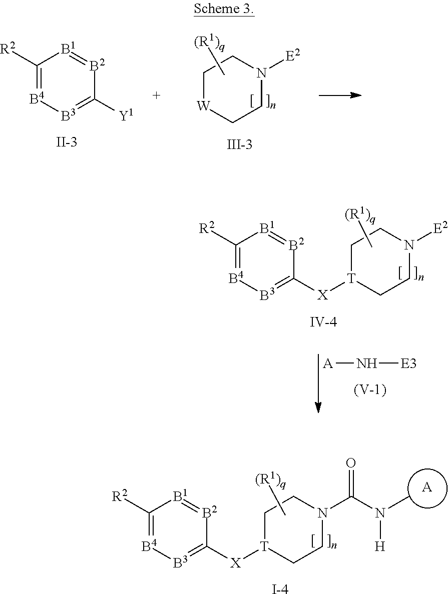

In Scheme 3, all the symbols have the same meanings as previously described in process (c).

Compounds of formula IV-4 wherein X is —C(R6a)(R6b) can be obtained via substitution reaction as described above between a compound of formula III-3 wherein T is >NH and a compound of formula II-3 wherein Z is a halogen atom or a leaving group such as mesylate, tosylate or triflate.

An alternative route to form compounds of formula IV-4 wherein X is —CH(R6a) consists to perform reductive amination as previously described from a compound of formula III-3 wherein T is >NH and a compound of formula II-3 wherein Y1 is —C(O)(R6a).

Further conversion of compounds of formula IV-4 into compounds of formula I-4 is performed following methods described above in Scheme 1 for the preparation of compounds of formula I-1 and I-2.

In Scheme 3, the amino protecting groups can be removed at any convenient step of the process.

The compounds of formula I wherein T is >CH— and X is —C(O)—, can be obtained as summarized in Scheme 4.

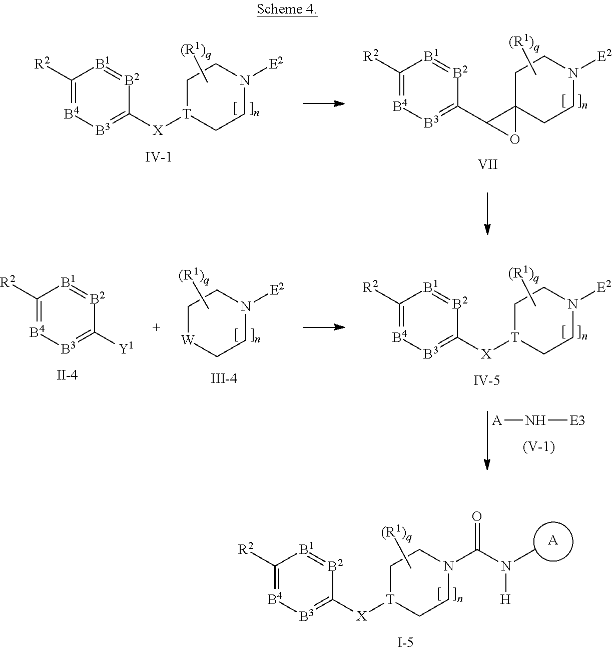

In Scheme 4, all the symbols have the same meanings as previously described in process (c), except for compounds of formula IV-1, for which X is —CH═ and T is >C═.

Compounds of formula IV-5 can be obtained from a compound of formula II-4 wherein Y1 is a hydrogen atom by Friedel-Crafts acylation with a compound of formula III-4 wherein Z is —C(O)C1 and E3 is preferentially N-acetyl group. Friedel-Crafts acylation is the acylation of aromatic rings with an acyl chloride using a strong Lewis acid catalyst such as ferric chloride or aluminium chloride (more frequently aluminium chloride). Friedel-Crafts acylation is also possible with acid anhydrides. Normally, a stoichiometric amount of the Lewis acid catalyst is required, because both the substrate and the product form complexes. The reaction is generally performed under anhydrous conditions in an inert solvent such as acetonitrile, tetrahydrofuran, dichloromethane, 1,2-dichloroethane or in neat mixture at a wide range of temperature (−20° C. to 100° C.).

Alternatively, compound of formula IV-5 can be obtained from a compound of formula II-4 wherein Y1 is —Mg-halogen, via Grignard reaction with a compound of formula III-4 wherein Z is —C(O)E5 and E5 is a leaving group such as —N(CH₃)O(CH₃). The Grignard reaction is typically performed under anhydrous conditions in an organic solvent such as tetrahydrofuran. The reaction are usually run between −78° C. and 60° C. (0° C. preferably). The activation of a compound of formula III-4 is generally obtained from a compound of formula III-4 wherein Z is —COOH and N,O-dimethylhydroxylamine via peptidic coupling reaction as previously described above. Other leaving groups can be used in place of N,O-dimethylhydroxylamine in order to activate the acid function by an ester group using activating ester reagent such as N-hydroxysuccinimidyl, 1-hydroxybenzotriazole or the like. The Grignard reagent is generally obtained from the reaction of an aryl halide and magnesium metal using classical methods widely described in literature (J. Am. Chem. Soc., 1980, 217).

In addition, an alternative route to prepare compounds IV-5 consists to the conversion of a compound of formula VII via epoxide rearrangement. The reaction is typically performed under strong acidic conditions such as neat sulfuric acid in a range of temperature of 0° C. to 100° C. (J. Chem. Soc., Transactions, 1924, 125, 2148). The reaction generally leads at the same time to the deprotection of the amino protecting group such as tert-butoxy carbonyl to generate compounds of formula IV-5, wherein E3 is only hydrogen atom and the subsequent coupling reaction with a compound of formula V-1 can be directly performed using conditions described above to afford the corresponding compounds of formula I-5.

The formation of compound VII is obtained by the oxidation of the olefin bond of a compound of formula IV-1, wherein T is >C═ and X ix —CH═ using classical methods of epoxidation of alkenes in presence of a peroxide reagent such as dihydrogen peroxide, tert-butyl hydroperoxide or meta-chloroperbenzoic acid or the like in a solvent such as dichloromethane, acetonitrile or ethyl acetate at a temperature ranging between −20° C. and 60° C.

Further conversion of compounds of formula IV-5 into compounds of formula I-5 is performed following methods described above in Scheme 1 for the preparation of compounds of formula I-1 and I-2.

In Scheme 4, the amino protecting groups can be removed at any convenient step of the process.

Unless otherwise stated the required starting compounds of formula II, III, V and VI are prepared following or adapting procedures described in the scientific literature.

Whenever required, the substituents R1, R2, R3, R4a, R4aa, R4b and/or R4c can be present as precursors in the starting material, and can be transformed by additional routine transformations during the synthetic pathways described herein.

Whenever an optically active form of a compound of the invention is required, it may be obtained by carrying out one of the above procedures using a pure enantiomer or diastereomer as a starting material, or by resolution of a mixture of the enantiomers or diastereomers of the final product or intermediate using a standard procedure. The resolution of enantiomers may be achieved by chromatography on a chiral stationary phase, such as for example REGIS PIRKLE COVALENT (R-R) WHELK-02, 10 μm, 100 Å, 250×21.1 mm column. Alternatively, resolution of stereoisomers may be obtained by preparation and selective crystallization of a diastereomeric salt of a chiral intermediate or chiral product with a chiral acid, such as camphorsulfonic acid or with a chiral base such as phenylethylamine. Alternatively a method of stereoselective synthesis may be employed, for example by using a chiral variant of a protecting group, a chiral catalyst or a chiral reagent where appropriate in the reaction sequence. Enzymatic techniques may also be used for the preparation of optically active compounds and/or intermediates.

### FIGURES

FIG. 1: FIG. 1 shows the results of the cell growth assays (crystal violet) in HeLa galactose and HeLa glucose cells treated with mitochondrial inhibitors Antimycin A (FIG. 1a) and Example 41 (FIG. 1b) or the cytotoxic drug Paclitaxel (FIG. 1c).

### EXAMPLES

Particular embodiments of the invention are described in the following Examples, which serve to illustrate the invention in more detail.

All reagents and solvents are generally used as received from the commercial supplier;

reactions are routinely performed with anhydrous solvents in well-dried glassware under argon or nitrogen atmosphere;

evaporations are carried out by rotary evaporation under reduced pressure and work-up procedures are carried out after removal of residual solids by filtration;

all temperatures are given in degree Celcius (° C.) and are approximate temperatures; unless otherwise noted, operations are carried out at room temperature, that is typically in the range 18-25° C.;

column chromatography (by the flash procedure) is used to purify compounds and is performed using Merck silica gel 60 (70-230 mesh ASTM) unless otherwise stated;

classical flash chromatography is often replaced by automated systems. This does not change the separation process per se. A person skilled in the art will be able to replace a classical flash chromatography process by an automated one, and vice versa. Typical automated systems can be used, as they are provided by Büchi or Isco (combiflash) for instance;

reaction mixture can often be separated by preparative HPLC. A person skilled in the art will find suitable conditions for each separation;

reactions, which required higher temperature, are usually performed using classical heating instruments;

but can also be performed using microwave apparatus (CEM Explorer) at a power of 250 W, unless otherwise noted;

hydrogenation or hydrogenolysis reactions can be performed using hydrogen gas in balloon or using Parr-apparatus system or other suitable hydrogenation equipment;

concentration of solutions and drying of solids was performed under reduced pressure unless otherwise stated;

in general, the course of reactions is followed by TLC, HPLC, or LC/MS and reaction times are given for illustration only; yields are given for illustration only and are not necessarily the maximum attainable;

the structure of the final products of the invention is generally confirmed by NMR, HPLC and mass spectral techniques.

HPLC of final products are generated using an Agilent 1200 series instrument and the following conditions:

Mobile Phase A: Water+0.1% trifluoroacetic acid

Mobile Phase B: Acetonitrile+0.1% trifluoroacetic acid

Column: SunFire™ C18 (3.5 μm), 150×4.6 mm

Column Temperature: 30° C.

Detection: UV λ=254 nm, 230 nm and 210 nm/DAD

Sample Preparation: 0.4 mg/mL

Injection: 8 μL

Flow: 1.0 mL/min

Gradient:

| Time (min) | % Mobile Phase B |
|---|---|
| 0 | 5 |
| 2 | 5 |
| 5 | 30 |
| 20 | 75 |
| 30 | 95 |
| 35 | 95 |

3 min equilibration linear gradient at 5% Mobile Phase B

Proton NMR spectra are recorded on a Brucker 400 MHz spectrometer. Chemical shifts (δ) are reported in ppm relative to Me₄Si as internal standard, and J values are in Hertz (Hz). Each peak is denoted as a broad singlet (br), singlet (s), doublet (d), triplet (t), quadruplet (q), doublet of doublets (dd), triplet of doublets (td) or multiplet (m). Mass spectra are generated using a q-Tof Ultima (Waters AG or Thermo Scientific MSQ Plus) mass spectrometer in the positive ESI mode. The system is equipped with the standard Lockspray interface;

each intermediate is purified to the standard required for the subsequent stage and is characterized in sufficient detail to confirm that the assigned structure is correct;

analytical and preparative HPLC on non-chiral phases are performed using RP-C18 based columns;

the following abbreviations may be used (reference can also be made to The Journal of Organic Chemistry Guidelines for Authors for a comprehensive list of standard abbreviations):
Boc: tert-butoxy carbonyl group
Cat. no.: Catolog number
CDCl₃: Deuterated chloroform
DMSO-d6: Deuterated dimethyl sulfoxide
D₂O: Deuterated water
ELSD: Evaporative light scattering detection
ESI: Electrospray ionization
Ex: Example
HATU: O-(7-azabenzotriazol-1-yl)-N,N,N′,N′-tetramethyluronium hexafluorophosphate
HPLC: High performance liquid chromatography
J: Coupling constant
LC/MS: Liquid chromatography coupled to mass spectroscopy
Me₄Si: Tetramethylsilane
MCI: Mitsubishi gel with high porous polymer for reverse phase column chromatography
MS: Mass spectroscopy
NMR: Nuclear magnetic resonance
nt: Not Tested
TLC: Thin layer chromatography
v/v: volume ratio
W: Watt
X-Phos: 2-Dicyclohexylphosphino-2′,4′,6′-triisopropylbiphenyl

The following Examples refer to the compounds of formula I as indicated in Table 1.

The Examples listed in the following table can be prepared using procedures described above, and detailed synthesis methodology is described in detail below. The Example numbers used in the leftmost column are used in the application text for identifying the respective compounds.

TABLE 1
Exemplified compounds

| | | | Refer- | | |
|---|---|---|---|---|---|
| | | | ence | | |
| Ex- | | Refer- | for | | |
| am- | | ence | Prepa- | ¹H-NMR | MS m/z |
| ple | Formula | Scheme | ration | (400 MHz) δ ppm | (+ESI) |
| 1 | 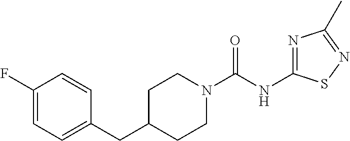 [SMILES: Cc1nsc(NC(=O)N2CCC(Cc3ccc(F)cc3)CC2)n1] | 1 | Ex 27 | Commercially Available [1244911-24-7] | 335.1 [M + H]⁺ |
| 2 | 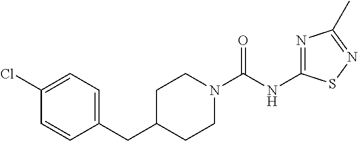 [SMILES: Cc1nsc(NC(=O)N2CCC(Cc3ccc(Cl)cc3)CC2)n1] | 1 | Ex 27 | DMSO-d₆ + D₂O: 7.32 (dd, J1 = 2.4 Hz, = 8.4 Hz, 2H), 7.20 (dd, J1 = 2.4 Hz, J2 = 8.4 Hz, 2H), 4.12 (m, 2H), 2.82 (m, 2H), 2.51 (d, J = 6.8 Hz, 2H), 2.36 (s, 3H), 1.75 (m, 1H), 1.55 (m, 2H), 1.05 (m, 2H) | 351.4, 353.4 [M + H]⁺ |
| 3 | 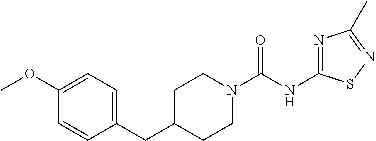 [SMILES: COc1ccc(CC2CCN(C(=O)Nc3nc(C)ns3)CC2)cc1] | 1 | Ex 27 | DMSO-d₆: 11.66 (br, 1H), 7.08 (d, J = 8.4 Hz, 2H), 6.85 (d, J = 8.4 Hz, 2H), 4.15 (m, 2H), 3.72 (s, 3H), 2.78 (m, 2H), 2.45 (d, J = 7.2 Hz, 2H), 2.38 (s, 3H), 1.72 (m, 1H), 1.57 (m, 2H), 1.05 (m, 2H) | 347.1 [M + H]⁺ |
| 4 | 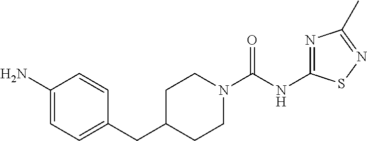 [SMILES: Cc1nsc(NC(=O)N2CCC(Cc3ccc(N)cc3)CC2)n1] | 1 | Ex 95, 27 | DMSO-d₆: 11.61 (br, 1H), 8.50-9.50 (br, 2H), 7.20 (d, J = 8.0 Hz, 2H), 7.10 (d, J = 8.0 Hz, 2H), 4.16 (m, 2H), 2.80 (m, 2H), 2.50 (d, J = 6.8 Hz, 2H), 2.38 (s, 3H), 1.75 (m, 1H), 1.55 (m, 2H), 1.10 (m, 2H) | 332.4 [M + H]⁺ |
| 5 | 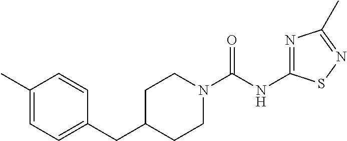 [SMILES: Cc1ccc(CC2CCN(C(=O)Nc3nc(C)ns3)CC2)cc1] | 1 | Ex 27 | DMSO-d₆: 11.57 (s, 1H), 7.06 (m, 4H), 4.15 (m, 2H), 2.81 (m, 2H), 2.50 (m, 2H), 2.46 (s, 3H), 2.33 (s, 3H), 1.74 (m, 1H), 1.70 (m, 2H), 1.16 (m, 2H) | 331.0 [M + H]⁺ |
| 6 | 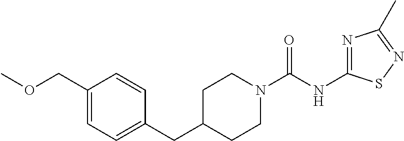 [SMILES: COCc1ccc(CC2CCN(C(=O)Nc3nc(C)ns3)CC2)cc1] | 1 | Ex 27 | DMSO-d₆: 11.57 (s, 1H), 7.23 (m, 2H), 7.15 (m, 2H), 4.37 (s, 2H), 4.15 (m, 2H), 3.27 (s, 3H), 2.80 (m, 2H), 2.51 (m, 2H), 2.38 (s, 3H), 1.76 (m, 1H), 1.57 (m, 2H), 1.13 (m, 2H) | 361.1 [M + H]⁺ |
| 7 | 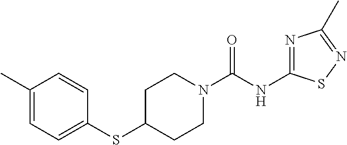 [SMILES: Cc1ccc(SC2CCN(C(=O)Nc3nc(C)ns3)CC2)cc1] | 2 | Ex 27 | DMSO-d₆: 11.62 (s, 1H), 7.33 (m, 2H), 7.18 (m, 2H), 4.05 (m, 2H), 3.41 (m, 1H), 3.10 (m, 2H), 2.33 (s, 3H) 2.29 (s, 3H), 1.92 (m, 2H), 1.44 (m, 2H) | 349.0 [M + H]⁺ |
| 8 | 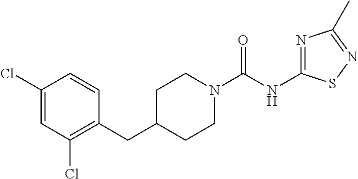 [SMILES: Cc1nsc(NC(=O)N2CCC(Cc3ccc(Cl)cc3Cl)CC2)n1] | 1 | Ex 27 | DMSO-d₆: 11.60 (s, 1H), 7.58 (d, J = 1.6 Hz, 1H), 7.33-7.39 (m, 2H), 4.16 (d, J = 13.2 Hz, 2H), 2.82 (t, J = 13.2 Hz, 2H), 2.65 (d, J = 7.2 Hz, 2H), 2.38 (s, 3H), 1.82 (m, 1H), 1.55-1.58 (m, 2H), 1.16-1.22 (m, 2H) | 385.1, 387.1 [M + H]⁺ |
| 9 | 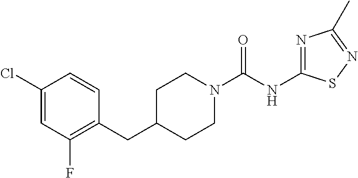 [SMILES: Cc1nsc(NC(=O)N2CCC(Cc3ccc(Cl)cc3F)CC2)n1] | 1 | Ex 27 | DMSO-d₆: 11.59 (s, 1H), 7.37 (dd, J1 = 10.0 Hz, J2 = 2.0 Hz, 1H), 7.32 (t, J = 8.4 Hz, 1H), 7.23 (dd, J1 = 8.4 Hz, J2 = 2.0 Hz, 1H), 4.16 (d, J = 12.8 Hz, 2H), 2.82 (t, J = 12.8 Hz, 2H), 2.56 (d, J = 7.2 Hz, 2H), 2.38 (s, 3H), 1.75 (m, 1H), 1.56-1.60 (m, 2H), 1.08-1.17 (m, 2H) | 369.0, 371.0 [M + H]⁺ |
| 10 | 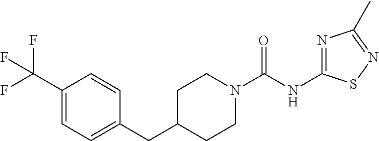 [SMILES: Cc1nsc(NC(=O)N2CCC(Cc3ccc(C(F)(F)F)cc3)CC2)n1] | 1 | Ex 27 | DMSO-d₆: 11.59 (s, 1H), 7.65 (d, J = 8.0 Hz, 2H), 7.42 (d, J = 8.0 Hz, 2H), 4.16 (d, J = 13.2 Hz, 2H), 2.82 (t, J = 13.2 Hz, 2H), 2.63 (d, J = 7.2 Hz, 2H), 2.38 (s, 3H), 1.81 (m, 1H), 1.58 (m, 2H), 1.13-1.17 (m, 2H) | 385.1 [M + H]⁺ |
| 11 | 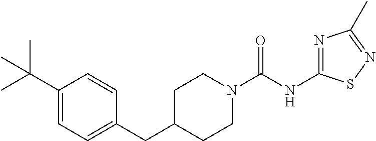 [SMILES: Cc1nsc(NC(=O)N2CCC(Cc3ccc(C(C)(C)C)cc3)CC2)n1] | 1 | Ex 27 | DMSO-d₆: 11.58 (s, 1H), 7.30 (d, J = 8.0 Hz, 2H), 7.09 (d, J = 8.0 Hz, 2H), 4.15 (d, J = 13.2 Hz, 2H), 2.81 (t, J = 13.2 Hz, 2H), 2.48 (d, J = 6.8 Hz, 2H), 2.38 (s, 3H), 1.74 (m, 1H), 1.58-1.61 (m, 2H), 1.27 (s, 9H), 1.10-1.14 (m, 2H) | 373.1 [M + H]⁺ |
| 12 | 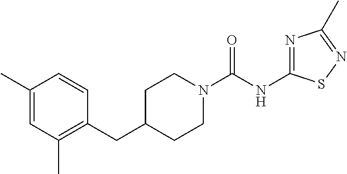 [SMILES: Cc1ccc(CC2CCN(C(=O)Nc3nc(C)ns3)CC2)c(C)c1] | 1 | Ex 27 | DMSO-d₆: 11.58 (s, 1H), 6.97 (m, 2H), 6.91 (d, J = 8.0 Hz, 2H,), 4.16 (d, J = 13.2 Hz, 2H), 2.80 (t, J = 12.4 Hz, 2H), 2.47 (d, J = 7.2 Hz, 2H), 2.38 (s, 3H), 2.22 (s, 6H), 1.70 (m, 1H), 1.57-1.61 (m, 2H), 1.15-1.18 (m, 2H) | 345.1 [M + H]⁺ |
| 13 | 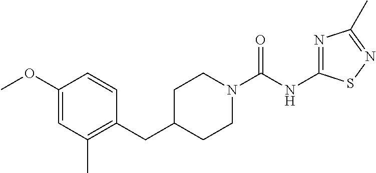 [SMILES: COc1ccc(CC2CCN(C(=O)Nc3nc(C)ns3)CC2)c(C)c1] | 1 | Ex 14, 27 | DMSO-d₆ + D₂O: 6.98 (d, J = 8.4 Hz, 1H), 6.64-6.71 (m, 2H), 4.11 (d, J = 13.2 Hz, 2H), 3.68 (s, 3H), 2.75-2.81 (m, 2H), 2.42 (d, J = 7.2 Hz, 2H), 2.35 (s, 3H), 2.21 (s, 3H), 1.66 (m, 1H), 1.56-1.59 (m, 2H), 1.02-1.14 (m, 2H) | 361.2 [M + H]⁺ |
| 14 | 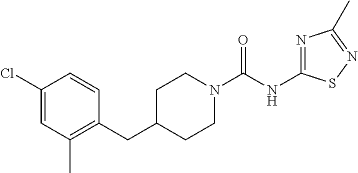 [SMILES: Cc1nsc(NC(=O)N2CCC(Cc3ccc(Cl)cc3C)CC2)n1] | 1 | Ex 27 | DMSO-d₆ + D₂O: 7.20 (d, J = 2.0 Hz, 1H), 7.14 (dd, J1 = 8.4 Hz, J2 = 2.0 Hz, 1H), 7.10 (d, J = 8.4 Hz, 1H), 4.13 (d, J = 13.6 Hz, 2H), 2.75-2.81 (m, 2H), 2.47-2.51 (m, 2H), 2.35 (s, 3H), 2.24 (s, 3H), 1.68-1.72 (m, 1H), 1.56 (d, J = 11.2 Hz, 2H), 1.07-1.16 (m, 2H) | 365.2, 367.2 [M + H]⁺ |
| 15 | 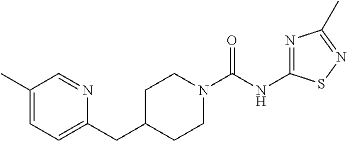 [SMILES: Cc1ccc(CC2CCN(C(=O)Nc3nc(C)ns3)CC2)nc1] | 1 | Ex 27 | DMSO-d₆ + D₂O: 8.29 (d, J = 2.0 Hz, 1H), 7.51 (dd, J1 = 8.0 Hz, J2 = 1.6 Hz, 1H), 7.12 (d, J = 8.0 Hz, 1H), 4.11 (d, J = 13.2 Hz, 2H), 2.79-2.85 (m, 2H), 2.50-2.61 (m, 2H), 2.36 (s, 3H), 2.24 (s, 3H), 1.92-1.97 (m, 1H), 1.56 (d, J = 10.8 Hz, 2H), 1.07-1.17 (m, 2H) | 332.2 [M + H]⁺ |
| 16 | 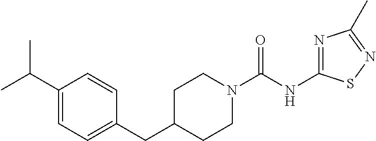 [SMILES: Cc1nsc(NC(=O)N2CCC(Cc3ccc(C(C)C)cc3)CC2)n1] | 1 | Ex 27 | DMSO-d₆: 11.56 (br, 1H), 7.15 (d, J = 8.0 Hz, 2H), 7.08 (d, J = 8.0 Hz, 2H), 4.15 (d, J = 12.8 Hz, 2H), 2.84 (m, 3H), 2.47 (d, J = 7.2 Hz, 2H), 2.37 (s, 3H), 1.72 (m, 1H), 1.59 (m, 2H), 1.18 (d, J = 7.2 Hz, 6H), 1.07 (m, 2H) | 359.2 [M + H]⁺ |
| 17 | 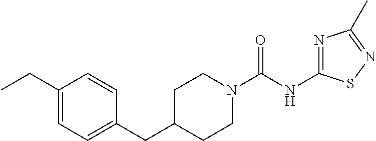 [SMILES: CCc1ccc(CC2CCN(C(=O)Nc3nc(C)ns3)CC2)cc1] | 1 | Ex 27 | DMSO-d₆: 11.56 (br, 1H), 7.12 (d, J = 8.0 Hz, 2H), 7.07 (d, J = 8.0 Hz, 2H), 4.15 (d, J = 13.2 Hz, 2H), 2.81 (t, J = 12.0 Hz, 2H), 2.56 (q, J = 7.6 Hz, 2H), 2.48 (d, J = 7.2 Hz, 2H), 2.38 (s, 3H), 1.72 (m, 1H), 1.59 (m, 2H), 1.16 (t, J = 7.6 Hz, 3H), 1.07 (m, 2H) | 345.1 [M + H]⁺ |
| 18 | 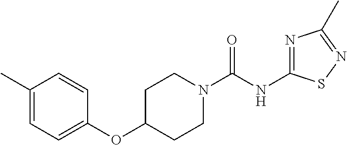 [SMILES: Cc1ccc(OC2CCN(C(=O)Nc3nc(C)ns3)CC2)cc1] | 2 | Ex 19 | DMSO-d₆: 11.68 (br, 1H), 7.08 (d, J = 8.0 Hz, 2H), 6.88 (m, 2H), 4.55 (m, 1H), 3.84 (m, 2H), 3.41 (m, 2H), 2.38 (s, 3H), 2.23 (s, 3H), 1.94 (m, 2H), 1.58 (m, 2H) | 333.1 [M + H]⁺ |
| 19 | 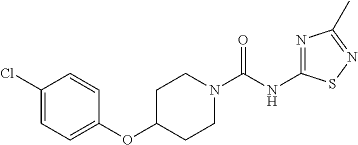 [SMILES: Cc1nsc(NC(=O)N2CCC(Oc3ccc(Cl)cc3)CC2)n1] | 2 | Ex 27 | DMSO-d₆: 11.75 (br, 1H), 7.32 (m, 2H), 7.02 (m, 2H), 4.61 (m, 1H), 3.87 (m, 2H), 3.38 (m, 2H), 2.36 (s, 3H), 1.95 (m, 2H), 1.58 (m, 2H) | 353.0, 355.0 [M + H]⁺ |
| 20 | 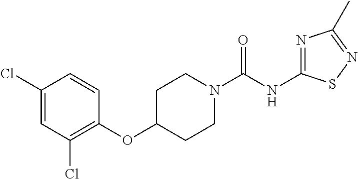 [SMILES: Cc1nsc(NC(=O)N2CCC(Oc3ccc(Cl)cc3Cl)CC2)n1] | 2 | Ex 19 | DMSO-d₆: 11.67 (br, 1H), 7.59 (d, J = 2.8 Hz, 1H), 7.37 (dd, J1 = 8.8 Hz, J2 = 2.4 Hz, 1H), 7.30 (d, J = 9.2 Hz, 1H), 4.75 (m, 1H), 3.75 (m, 2H), 3.53 (m, 2H), 2.39 (s, 3H), 1.95 (m, 2H), 1.66 (m, 2H) | 387.0, 389.0 [M + H]⁺ |
| 21 | 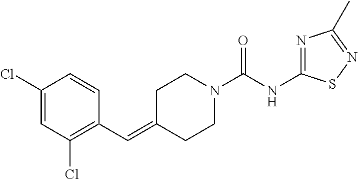 [SMILES: Cc1nsc(NC(=O)N2CCC(=Cc3ccc(Cl)cc3Cl)CC2)n1] | 1 | Ex 95 | DMSO-d₆: 11.74 (s, 1H), 7.66 (d, J = 2.1 Hz, 1H), 7.43 (dd, J1 = 8.3, J = 2.1 Hz, 1H), 7.35 (d, J = 8.3 Hz, 1H), 6.35 (s, 1H), 3.66 (m, 2H), 3.57 (m, 2H), 2.41 (m, 5H), 2.30 (m, 2H) | 383.0, 385.0 [M + H]⁺ |
| 22 | 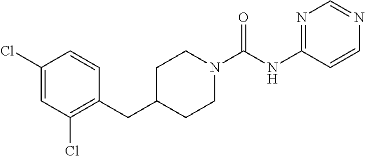 [SMILES: O=C(Nc1ccncn1)N1CCC(Cc2ccc(Cl)cc2Cl)CC1] | 1 | Ex 8, 27 | DMSO-d₆: 10.01 (s, 1H), 8.82 (s, 1H), 8.52 (m, 1H), 7.80 (m, 1H), 7.60 (s, 1H), 7.38 (m, 2H), 4.12 (m, 2H), 2.78 (m, 2H), 2.66 (d, 2H, J = 8 Hz), 1.81 (m, 1H), 1.57 (m, 2H), 1.21 (m, 2H) | 365.1, 367.0 [M + H]⁺ |
| 23 | 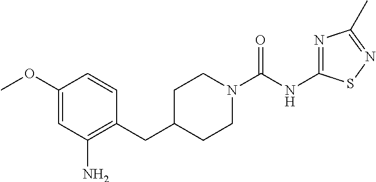 [SMILES: COc1ccc(CC2CCN(C(=O)Nc3nc(C)ns3)CC2)c(N)c1] | 1 | Ex 95, 4, 27 | DMSO-d₆: 11.56 (br, 1H), 6.74 (d, J = 8.0 Hz, 1H), 6.19 (d, J = 2.8 Hz, 1H), 6.06 (dd, J1 = 8.4 Hz, J2 = 2.8 Hz, 1H), 4.86 (s, 2H), 4.15 (d, J = 12.8 Hz, 2H), 3.63 (s, 3H), 2.79 (t, J = 12.4 Hz, 2H), 2.38 (s, 3H), 2.31 (d, J = 6.8 Hz, 2H), 1.71 (m, 1H), 1.62 (d, J = 12.8 Hz, 2H), 1.09 (m, 2H) | 362.3 [M + H]⁺ |
| 24 | 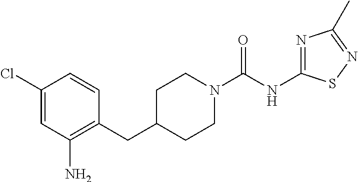 [SMILES: Cc1nsc(NC(=O)N2CCC(Cc3ccc(Cl)cc3N)CC2)n1] | 1 | Ex 95, 4, 27 | DMSO-d₆ + D₂O: 6.93 (d, J = 8.4 Hz, 1H), 6.74 (d, J = 2.4 Hz, 1H), 6.62 (dd, J1 = 8.0 Hz, J2 = 2.4 Hz, 1H), 4.12 (d, J = 13.6 Hz, 2H), 2.78 (t, J = 12.8 Hz, 2H), 2.37 (m, 5H), 1.76 (m, 1H), 1.59 (d, J = 10.8 Hz, 2H), 1.08 (m, 2H) | 366.0, 368.0 [M + H]⁺ |
| 25 | 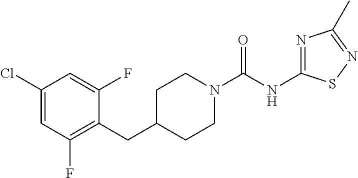 [SMILES: Cc1nsc(NC(=O)N2CCC(Cc3c(F)cc(Cl)cc3F)CC2)n1] | 1 | Ex 27 | DMSO-d₆ + D₂O: 7.26 (m, 2H), 4.10 (d, J = 13.6 Hz, 2H), 2.80 (t, J = 12.4 Hz, 2H), 2.53 (d, J = 7.2 Hz, 2H), 2.35 (s, 3H), 1.75 (m, 1H), 1.57 (d, J = 11.6 Hz, 2H), 1.08 (m, 2H) | 387.0, 389.0 [M + H]⁺ |
| 26 | 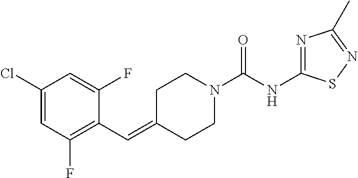 [SMILES: Cc1nsc(NC(=O)N2CCC(=Cc3c(F)cc(Cl)cc3F)CC2)n1] | 1 | Ex 95 | DMSO-d₆ + D₂O: 7.32 (d, J = 7.2 Hz, 2H), 6.01 (s, 1H), 3.60 (t, J = 5.6 Hz, 2H), 3.50 (t, J = 5.6 Hz, 2H), 2.39 (t, J = 5.6 Hz, 2H), 2.36 (s, 3H), 2.07 (m, 2H) | 385.1, 387.0 [M + H]⁺ |
| 27 | 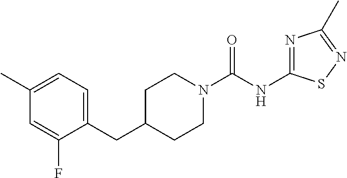 [SMILES: Cc1ccc(CC2CCN(C(=O)Nc3nc(C)ns3)CC2)c(F)c1] | 1 | — | DMSO-d₆ + D₂O: 7.10 (dd, J1 = J2 = 7.6 Hz, 1H), 6.90-6.96 (m, 2H), 4.05-4.14 (m, 2H), 2.73-2.85 (m, 2H), 2.48 (d, J = 6.8 Hz, 2H), 2.35 (s, 3H), 2.25 (s, 3H), 1.67-1.79 (m, 1H), 1.52-1.60 (m, 2H), 1.00-1.13 (m, 2H). | 349.1 [M + H]⁺ |
| 28 | 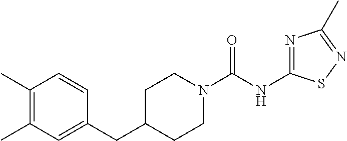 [SMILES: Cc1nsc(NC(=O)N2CCC(Cc3ccc(C)c(C)c3)CC2)n1] | 1 | Ex 27 | DMSO-d₆ + D₂O: 7.02 (d, J = 7.6 Hz, 1H), 6.91 (s, 1H), 6.86 (d, J = 7.6 Hz, 1H), 4.05-4.15 (m, 2H), 2.73-2.86 (m, 2H), 2.42 (d, J = 7.2 Hz, 2H), 2.36 (s, 3H), 2.16 (s, 3H), 2.15 (s, 3H), 1.63-1.78 (m, 1H), 1.52-1.61 (m, 2H), 0.98-1.11 (m, 2H) | 345.2 [M + H]⁺ |
| 29 | 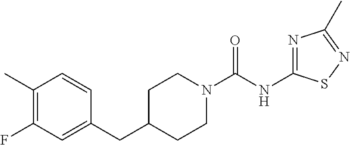 [SMILES: Cc1nsc(NC(=O)N2CCC(Cc3ccc(C)c(F)c3)CC2)n1] | 1 | Ex 27 | DMSO-d₆ + D₂O: 7.14 (dd, J1 = J2 = 8.0 Hz, 1H), 6.91 (d, J = 10.0 Hz, 1H), 6.87 (d, J = 8.0 Hz, 1H), 4.04-4.13 (m, 2H), 2.73-2.84 (m, 2H), 2.46 (d, J = 7.2 Hz, 2H), 2.34 (s, 3H), 2.15 (s, 3H), 1.65-1.78 (m, 1H), 1.50-1.60 (m, 2H), 0.96-1.10 (m, 2H) | 349.1 [M + H]⁺ |
| 30 | 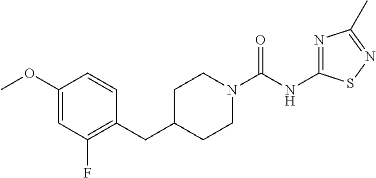 [SMILES: COc1ccc(CC2CCN(C(=O)Nc3nc(C)ns3)CC2)c(F)c1] | 1 | Ex 27, 28 | DMSO-d₆ + D₂O: 7.13 (dd, J1 = J2 = 8.4 Hz, 1H), 6.66-6.75 (m, 2H), 4.04-4.15 (m, 2H), 3.70 (s, 3H), 2.73-2.86 (m, 2H), 2.45 (d, J = 6.8 Hz, 2H), 2.34 (s, 3H), 1.62-1.75 (m, 1H), 1.51-1.60 (m, 2H), 0.99-1.11 (m, 2H) | 365.1 [M + H]⁺ |
| 31 | 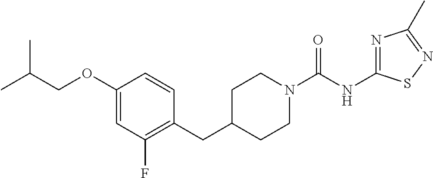 [SMILES: Cc1nsc(NC(=O)N2CCC(Cc3ccc(OCC(C)C)cc3F)CC2)n1] | 1 | Ex 27 | DMSO-d₆ + D₂O: 7.12 (dd, J1 = J2 = 8.8 Hz, 1H), 6.66-6.75 (m, 2H), 4.06-4.16 (m, 2H), 3.69 (d, J = 6.8 Hz, 2H), 2.75-2.87 (m, 2H), 2.46 (d, J = 7.2 Hz, 2H), 2.36 (s, 3H), 1.92-2.02 (m, 1H), 1.64-1.77 (m, 1H), 1.53-1.61 (m, 2H), 1.00-1.12 (m, 2H), 0.94 (d, J = 6.8 Hz, 6H) | 407.2 [M + H]⁺ |
| 32 | 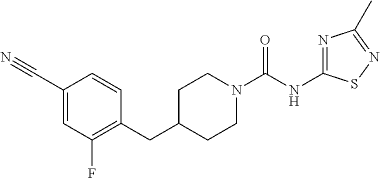 [SMILES: Cc1nsc(NC(=O)N2CCC(Cc3ccc(C#N)cc3F)CC2)n1] | 1 | Ex 27 | DMSO-d₆ + D₂O: 7.75 (dd, J1 = 1.6 Hz, J2 = 9.6 Hz, 1H), 7.62 (dd, J1 = 1.6 Hz, J2 = 8.0 Hz, 1H), 7.49 (dd, J1 = J2 = 8.0 Hz, 1H), 4.08-4.17 (m, 2H), 2.75-2.85 (m, 2H), 2.63 (d, J = 6.8 Hz, 2H), 2.36 (s, 3H), 1.75-1.88 (m, 1H), 1.52-1.60 (m, 2H), 1.06-1.18 (m, 2H) | 360.1 [M + H]⁺ |
| 33 | 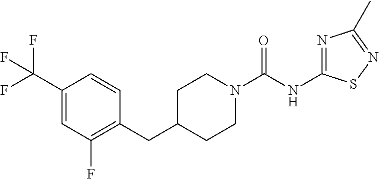 [SMILES: Cc1nsc(NC(=O)N2CCC(Cc3ccc(C(F)(F)F)cc3F)CC2)n1] | 1 | Ex 27 | DMSO-d₆ + D₂O: 7.58 (d, J = 10.4 Hz, 1H), 7.51 (m, 2H), 4.08-4.17 (m, 2H), 2.74-2.87 (m, 2H), 2.63 (d, J = 7.2 Hz, 2H), 2.35 (s, 3H), 1.75-1.88 (m, 1H), 1.52-1.62 (m, 2H), 1.06-1.18 (m, 2H) | 403.1 [M + H]⁺ |
| 34 | 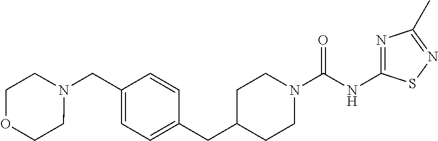 [SMILES: Cc1nsc(NC(=O)N2CCC(Cc3ccc(CN4CCOCC4)cc3)CC2)n1] | 1 | Ex 27, 28 | DMSO-d₆ + D₂O: 7.39 (d, J = 8.0 Hz, 2H), 7.27 (d, J = 8.0 Hz, 2H), 4.27 (s, 2H), 4.06-4.15 (m, 2H), 3.90-4.00 (m, 2H), 3.53-3.67 (m, 2H), 3.16-3.26 (m, 2H), 3.03-3.16 (m, 2H), 2.75-2.89 (m, 2H), 2.54 (d, J = 7.2 Hz, 2H), 2.36 (s, 3H), 1.73-1.85 (m, 1H), 1.53-1.63 (m, 2H), 1.01-1.15 (m, 2H) | 416.2 [M + H]⁺ |
| 35 | 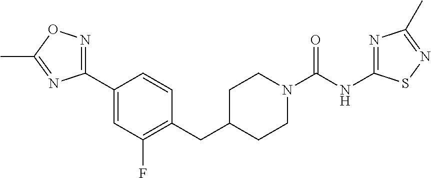 [SMILES: Cc1nsc(NC(=O)N2CCC(Cc3ccc(-c4noc(C)n4)cc3F)CC2)n1] | 1 | Ex 27, 32 | DMSO-d₆ + D₂O: 7.76 (dd, J1 = 1.6 Hz, J2 = 7.6 Hz, 1H), 7.67 (dd, J1 = 1.6 Hz, J2 = 10.4 Hz, 1H), 7.47 (dd, J1 = J2 = 7.6 Hz, 1H), 4.10-4.18 (m, 2H), 2.77-2.89 (m, 2H), 2.64 (s, 3H), 2.62 (d, J = 6.8 Hz, 2H), 2.36 (s, 3H), 1.77-1.89 (m, 1H), 1.55-1.63 (m, 2H), 1.07-1.20 (m, 2H) | 417.1 [M + H]⁺ |
| 36 | 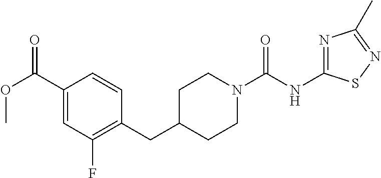 [SMILES: COC(=O)c1ccc(CC2CCN(C(=O)Nc3nc(C)ns3)CC2)c(F)c1] | 1 | Ex 27 | DMSO-d₆ + D₂O: 7.71 (dd, J1 = 2.0 Hz, J2 = 7.6 Hz, 1H), 7.63 (dd, J1 = 2.0 Hz, J2 = 10.4 Hz, 1H), 7.42 (dd, J1 = J2 = 7.6 Hz, 1H), 4.07-4.17 (m, 2H), 3.82 (s, 3H), 2.73-2.87 (m, 2H), 2.61 (d, J = 7.2 Hz, 2H), 2.34 (s, 3H), 1.73-1.86 (m, 1H), 1.52-1.61 (m, 2H), 1.04-1.17 (m, 2H) | 393.1 [M + H]⁺ |
| 37 | 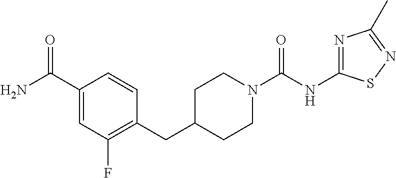 [SMILES: Cc1nsc(NC(=O)N2CCC(Cc3ccc(C(N)=O)cc3F)CC2)n1] | 1 | Ex 36 | DMSO-d₆ + D₂O: 7.55-7.64 (m, 2H), 7.34 (dd, J1 = J2 = 8.0 Hz, 1H), 4.05-4.14 (m, 2H), 2.72-2.88 (m, 2H), 2.58 (d, J = 6.8 Hz, 2H), 2.34 (s, 3H), 1.73-1.85 (m, 1H), 1.53-1.61 (m, 2H), 1.03-1.16 (m, 2H) | 378.0 [M + H]⁺ |
| 38 | 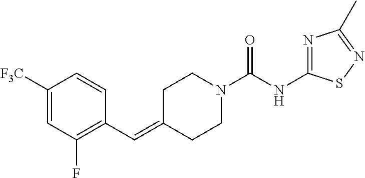 [SMILES: Cc1nsc(NC(=O)N2CCC(=Cc3ccc(C(F)(F)F)cc3F)CC2)n1] | 1 | Ex 95 | DMSO-d₆ + D₂O: 7.63 (d, J = 10.0 Hz, 1H), 7.48-7.58 (m, 2H), 6.36 (s, 1H), 3.63 (t, J = 5.6 Hz, 2H), 3.54 (t, J = 5.6 Hz, 2H), 2.42 (t, J = 5.6 Hz, 2H), 2.37 (s, 3H), 2.32 (t, J = 5.6 Hz, 2H) | 401.1 [M + H]⁺ |
| 39 | 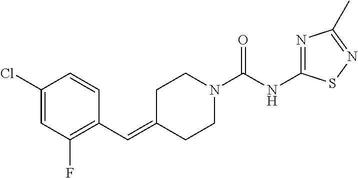 [SMILES: Cc1nsc(NC(=O)N2CCC(=Cc3ccc(Cl)cc3F)CC2)n1] | 1 | Ex 95 | DMSO-d₆ + D₂O: 7.41 (dd, J1 = 2.0 Hz, J2 = 10.0 Hz, 1H), 7.31 (dd, J1 = J2 = 8.4 Hz, 1H), 7.27 (dd, J1 = 2.0 Hz, J2 = 8.4 Hz, 1H), 6.27 (s, 1H), 3.62 (t, J = 5.6 Hz, 2H), 3.53 (t, J = 5.6 Hz, 2H), 2.40 (t, J = 5.6 Hz, 2H), 2.37 (s, 3H), 2.29 (t, J = 5.6 Hz, 2H) | 367.1, 369.0 [M + H]⁺ |
| 40 | 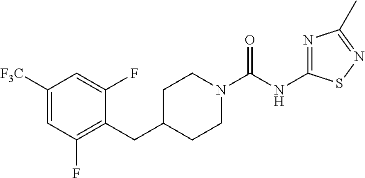 [SMILES: Cc1nsc(NC(=O)N2CCC(Cc3c(F)cc(C(F)(F)F)cc3F)CC2)n1] | 1 | Ex 88, 27 | DMSO-d₆ + D₂O: 7.52-7.62 (m, 2H), 4.08-4.18 (m, 2H), 2.86-2.90 (m, 2H), 2.64 (d, J = 7.2 Hz, 2H), 2.36 (s, 3H), 1.73-1.86 (m, 1H), 1.55-1.65 (m, 2H), 1.10-1.24 (m, 2H) | 421.1 [M + H]⁺ |
| 41 | 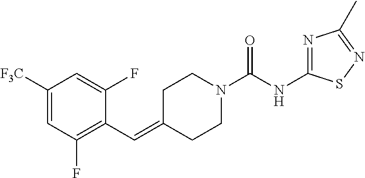 [SMILES: Cc1nsc(NC(=O)N2CCC(=Cc3c(F)cc(C(F)(F)F)cc3F)CC2)n1] | 1 | Ex 88 | DMSO-d₆ + D₂O: 7.56-7.64 (m, 2H), 6.11 (s, 1H), 3.63 (t, J = 5.6 Hz, 2H), 3.52 (t, J = 5.6 Hz, 2H), 2.43 (t, J = 5.6 Hz, 2H), 2.37 (s, 3H), 2.11 (t, J = 5.6 Hz, 2H) | 419.1 [M + H]⁺ |
| 42 | 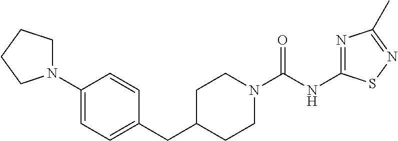 [SMILES: Cc1nsc(NC(=O)N2CCC(Cc3ccc(N4CCCC4)cc3)CC2)n1] | 1 | Ex 27, 45 | DMSO-d₆ + D₂O: 7.03 (d, J = 8.4 Hz, 2H), 6.66 (d, J = 8.4 Hz, 2H), 4.08-4.17 (m, 2H), 3.22-3.30 (m, 4H), 2.75-2.85 (m, 2H), 2.42 (d, J = 6.8 Hz, 2H), 2.36 (s, 3H), 1.90-2.00 (m, 4H), 1.62-1.73 (m, 1H), 1.53-1.62 (m, 2H), 0.99-1.11 (m, 2H) | 386.2 [M + H]⁺ |
| 43 | 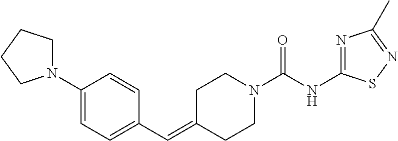 [SMILES: Cc1nsc(NC(=O)N2CCC(=Cc3ccc(N4CCCC4)cc3)CC2)n1] | 1 | Ex 27, 45 | DMSO-d₆ + D₂O: 7.04 (d, J = 8.4 Hz, 2H), 6.52 (d, J = 8.4 Hz, 2H), 6.24 (s, 1H), 3.40-3.60 (m, 4H), 3.20 (m, 4H), 2.43 (m, 2H), 2.35 (s, 3H), 2.29 (m, 2H), 1.90 (m, 4H) | 384.2 [M + H]⁺ |
| 44 | 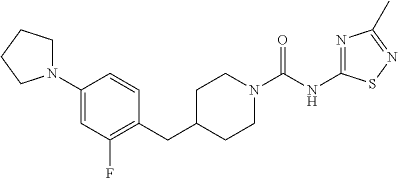 [SMILES: Cc1nsc(NC(=O)N2CCC(Cc3ccc(N4CCCC4)cc3F)CC2)n1] | 1 | Ex 27, 45 | DMSO-d₆ + D₂O: 6.98 (dd, J1 = J2 = 8.4 Hz, 1H), 6.20-6.32 (m, 2H), 4.08-4.16 (m, 2H), 3.12-3.23 (m, 4H), 2.74-2.86 (m, 2H), 2.41 (d, J = 7.2 Hz, 2H), 2.36 (s, 3H), 1.86-1.97 (m, 4H), 1.62-1.73 (m, 1H), 1.55-1.62 (m, 2H), 0.99-1.11 (m, 2H) | 404.2 [M + H]⁺ |
| 45 | 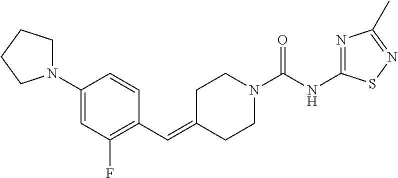 [SMILES: Cc1nsc(NC(=O)N2CCC(=Cc3ccc(N4CCCC4)cc3F)CC2)n1] | 1 | Ex 27 | DMSO-d₆ + D₂O: 7.02 (dd, J1 = J2 = 8.4 Hz, 1H), 6.22-6.35 (m, 2H), 6.16 (s, 1H), 3.56 (m, 2H), 3.48 (m, 2H), 3.10-3.20 (m, 4H), 2.35 (s, 3H), 2.25-2.34 (m, 4H), 1.83-1.96 (m, 4H) | 402.2 [M + H]⁺ |
| 46 | 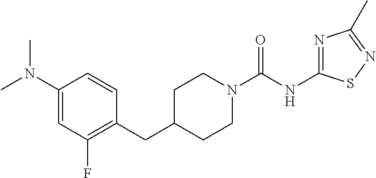 [SMILES: Cc1nsc(NC(=O)N2CCC(Cc3ccc(N(C)C)cc3F)CC2)n1] | 1 | Ex 27, 45, 47 | DMSO-d₆ + D₂O: 7.06 (dd, J1 = J2 = 8.4 Hz, 1H), 6.54-6.62 (m, 2H), 4.07-4.17 (m, 2H), 2.88 (s, 6H), 2.75-2.83 (m, 2H), 2.43 (d, J = 6.8 Hz, 2H), 2.36 (s, 3H), 1.63-1.76 (m, 1H), 1.53-1.62 (m, 2H), 1.00-1.12 (m, 2H) | 378.2 [M + H]⁺ |
| 47 | 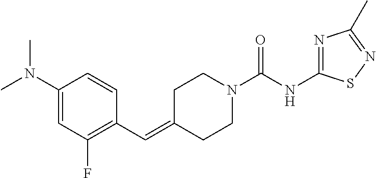 [SMILES: Cc1nsc(NC(=O)N2CCC(=Cc3ccc(N(C)C)cc3F)CC2)n1] | 1 | Ex 27, 45 | DMSO-d₆ + D₂O: 7.08 (dd, J1 = J2 = 8.4 Hz, 1H), 6.47-6.56 (m, 2H), 6.20 (s, 1H), 3.60 (t, J = 5.6 Hz, 2H), 3.52 (t, J = 5.6 Hz, 2H), 2.89 (s, 6H), 2.38 (s, 3H), 2.28-2.37 (m, 4H) | 376.2 [M + H]⁺ |
| 48 | 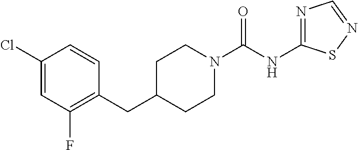 [SMILES: O=C(Nc1ncns1)N1CCC(Cc2ccc(Cl)cc2F)CC1] | 1 | Ex 9, 27 | DMSO-d₆ + D₂O: 8.28 (s, 1H), 7.25-7.35 (m, 2H), 7.20 (d, J = 8.0 Hz, 1H), 4.08-4.18 (m, 2H), 2.77-2.90 (m, 2H), 2.54 (d, J = 6.8 Hz, 2H), 1.70-1.82 (m, 1H), 1.53-1.62 (m, 2H), 1.04-1.16 (m, 2H) | 355.1 357.0 [M + H]⁺ |
| 49 | 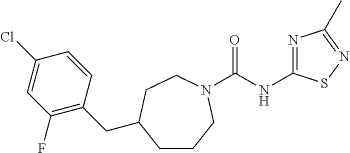 [SMILES: Cc1nsc(NC(=O)N2CCCC(Cc3ccc(Cl)cc3F)CC2)n1] | 1 | Ex 9, 27 | DMSO-d₆ + D₂O: 7.31 (dd, J1 = 2.0 Hz, J2 = 10.0 Hz, 1H), 7.27 (dd, J1 = J2 = 8.0 Hz, 1H), 7.19 (dd, J1 = 2.0 Hz, J2 = 8.0 Hz, 1H), 3.60-3.72 (m, 1H), 3.38-3.60 (m, 2H), 3.15-3.38 (m, 1H), 2.49 (m, 2H), 2.36 (s, 3H), 1.78-1.88 (m, 1H), 1.57-1.75 (m, 3H), 1.43-1.58 (m, 1H), 1.20-1.36 (m, 1H), 1.06-1.19 (m, 1H) | 383.1, 385.1 [M + H]⁺ |
| 50 | 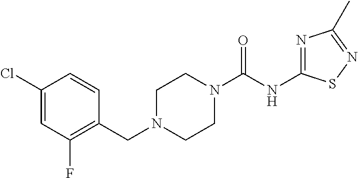 [SMILES: Cc1nsc(NC(=O)N2CCN(Cc3ccc(Cl)cc3F)CC2)n1] | 3 | Ex 27 | DMSO-d₆ + D₂O: 7.57-7.61 (m, 2H), 7.43 (dd, J1 = 8.4 Hz, J2 = 2.0 Hz, 1H), 4.35 (s, 2H), 3.58-4.15 (overlap, 4H), 3.26 (br, 4H), 2.37 (s, 3H) | 370.1, 372.0 [M + H]⁺ |
| 51 | 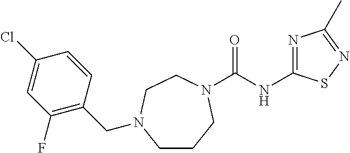 [SMILES: Cc1nsc(NC(=O)N2CCCN(Cc3ccc(Cl)cc3F)CC2)n1] | 3 | Ex 50, 27 | DMSO-d₆ + D₂O: 7.57-763 (m, 2H), 7.43 (dd, J1 = 8.4 Hz, J2 = 2.0 Hz, 1H), 4.39 (s, 2H), 3.56-3.97 (m, 4H), 3.32 (m, 4H), 2.37 (s, 3H), 2.11 (m, 2H) | 384.1 [M + H]⁺ |
| 52 | 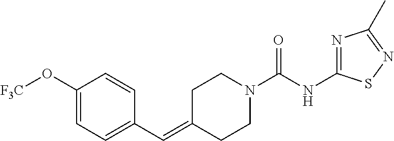 [SMILES: Cc1nsc(NC(=O)N2CCC(=Cc3ccc(OC(F)(F)F)cc3)CC2)n1] | 1 | Ex 88 | DMSO-d₆: 11.72 (s, 1H), 7.32-7.37 (m, 4H), 6.42 (s, 1H), 3.63 (t, J = 5.6 Hz, 2H), 3.56 (t, J = 5.6 Hz, 2H), 2.45 (t, J = 5.6 Hz, 2H), 2.36-2.38 (m, 5H) | 399.1 [M + H]⁺ |
| 53 | 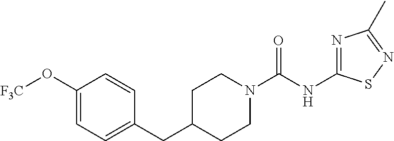 [SMILES: Cc1nsc(NC(=O)N2CCC(Cc3ccc(OC(F)(F)F)cc3)CC2)n1] | 1 | Ex 88, 27 | DMSO-d₆ + D₂O: 7.23-7.30 (m, 4H), 4.09-4.13 (m, 2H), 2.77-2.83 (m, 2H), 2.50-2.54 (m, 2H), 2.35 (s, 3H), 1.73-1.77 (m, 1H), 1.55-1.58 (m, 2H), 1.06-1.08 (m, 2H) | 401.1 [M + H]⁺ |
| 54 | 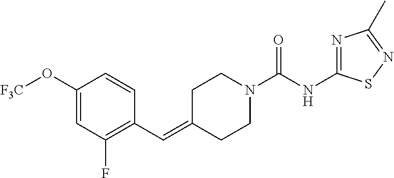 [SMILES: Cc1nsc(NC(=O)N2CCC(=Cc3ccc(OC(F)(F)F)cc3F)CC2)n1] | 1 | Ex 88 | DMSO-d₆ + D₂O: 7.41 (t, J = 8.4 Hz, 1H), 7.35 (t, J = 10.4 Hz, 1H), 7.21 (t, J = 8.4 Hz, 1H), 6.29 (s, 1H), 3.64 (t, J = 4.2 Hz, 2H), 3.55 (t, J = 4.2 Hz, 2H), 2.40 (t, J = 4.2 Hz, 2H), 2.38 (s, 3H), 2.31 (t, J = 4.2 Hz, 2H) | 417.1 [M + H]⁺ |
| 55 | 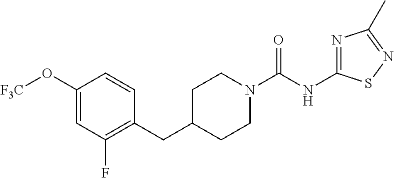 [SMILES: Cc1nsc(NC(=O)N2CCC(Cc3ccc(OC(F)(F)F)cc3F)CC2)n1] | 1 | Ex 88, 27 | DMSO-d₆ + D₂O: 7.34 (t, J = 8.4 Hz, 1H), 7.28 (d, J = 10.4 Hz, 1H), 7.15 (d, J = 8.0 Hz, 1H), 4.10-4.13 (m, 2H), 2.77-2.84 (m, 2H), 2.56 (d, J = 7.2 Hz, 2H), 2.35 (s, 3H), 1.74-1.78 (m, 1H), 1.55-1.58 (m, 2H), 1.04-1.14 (m, 2H) | 419.4 [M + H]⁺ |
| 56 | 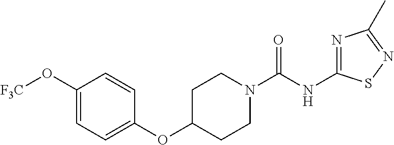 [SMILES: Cc1nsc(NC(=O)N2CCC(Oc3ccc(OC(F)(F)F)cc3)CC2)n1] | 2 | Ex 19 | DMSO-d₆ + D₂O: 7.27 (d, J = 8.8 Hz, 2H), 7.05-7.09 (m, 2H), 4.60-4.64 (m, 1H), 3.80-3.84 (m, 2H), 3.37-3.42 (m, 2H), 2.36 (s, 3H), 1.93-1.97 (m, 2H), 1.54-1.62 (m, 2H) | 403.0 [M + H]⁺ |
| 57 | 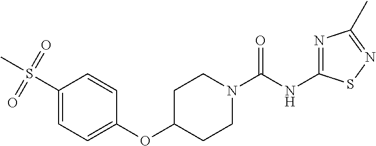 [SMILES: Cc1nsc(NC(=O)N2CCC(Oc3ccc(S(C)(=O)=O)cc3)CC2)n1] | 1 | Ex 88, 27 | DMSO-d₆ + D₂O: 7.82 (d, J = 8.0 Hz, 2H), 7.45 (d, J = 8.0 Hz, 2H), 4.10-4.13 (m, 2H), 3.16 (s, 3H), 2.78-2.84 (m, 2H), 2.62 (d, J = 7.2 Hz, 2H), 2.35 (s, 3H), 1.78-1.84 (m, 1H), 1.55-1.58 (m, 2H), 1.05-1.15 (m, 2H) | 395.6 [M + H]⁺ |
| 58 | 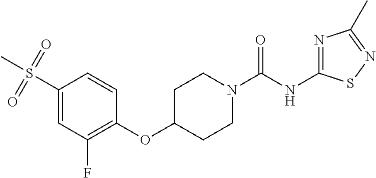 [SMILES: Cc1nsc(NC(=O)N2CCC(Oc3ccc(S(C)(=O)=O)cc3F)CC2)n1] | 1 | Ex 88, 27 | DMSO-d₆ + D₂O: 7.67-7.71 (m, 2H), 7.55-7.58 (m, 1H), 4.10-4.13 (m, 2H), 3.21 (s, 3H), 2.78-2.84 (m, 2H), 2.65 (d, J = 7.2 Hz, 2H), 2.35 (s, 3H), 1.78-1.83 (m, 1H), 1.56-1.59 (m, 2H), 1.11-1.13 (m, 2H) | 413.1 [M + H]⁺ |
| 59 | 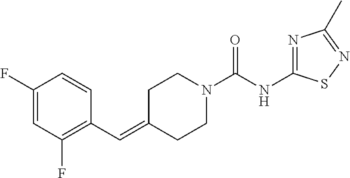 [SMILES: Cc1nsc(NC(=O)N2CCC(=Cc3ccc(F)cc3F)CC2)n1] | 1 | Ex 88 | DMSO-d₆ + D₂O: 7.27-7.33 (m, 1H), 7.15-7.20 (m, 1H), 7.02-7.06 (m, 1H), 6.24 (s, 1H), 3.60 (t, J = 5.6 Hz, 2H), 3.51 (t, J = 5.6 Hz, 2H), 2.36-2.38 (m, 5H), 2.27 (t, J = 5.6 Hz, 2H) | 351.0 [M + H]⁺ |
| 60 | 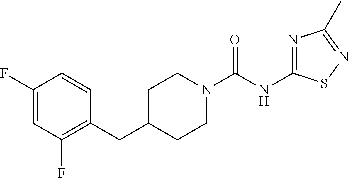 [SMILES: Cc1nsc(NC(=O)N2CCC(Cc3ccc(F)cc3F)CC2)n1] | 1 | Ex 88, 27 | DMSO-d₆ + D₂O: 7.26-7.32 (m, 1H), 7.10-7.15 (m, 1H), 6.96-7.01 (m, 1H), 4.12 (d, J = 13.2 Hz, 2H), 2.80 (t, J = 12.4 Hz, 2H), 2.52 (s, 2H), 2.35 (s, 3H), 1.70-1.75 (m, 1H), 1.56 (d, J = 11.2 Hz, 2H), 1.03-1.13 (m, 2H) | 353.0 [M + H]⁺ |
| 61 | 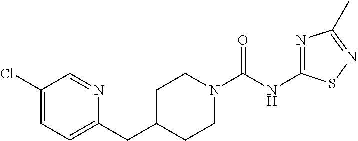 [SMILES: Cc1nsc(NC(=O)N2CCC(Cc3ccc(Cl)cn3)CC2)n1] | 1 | Ex 88, 27 | DMSO-d₆ + D₂O: 8.50 (t, J = 2.4 Hz, 1H), 7.81 (dd, J1 = 8.4 Hz, J2 = 2.4 Hz, 1H), 7.30 (d, J = 8.4 Hz, 1H), 4.09-4.12 (m, 2H), 2.79-2.85 (m, 2H), 2.66 (d, J = 7.6 Hz, 2H), 2.35 (s, 3H), 1.93-1.98 (m, 1H), 1.54-1.57 (m, 2H), 1.06-1.16 (m, 2H) | 352.3, 354.3 [M + H]⁺ |
| 62 | 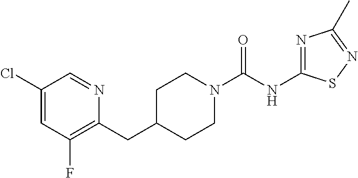 [SMILES: Cc1nsc(NC(=O)N2CCC(Cc3ncc(Cl)cc3F)CC2)n1] | 1 | Ex 88, 27 | DMSO-d₆ + D₂O: 8.43 (s, 1H), 7.95-7.97 (m, 1H), 4.10-4.13 (m, 2H), 2.80-2.86 (m, 2H), 2.71 (d, J = 6.8 Hz, 2H), 2.35 (s, 3H), 1.93-2.03 (m, 1H), 1.58-1.61 (m, 2H), 1.09-1.18 (m, 2H) | 370.1, 372.1 [M + H]⁺ |
| 63 | 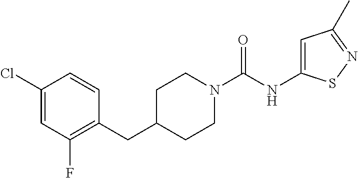 [SMILES: Cc1cc(NC(=O)N2CCC(Cc3ccc(Cl)cc3F)CC2)sn1] | 1 | Ex 70 | DMSO-d₆ + D₂O: 7.28-7.34 (m, 2H), 7.20 (dd, J1 = 2.0 Hz, J2 = 8.4 Hz, 1H), 6.63 (s, 1H), 4.02-4.05 (m, 2H), 2.78-2.84 (m, 2H), 2.50-2.55 (m, 2H), 2.27 (s, 3H), 1.71-1.77 (m, 1H), 1.56-1.59 (m, 2H), 1.04-1.15 (m, 2H) | 368.2, 370.2 [M + H]⁺ |
| 64 | 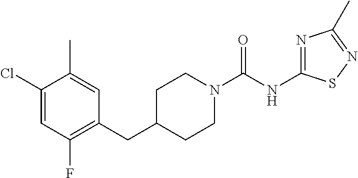 [SMILES: Cc1nsc(NC(=O)N2CCC(Cc3cc(C)c(Cl)cc3F)CC2)n1] | 1 | Ex 88, 27 | DMSO-d₆ + D₂O: 7.27 (d, J = 9.6 Hz, 1H), 7.23 (d, J = 8.4 Hz, 1H), 4.09-4.12 (m, 2H), 2.77-2.83 (m, 2H), 2.43-2.49 (m, 2H), 2.35 (s, 3H), 2.24 (s, 3H), 1.71-1.75 (m, 1H), 1.55-1.58 (m, 2H), 1.02-1.12 (m, 2H) | 383.1, 385.1 [M + H]⁺ |
| 65 | 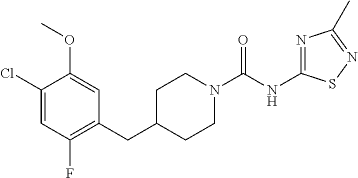 [SMILES: COc1cc(CC2CCN(C(=O)Nc3nc(C)ns3)CC2)c(F)cc1Cl] | 1 | Ex 88, 27 | DMSO-d₆ + D₂O: 7.32 (d, J = 9.2 Hz, 1H), 7.03 (d, J = 6.4 Hz, 1H), 4.13 (d, J = 13.6 Hz, 2H), 3.80 (s, 3H), 2.80 (t, J = 12.8 Hz, 2H), 2.53 (d, J = 7.6 Hz, 2H), 2.35 (s, 3H), 1.75-1.80 (m, 1H), 1.58 (d, J = 11.2 Hz, 2H), 1.06-1.15 (m, 2H) | 399.2, 401.2 [M + H]⁺ |
| 66 | 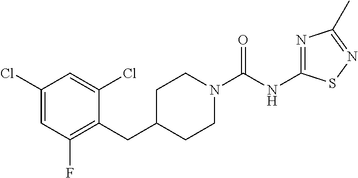 [SMILES: Cc1nsc(NC(=O)N2CCC(Cc3c(F)cc(Cl)cc3Cl)CC2)n1] | 1 | Ex 88, 27 | DMSO-d₆ + D₂O: 7.49 (t, J = 1.6 Hz, 1H), 7.43 (dd, J1 = 9.4 Hz, J2 = 1.6 Hz, 1H), 4.11-4.14 (m, 2H), 2.77-2.83 (m, 2H), 2.65 (dd, J1 = 7.2 Hz, J2 = 1.6 Hz, 2H), 2.35 (s, 3H), 1.78-1.79 (m, 1H), 1.55-1.58 (m, 2H), 1.15-1.22 (m, 2H) | 403.2, 405.1 [M + H]⁺ |
| 67 | 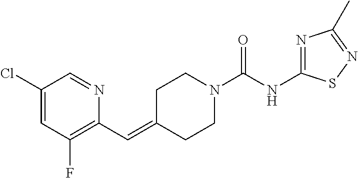 [SMILES: Cc1nsc(NC(=O)N2CCC(=Cc3ncc(Cl)cc3F)CC2)n1] | 1 | Ex 88 | DMSO-d₆ + D₂O: 8.47 (d, J = 2.0 Hz, 1H), 7.99 (dd, J1 = 10.0 Hz, J2 = 2.0 Hz, 1H), 6.43 (s, 1H), 3.62-3.65 (m, 2H), 3.55-3.58 (m, 2H), 2.85 (t, J = 5.6 Hz, 2H), 2.43 (t, J = 4.2 Hz, 2H), 2.37 (s, 3H) | 368.2, 370.2 [M + H]⁺ |
| 68 | 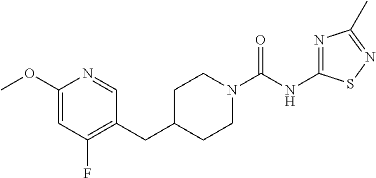 [SMILES: COc1cc(F)c(CC2CCN(C(=O)Nc3nc(C)ns3)CC2)cn1] | 1 | Ex 88, 27 | DMSO-d₆ + D₂O: 8.03 (t, J = 10.8 Hz, 1H), 6.68 (t, J = 10.8 Hz, 1H), 4.10-4.14 (m, 2H), 3.82 (s, 3H), 2.77-2.85 (m, 2H), 2.48-2.49 (m, 2H), 2.35 (s, 3H), 1.69-1.75 (m, 1H), 1.56-1.58 (m, 2H), 1.02-1.14 (m, 2H) | 366.0 [M + H]⁺ |
| 69 | 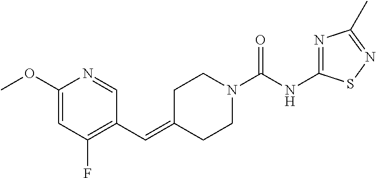 [SMILES: COc1cc(F)c(C=C2CCN(C(=O)Nc3nc(C)ns3)CC2)cn1] | 1 | Ex 88 | DMSO-d₆ + D₂O: 8.09 (d, J = 10.8 Hz, 1H), 6.78 (d, J = 10.8 Hz, 1H), 6.19 (s, 1H), 3.87 (s, 3H), 3.63 (t, J = 4.2 Hz, 2H), 3.56 (t, J = 4.2 Hz, 2H), 2.33-2.41 (m, 5H), 2.30 (t, J = 4.2 Hz, 2H) | 364.0 [M + H]⁺ |
| 70 | 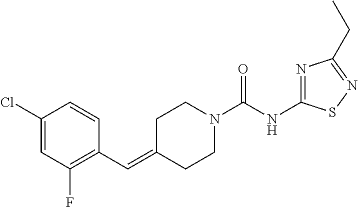 [SMILES: CCc1nsc(NC(=O)N2CCC(=Cc3ccc(Cl)cc3F)CC2)n1] | 1 | — | DMSO-d₆ + D₂O: 7.28-7.35 (m, 2H), 7.20 (dd, J1 = 2.0 Hz, J2 = 8.4 Hz, 1H), 4.11-4.14 (m, 2H), 2.77-2.83 (m, 2H), 2.69 (q, J = 7.6 Hz, 2H), 2.52-2.54 (m, 2H), 1.72-1.76 (m, 1H), 1.55-1.57 (m, 2H), 1.21 (t, J = 7.6 Hz, 3H), 1.07-1.11 (m, 2H) | 383.1, 385.1 [M + H]⁺ |
| 71 | 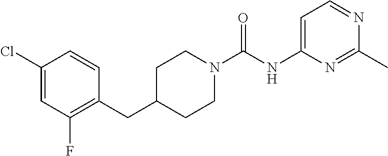 [SMILES: Cc1nccc(NC(=O)N2CCC(Cc3ccc(Cl)cc3F)CC2)n1] | 1 | 70 | DMSO-d₆ + D₂O: 8.51 (d, J = 6.8 Hz, 1H), 7.71 (d, J = 6.8 Hz, 1H), 7.28-7.36 (m, 2H), 7.21 (dd, J1 = 2.0 Hz, J2 = 8.4 Hz, 1H), 4.04-4.08 (m, 2H), 2.78-2.84 (m, 2H), 2.58 (s, 3H), 2.53-2.55 (m, 2H), 1.72-1.77 (m, 1H), 1.56-1.59 (m, 2H), 1.07-1.18 (m, 2H) | 363.2, 365.2 [M + H]⁺ |
| 72 | 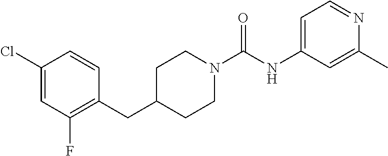 [SMILES: Cc1cc(NC(=O)N2CCC(Cc3ccc(Cl)cc3F)CC2)ccn1] | 1 | 70 | DMSO-d₆ + D₂O: 8.35 (t, J = 2.4 Hz, 1H), 7.67-7.69 (m, 2H), 7.28-7.34 (m, 2H), 7.20 (dd, J1 = 2.0 Hz, J2 = 8.4 Hz, 1H), 4.05-4.08 (m, 2H), 2.79-2.86 (m, 2H), 2.52-2.55 (m, 2H), 2.54 (s, 3H), 1.73-1.78 (m, 1H), 1.58-1.61 (m, 2H), 1.07-1.17 (m, 2H) | 362.3, 364.2 [M + H]⁺ |
| 73 | 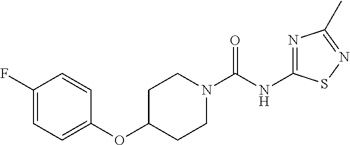 [SMILES: Cc1nsc(NC(=O)N2CCC(Oc3ccc(F)cc3)CC2)n1] | 2 | Ex 19 | DMSO-d₆: 11.69 (s, 1H), 7.14 (m, 2H), 7.02 (m, 2H), 4.57 (m, 1H), 3.85 (m, 2H), 3.42 (m, 2H), 2.40 (s, 3H), 1.94 (m, 2H), 1.61 (m, 2H) | 337.1 [M + H]⁺ |
| 74 | 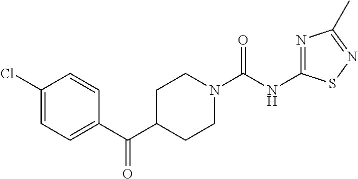 [SMILES: Cc1nsc(NC(=O)N2CCC(C(=O)c3ccc(Cl)cc3)CC2)n1] | 4 | Ex 77, 27 | DMSO-d₆: 11.66 (s, 1H), 8.04 (m, 2H), 7.63 (m, 2H), 4.22 (m, 2H), 3.72 (m, 1H), 3.11 (m, 2H), 2.40 (s, 3H), 1.85 (m, 2H), 1.49 (m, 2H) | 365.0, 367.0 [M + H]⁺ |
| 75 | 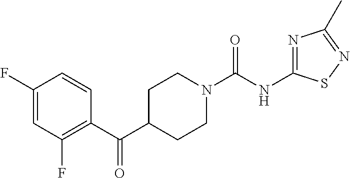 [SMILES: Cc1nsc(NC(=O)N2CCC(C(=O)c3ccc(F)cc3F)CC2)n1] | 4 | Ex 77, 27 | DMSO-d₆: 11.66 (s, 1H), 7.92 (m, 1H), 7.45 (m, 1H), 7.27 (m, 1H), 4.19 (m, 2H), 3.42 (m, 1H), 3.08 (m, 2H), 2.40 (s, 3H), 1.88 (m, 2H), 1.48 (m, 2H) | 367.1 [M + H]⁺ |
| 76 | 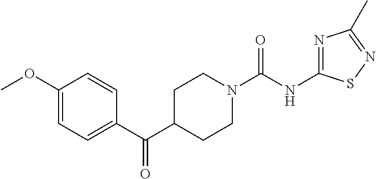 [SMILES: COc1ccc(C(=O)C2CCN(C(=O)Nc3nc(C)ns3)CC2)cc1] | 4 | Ex 77, 27 | DMSO-d₆: 11.66 (s, 1H), 8.01 (m, 2H), 7.07 (m, 2H), 4.23 (m, 2H), 3.69 (m, 1H), 3.11 (m, 2H), 2.40 (s, 3H), 1.82 (m, 2H), 1.45-1.54 (m, 2H) | 361.1 [M + H]⁺ |
| 77 | 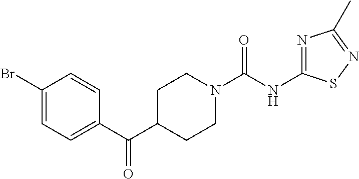 [SMILES: Cc1nsc(NC(=O)N2CCC(C(=O)c3ccc(Br)cc3)CC2)n1] | 4 | Ex 27 | DMSO-d₆: 11.66 (s, 1H), 7.96 (m, 2H), 7.77 (m, 2H), 4.22 (m, 2H), 3.71 (m, 2H), 3.11 (m, 2H), 2.40 (s, 3H), 1.84 (m, 2H), 1.45-154 (m, 2H) | 409.0, 411.0 [M + H]⁺ |
| 78 | 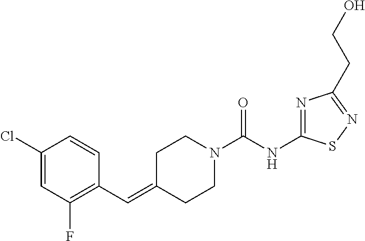 [SMILES: O=C(Nc1nc(CCO)ns1)N1CCC(=Cc2ccc(Cl)cc2F)CC1] | 1 | — | DMSO-d₆: 11.75 (s, 1H), 7.45 (m, 1H), 7.28-7.36 (m, 2H), 6.30 (s, 1H), 4.66 (m, 1H), 3.80 (m, 2H), 3.57-3.66 (m, 4H), 2.88 (m, 2H), 2.32-2.41 (m, 4H) | 397.0, 399.0 [M + H]⁺ |
| 79 | 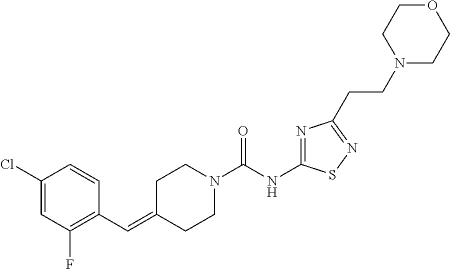 [SMILES: O=C(Nc1nc(CCN2CCOCC2)ns1)N1CCC(=Cc2ccc(Cl)cc2F)CC1] | 1 | Ex 78 | DMSO-d₆: 11.80 (s, 1H), 7.45 (m, 1H), 7.32 (m, 2H), 6.30 (s, 1H), 3.56-3.65 (m, 8H), 2.90 (m, 2H), 2.74 (m, 2H), 2.32-2.41 (m, 8H) | 466.1, 468.0 [M + H]⁺ |
| 80 | 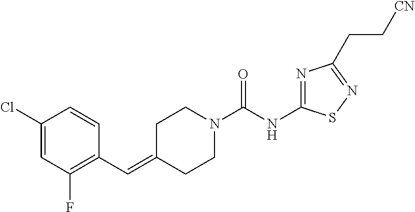 [SMILES: N#CCCc1nsc(NC(=O)N2CCC(=Cc3ccc(Cl)cc3F)CC2)n1] | 1 | Ex 78 | DMSO-d₆: 11.83 (s, 1H), 7.44 (m, 1H), 7.30 (m, 2H), 6.30 (s, 1H), 3.58-3.66 (m, 2H), 3.07 (m, 2H), 2.93 (m, 2H), 2.32-2.41 (m, 4H) | 406.0, 408.0 [M + H]⁺ |
| 81 | 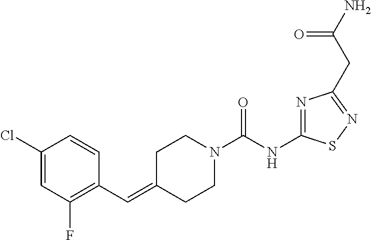 [SMILES: NC(=O)Cc1nsc(NC(=O)N2CCC(=Cc3ccc(Cl)cc3F)CC2)n1] | 1 | Ex 78 | DMSO-d₆: 11.82 (s, 1H), 7.43-7.50 (m, 2H), 7.28-7.34 (m, 2H), 7.04 (br, 1H), 6.30 (s, 1H), 3.58-3.66 (m, 6H), 2.32-2.41 (m, 4H) | 410.0, 412.0 [M + H]⁺ |
| 82 | 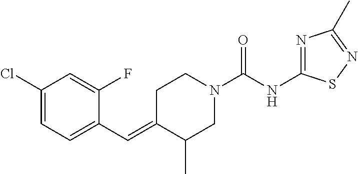 [SMILES: Cc1nsc(NC(=O)N2CC/C(=C\c3ccc(Cl)cc3F)C(C)C2)n1] | 1 | Ex 95 | DMSO-d₆ + D₂O: 7.39 (dd, J1 = 2.0 Hz, J2 = 8.4 Hz, 1H), 7.23-7.30 (m, 2H), 6.19 (s, 1H), 3.84 (m, 1H), 3.66 (m, 1H), 3.35 (m, 1H), 3.15 (m, 1H), 2.40-2.53 (m, 2H), 2.36 (s, 3H), 2.15 (m, 1H), 1.10 (d, J = 6.4 Hz, 3H) | 381.2, 383.2 [M + H]⁺ |
| 83 | 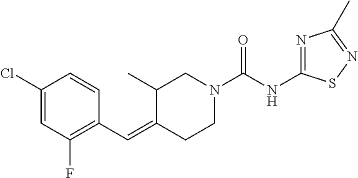 [SMILES: Cc1nsc(NC(=O)N2CC/C(=C/c3ccc(Cl)cc3F)C(C)C2)n1] | 1 | Ex 95 | DMSO-d₆ + D₂O: 7.41 (dd, J1 = 2.0 Hz, J2 = 8.4 Hz, 1H), 7.25-7.32 (m, 2H), 6.18 (s, 1H), 4.35 (m, 1H), 4.08 (m, 1H), 2.80-2.99 (m, 2H), 2.75 (m, 1H), 2.57 (m, 1H), 2.36 (s, 3H), 2.24 (m, 1H), 1.04 (d, J = 7.2 Hz, 3H) | 381.2, 383.2 [M + H]⁺ |
| 84a | 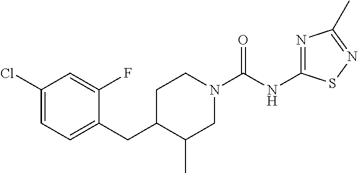 [SMILES: Cc1nsc(NC(=O)N2CCC(Cc3ccc(Cl)cc3F)C(C)C2)n1] | 1 | Ex 27 | DMSO-d₆ + D₂O: 7.27-7.39 (m, 2H), 7.20 (dd, J1 = 2.0 Hz, J2 = 8.0 Hz, 1H), 4.04 (m, 1H), 3.90 (m, 1H), 3.05 (m, 1H), 2.87 (m, 1H), 2.45-2.60 (m, 2H), 2.36 (s, 3H), 1.90 (m, 1H), 1.73 (m, 1H), 1.27-1.42 (m, 2H), 0.83 (d, J = 6.8 Hz, 3H) | 383.2, 385.2 [M + H]⁺ |
| 84b | 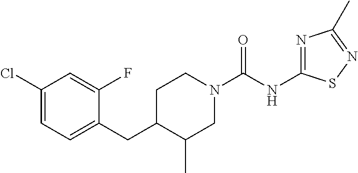 [SMILES: Cc1nsc(NC(=O)N2CCC(Cc3ccc(Cl)cc3F)C(C)C2)n1] | 1 | Ex 27 | DMSO-d₆ + D₂O: 7.34 (dd, J1 = 2.0 Hz, J2 = 10.0 Hz, 1H), 7.28 (dd, J1 = J2 = 8.4 Hz, 1H), 7.21 (dd, J1 = 2.0 Hz, J2 = 8.4 Hz, 1H), 4.02-4.13 (m, 2H), 2.88 (m, 1H), 2.69-2.70 (m, 1H), 2.50-2.60 (m, 1H), 2.36 (s, 3H), 2.24 (m, 1H), 1.26-1.48 (m, 3H), 1.05 (m, 1H), 0.99 (d, J = 6.4 Hz, 3H) | 383.2, 385.2 [M + H]⁺ |
| 85 | 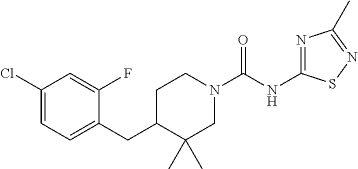 [SMILES: Cc1nsc(NC(=O)N2CCC(Cc3ccc(Cl)cc3F)C(C)(C)C2)n1] | 1 | Ex 95 | DMSO-d₆ + D₂O: 7.38 (dd, J1 = 2.0 Hz, J2 = 10.0 Hz, 1H), 7.22-7.28 (m, 2H), 6.21 (s, 1H), 3.49 (t, J = 5.6 Hz, 2H), 3.39 (s, 2H), 2.36 (s, 3H), 2.33 (t, J = 5.6 Hz, 2H), 1.13 (s, 6H) | 395.2, 397.2 [M + H]⁺ |
| 86 | 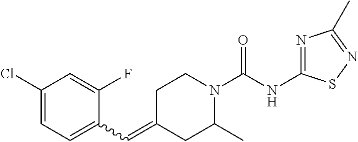 [SMILES: Cc1nsc(NC(=O)N2CCC(=Cc3ccc(Cl)cc3F)CC2C)n1] | 1 | Ex 95 | DMSO-d₆ + D₂O: 7.30-7.42 (m, 2H), 7.25 (m, 1H), 6.38 (s, 0.5H), 6.24 (s, 0.5H), 4.53-4.55 (m, 1H), 4.16 (m, 0.5H), 4.07 (m, 0.5H), 2.96-3.12 (m, 1H), 2.53 (m, 0.5H), 2.43 (m, 1H), 2.36 (s, 3H), 2.22-2.33 (m, 2H), 2.11 (m, 0.5H), 1.12 (d, J = 6.8 Hz, 1.5H), 0.99 (d, J = 6.8 Hz, 1.5H) | 381.2, 383.2 [M + H]⁺ |
| 87 | 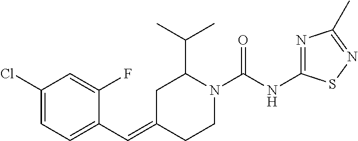 [SMILES: Cc1nsc(NC(=O)N2CC/C(=C/c3ccc(Cl)cc3F)CC2C(C)C)n1] | 1 | Ex 95 | DMSO-d₆ + D₂O: 7.41 (dd, J1 = 2.0 Hz, J2 = 10.0 Hz, 1H), 7.23-7.32 (m, 2H), 6.34 (s, 1H), 4.10-4.35 (m, 1H), 3.90-4.04 (m, 1H), 2.70-3.10 (m, 1H), 2.60 (m, 1H), 2.26-2.43 (m, 5H), 2.16 (m, 1H), 1.76 (m, 1H), 0.66 (d, J = 6.8 Hz, 3H), 0.49 (d, J = 6.8 Hz, 3H) | 409.3, 411.3 [M + H]⁺ |
| 88 | 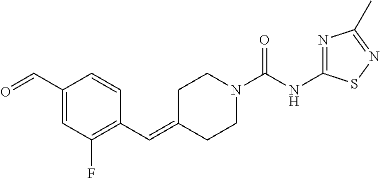 [SMILES: Cc1nsc(NC(=O)N2CCC(=Cc3ccc(C=O)cc3F)CC2)n1] | 1 | Ex 27 | DMSO-d₆: 11.75 (s, 1H), 10.00 (s, 1H), 7.71-7.78 (m, 2H), 7.57 (m, 1H), 6.42 (s, 1H), 3.59-3.67 (m, 4H), 2.41-2.46 (m, 7H) | 361.1 [M + H]⁺ |
| 89 | 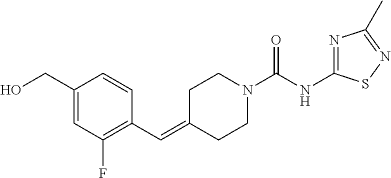 [SMILES: Cc1nsc(NC(=O)N2CCC(=Cc3ccc(CO)cc3F)CC2)n1] | 1 | Ex 88 | DMSO-d₆: 11.75 (s, 1H), 7.25 (m, 1H), 7.13 (m, 2H), 6.32 (s, 1H), 5.30 (t, J = 5.7 Hz, 1H), 4.51 (d, J = 5.7 Hz, 2H), 3.57-3.65 (m, 4H), 2.34-2.42 (m, 7H) | 363.1 [M + H]⁺ |
| 90 | 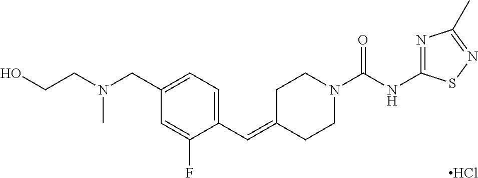 [SMILES: Cc1nsc(NC(=O)N2CCC(=Cc3ccc(CN(C)CCO)cc3F)CC2)n1 ; Cl] | 1 | Ex 88 | DMSO-d₆: 11.76 (s, 1H), 10.35 (br, 1H), 7.52 (m, 1H), 7.41 (m, 2H), 6.35 (s, 1H), 5.77 (br, 1H), 4.32-4.40 (m, 2H), 3.79 (m, 2H), 3.58-3.66 (m, 4H), 3.08-3.17 (m, 2H), 2.75 (m, 3H), 2.36-2.43 (m, 7H) | 420.2 [M + H]⁺ |
| 91 | 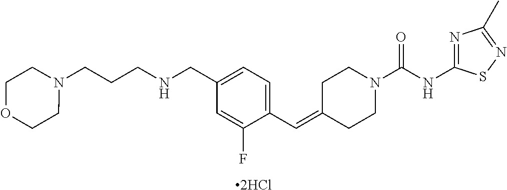 [SMILES: Cc1nsc(NC(=O)N2CCC(=Cc3ccc(CNCCCN4CCOCC4)cc3F)CC2)n1 ; "·2HCl"] | 1 | Ex 88, 90 | DMSO-d₆: 11.74 (s, 1H), 11.36 (br, 1H), 9.64 (br, 2H), 7.53 (m, 1H), 7.35-743 (m, 2H), 6.35 (s, 1H), 4.17 (m, 2H), 3.98 (m, 2H), 3.86 (m, 4H), 3.58-3.66 (m, 4H), 3.40 (m, 2H), 3.23 (m, 2H), 3.04 (m, 4H), 2.34-243 (m, 7H), 2.20 (m, 2H) | 489.2 [M + H]⁺ |
| 92 | 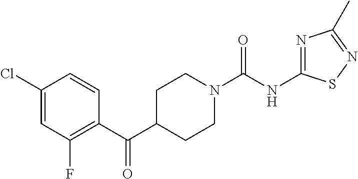 [SMILES: Cc1nsc(NC(=O)N2CCC(C(=O)c3ccc(Cl)cc3F)CC2)n1] | 4 | Ex 39, 77 | DMSO-d₆: 11.72 (br, 1H), 7.84 (m, 1H), 7.64 (m, 1H), 7.46 (m, 1H), 4.19 (m, 2H), 3.42 (m, 1H), 3.04-3.10 (m, 2H), 2.39 (s, 3H), 1.87 (m, 2H), 1.43-1.51 (m, 2H) | 382.9, 384.0 [M + H]⁺ |
| 93 | 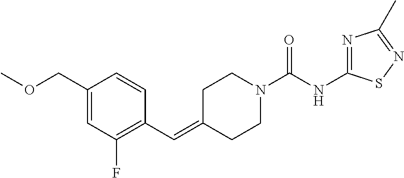 [SMILES: COCc1ccc(C=C2CCN(C(=O)Nc3nc(C)ns3)CC2)c(F)c1] | 1 | Ex 88, 89 | DMSO-d₆: 11.73 (s, 1H), 7.28 (m, 1H), 7.14 (m, 2H), 6.32 (s, 1H), 4.42 (s, 2H), 3.57-3.65 (m, 4H), 3.31 (s, 3H), 2.34-2.41 (m, 7H) | 377.0 [M + H]⁺ |
| 94 | 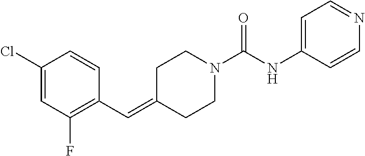 [SMILES: O=C(Nc1ccncc1)N1CCC(=Cc2ccc(Cl)cc2F)CC1] | 1 | Ex 70 | DMSO-d₆: 9.04 (s, 1H), 8.32 (m, 2H), 7.50 (m, 2H), 7.44 (m, 1H), 7.28-7.37 (m, 2H), 6.29 (s, 1H), 3.60 (m, 2H), 3.50 (m, 2H), 2.42 (m, 2H), 2.33 (m, 2H) | 346.1, 348.0 [M + H]⁺ |
| 95 | 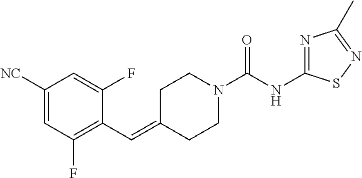 [SMILES: Cc1nsc(NC(=O)N2CCC(=Cc3c(F)cc(C#N)cc3F)CC2)n1] | 1 | Ex 27 | DMSO-d₆ + D₂O: 7.75 (s, 1H), 7.72 (s, 1H), 6.10 (s, 1H), 3.60-3.62 (m, 2H), 3.49-3.54 (m, 2H), 2.40-2.45 (m, 2H), 2.36 (s, 3H), 2.03-2.09 (m, 2H) | 376.0 [M + H]⁺ |
| 96 | 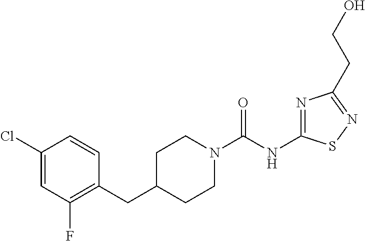 [SMILES: O=C(Nc1nc(CCO)ns1)N1CCC(Cc2ccc(Cl)cc2F)CC1] | 1 | Ex 27, 78 | DMSO-d₆ + D₂O: 7.28-7.35 (m, 2H), 7.20 (dd, J1 = 2.0 Hz, J2 = 8.4 Hz, 1H), 4.11-4.15 (m, 2H), 3.76 (t, J = 7.2 Hz, 2H), 2.81-2.86 (m, 4H), 2.52-2.54 (m, 2H), 1.72-1.78 (m, 1H), 1.55-1.58 (m, 2H), 1.04-1.14 (m, 2H). | 399.0, 401.0 [M + H]⁺ |
| 97 | 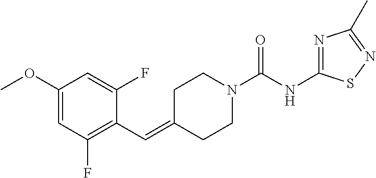 [SMILES: COc1cc(F)c(C=C2CCN(C(=O)Nc3nc(C)ns3)CC2)c(F)c1] | 1 | Ex 95 | DMSO-d₆ + D₂O: 6.74 (s, 1H), 6.71 (s, 1H), 5.97 (s, 1H), 3.76 (s, 3H), 3.57-3.60 (m, 2H), 3.45-3.52 (m, 2H), 2.34-2.39 (m, 5H), 2.03-2.09 (m, 2H) | 381.2 [M + H]⁺ |
| 98 | 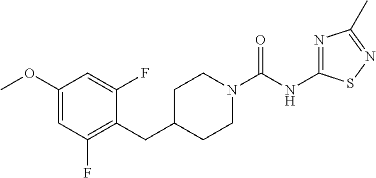 [SMILES: COc1cc(F)c(CC2CCN(C(=O)Nc3nc(C)ns3)CC2)c(F)c1] | 1 | Ex 27 | DMSO-d₆ + D₂O: 6.70 (s, 1H), 6.67 (s, 1H), 4.09-4.14 (m, 2H), 3.73 (s, 3H), 2.70-2.84 (m, 2H), 2.46-2.48 (m, 2H), 2.36 (s, 3H), 1.65-1.72 (m, 1H), 1.55-1.60 (m, 2H), 1.03-1.14 (m, 2H) | 383.2 [M + H]⁺ |
| 99 | 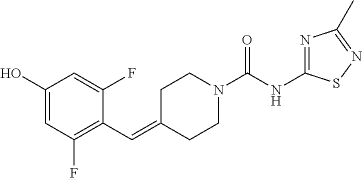 [SMILES: Cc1nsc(NC(=O)N2CCC(=Cc3c(F)cc(O)cc3F)CC2)n1] | 1 | Ex 95 | DMSO-d₆ + D₂O: 6.48 (s, 1H), 6.46 (s, 1H), 5.94 (s, 1H), 3.57-3.61 (m, 2H), 3.45-3.52 (m, 2H), 2.34-2.38 (m, 5H), 2.02-2.09 (m, 2H) | 367.2 [M + H]⁺ |
| 100 | 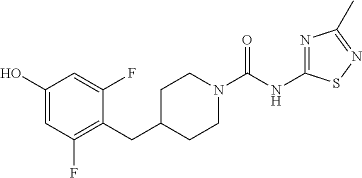 [SMILES: Cc1nsc(NC(=O)N2CCC(Cc3c(F)cc(O)cc3F)CC2)n1] | 1 | Ex 98, 99 | DMSO-d₆ + D₂O: 6.44 (s, 1H), 6.41 (s, 1H), 4.09-4.14 (m, 2H), 2.75-2.84 (m, 2H), 2.43 (d, J = 6.8 Hz, 2H), 2.36 (s, 3H), 1.64-1.70 (m, 1H), 1.54-1.60 (m, 2H), 1.03-1.12 (m, 2H) | 369.0 [M + H]⁺ |
| 101 | 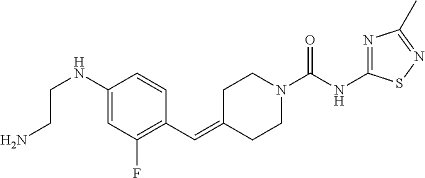 [SMILES: Cc1nsc(NC(=O)N2CCC(=Cc3ccc(NCCN)cc3F)CC2)n1] | 1 | — | DMSO-d₆ + D₂O: 7.01 (t, J = 8.8 Hz, 1H), 6.38-6.44 (m, 2H), 6.16 (s, 1H), 3.49-3.65 (m, 4H), 3.26 (t, J = 6.4 Hz, 2H), 2.92 (t, J = 6.4 Hz, 2H), 2.37 (s, 3H), 2.30-2.35 (m, 4H) | 391.0 [M + H]⁺ |
| 102 | 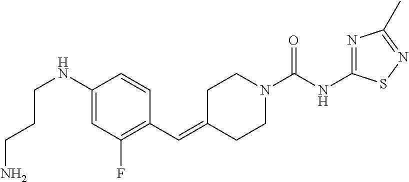 [SMILES: Cc1nsc(NC(=O)N2CCC(=Cc3ccc(NCCCN)cc3F)CC2)n1] | 1 | Ex 101 | DMSO-d₆ + D₂O: 6.97 (t, J = 8.4 Hz, 1H), 6.22-6.40 (m, 2H), 6.15 (s, 1H), 3.48-3.62 (m, 4H), 3.07 (t, J = 6.8 Hz, 2H), 2.85 (t, J = 7.6 Hz, 2H), 2.37 (s, 3H), 2.28-2.34 (m, 4H), 1.78 (m, 2H) | 405.2 [M + H]⁺ |
| 103 | 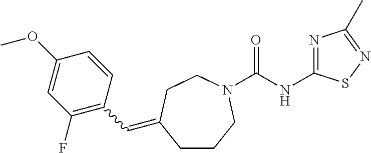 [SMILES: COc1ccc(C=C2CCCN(C(=O)Nc3nc(C)ns3)CC2)c(F)c1] | 1 | Ex 88 | DMSO-d₆ + D₂O: 7.07-7.11 (m, 1H), 6.70-6.78 (m, 2H), 6.13-6.20 (m, 1H), 3.72 (s, 3H), 3.57 (m, 4H), 2.25-2.55 (m, 4H), 2.37 (s, 3H), 1.66-1.70 (m, 2H) | 377.0 [M + H]⁺ |
| 104 | 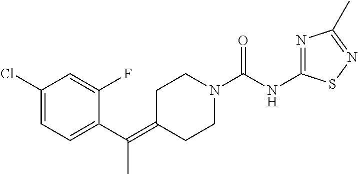 [SMILES: CC(=C1CCN(C(=O)Nc2nc(C)ns2)CC1)c1ccc(Cl)cc1F] | 1 | Ex 95 | DMSO-d₆ + D₂O: 7.40 (dd, J1 = 2.0 Hz, J2 = 9.6 Hz, 1H), 7.27 (dd, J1 = 2.0 Hz, J2 = 8.4 Hz, 1H), 7.21 (dd, J1 = J2 = 8.4 Hz, 1H), 3.54-3.62 (m, 2H), 3.38-3.46 (m, 2H), 2.40-2.46 (m, 2H), 2.36 (s, 3H), 1.93-2.02 (m, 2H), 1.90 (s, 3H) | 381.0, 383.0 [M + H]⁺ |
| 105 | 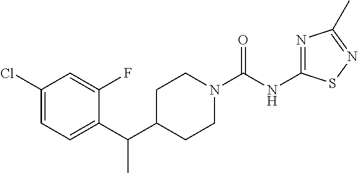 [SMILES: Cc1nsc(NC(=O)N2CCC(C(C)c3ccc(Cl)cc3F)CC2)n1] | 1 | Ex 27 | DMSO-d₆ + D₂O: 7.27-7.35 (m, 2H), 7.23 (dd, J1 = 2.0 Hz, J2 = 8.4 Hz, 1H), 4.20 (m, 1H), 4.07 (m, 1H), 2.66-2.89 (m, 3H), 2.35 (s, 3H), 1.84 (m, 1H), 1.62-1.73 (m, 1H), 1.30 (m, 1H), 1.19 and 1.17 (2s, 3H), 1.10-1.13 (m, 1H), 0.89-1.00 (m, 1H) | 383.0, 385.0 [M + H]⁺ |
| 106 | 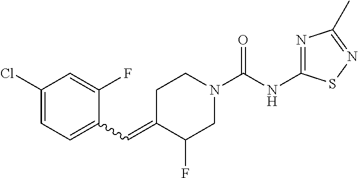 [SMILES: Cc1nsc(NC(=O)N2CCC(=Cc3ccc(Cl)cc3F)C(F)C2)n1] | 1 | Ex 95 | DMSO-d₆ + D₂O: 7.47 (dd, J1 = 2.0 Hz, J2 = 10.0 Hz, 1H), 7.28-7.40 (m, 2H), 6.64 (s, 0.8H), 6.59 (s, 0.2H), 5.30 and 5.19 (2m, 1H), 4.59 (m, 0.2H), 4.30-4.44 (m, 1H), 4.05 (m, 0.8H), 3.42-3.52 (m, 1H), 2.95-3.30 (m, 1.2H), 2.32-2.65 (m, 4.8H) | 385.0, 387.0 [M + H]⁺ |
| 107 | 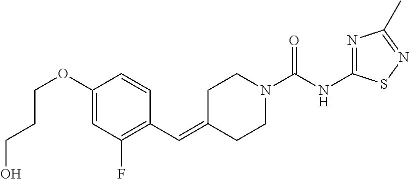 [SMILES: Cc1nsc(NC(=O)N2CCC(=Cc3ccc(OCCCO)cc3F)CC2)n1] | 1 | Ex 101 | DMSO-d₆ + D₂O: 7.16 (t, J = 8.8 Hz, 1H), 6.73-6.80 (m, 2H), 6.21 (s, 1H), 4.02 (t, J = 6.4 Hz, 2H), 3.51-3.62 (m, 6H), 2.37 (s, 3H), 2.27-2.36 (m, 4H), 1.83 (m, 2H) | 407.1 [M + H]⁺ |
| 108 | 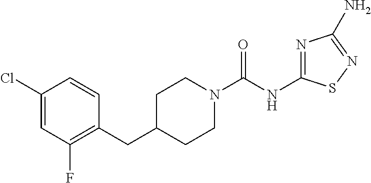 [SMILES: Nc1nsc(NC(=O)N2CCC(Cc3ccc(Cl)cc3F)CC2)n1] | 1 | Ex 70 | DMSO-d₆ + D₂O: 7.27-7.34 (m, 2H), 7.20 (dd, J1 = 2.0 Hz, J2 = 8.4 Hz, 1H), 4.07-4.11 (m, 2H), 2.78-2.84 (m, 2H), 2.52-2.54 (m, 2H), 1.72-1.75 (m, 1H), 1.55-1.58 (m, 2H), 1.04-1.14 (m, 2H) | 370.2, 372.2 [M + H]⁺ |
| 109 | 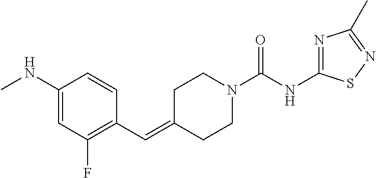 [SMILES: CNc1ccc(C=C2CCN(C(=O)Nc3nc(C)ns3)CC2)c(F)c1] | 1 | Ex 101 | DMSO-d₆ + D₂O: 6.99 (t, J = 8.4 Hz, 1H), 6.31-6.41 (m, 2H), 6.17 (s, 1H), 3.49-3.62 (m, 4H), 2.66 (s, 3H), 2.37 (s, 3H), 2.30-2.36 (m, 4H) | 362.0 [M + H]⁺ |
| 110 | 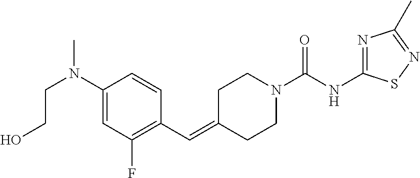 [SMILES: Cc1nsc(NC(=O)N2CCC(=Cc3ccc(N(C)CCO)cc3F)CC2)n1] | 1 | Ex 101 | DMSO-d₆ + D₂O: 7.04 (t, J = 8.8 Hz, 1H), 6.44-6.51 (m, 2H), 6.17 (s, 1H), 3.49-3.64 (m, 6H), 3.38 (t, J = 6.0 Hz, 2H), 2.90 (s, 3H), 2.36 (s, 3H), 2.31-2.34 (m, 4H) | 406.2 [M + H]⁺ |
| 111 | 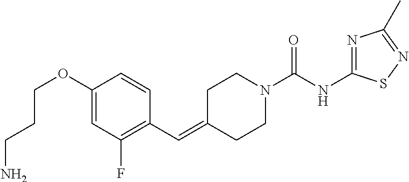 [SMILES: Cc1nsc(NC(=O)N2CCC(=Cc3ccc(OCCCN)cc3F)CC2)n1] | 1 | Ex 101 | DMSO-d₆ + D₂O: 7.19 (t, J = 8.8 Hz, 1H), 6.75-6.84 (m, 2H), 6.22 (s, 1H), 4.05 (t, J = 6.0 Hz, 2H), 3.49-3.62 (m, 4H), 2.94 t, J = 7.4 Hz, 2H), 2.37 (s, 3H), 2.27-2.35 (m, 4H), 1.98 (m, 2H) | 406.2 [M + H]⁺ |
| 112 | 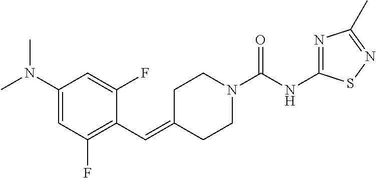 [SMILES: Cc1nsc(NC(=O)N2CCC(=Cc3c(F)cc(N(C)C)cc3F)CC2)n1] | 1 | Ex 88 | DMSO-d₆ + D₂O: 6.36 (s, 1H), 6.32 (s, 1H), 5.94 (s, 1H), 3.57-3.59 (m, 2H), 3.48-3.51 (m, 2H), 2.88 (s, 6H), 2.30-2.40 (m, 5H), 2.06-2.10 (m, 2H) | 394.1 [M + H]⁺ |
| 113 | 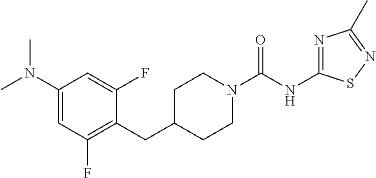 [SMILES: Cc1nsc(NC(=O)N2CCC(Cc3c(F)cc(N(C)C)cc3F)CC2)n1] | 1 | Ex 88, 27 | DMSO-d₆ + D₂O: 6.33 (s, 1H), 6.29 (s, 1H), 4.09-4.13 (m, 2H), 2.85 (s, 6H), 2.70-2.83 (m, 2H), 2.41 (d, J = 6.8 Hz, 2H), 2.35 (s, 3H), 1.65-1.68 (m, 1H), 1.56-1.60 (m, 2H), 0.95-1.15 (m, 2H) | 396.1 [M + H]⁺ |
| 114 | 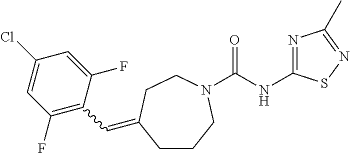 [SMILES: Cc1nsc(NC(=O)N2CCCC(=Cc3c(F)cc(Cl)cc3F)CC2)n1] | 1 | Ex 88 | DMSO-d₆ + D₂O: 7.00-7.30 (m, 2H), 5.85-5.95 (m, 1H), 3.51-3.64 (m, 4H), 2.10-2.60 (m, 7H), 1.54-17.1 (m, 2H) | 399.1, 401.1 [M + H]⁺ |
| 115 | 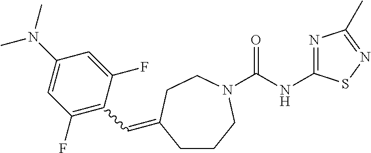 [SMILES: Cc1nsc(NC(=O)N2CCCC(=Cc3c(F)cc(N(C)C)cc3F)CC2)n1] | 1 | Ex 88 | DMSO-d₆ + D₂O: 6.21-6.31 (m, 2H), 5.75-5.89 (m, 1H), 3.51-3.63 (m, 4H), 2.85 (s, 6H), 2.09-2.56 (m, 7H), 1.55-1.70 (m, 2H) | 408.0 [M + H]⁺ |
| 116 | 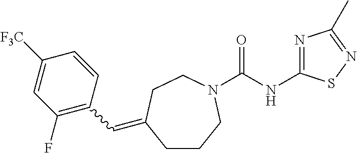 [SMILES: Cc1nsc(NC(=O)N2CCCC(=Cc3ccc(C(F)(F)F)cc3F)CC2)n1] | 1 | Ex 88 | DMSO-d₆: 11.29-11.58 (m, 1H), 7.33-7.64 (m, 3H), 6.28-6.33 (m, 1H), 3.60-3.66 (m, 4H), 2.30-2.63 (m, 7H), 1.69-1.74 (m, 2H) | 415.0 [M + H]⁺ |
| 117 | 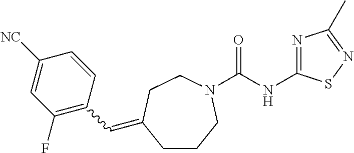 [SMILES: Cc1nsc(NC(=O)N2CCCC(=Cc3ccc(C#N)cc3F)CC2)n1] | 1 | Ex 88 | DMSO-d₆ + D₂O: 7.36-7.74 (m, 3H), 6.24-6.31 (m, 1H), 3.49-3.65 (m, 4H), 2.29-2.61 (m, 7H), 1.65-1.71 (m, 2H) | 372.1 [M + H]⁺ |
| 118 | 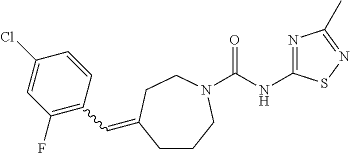 [SMILES: Cc1nsc(NC(=O)N2CCCC(=Cc3ccc(Cl)cc3F)CC2)n1] | 1 | Ex 88 | DMSO-d₆ + D₂O: 7.17-7.35 (m, 3H), 6.15-6.22 (m, 1H), 3.65 (m, 4H), 2.23-2.56 (m, 7H) 1.64-1.71 (m, 2H) | 381.0, 383.0 [M + H]⁺ |
| 119 | 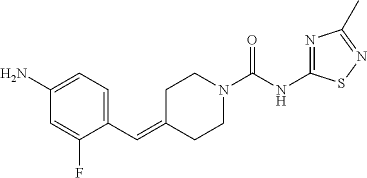 [SMILES: Cc1nsc(NC(=O)N2CCC(=Cc3ccc(N)cc3F)CC2)n1] | 1 | Ex. 101 | DMSO-d₆: 11.74 (s, 1H), 6.97 (t, J = 8.4 Hz, 1H), 6.41-6.47 (m, 2H), 6.16 (s, 1H), 3.60 (t, J = 5.6 Hz, 2H), 3.53 (t, J = 5.6 Hz, 2H), 2.38 (s, 3H), 2.30-2.34 (m, 4H) | 348.5 [M + H]⁺ |
| 120 | 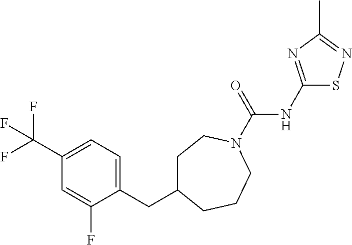 [SMILES: Cc1nsc(NC(=O)N2CCCC(Cc3ccc(C(F)(F)F)cc3F)CC2)n1] | 1 | Ex. 88, 27 | DMSO-d₆: 11.47 (s, 1H), 7.61 (d, J = 10 Hz, 1H), 7.50-7.55 (m, 2H), 3.25-3.70 (m, 4H), 2.63 (d, J = 7.2 Hz), 2.37 (s, 3H), 1.63-1.85 (m, 4H), 1.54 (m, 1H), 1.17-1.31 (m, 2H) | 417.6 [M + H]⁺ |
| 121 | 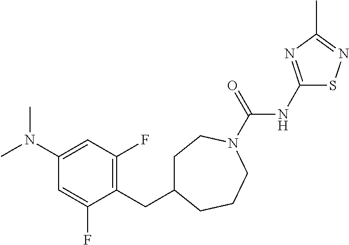 [SMILES: Cc1nsc(NC(=O)N2CCCC(Cc3c(F)cc(N(C)C)cc3F)CC2)n1] | 1 | Ex. 88, 27 | DMSO-d₆ + D₂O: 6.31 (d, J = 11.2 Hz, 2H), 3.26-3.67 (m, 4H), 2.85 (s, 6H), 2.36-2.39 (m, 5H), 1.54-1.83 (m, 4H), 1.50 (m, 1H), 1.11-1.25 (m, 2H) | 410.4 [M + H]⁺ |
| 122 | 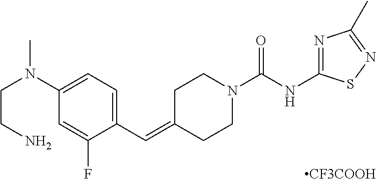 [SMILES: Cc1nsc(NC(=O)N2CCC(=Cc3ccc(N(C)CCN)cc3F)CC2)n1 ; "·CF3COOH"] | 1 | Ex. 101 | DMSO-d₆: 11.69 (s, 1H), 7.76 (br, 3H), 7.09 (t, J = 8.8 Hz, 1H), 6.55-6.60 (m, 2H), 6.20 (s, 1H), 3.62 (t, J = 5.2 Hz, 2H), 3.49-3.53 (m, 4H), 2.96 (dd, J1 = 12.4 Hz, J2 = 6.0 Hz, 2H), 2.91 (s, 3H), 2.38 (s, 3H), 2.32-2.36 (m, 4H) | 405.5 [M + H]⁺ |
| 123 | 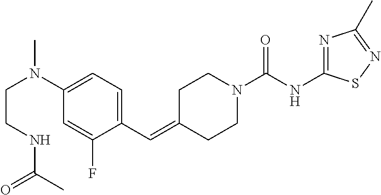 [SMILES: CC(=O)NCCN(C)c1ccc(C=C2CCN(C(=O)Nc3nc(C)ns3)CC2)c(F)c1] | 1 | Ex. 122 | DMSO-d₆: 11.70 (s, 1H), 7.93 (t, J = 5.6 Hz, 1H), 7.05 (t, J = 8.8 Hz), 6.49-6.53 (m, 2H), 6.19 (s, 1H), 3.61 (t, J = 5.2 Hz, 2H), 3.53 (t, J = 5.2 Hz, 2H), 3.34 (t, J = 6.4 Hz, 2H), 3.17 (dd, J1 = 12.8 Hz, J2 = 6.4 Hz, 2H), 2.90 (s, 3H), 2.49 (s, 3H), 2.33-2.38 (m, 4H), 1.76 (s, 3H) | 447.2 [M + H]⁺ |
| 124 | 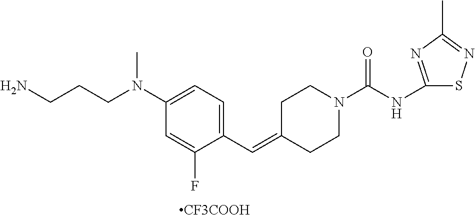 [SMILES: Cc1nsc(NC(=O)N2CCC(=Cc3ccc(N(C)CCCN)cc3F)CC2)n1 ; "·CF3COOH"] | 1 | Ex. 101, 122 | DMSO-d₆: 11.69 (s, 1H), 7.67 (br., 3H), 7.06 (t, J = 8.8 Hz, 1H), 6.51-6.54 (m, 2H), 6.19 (s, 1H), 3.61 (t, J = 5.6 Hz, 2H), 3.53 (t, J = 5.6 Hz, 2H), 3.39 (t, J = 7.2 Hz, 2H), 2.88 (s, 3H), 2.82 (dd, J1 = 15.2 Hz, J2 = 6.4 Hz, 2H), 2.38 (s, 3H), 2.33-2.36 (m, 4H), 1.72-1.80 (m, 2H) | 419.4 [M + H]⁺ |
| 125 | 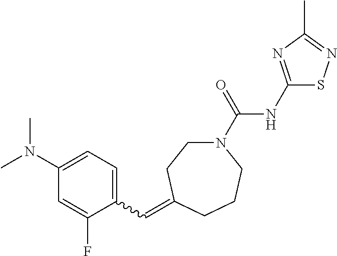 [SMILES: Cc1nsc(NC(=O)N2CCCC(=Cc3ccc(N(C)C)cc3F)CC2)n1] | 1 | Ex. 88 | DMSO-d₆ + D₂O: 6.92-7.05 (m, 1H), 6.34-6.48 (m, 2H), 6.10-6.17 (m, 1H), 3.51-3.60 (m, 4H), 2.85 (s, 6H), 2.27-2.37 (m, 7H), 1.68 (m, 2H) | 390.4 [M + H]⁺ |
| 126 | 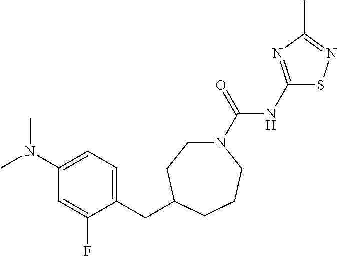 [SMILES: Cc1nsc(NC(=O)N2CCCC(Cc3ccc(N(C)C)cc3F)CC2)n1] | 1 | Ex. 88, 27 | DMSO-d₆ + D₂O: 6.98 (t, J = 8.8 Hz, 1H), 6.37-6.45 (m, 2H), 3.24-3.67 (m, 4H), 2.82 (s, 6H), 2.38-2.45 (m, 5H), 1.57-1.82 (m, 4H), 1.55 (m, 1H), 1.08-1.26 (m, 2H) | 392.1 [M + H]⁺ |
| 127 | 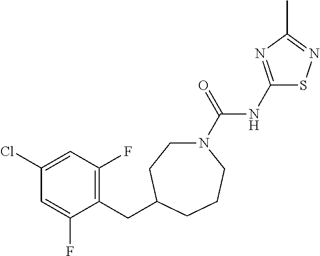 [SMILES: Cc1nsc(NC(=O)N2CCCC(Cc3c(F)cc(Cl)cc3F)CC2)n1] | 1 | Ex. 88, 27 | DMSO-d₆: 11.47 (s, 1H), 7.30-7.34 (m, 2H), 3.37-3.71 (m, 4H), 2.52 (m, 1H), 2.38 (s, 3H), 1.65-1.86 (m, 4H), 1.52 (m, 1H), 1.17-1.33 (m, 2H) | 401.0, 402.8 [M + H]⁺ |
| 128 | 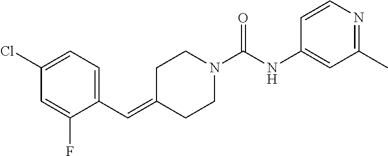 [SMILES: Cc1cc(NC(=O)N2CCC(=Cc3ccc(Cl)cc3F)CC2)ccn1] | 1 | Ex. 39, 138 | DMSO-d₆ + D₂O: 8.39 (d, J = 6.8 Hz, 1H), 7.70-7.76 (m, 2H), 7.40 (dd, J1 = 2.0 Hz, J2 = 10.0 Hz, 1H), 7.31 (dd, J1 = J2 = 8.0 Hz, 1H), 7.26 (dd, J1 = 8.4 Hz, J2 = 2.0 Hz, 1H), 6.28 (s, 1H), 3.60 (m, 2H), 3.51 (m, 2H), 2.56 (s, 3H), 2.42 (m, 2H), 2.33 (m, 2H) | 360.3, 362.2 [M + H]⁺ |
| 129 | 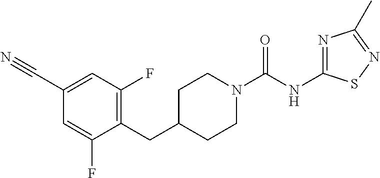 [SMILES: Cc1nsc(NC(=O)N2CCC(Cc3c(F)cc(C#N)cc3F)CC2)n1] | 1 | Ex. 95, 37, 27 | DMSO-d₆ + D₂O: 7.69-7.71 (m, 2H), 4.09-4.13 (m, 2H), 2.80-2.83 (m, 2H), 2.63 (d, J = 7.2 Hz, 2H), 2.35 (s, 3H), 1.76-1.78 (m, 1H), 1.56-1.59 (m, 2H), 1.10-1.16 (m, 2H) | 378.3 [M + H]⁺ |
| 130 | 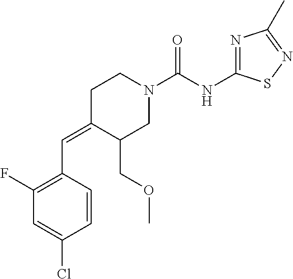 [SMILES: COCC1CN(C(=O)Nc2nc(C)ns2)CC/C1=C/c1ccc(Cl)cc1F] | 1 | Ex. 95 | DMSO-d₆ + D₂O: 7.38-7.47 (m, 2H), 7.27 (dd, J1 = 2.4 Hz, J2 = 8.4 Hz, 1H), 6.34 (s, 1H), 4.25-4.36 (m, 2H), 3.28-3.42 (m, 2H), 3.13 (s, 3H), 2.86-2.89 (m, 3H), 2.44 (m, 1H), 2.37 (s, 3H), 2.28 (m, 1H) | 411.2, 413.2 [M + H]⁺ |
| 131 | 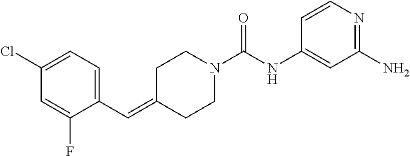 [SMILES: Nc1cc(NC(=O)N2CCC(=Cc3ccc(Cl)cc3F)CC2)ccn1] | 1 | Ex. 39, 138 | DMSO-d₆ + D₂O: 7.70 (d, J = 7.6 Hz, 1H), 7.40 (dd, J1 = 2.0 Hz, J2 = 10.0 Hz, 1H), 7.21-7.33 (m, 3H), 6.84 (dd, J = 2.0 Hz, J2 = 7.6 Hz, 1H), 6.27 (s, 1H), 3.56 (m, 2H), 3.47 (m, 2H), 2.39 (m, 2H), 2.30 (m, 2H) | 361.2, 363.1 [M + H]⁺ |
| 132 | 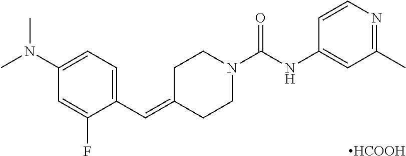 | 1 | Ex. 47, 138 | DMSO-d₆ + D₂O: 8.15 (d, J = 6.0 Hz, 1H), 8.12 (s, 1H), 7.36 (s, 1H), 7.30 (dd, J1 = 2.0 Hz, J2 = 5.6 Hz, 1H), 7.06 (dd, J1 = J2 = 8.8 Hz, 1H), 6.42-6.51 (m, 2H), 6.17 (s, 1H), 3.52 (m, 2H), 3.44 (m, 2H), 2.87 (s, 6H), 2.36 (s, 3H), 2.30-2.35 (m, 4H) | 369.3 [M + H]⁺ |
| 133 | 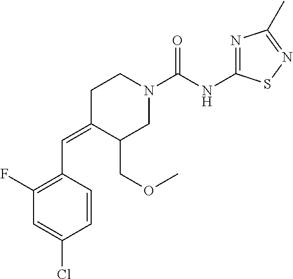 [SMILES: COCC1CN(C(=O)Nc2nc(C)ns2)CC/C1=C/c1ccc(Cl)cc1F] | 1 | Ex: 39, 95 | DMSO-d₆ + D₂O: 7.42 (dd, J1 = 10.4 Hz, J2 = 2.0 Hz, 1H), 7.23-7.31 (m, 2H), 6.42 (s, 1H), 4.45 (d, J = 13.6 Hz, 1H), 4.29 (d, J = 11.2 Hz, 1H), 4.05 (s, 1H), 3.18-3.26 (m, 2H), 2.91-3.02 (m, 2H), 2.56 (m, 1H), 2.36 (s, 3H), 2.26 (d, J = 13.6 Hz, 1H), 0.87 (t, J = 6.4 Hz, 3H) | 411.2, 413.0 [M + H]⁺ |
| 134 | 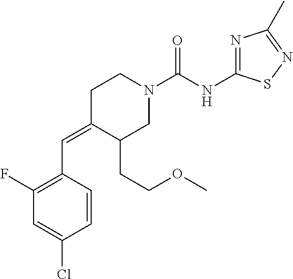 [SMILES: COCCC1CN(C(=O)Nc2nc(C)ns2)CC/C1=C/c1ccc(Cl)cc1F] | 1 | Ex: 39, 95 | DMSO-d₆ + D₂O: 7.39 (dd, J1 = 10.0 Hz, J2 = 2.0 Hz, 1H), 7.34 (dd, J1 = J2 = 8.4 Hz, 1H), 7.25 (dd, J1 = 8.4 Hz, J2 = 2.0 Hz, 1H), 6.26 (s, 1H), 4.19-4.37 (m, 2H), 2.79-3.26 (m, 8H), 2.25-2.49 (m, 2H), 2.36 (s, 3H), 1.60 (m, 2H) | 425.2, 427.0 [M + H]⁺ |
| 135 | 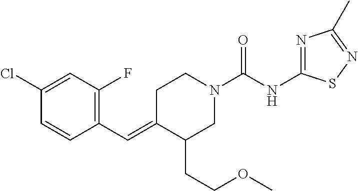 [SMILES: COCCC1CN(C(=O)Nc2nc(C)ns2)CC/C1=C\c1ccc(Cl)cc1F] | 1 | Ex: 39, 95 | DMSO-d₆ + D₂O: 7.39 (dd, J1 = 10.4 Hz, J2 = 2.0 Hz, 1H), 7.24-7.32 (m, 2H), 6.21 (s, 1H), 3.90-4.01 (m, 2H), 3.03-3.59 (m, 4H), 3.20 (s, 3H), 2.26-2.56 (m, 3H, H), 2.37 (s, 3H, H-1), 1.67 (m, 2H) | 425.3, 427.1 [M + H]⁺ |
| 136 | 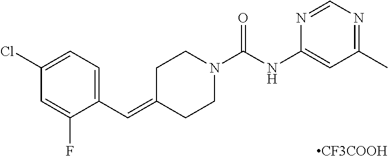 [SMILES: Cc1cc(NC(=O)N2CCC(=Cc3ccc(Cl)cc3F)CC2)ncn1 ; "·CF3COOH"] | 1 | Ex. 39, 138 | DMSO-d₆ + D₂O: 8.84 (s, 1H), 7.73 (s, 1H), 7.40 (dd, J1 = 2.0 Hz, J2 = 10.0 Hz, 1H), 7.24-7.33 (m, 2H), 6.26 (s, 1H), 3.53-3.56 (m, 2H), 3.48 (t, J = 5.6 Hz, 2H), 2.45 (s, 3H), 2.39 (t, J = 5.6 Hz, 2H), 2.30 (t, J = 5.6 Hz, 2H) | 361.5, 363.2 [M + H]⁺ |
| 137 | 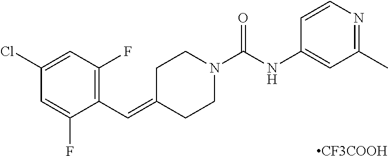 [SMILES: Cc1cc(NC(=O)N2CCC(=Cc3c(F)cc(Cl)cc3F)CC2)ccn1 ; "·CF3COOH"] | 1 | Ex. 26, 144 | DMSO-d₆ + D₂O: 8.38 (d, J = 6.8 Hz, 1H), 7.70-7.74 (m, 2H), 7.35 (d, J = 7.2 Hz, 2H), 6.04 (s, 1H), 3.62 (m, 2H), 3.51 (m, 2H), 2.55 (s, 3H), 2.45 (m, 2H), 2.12 (m, 2H) | 378.0, 380.0 [M + H]⁺ |
| 138 | 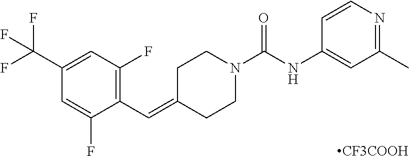 [SMILES: Cc1cc(NC(=O)N2CCC(=Cc3c(F)cc(C(F)(F)F)cc3F)CC2)ccn1 ; "·CF3COOH"] | 1 | Ex. 41 | DMSO-d₆ + D₂O: 8.39 (d, J = 6.8 Hz, 1H), 7.70-7.77 (m, 2H), 7.62 (d, J = 6.8 Hz, 2H), 6.13 (s, 1H), 3.62 (m, 2H), 3.50 (m, 2H), 2.56 (s, 3H), 2.46 (m, 2H), 2.15 (m, 2H) | 412.1 [M + H]⁺ |
| 139 | 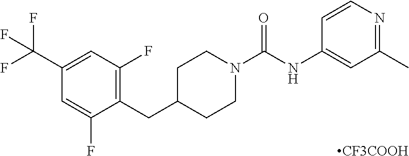 [SMILES: Cc1cc(NC(=O)N2CCC(Cc3c(F)cc(C(F)(F)F)cc3F)CC2)ccn1 ; "·CF3COOH"] | 1 | Ex. 40, 138 | DMSO-d₆ + D₂O: 8.38 (d, J = 6.8 Hz, 1H), 7.69-7.72 (m, 2H), 7.58 (d, J = 6.8 Hz, 2H), 4.07 (m, 2H), 2.84 (m, 2H), 2.65 (d, J = 7.2 Hz, 2H), 2.55 (s, 3H), 1.81 (m, 1H), 1.64 (m, 2H), 1.19 (m, 2H). | 414.1 [M + H]⁺ |
| 140 | 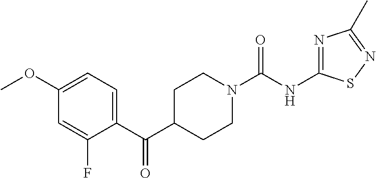 [SMILES: COc1ccc(C(=O)C2CCN(C(=O)Nc3nc(C)ns3)CC2)c(F)c1] | 4 | Ex. 141 | DMSO-d₆ + D₂O: 7.78 (dd, J1 = J2 = 8.8 Hz, 1H), 6.88-6.94 (m, 2H), 4.16 (m, 2H), 3.83 (s, 3H), 3.37 (m, 1H), 3.04 (m, 2H), 2.36 (s, 3H), 1.83 (m, 2H), 1.44 (m, 2H) | 379.0 [M + H]⁺ |
| 141 | 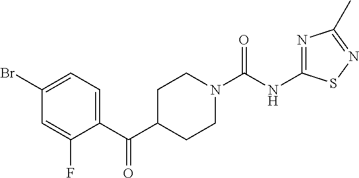 [SMILES: Cc1nsc(NC(=O)N2CCC(C(=O)c3ccc(Br)cc3F)CC2)n1] | 4 | Ex. 77, 27 | DMSO-d₆ + D₂O: 7.69-7.75 (m, 2H), 7.56 (dd, J1 = 2.0 Hz, J2 = 8.4 Hz, 1H), 4.14 (m, 2H), 3.38 (m, 1H), 3.04 (m, 2H), 2.36 (s, 3H), 1.84 (m, 2H), 1.43 (m, 2H) | 427.2, 429.4 [M + H]⁺ |
| 142 | 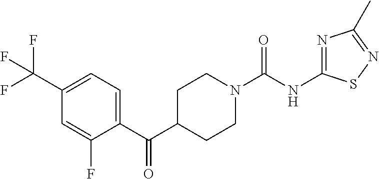 [SMILES: Cc1nsc(NC(=O)N2CCC(C(=O)c3ccc(C(F)(F)F)cc3F)CC2)n1] | 4 | Ex. 77, 27 | DMSO-d₆ + D₂O: 7.95 (dd, J1 = J2 = 8.0 Hz, 1H), 7.84 (d, J = 10.8 Hz, 1H), 7.72 (d, J = 8.0 Hz, 1H), 4.14 (m, 2H), 3.41 (m, 1H), 3.05 (m, 2H), 2.36 (s, 3H), 1.87 (m, 2H), 1.45 (m, 2H) | 417.3 [M + H]⁺ |
| 143 | 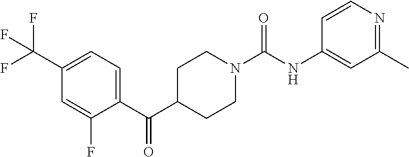 [SMILES: Cc1cc(NC(=O)N2CCC(C(=O)c3ccc(C(F)(F)F)cc3F)CC2)ccn1] | 4 | Ex. 142, 144 | DMSO-d₆ + D₂O: 8.19 (d, J = 6.0 Hz, 1H), 7.96 (dd, J1 = J2 = 8.0 Hz, 1H), 7.85 (d, J = 10.4 Hz, 1H), 7.72 (d, J = 8.0 Hz, 1H), 7.40 (d, J = 2.0 Hz, 1H), 7.36 (dd, J1 = 2.0 Hz, J2 = 6.0 Hz, 1H), 4.08 (m, 2H), 3.40 (m, 1H), 2.99 (m, 2H), 2.39 (s, 3H), 1.87 (m, 2H), 1.46 (m, 2H) | 410.3 [M + H]⁺ |
| 144 | 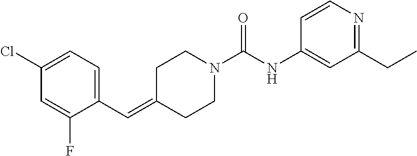 [SMILES: CCc1cc(NC(=O)N2CCC(=Cc3ccc(Cl)cc3F)CC2)ccn1] | 1 | Ex. 39 | DMSO-d₆ + D₂O: 8.16 (d, J = 5.6 Hz, 1H), 7.38 (dd, J1 = 2.0 Hz, J2 = 10.0 Hz, 1H), 7.23-7.33 (m, 4H), 6.25 (s, 1H), 3.54 (t, J = 5.6 Hz, 2H), 3.45 (t, J = 5.6 Hz, 2H), 2.62 (q, J = 7.6 Hz, 2H), 2.37 (t, J = 5.6 Hz, 2H), 2.28 (t, J = 5.6 Hz, 2H), 1.16 (t, J = 7.6 Hz, 3H) | 374.3, 376.2 [M + H]⁺ |
| 145 | 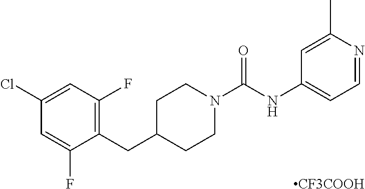 [SMILES: Cc1cc(NC(=O)N2CCC(Cc3c(F)cc(Cl)cc3F)CC2)ccn1 ; "·CF3COOH"] | 1 | Ex. 25, 144 | DMSO-d₆ + D₂O: 8.35 (d, J = 7.2 Hz, 1H, H-f), 7.65-7.71 (m, 2H), 7.31 (d, J = 7.2 Hz, 2H), 4.07 (m, 2H), 2.83 (m, 2H), 2.52-2.57 (m, 5H), 1.75 (m, 1H), 1.62 (m, 2H), 1.08-1.21 (m, 2H) | 380.5, 382.4 [M + H]⁺ |
| 146 | 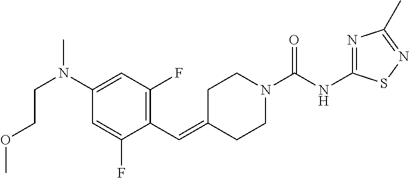 [SMILES: COCCN(C)c1cc(F)c(C=C2CCN(C(=O)Nc3nc(C)ns3)CC2)c(F)c1] | 1 | Ex. 26, 101, 122 | DMSO-d₆ + D₂O: 6.32-6.39 (m, 2H), 5.93 (s, 1H), 3.58-3.60 (m, 2H), 3.45-3.52 (m, 6H), 3.22 (s, 3H), 2.89 (s, 3H), 2.37 (s, 3H), 2.33-2.36 (m, 2H), 2.07-2.10 (m, 2H) | 438.4 [M + H]⁺ |
| 147 | 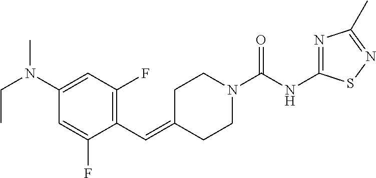 | 1 | Ex. 26, 101 | DMSO-d₆ + D₂O: : 6.17-6.22 (m, 2H), 5.90 (s, 1H), 3.56-3.59 (m, 2H), 3.48-3.50 (m, 2H), 3.00 (q, J = 7.2 Hz, 2H), 2.36 (s, 3H), 2.32-2.35 (m, 2H), 2.06-2.09 (m, 2H), 1.11 (t, J = 7.2 Hz, 3H) | 394.3 [M + H]⁺ |
| 148 | 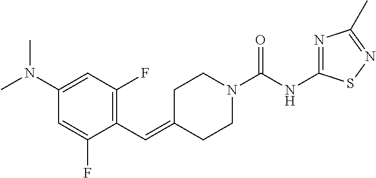 [SMILES: Cc1nsc(NC(=O)N2CCC(=Cc3c(F)cc(N(C)C)cc3F)CC2)n1] | 1 | Ex. 112, 70 | DMSO-d₆ + D₂O: 6.62 (s, 1H), 6.35 (d, J = 11.6 Hz, 2H), 5.94 (s, 1H), 3.53-3.55 (m, 2H), 3.44-3.46 (m, 2H), 2.88 (s, 6H), 2.34-2.36 (m, 2H), 2.08-2.10 (m, 2H), 2.24 (s, 3H) | 393.2 [M + H]⁺ |
| 149 | 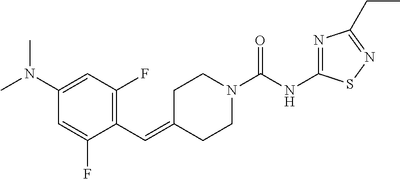 [SMILES: CCc1nsc(NC(=O)N2CCC(=Cc3c(F)cc(N(C)C)cc3F)CC2)n1] | 1 | Ex. 112, 70 | DMSO-d₆ + D₂O: 6.34 (d, J = 11.2 Hz, 2H), 5.93 (s, 1H), 3.57-3.59 (m, 2H), 3.51-3.53 (m, 2H), 2.88 (s, 6H), 2.70 (q, J = 7.6 Hz, 2H), 2.33-2.35 (m, 2H), 2.08-2.09 (m, 2H), 1.21 (t, J = 7.6 Hz, 3H) | 408.5 [M + H]⁺ |
| 150 | 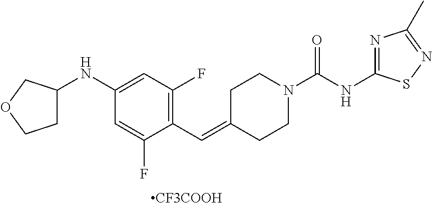 [SMILES: Cc1nsc(NC(=O)N2CCC(=Cc3c(F)cc(NC4CCOC4)cc3F)CC2)n1 ; "·CF3COOH"] | 1 | Ex. 26, 101 | DMSO-d₆ + D₂O: 6.21-6.27 (m, 2H), 5.90 (s, 1H), 3.92-3.97 (m, 1H), 3.75-3.85 (m, 2H), 3.67-3.72 (m, 1H), 3.55-3.61 (m, 2H), 3.47-3.51 (m, 3H), 2.31-2.37 (m, 5H), 2.11-2.20 (m, 1H), 2.05-2.10 (m, 2H), 1.67-1.74 (m, 1H) | 436.5 [M + H]⁺ |
| 151 | 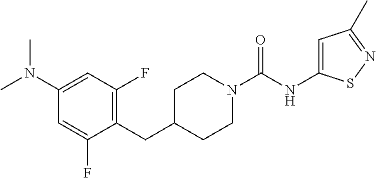 [SMILES: Cc1cc(NC(=O)N2CCC(Cc3c(F)cc(N(C)C)cc3F)CC2)sn1] | 1 | Ex. 113, 70 | DMSO-d₆ + D₂O: 6.66 (s, 1H), 6.31 (d, J = 11.2 Hz, 2H), 4.01-4.05 (m, 2H), 2.85 (s, 6H), 2.78-2.84 (m, 2H), 2.42 (d, J = 6.8 Hz, 2H), 2.29 (s, 3H), 1.58-1.67 (m, 3H), 1.05-1.08 (m, 2H) | 395.3 [M + H]⁺ |
| 152 | 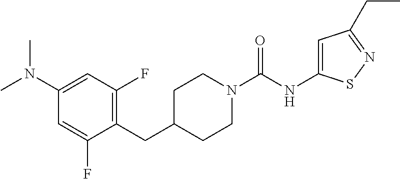 [SMILES: CCc1cc(NC(=O)N2CCC(Cc3c(F)cc(N(C)C)cc3F)CC2)sn1] | 1 | Ex. 113, 70 | DMSO-d₆ + D₂O: 6.32 (d, J = 11.6 Hz, 2H), 4.11-4.14 (m, 2H), 2.86 (s, 6H), 2.77-2.84 (m, 2H), 2.70 (q, J = 7.6 Hz, 2H), 2.41 (d, J = 6.8 Hz, 2H), 1.56-1.63 (m, 3H), 1.21 (t, J = 7.6 Hz, 3H), 1.05-1.08 (m, 2H) | 410.2 [M + H]⁺ |
| 153 | 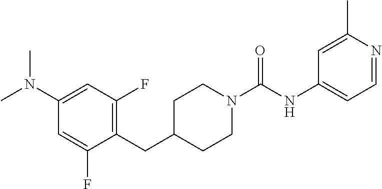 [SMILES: Cc1cc(NC(=O)N2CCC(Cc3c(F)cc(N(C)C)cc3F)CC2)ccn1] | 1 | Ex. 113, 70 | DMSO-d₆ + D₂O: 8.35 (d, J = 6.8 Hz, 1H), 7.67-7.69 (m, 2H), 6.30 (d, J = 11.6 Hz, 2H), 4.05-4.08 (m, 2H), 2.85 (s, 6H), 2.78-2.84 (m, 2H), 2.54 (s, 3H), 2.42 (d, J = 6.8 Hz, 2H), 1.60-1.68 (m, 3H), 1.08-1.12 (m, 2H) | 389.4 [M + H]⁺ |
| 154 | 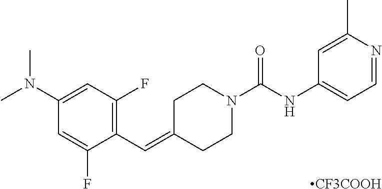 [SMILES: Cc1cc(NC(=O)N2CCC(=Cc3c(F)cc(N(C)C)cc3F)CC2)ccn1 ; "·CF3COOH"] | 1 | Ex. 112, 70 | DMSO-d₆ + D₂O: 8.37 (d, J = 11.2 Hz, 1H), 7.70-7.72 (m, 2H), 6.35 (d, J = 11.6 Hz, 2H), 5.96 (s, 1H), 3.56-3.57 (m, 2H), 3.46-3.49 (m, 2H), 2.88 (s, 6H), 2.55 (s, 3H), 2.37-2.39 (m, 2H), 2.21-2.23 (m, 2H) | 387.3 [M + H]⁺ |
| 155 | 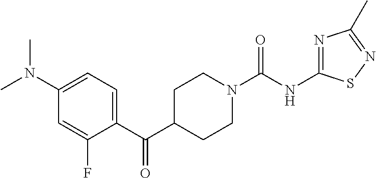 [SMILES: Cc1nsc(NC(=O)N2CCC(C(=O)c3ccc(N(C)C)cc3F)CC2)n1] | 4 | Ex. 141, 27 | DMSO-d₆ + D₂O: 7.68 (dd, J1 = J2 = 9.2 Hz, 1H), 6.58 (dd, J1 = 2.4 Hz, J2 = 9.2 Hz, 1H), 6.45 (dd, J1 = 2.4 Hz, J2 = 16.4 Hz, 1H), 4.17 (m, 2H), 3.32 (m, 1H), 2.95-3.06 (m, 8H), 2.36 (s, 3H), 1.80 (m, 2H), 1.44 (m, 2H) | 392.3 [M + H]⁺ |
| 156 | 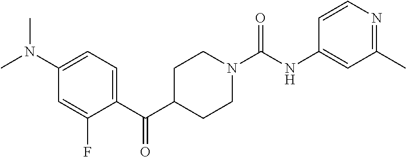 | 4 | Ex. 155, 144 | DMSO-d₆ + D₂O: 8.14 (d, J = 5.2 Hz, 1H), 7.68 (dd, J1 7--,' J2 = 8.8 Hz, 1H), 7.32 (d, J = 2.0 Hz, 1H), 7.28 (dd, J1 = 2.0 Hz, J2 = 5.2 Hz, 1H), 6.58 (dd, J1 = 2.4 Hz, J2 = 8.8 Hz, 1H), 6.44 (dd, J1 = 2.4 Hz, J2 = 16.0 Hz, 1H), 4.10 (m 2H), 3.30 (m, 1H), 2.99 (s, 6H), 2.94 (m, 2H), 2.34 (s, 3H), 1.78 (m, 2H), 1.44 (m, 2H) | 385.3 [M + H]⁺ |
| 157 | 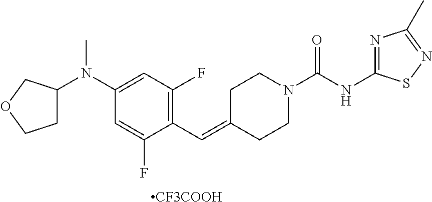 [SMILES: Cc1nsc(NC(=O)N2CCC(=Cc3c(F)cc(N(C)C4CCOC4)cc3F)CC2)n1 ; "·CF3COOH"] | 1 | Ex. 26, 101, 122 | DMSO-d₆ + D₂O: 6.45-6.52 (m, 2H), 5.94 (s, 1H), 4.52 (m, 1H), 3.89-3.95 (m, 1H), 3.68-3.72 (m, 2H), 3.55-3.60 (m, 3H), 3.48-3.52 (m, 2H), 2.75 (s, 3H), 2.33-2.38 (m, 5H), 2.16-2.26 (m, 1H), 2.05-2.12 (m, 2H), 1.72-1.80 (m, 1H) | 450.4 [M + H]⁺ |
| 158 | 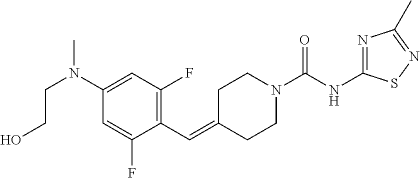 [SMILES: Cc1nsc(NC(=O)N2CCC(=Cc3c(F)cc(N(C)CCO)cc3F)CC2)n1] | 1 | Ex. 26, 101, 122 | DMSO-d₆ + D₂O: 6.31-6.38 (m, 2H), 5.93 (s, 1H), 3.57-3.60 (m, 2H), 3.49-3.52 (m, 4H), 3.34-3.37 (m, 2H), 2.90 (s, 3H), 2.37 (s, 3H), 2.33-2.36 (m, 2H), 2.07-2.10 (m, 2H) | 424.0 [M + H]⁺ |
| 159 | 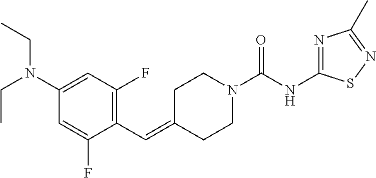 [SMILES: CCN(CC)c1cc(F)c(C=C2CCN(C(=O)Nc3nc(C)ns3)CC2)c(F)c1] | 1 | Ex. 147, 122 | DMSO-d₆ + D₂O: 6.25-6.32 (m, 2H), 5.92 (s, 1H), 3.57-3.59 (m, 2H), 3.48-3.50 (m, 2H), 3.28 (q, J = 7.2 Hz, 4H), 2.36 (s, 3H), 2.32-2.35 (m, 2H), 2.07-2.10 (m, 2H), 1.05 (t, J = 7.2 Hz, 6H) | 422.0 [M + H]⁺ |
| 160 | 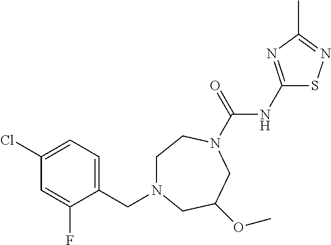 [SMILES: COC1CN(Cc2ccc(Cl)cc2F)CCN(C(=O)Nc2nc(C)ns2)C1] | 3 | Ex. 50, 27 | DMSO-d₆ + D₂O: 7.40-7.54 (m, 1H), 7.31 (d, J = 10 Hz, 1H), 7.12-7.28 (m, 1H), 3.85-3.94 (m, 1H), 3.70 (s, 2H), 3.37-3.61 (m, 4H), 3.18 (s, 3H), 2.65 (br., 4H), 2.36 (s, 3H) | 413.9, 415.9 [M + H]⁺ |
| 161 | 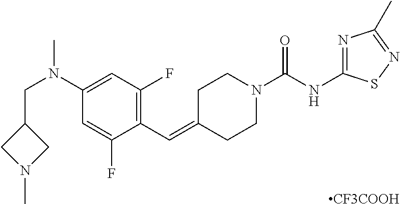 [SMILES: Cc1nsc(NC(=O)N2CCC(=Cc3c(F)cc(N(C)CC4CN(C)C4)cc3F)CC2)n1 ; "·CF3COOH"] | 1 | Ex. 26, 101, 122 | DMSO-d₆ + D₂O: 6.40-6.48 (m, 2H), 5.93 (s, 1H), 4.12-4.17 (m, 1H), 3.89-3.99 (m, 2H), 3.80 (m, 1H), 3.55-3.60 (m, 4H), 3.48-3.50 (m, 2H), 2.98-3.11 (m, 1H), 2.86 (m, 3H), 2.80 (m, 3H), 2.33-2.37 (m, 5H), 2.06-2.10 (m, 2H) | 463.5 [M + H]⁺ |
| 162 | 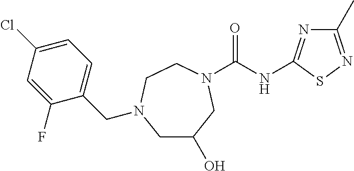 [SMILES: Cc1nsc(NC(=O)N2CCN(Cc3ccc(Cl)cc3F)CC(O)C2)n1] | 3 | Ex. 50, 27 | DMSO-d₆ + D₂O: 7.44 (br., 1H), 7.30 (d, J = 9.2 Hz, 1H), 7.19 (br, 1H), 3.68 (s, 2H), 3.30-3.92 (m, 5H), 2.49-2.75 (m, 4H), 2.35 (s, 3H) | 400.5, 402.2 [M + H]⁺ |
| 163 | 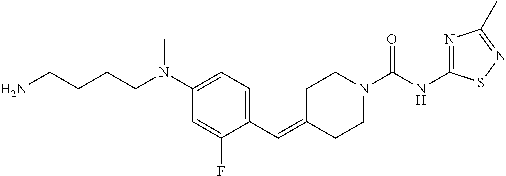 [SMILES: Cc1nsc(NC(=O)N2CCC(=Cc3ccc(N(C)CCCCN)cc3F)CC2)n1] | 1 | Ex. 101, 122 | DMSO-d₆: 7.06 (t, J = 8.8 Hz, 1H), 6.82 (br., 2H), 6.41-6.54 (m, 2H), 6.16 (s, 1H), 3.57 (m, 4H), 3.33 (t, J = 7.1 Hz, 2H), 2.90 (s, 3H), 2.72 (t, J = 6.9 Hz, 2H), 2.24-2.36 (m, 7H), 1.39-1.60 (m, 4H) | 433.1 [M + H]⁺ |
| 164 | 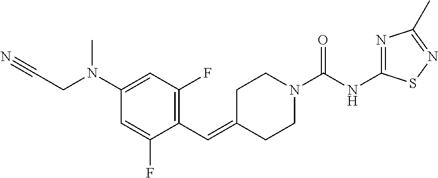 [SMILES: Cc1nsc(NC(=O)N2CCC(=Cc3c(F)cc(N(C)CC#N)cc3F)CC2)n1] | 1 | Ex. 88 | DMSO-d₆ + D₂O: 6.61-6.68 (m, 2H), 5.97 (s, 1H), 4.54 (s, 2H), 3.58-3.62 (m, 2H), 3.49-3.53 (m, 2H), 2.93 (s, 3H), 2.35-2.39 (m, 5H), 2.07-2.11 (m, 2H) | 419.4 [M + H]⁺ |
| 165 | 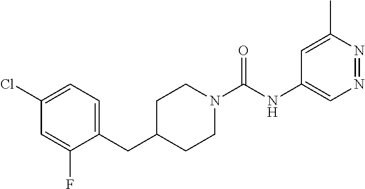 [SMILES: Cc1cc(NC(=O)N2CCC(Cc3ccc(Cl)cc3F)CC2)cnn1] | 1 | Ex. 9, 22 | DMSO-d₆ + D₂O: 9.07 (d, J = 2.4 Hz, 1H), 8.08 (d, J = 2.4 Hz, 1H), 7.34 (dd, J1 = 10.0 Hz, J2 = 2.0 Hz, 1H), 7.30 (dd, J1 = J2 = 8.4 Hz, 1H), 7.21 (dd, J1 = 8.4 Hz, J2 = 2.0 Hz, 1H), 4.09 (m, 2H), 2.86 (m, 2H), 2.63 (s, 3H), 2.55 (d, J = 6.8 Hz, 2H), 1.78 (m, 1H), 1.61 (m, 2H), 1.09 (m, 2H) | 363.5, 365.3 [M + H]⁺ |
| 166 | 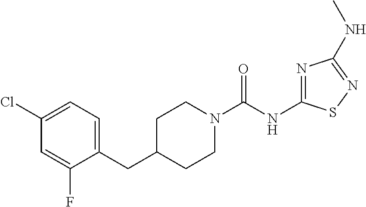 [SMILES: CNc1nsc(NC(=O)N2CCC(Cc3ccc(Cl)cc3F)CC2)n1] | 1 | Ex. 9, 22 | DMSO-d₆ + D₂O: 7.32 (dd, J1 = 10.0 Hz, J2 = 2.0 Hz, 1H), 7.29 (dd, J1 = J2 = 8.0 Hz, 1H), 7.20 (dd, J1 = 8.0 Hz, J2 = 2.0 Hz, 1H), 4.08 (m, 2H), 2.72-2.86 (m, 5H), 2.53 (m, 2H), 1.75 (m, 1H), 1.56 (m, 2H), 1.10 (m, 2H) | 384.2, 386.1 [M + H]⁺ |
| 167 | 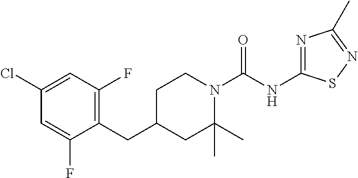 [SMILES: Cc1nsc(NC(=O)N2CCC(Cc3c(F)cc(Cl)cc3F)CC2(C)C)n1] | 1 | Ex. 27 | DMSO-d₆ + D₂O: 7.32 (d, J = 7.2 Hz, 2H), 3.72 (m, 1H), 3.05 (m, 1H), 2.45-2.56 (m, 2H), 2.34 (s, 3H), 1.83 (m, 1H), 1.62 (m, 1H), 1.39-1.49 (m, 4H), 1.14-1.32 (m, 5H) | 415.5, 417.3 [M + H]⁺ |
| 168 | 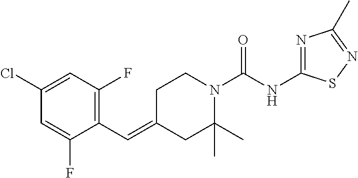 [SMILES: Cc1nsc(NC(=O)N2CC/C(=C\c3c(F)cc(Cl)cc3F)CC2(C)C)n1] | 1 | Ex. 88 | DMSO-d₆ + D₂O: 7.34 (d, J = 7.2 Hz, 2H), 6.05 (s, 1H), 3.61 (t, J = 6.0 Hz, 2H), 2.54 (s, 2H), 2.34 (s, 3H), 2.27 (t, J = 6.0 Hz, 2H), 1.44 (s, 6H) | 413.5, 415.4 [M + H]⁺ |
| 169 | 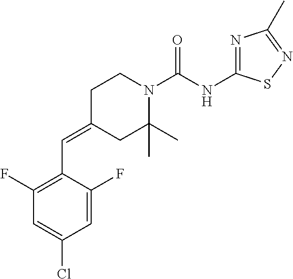 [SMILES: Cc1nsc(NC(=O)N2CC/C(=C/c3c(F)cc(Cl)cc3F)CC2(C)C)n1] | 1 | Ex. 88 | DMSO-d₆ + D₂O: 7.33 (d, J = 7.2 Hz, 2H), 6.06 (s, 1H), 3.63 (t, J = 6.0 Hz, 2H), 2.62 (t, J = 6.0 Hz, 2H), 2.36 (s, 3H), 2.23 (s, 2H), 1.29 (s, 6H) | 413.5, 415.4 [M + H]⁺ |
| 170 | 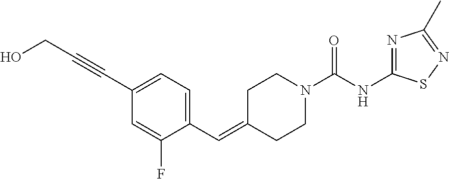 [SMILES: Cc1nsc(NC(=O)N2CCC(=Cc3ccc(C#CCO)cc3F)CC2)n1] | 1 | Ex. 39, 95 | DMSO-d₆ + D₂O: 11.74 (s, 1H), 7.21-7.40 (m, 3H), 6.33 (s, 1H), 5.38 (t, J = 6.0 Hz, 1H), 4.32 (d, J = 6.0 Hz, 2H), 3.65 (t, J = 5.9 Hz, 2H), 3.58 (t, J = 5.9 Hz, 2H), 2.30-2.47 (m, 7H) | 387.0 [M + H]⁺ |
| 171 | 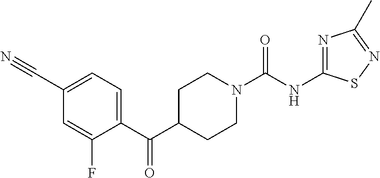 [SMILES: Cc1nsc(NC(=O)N2CCC(C(=O)c3ccc(C#N)cc3F)CC2)n1] | 4 | Ex. 142, 77, 27 | DMSO-d₆ + D₂O: 7.96 (dd, J1 = 10.4 Hz, J2 = 0.8 Hz, 1H), 7.89 (dd, J1 = 8.0 Hz, J2 = 7.2 Hz, 1H), 7.80 (dd, J1 = 8.0 Hz, J2 = 1.2 Hz, 1H), 4.12 (m, 2H), 3.39 (m, 1H), 3.04 (m, 2H), 2.35 (s, 3H), 1.85 (m, 2H), 1.43 (m, 2H) | 374.2 [M + H]⁺ |
| 172 | 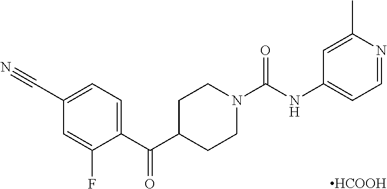 [SMILES: Cc1cc(NC(=O)N2CCC(C(=O)c3ccc(C#N)cc3F)CC2)ccn1 ; "·HCOOH"] | 4 | Ex. 142, 144 | DMSO-d₆ + D₂O: 8.14 (d, J = 6.0 Hz, 1H), 8.13 (s, 1H), 7.99 (dd, J1 = 10.4 Hz, J2 = 1.6 Hz, 1H), 7.90 (dd, J1 = 8.0 Hz, J2 = 7.2 Hz, 1H), 7.81 (dd, J1 = 8.0 Hz, J2 = 1.6 Hz, 1H), 7.32 (d, J = 2.0 Hz, 1H), 7.27 (dd, J1 = 6.0 Hz, J2 = 2.0 Hz, 1H), 4.06 (m, 2H), 3.37 (m, 1H), 2.97 (m, 2H), 2.35 (s, 3H), 1.84 (m, 2H), 1.43 (m, 2H) | 367.5 [M + H]⁺ |
| 173 | 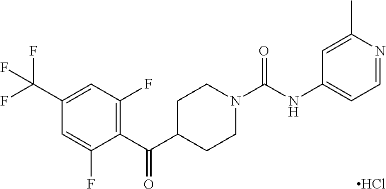 [SMILES: Cc1cc(NC(=O)N2CCC(C(=O)c3c(F)cc(C(F)(F)F)cc3F)CC2)ccn1 ; Cl] | 4 | Ex. 142, 144 | DMSO-d₆ + D₂O: 8.36 (d, J = 7.2 Hz, 1H), 7.79 (d, J = 7.2 Hz, 2H), 7.70 (s, 1H), 7.69 (dd, J1 = 7.2 Hz, J2 = 2.4 Hz, 1H), 4.10 (m, 2H), 3.19 (m, 1H), 3.03 (m, 2H), 2.54 (s, 3H), 1.93 (m, 2H), 1.49 (m, 2H) | 428.2 [M + H]⁺ |
| 174 | 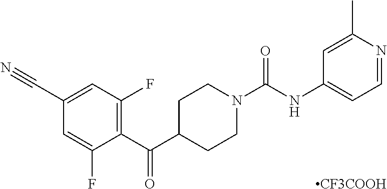 [SMILES: Cc1cc(NC(=O)N2CCC(C(=O)c3c(F)cc(C#N)cc3F)CC2)ccn1 ; "·CF3COOH"] | 4 | Ex. 142, 144 | DMSO-d₆ + D₂O: 8.37 (d, J = 6.8 Hz, 1H), 7.93 (d, J = 7.2 Hz, 2H), 7.67-7.70 (m, 2H), 4.08 (m, 2H), 3.18 (m, 1H), 3.03 (m, 2H), 2.55 (s, 3H), 1.92 (m, 2H), 1.49 (m, 2H) | 385.3 [M + H]⁺ |
| 175 | 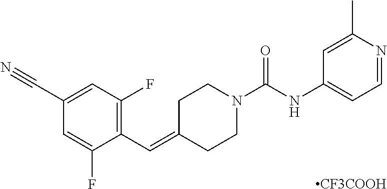 [SMILES: Cc1cc(NC(=O)N2CCC(=Cc3c(F)cc(C#N)cc3F)CC2)ccn1 ; "·CF3COOH"] | 1 | Ex. 95, 149 | DMSO-d₆ + D₂O: 8.38 (d, J = 7.2 Hz, 1H), 7.76 (d, J = 7.2 Hz), 7.72 (s, 1H), 7.71 (dd, J1 = 7.2 Hz, J2 = 2.4 Hz, 1H), 6.12 (s, 1H), 3.64 (m, 2H), 3.49 (m, 2H), 2.55 (s, 3H), 2.42-2.52 (m, 2H), 2.14 (m, 2H) | 369.3 [M + H]⁺ |
| 176 | 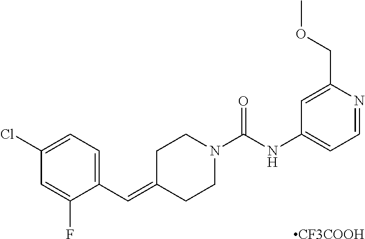 [SMILES: COCc1cc(NC(=O)N2CCC(=Cc3ccc(Cl)cc3F)CC2)ccn1 ; "·CF3COOH"] | 1 | Ex. 39, 144 | DMSO-d₆ + D₂O: 8.43 (d, J = 6.8 Hz, 1H), 7.86 (d, J = 2.0 Hz), 7.80 (dd, J1 = 2.0 Hz, J2 = 6.8 Hz, 1H), 7.39 (dd, J1 = 10.0 Hz, J2 = 2.0 Hz, 1H), 7.24-7.33 (m, 2H), 6.28 (s, 1H), 4.64 (s, 2H), 3.60 (m, 2H), 3.51 (m, 2H), 3.40 (s, 3H), 2.42 (m, 2H), 2.32 (m, 2H) | 390.0, 392.0 [M + H]⁺ |
| 177 | 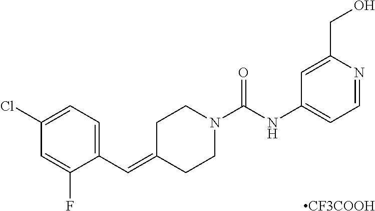 [SMILES: O=C(Nc1ccnc(CO)c1)N1CCC(=Cc2ccc(Cl)cc2F)CC1 ; "·CF3COOH"] | 1 | Ex. 144 | DMSO-d₆ + D₂O: 8.40 (d, J = 6.8 Hz, 1H), 7.89 (d, J = 2.0 Hz, 1H), 7.79 (dd, J1 = 6.8 Hz, J2 = 2.0 Hz, 1H), 7.40 (dd, J1 = 10.0 Hz, J2 = 2.0 Hz, 1H), 7.31 (dd, J1 = J2 = 8.4 Hz, 1H), 7.26 (dd, J1 = 8.4 Hz, J2 = 2.0 Hz, 1H), 6.28 (s, 1H), 4.71 (s, 2H), 3.55-3.66 (m, 2H), 3.51 (m, 2H), 2.42 (m, 2H), 2.32 (m, 2H) | 376.5, 378.2 [M + H]⁺ |
| 178 | 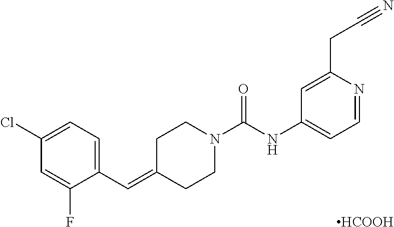 [SMILES: N#CCc1cc(NC(=O)N2CCC(=Cc3ccc(Cl)cc3F)CC2)ccn1 ; "·HCOOH"] | 1 | Ex. 177 | DMSO-d₆ + D₂O: 8.26 (d, J = 5.6 Hz, 1H), 7.57 (d, J = 2.0 Hz, 1H), 7.42 (dd, J1 = 5.6 Hz, J2 = 2.0 Hz, 1H), 7.39 (dd, J1 = 10.0 Hz, J2 = 2.0 Hz, 1H), 7.31 (dd, J1 = J2 = 8.4 Hz, 1H), 7.25 (d, J1 = 8.4 Hz, J2 = 2.0 Hz, 1H), 6.25 (s, 1H), 4.71 (s, 2H), 3.51-3.65 (m, 2H), 3.47 (m, 2H), 2.38 (m, 2H), 2.29 (m, 2H) | 385.1, 387.1 [M + H]⁺ |
| 179 | 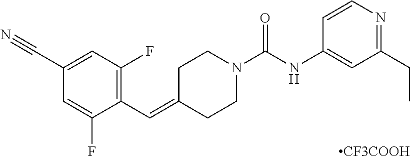 [SMILES: CCc1cc(NC(=O)N2CCC(=Cc3c(F)cc(C#N)cc3F)CC2)ccn1 ; "·CF3COOH"] | 1 | Ex. 95, 144 | DMSO-d₆ + D₂O: 8.38 (d, J = 7.2 Hz, 1H), 7.73-7.77 (m, 4H), 6.12 (s, 1H), 3.60 (t, J = 5.8 Hz, 2H), 3.49 (t, J = 5.8 Hz, 2H), 2.84 (q, J = 7.6 Hz, 2H), 2.49 (m, 2H, overlap DMSO), 2.14 (m, 2H), 1.24 (t, J = 7.6 Hz, 3H) | 383.4 [M + H]⁺ |
| 180 | 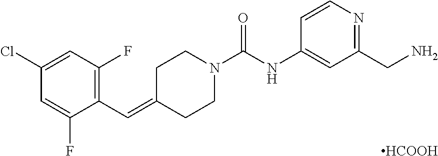 [SMILES: NCc1cc(NC(=O)N2CCC(=Cc3c(F)cc(Cl)cc3F)CC2)ccn1 ; "·HCOOH"] | 1 | Ex. 177, 144 | DMSO-d₆ + D₂O: 9.15 (s, 1H), 8.20-8.35 (m, 2H), 7.57 (s, 1H), 7.36-7.41 (m, 3H), 6.02 (s, 1H), 3.89 (s, 2H), 3.58 (t, J = 5.6 Hz, 2H), 3.47 (t, J = 5.6 Hz, 2H), 2.41 (t, J = 5.6 Hz, 2H), 2.10 (m, 2H) | 393.3, 395.2 [M + H]⁺ |
| 181 | 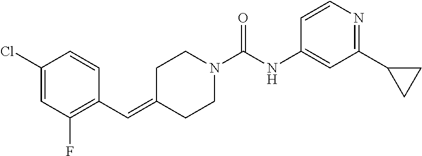 [SMILES: O=C(Nc1ccnc(C2CC2)c1)N1CCC(=Cc2ccc(Cl)cc2F)CC1] | 1 | Ex. 39, 144 | DMSO-d₆: 9.41 (s, 1H), 8.22 (m, 1H), 7.44 (m, 1H), 7.12-7.25 (m, 3H), 6.20 (s, 1H), 3.47-3.63 (m, 4H), 2.31-2.45 (m, 4H), 1.97-2.09 (m, 1H), 0.89-1.09 (m, 4H) | 387.1, 389.0 [M + H]⁺ |
| 182 | 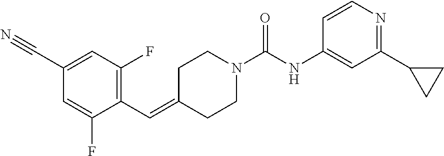 [SMILES: N#Cc1cc(F)c(C=C2CCN(C(=O)Nc3ccnc(C4CC4)c3)CC2)c(F)c1] | 1 | Ex. 95, 144 | DMSO-d₆: 8.98 (s, 1H), 8.15 (d, J = 5.7 Hz, 1H), 7.78-7.90 (m, 2H), 7.40 (d, J = 2.0 Hz, 1H), 7.27 (dd, J = 5.7, 2.1 Hz, 1H), 6.13 (s, 1H), 3.60 (t, J = 5.7 Hz, 2H), 3.48 (t, J = 5.7 Hz, 2H), 2.46 (t, J = 5.8 Hz, 2H), 2.13 (m, 2H), 1.96 (m, 1H), 0.83-0.95 (m, 4H) | 395.1 [M + H]⁺ |
| 183 | 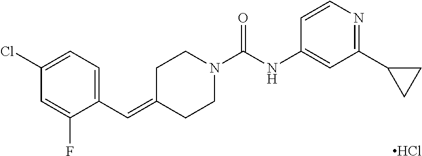 [SMILES: O=C(Nc1ccnc(C2CC2)c1)N1CCC(=Cc2ccc(Cl)cc2F)CC1 ; Cl] | 1 | Ex. 39, 144 | DMSO-d₆: 14.59 (s, 1H), 10.25 (s, 1H), 8.36 (d, J = 6.9 Hz, 1H), 7.83 (dd, J1 = 6.9, J2 = 2.3 Hz, 1H), 7.63 (d, J = 2.3 Hz, 1H), 7.45 (dd, J1 = 10.0, J2 = 2.0 Hz, 1H), 7.25-7.38 (m, 2H), 6.31 (s, 1H), 3.65 (t, J = 5.8 Hz, 2H), 3.57 (t, J = 5.8 Hz, 2H), 2.44 (t, J = 5.7 Hz, 2H), 2.35 (t, J = 5.7 Hz, 2H), 2.29 (m, 1H), 1.24-1.36 (m, 2H), 0.98-1.08 (m, 2H) | 385.9, 387.8 [M + H]⁺ |

### Preparation of Example 4: 4-[(4-aminophenyl)methyl]-N-(3-methyl-1,2,4-thiadiazol-5-yl)piperidine-1-carboxamide

### Preparation of N-(3-methyl-1,2,4-thiadiazol-5-yl)-4-[(4-nitrophenyl)methylene]piperidine-1-carboxamide

The title compound was prepared as a white solid following scheme 1 and in analogy to Example 95 and 27 using 1-(diethoxyphosphorylmethyl)-4-nitro-benzene [CAS 2609-49-6], tert-butyl 4-oxopiperidine-1-carboxylate [CAS 79099-07-3] and (4-nitrophenyl) N-(3-methyl-1,2,4-thiadiazol-5-yl)carbamate as starting materials.

¹H-NMR (400 MHz, CDCl₃) δ ppm: 8.21 (d, J=8.8 Hz, 2H), 7.34 (d, J=8.8 Hz, 2H), 6.49 (s, 1H), 3.69 (m, 2H), 3.59 (m, 2H), 2.61 (m, 2H), 2.54 (m, 2H), 2.51 (s, 3H).

### Preparation of 4-[(4-aminophenyl)methyl]-N-(3-methyl-1,2,4-thiadiazol-5-yl)piperidine-1-carboxamide

A mixture of N-(3-methyl-1,2,4-thiadiazol-5-yl)-4-[(4-nitrophenyl)methylene]piperidine-1-carboxamide (90 mg; 0.25 mmol) and 10% palladium on activated carbon (27 mg) in ethyl acetate (10 mL) was stirred under hydrogen atmosphere (1 bar) for 3 hours. The mixture was filtered and the filtrate was concentrated under vacuum. The resulting residue was purified by preparative HPLC to afford 4-[(4-aminophenyl)methyl]-N-(3-methyl-1,2,4-thiadiazol-5-yl)piperidine-1-carboxamide (60 mg) as a white solid.

### Preparation of Example 7: N-(3-methyl-1,2,4-thiadiazol-5-yl)-4-(p-tolylsulfanyl)piperidine-1-carboxamide

### Preparation of tert-butyl 4-methylsulfonyloxypiperidine-1-carboxylate

To a solution of tert-butyl 4-hydroxypiperidine-1-carboxylate (2000 mg; 9.94 mmol) [CAS 109384-19-2] in dichloromethane (20 mL) were slowly added triethylamine (1500 mg; 14.9 mmol) and methane sulfonyl chloride (1600 mg: 13.9 mmol) at 0° C. The reaction mixture was allowed to warm up to 15° C. and stirring was prolonged for 2 hours. Then, water (30 mL) was added and the mixture was extracted with dichloromethane (3×30 mL). The combined organic layers were washed with brine, dried over anhydrous Na₂SO₄, filtered and concentrated to dryness to give tert-butyl 4-methylsulfonyloxypiperidine-1-carboxylate (3000 mg) as a white solid.

MS m/z (+ESI): 280.1 [M+H]⁺.

### Preparation of tert-butyl 4-methylsulfonyloxypiperidine-1-carboxylate

To a stirred solution of tert-butyl 4-methylsulfonyloxypiperidine-1-carboxylate (3000 mg; 10.74 mmol) in acetonitrile (20 mL) was added 4-methylbenzenethiol (1600 mg; 12.89 mmol) [CAS 106-45-6] and potassium carbonate (2200 mg; 16.1 mmol). The reaction mixture was then heated to 75° C. and stirred for 12 hours. Then water (30 mL) was added and the mixture was extracted with dichloromethane (3×30 mL). The combined organic layers were washed with brine, dried with Na₂SO₄, filtered and concentrated to give the crude product, which was purified by column chromatography (silica gel; petroleum ether:ethyl acetate; 60:1 to 10:1; v/v) to afford tert-butyl 4-methylsulfonyloxypiperidine-1-carboxylate (2000 mg) as a colorless oil.

MS m/z (+ESI): 308.2 [M+H]⁺.

### Preparation of 4-methylsulfonyloxypiperidine

The title compound was prepared as a white solid following scheme 1 and 2 and in analogy to Example 27 using tert-butyl 4-methylsulfonyloxypiperidine-1-carboxylate as starting material.

MS m/z (+ESI): 208.1 [M+H]⁺.

### Preparation of N-(3-methyl-1,2,4-thiadiazol-5-yl)-4-(p-tolylsulfanyl)piperidine-1-carboxamide

The title compound was prepared as a white solid following scheme 1 and 2 and in analogy to Example 27 using 4-methylsulfonyloxypiperidine and (4-nitrophenyl) N-(3-methyl-1,2,4-thiadiazol-5-yl)carbamate as starting materials and after purification by column chromatography (silica gel; petroleum ether:ethyl acetate; 3:1; v/v).

### Preparation of Example 13: 4-[(4-methoxy-2-methyl-phenyl)methyl]-N-(3-methyl-1,2,4-thiadiazol-5-yl)piperidine-1-carboxamide

### Preparation of tert-butyl 4-[(4-chloro-2-methyl-phenyl)methyl]piperidine-1-carboxylate

The title compound was prepared as a white solid following scheme 1 and in analogy to Example 14 and 27 using 4-chloro-1-(chloromethyl)-2-methylbenzene [CAS 16470-09-0] and tert-butyl 4-oxopiperidine-1-carboxylate [CAS 79099-07-3] as starting materials.

MS m/z (+ESI): 324.1 [M+H]⁺.

### Preparation of tert-butyl 4-[(4-hydroxy-2-methyl-phenyl)methyl]piperidine-1-carboxylate

To a stirred solution of tert-butyl 4-[(4-chloro-2-methyl-phenyl)methyl]piperidine-1-carboxylate (100 mg; 0.28 mmol) in dioxane (4 mL) were added palladium(II) acetate (13 mg; 0.06 mmol), X-Phos (7 mg; 0.04 mmol) and potassium hydroxide (156 mg; 2.78 mmol). The reaction mixture was stirred for 1 hour at 125° C. in a microwave apparatus. The reaction mixture was treated with 1N HCl aqueous solution to adjust the pH at about 7. The product was extracted with ethyl acetate (3×20 mL), and the combined organic layers were washed with brine, dried over Na₂SO₄, filtered and concentrated to dryness. The residue was purified by column chromatography (silica gel; dichloromethane:methanol; 30:1; v/v) to afford tert-butyl 4-[(4-hydroxy-2-methyl-phenyl)methyl]piperidine-1-carboxylate (70 mg) as a light yellow solid.

MS m/z (+ESI): 306.2 [M+H]⁺.

### Preparation of tert-butyl 4-[(4-methoxy-2-methyl-phenyl)methyl]piperidine-1-carboxylate

Under nitrogen atmosphere, to a stirred solution of tert-butyl 4-[(4-hydroxy-2-methyl-phenyl)methyl]piperidine-1-carboxylate (500 mg; 1.56 mmol) in tetrahydrofuran (20 mL) was added methyl iodide (270 mg; 1.87 mmol), followed by the addition of 60% sodium hydride in mineral oil (125 mg; 3.6 mmol). After stirring for 2 hours, the reaction mixture was deactivated with a saturated aqueous solution of ammonium chloride (10 mL) and the product was extracted with ethyl acetate (3×30 mL). The combined organic layers were washed with brine, dried over Na₂SO₄, filtered and concentrated to dryness to afford crude tert-butyl 4-[(4-methoxy-2-methyl-phenyl)methyl]piperidine-1-carboxylate (500 mg) as a light yellow oil, which was used directly in the next step.

MS m/z (+ESI): 320.2 [M+H]⁺.

### Preparation 4-[(4-methoxy-2-methyl-phenyl)methyl]piperidine

The title compound was prepared as a white solid following scheme 1 and in analogy to Example 27 using tert-butyl 4-[(4-methoxy-2-methyl-phenyl)methyl]piperidine-1-carboxylate as starting material.

MS m/z (+ESI): 220.2 [M+H]⁺.

### Preparation of 4-[(4-methoxy-2-methyl-phenyl)methyl]-N-(3-methyl-1,2,4-thiadiazol-5-yl)piperidine-1-carboxamide

The title compound was prepared as a white solid following scheme 1 and in analogy to Example 27 using 4-[(4-methoxy-2-methyl-phenyl)methyl]piperidine and (4-nitrophenyl) N-(3-methyl-1,2,4-thiadiazol-5-yl)carbamate as starting materials and after purification by column chromatography (silica gel; petroleum ether:ethyl acetate; 2:1; v/v).

### Preparation of Example 19: 4-(4-chlorophenoxy)-N-(3-methyl-1,2,4-thiadiazol-5-yl)piperidine-1-carboxamide

### Preparation of tert-butyl 4-(4-chlorophenoxy)piperidine-1-carboxylate

To a solution of tert-butyl 4-hydroxypiperidine-1-carboxylate (1000 mg; 4.97 mmol) [CAS 109384-19-2], 4-chlorophenol (640 mg; 4.97 mmol) [CAS 106-48-9] and triphenylphosphine (1430 mg; 5.47 mmol) in tetrahydrofuran (20 mL) was added diethylazodicarboxylate (0.86 mL; 5.47 mmol) at 15° C. The mixture was stirred at 15° C. for 5 hours. The reaction mixture was partitioned between diethylether (60 mL) and 1M NaOH aqueous solution (30 mL). After decantation, the organic layer was washed with water and brine, dried over anhydrous Na₂SO₄, filtered and concentrated to dryness. The residue was purified by column chromatography (silica gel; petroleum ether:ethyl acetate; 30:1; v/v) to afford tert-butyl 4-(4-chlorophenoxy)piperidine-1-carboxylate (930 mg) as a white solid.

MS m/z (+ESI): 256.1 [M-t-Bu+H]⁺.

### Preparation of 4-(4-chlorophenoxy)piperidine

The title compound was prepared as a white solid following scheme 1 and 2 and in analogy to Example 27 using tert-butyl 4-(4-chlorophenoxy)piperidine-1-carboxylate as starting material.

MS m/z (+ESI): 212.1 [M+H]⁺.

### Preparation of 4-(4-chlorophenoxy)-N-(3-methyl-1,2,4-thiadiazol-5-yl)piperidine-1-carboxamide

The title compound was prepared as a white solid following scheme 1 and 2 and in analogy to Example 27 using 4-(4-chlorophenoxy)piperidine and (4-nitrophenyl) N-(3-methyl-1,2,4-thiadiazol-5-yl)carbamate as starting materials and after purification by preparative HPLC.

### Preparation of Example 22: 4-[(2,4-dichlorophenyl)methyl]-N-pyrimidin-4-yl-piperidine-1-carboxamide

### Preparation of (4-nitrophenyl) 4-[(2,4-dichlorophenyl)methyl]piperidine-1-carboxylate

To an ice-cold solution of 4-nitrophenyl chloroformate (190 mg; 0.91 mmol) in dichloromethane (8 mL) was added dropwise a solution of 4-[(2,4-dichlorophenyl)methyl]piperidine trifluoroacetic acid salt (300 mg; 0.83 mmol) (intermediate of Example 8) and triethylamine (0.26 mL; 1.82 mmol) in dichloromethane (3 mL). The reaction mixture was stirred for 0.5 hour at 0° C. and then concentrated to dryness. The residue was purified by column chromatography (silica gel; cyclohexane:ethyl acetate; 4:1 to 4:6; v/v) to afford (4-nitrophenyl) 4-[(2,4-dichlorophenyl)methyl]piperidine-1-carboxylate (155 mg) as an off-white semi-solid.

MS m/z (+ESI): 450.9, 452.8 [M+HCOOH]⁺.

### Preparation of 4-[(2,4-dichlorophenyl)methyl]-N-pyrimidin-4-yl-piperidine-1-carboxamide

To a slurry of 60% sodium hydride (13 mg; 0.30 mmol) in dry N,N-dimethylformamide (2 mL) was added pyrimidin-4-amine (23 mg; 0.24 mmol) [CAS 591-54-8] and the mixture was stirred for 0.5 hour. The reaction mixture was then heated to 80° C. and treated with a solution of (4-nitrophenyl) 4-[(2,4-dichlorophenyl)methyl]piperidine-1-carboxylate (50 mg; 0.12 mmol) in dry N,N-dimethylformamide (1 mL). The reaction mixture was stirred for 0.5 hour at 80° C. After cooling to room temperature, ethyl acetate (20 mL) was added and the mixture was successively washed with water, 8% NaHCO₃ aqueous solution (3×10 mL), brine, dried over MgSO₄, filtered and concentrated to dryness. The residue was purified by column chromatography (MCI gel; water:acetonitrile; 4:6; v/v) to afford 4-[(2,4-dichlorophenyl)methyl]-N-pyrimidin-4-yl-piperidine-1-carboxamide (33 mg) as a white powder.

### Preparation of Example 27: 4-[(2-fluoro-4-methyl-phenyl)methyl]-N-(3-methyl-1,2,4-thiadiazol-5-yl)piperidine-1-carboxamide

### Preparation of 1-(diethoxyphosphorylmethyl)-2-fluoro-4-methyl-benzene

A mixture of 1-(bromomethyl)-2-fluoro-4-methyl-benzene (450 mg, 2.22 mmol) [CAS 118745-63-4] in triethylphosphite (1.11 mL, 6.65 mmol) was heated to 100° C. After stirring for 5 hours, the reaction mixture was cooled to 20° C. and then concentrated under vacuum to afford 1-(di-ethoxyphosphorylmethyl)-2-fluoro-4-methyl-benzene (575 mg) as colorless oil.

MS m/z (+ESI): 261.1 [M+H]⁺.

### Preparation of tert-butyl 4-[(2-fluoro-4-methyl-phenyl)methylene]piperidine-1-carboxylate

To a mixture of 1-(diethoxyphosphorylmethyl)-2-fluoro-4-methyl-benzene (500 mg, 1.92 mmol) in tetrahydrofuran (20 mL) was added sodium hydride 60% in mineral oil (307 mg, 7.69 mmol) at 0° C. The reaction mixture was stirred at 0° C. for 30 minutes and then treated with tert-butyl 4-oxopiperidine-1-carboxylate (383 mg, 1.92 mmol) [CAS 79099-07-3]. The reaction mixture was allowed to warm up to room temperature. After stirring for 2 hours, methanol (3 mL) was added to deactivate the reaction mixture and volatiles were removed under reduced pressure. The residue was purified by column chromatography (silica gel; petroleum ether:ethyl acetate; 20:1; v/v) to afford tert-butyl 4-[(2-fluoro-4-methyl-phenyl)methylene]piperidine-1-carboxylate (170 mg) as a white solid.

¹H-NMR (400 MHz, DMSO-d₆) δ ppm: 7.05 (dd, J1=J2=8.0 Hz, 1H), 6.85-6.91 (m, 2H), 6.24 (s, 1H), 3.52 (t, J=6.0 Hz, 2H), 3.41 (t, J=6.0 Hz, 2H), 2.30-2.40 (m, 7H), 1.46 (s, 9H).

### Preparation of tert-butyl 4-[(2-fluoro-4-methyl-phenyl)methyl]piperidine-1-carboxylate

A mixture of tert-butyl 4-[(2-fluoro-4-methyl-phenyl)methylene]piperidine-1-carboxylate (150 mg, 0.49 mmol) and 10% palladium on activated carbon (10 mg) in ethyl acetate (15 mL) was stirred under hydrogen atmosphere for 10 hours. The reaction mixture was filtered and the filtrate was concentrated under vacuum to afford tert-butyl 4-[(2-fluoro-4-methyl-phenyl)methyl]piperidine-1-carboxylate (150 mg) as a colorless oil.

¹H-NMR (400 MHz, CDCl₃) δ ppm: 6.99 (dd, J1=J2=8.0 Hz, 1H), 6.80-6.88 (m, 2H), 3.98-4.17 (m, 2H), 2.57-2.70 (m, 2H), 2.53 (d, J=7.2 Hz, 2H), 2.31 (s, 3H), 1.57-1.73 (m, 3H), 1.45 (s, 9H), 1.10-1.23 (m, 2H).

### Preparation of 4-[(2-fluoro-4-methyl-phenyl)methyl]piperidine

A solution of tert-butyl 4-[(2-fluoro-4-methyl-phenyl)methyl]piperidine-1-carboxylate (150 mg, 0.49 mmol) in 2.0 M HCl in ethyl acetate (10 mL) was stirred at 20° C. for 5 hours and the reaction mixture was concentrated to dryness to afford 4-[(2-fluoro-4-methyl-phenyl)methyl]piperidine as a white solid in quantitative yield.

MS m/z (+ESI): 208.1 [M+H]⁺.

### Preparation of (4-nitrophenyl) N-(3-methyl-1,2,4-thiadiazol-5-yl)carbamate

3-Methyl-1,2,4-thiadiazol-5-amine (1000 mg; 8.68 mmol) [CAS 17467-35-5] and 4-dimethylaminopyridine (106 mg; 0.87 mmol) were dissolved in pyridine (50 mL). The mixture was cooled to 0° C. followed by addition of 4-nitrophenyl chloroformate (1750 mg; 8.68 mmol) [CAS 7693-46-1]. The resulting mixture was stirred at 0° C. for 0.5 hour. The reaction mixture was allowed to warm to room temperature and further stirred for 20 hours at. Water (100 mL) was added and the resulting suspension was filtered. The filter cake was washed with water (2×20 mL) and diethylether (2×20 mL) successively. The white solid was dried to afford (4-nitrophenyl) N-(3-methyl-1,2,4-thiadiazol-5-yl)carbamate (1300 mg).

MS m/z (+ESI): 280.0 [M+H]⁺.

### Preparation of 4-[(2-fluoro-4-methyl-phenyl)methyl]-N-(3-methyl-1,2,4-thiadiazol-5-yl)piperidine-1-carboxamide

To a solution of 4-[(2-fluoro-4-methyl-phenyl)methyl]piperidine (101 mg, 0.49 mmol) and trimethylamine (0.2 mL, 1.46 mmol) in N,N-dimethylformamide (15 mL) was added (4-nitrophenyl) N-(3-methyl-1,2,4-thiadiazol-5-yl)carbamate (273 mg, 0.98 mmol). The mixture was stirred for 16 hours. The reaction mixture was concentrated and the residue was purified by preparative HPLC to afford 4-[(2-fluoro-4-methyl-phenyl)methyl]-N-(3-methyl-1,2,4-thiadiazol-5-yl)piperidine-1-carboxamide (85 mg) as a white solid.

### Preparation of Example 28: 4-[(3,4-dimethylphenyl)methyl]-N-(3-methyl-1,2,4-thiadiazol-5-yl)piperidine-1-carboxamide

### Preparation of N-(3-methyl-1,2,4-thiadiazol-5-yl)-4-oxo-piperidine-1-carboxamide

The title compound was prepared as a white solid following scheme 1 and in analogy to Example 27 using 4-oxopiperidinium chloride [CAS 41979-39-9] and (4-nitrophenyl) N-(3-methyl-1,2,4-thiadiazol-5-yl)carbamate as starting materials and after purification by column chromatography (silica gel; petroleum ether:ethyl acetate; 1:1; v/v).

MS m/z (+ESI): 241.1 [M+H]⁺.

¹H-NMR (400 MHz, CDCl₃) δ ppm: 3.91 (t, J=6.4 Hz, 4H), 2.60 (t, J=6.4 Hz, 4H), 2.49 (s, 3H).

### Preparation of 4-[(3,4-dimethylphenyl)methylene]-N-(3-methyl-1,2,4-thiadiazol-5-yl)piperidine-1-carboxamide

To a mixture of 4-(diethoxyphosphorylmethyl)-1,2-dimethyl-benzene (400 mg; 1.56 mmol) in tetrahydrofuran (25 mL) was added sodium hydride 60% in mineral oil (250 mg; 6.24 mmol) at 0° C. The reaction mixture was stirred at 0° C. for 0.5 hour and N-(3-methyl-1,2,4-thiadiazol-5-yl)-4-oxo-piperidine-1-carboxamide (375 mg; 1.56 mmol) was added. The reaction mixture was allowed to warm up to room temperature and was stirred for 3 hours. Methanol (2 mL) was added dropwise to deactivate the reaction and volatiles were removed under reduced pressure. The residue was purified column chromatography (silica gel; petroleum ether:ethyl acetate; 2:1; v/v) to afford 4-[(3,4-dimethylphenyl)methylene]-N-(3-methyl-1,2,4-thiadiazol-5-yl)piperidine-1-carboxamide (50 mg) as a light grey solid.

MS m/z (+ESI): 343.2 [M+H]⁺.

¹H-NMR (400 MHz, CDCl₃) δ ppm: 7.12 (d, J=7.6 Hz, 1H), 6.88-6.98 (m, 2H), 6.39 (s, 1H), 3.64 (t, J=6.0 Hz, 2H), 3.54 (t, J=6.0 Hz, 2H), 2.60 (t, J=6.0 Hz, 2H), 2.59 (s, 3H), 2.51 (s, 3H), 2.46 (t, J=6.0 Hz, 2H), 2.27 (s, 3H).

### Preparation of 4-[(3,4-dimethylphenyl)methyl]-N-(3-methyl-1,2,4-thiadiazol-5-yl)piperidine-1-carboxamide

A mixture 4-[(3,4-dimethylphenyl)methylene]-N-(3-methyl-1,2,4-thiadiazol-5-yl)piperidine-1-carboxamide (500 mg; 1.46 mmol) and 10% palladium on activated carbon (50 mg) in ethanol (20 mL) was stirred under hydrogen atmosphere (4 bar) using Parr apparatus for 12 hours. The mixture was filtered and the filtrate was concentrated. The residue was purified by preparative HPLC to afford 4-[(3,4-dimethylphenyl)methyl]-N-(3-methyl-1,2,4-thiadiazol-5-yl)piperidine-1-carboxamide (80 mg) as a white solid.

### Preparation of Example 34: N-(3-methyl-1,2,4-thiadiazol-5-yl)-4-[[4-(morpholinomethyl)phenyl]methyl]piperidine-1-carboxamide

### Preparation of 4-[[1-[(3-methyl-1,2,4-thiadiazol-5-yl)carbamoyl]-4-piperidylidene]methyl]benzoic acid

The title compound was prepared as a white solid following scheme 1 and in analogy to Example 27 and 28 using methyl 4-(bromomethyl)benzoate [CAS 2417-72-3] and N-(3-methyl-1,2,4-thiadiazol-5-yl)-4-oxo-piperidine-1-carboxamide as starting materials and after purification by column chromatography (silica gel; ethanol).

MS m/z (+ESI): 359.1 [M+H]⁺.

### Preparation of 4-[[4-(hydroxymethyl)phenyl]methylene]-N-(3-methyl-1,2,4-thiadiazol-5-yl)piperidine-1-carboxamide

To a mixture of 4-[[1-[(3-methyl-1,2,4-thiadiazol-5-yl)carbamoyl]-4-piperidylidene]methyl]benzoic acid (580 mg; 1.62 mmol) in tetrahydrofuran (25 mL) was added a solution of 2M borane dimethyl sulfide complex in tetrahydrofuran (1.6 mL; 3.20 mmol) at 0° C. The reaction mixture was allowed to warm up to room temperature and was further stirred for 3 hours. Methanol (3 mL) was added dropwise to quench the reaction and volatiles were removed under reduced pressure. The residue was purified by column chromatography (silica gel; ethanol) to afford 4-[[4-(hydroxymethyl)phenyl]methylene]-N-(3-methyl-1,2,4-thiadiazol-5-yl)piperidine-1-carboxamide (350 mg) as a white solid.

MS m/z (+ESI): 345.1 [M+H]⁺.

### Preparation of 4-[[1-[(3-methyl-1,2,4-thiadiazol-5-yl)carbamoyl]-4-piperidylidene]methyl]phenyl]methyl methanesulfonate

To a mixture of 4-[[4-(hydroxymethyl)phenyl]methylene]-N-(3-methyl-1,2,4-thiadiazol-5-yl)piperidine-1-carboxamide (320 mg; 0.93 mmol) in dichloromethane (25 mL) were added triethylamine (0.39 mL; 2.79 mmol) and methane sulfonyl chloride (0.09 mL; 1.11 mmol) at 0° C. The reaction mixture was allowed to warm up to room temperature and was further stirred for 2 hours. The reaction mixture was concentrated to dryness to afford 4-[[1-[(3-methyl-1,2,4-thiadiazol-5-yl)carbamoyl]-4-piperidylidene]methyl]phenyl]methyl methanesulfonate in quantitative yield as a light yellow oil, which was used directly in the next step without purification.

MS m/z (+ESI): 423.1 [M+H]⁺.

### Preparation of N-(3-methyl-1,2,4-thiadiazol-5-yl)-4-[[4-(morpholinomethyl)phenyl]methylene]piperidine-1-carboxamide

To a mixture of [4-[[1-[(3-methyl-1,2,4-thiadiazol-5-yl)carbamoyl]-4-piperidylidene]methyl]phenyl]methyl methanesulfonate (392 mg; 0.93 mmol) in dichloromethane (25 mL) were added morpholine (162 mg; 1.86 mmol) and triethylamine (0.39 mL; 2.78 mmol). After stirring for 5 hours, the reaction mixture was concentrated to dryness and the residue was purified by column chromatography (silica gel; ethanol) to afford N-(3-methyl-1,2,4-thiadiazol-5-yl)-4-[[4-(morpholinomethyl)phenyl]methylene]piperidine-1-carboxamide (80 mg) as a light yellow oil.

MS m/z (+ESI): 414.1 [M+H]⁺.

### Preparation of N-(3-methyl-1,2,4-thiadiazol-5-yl)-4-[[4-(morpholinomethyl)phenyl]methyl]piperidine-1-carboxamide

The title compound was prepared as a white solid following scheme 1 and in analogy to Example 28 using N-(3-methyl-1,2,4-thiadiazol-5-yl)-4-[[4-(morpholinomethyl)phenyl]methylene]piperidine-1-carboxamide as starting material and after purification by preparative HPLC.

### Preparation of Example 35: 4-[[2-fluoro-4-(5-methyl-1,2,4-oxadiazol-3-yl)phenyl]methyl]-N-(3-methyl-1,2,4-thiadiazol-5-yl)piperidine-1-carboxamide

### Preparation of tert-butyl 4-[[2-fluoro-4-(N-hydroxycarbamimidoyl)phenyl]methyl]piperidine-1-carboxylate

A mixture of tert-butyl 4-[(4-cyano-2-fluoro-phenyl)methyl]piperidine-1-carboxylate (100 mg; 0.31 mmol) (intermediate of Example 32), hydroxylamine hydrochloride (66 mg; 0.94 mmol) and sodium hydrogen carbonate (90 mg; 0.94 mmol) in ethanol (10 mL) was stirred at 60° C. for 5 hours. Insolubles were removed by filtration and the filtrate was concentrated to dryness to give tert-butyl 4-[[2-fluoro-4-(N-hydroxycarbamimidoyl)phenyl]methyl]piperidine-1-carboxylate (110 mg) as a white solid, which was used directly in the next step without purification.

MS m/z (+ESI): 352.2 [M+H]⁺.

### Preparation of 1-[4-[[2-fluoro-4-(5-methyl-1,2,4-oxadiazol-3-yl)phenyl]methyl]-1-piperidyl]ethanone

Under nitrogen atmosphere, a mixture of tert-butyl 4-[[2-fluoro-4-(N-hydroxycarbamimidoyl)phenyl]-methyl]piperidine-1-carboxylate (550 mg; 1.57 mmol) in a mixture of acetic anhydride (1.47 mL; 15.7 mmol) and acetic acid (15 mL) was stirred at 100° C. for 5 hours. The mixture was concentrated and the residue was dissolved in ethyl acetate (30 mL). The mixture was washed with water and brine, dried over N₂SO₄, filtered and concentrated to dryness to afford 1-[4-[[2-fluoro-4-(5-methyl-1,2,4-oxadiazol-3-yl)phenyl]methyl]-1-piperidyl]ethanone (490 mg) as a light yellow oil, which was used directly in the next step without purification.

MS m/z (+ESI): 318.2 [M+H]⁺.

### Preparation of 3-[3-fluoro-4-(4-piperidylmethyl)phenyl]-5-methyl-1,2,4-oxadiazole

1-[4-[[2-fluoro-4-(5-methyl-1,2,4-oxadiazol-3-yl)phenyl]methyl]-1-piperidyl]ethanone (450 mg; 1.42 mmol) was dissolved in ethanol (20 mL) followed by addition of 1M sodium hydroxide aqueous solution (10 mL). The reaction solution was heated to 90° C. and stirred for 3 hours. After cooling to room temperature, ethanol was removed under reduced pressure and the residue was dissolved in ethyl acetate (20 mL). The organic solution was washed with water, dried over Na₂SO₄, filtered and concentrated to afford 3-[3-fluoro-4-(4-piperidylmethyl)phenyl]-5-methyl-1,2,4-oxadiazole (350 mg) as a light yellow oil.

MS m/z (+ESI): 276.1 [M+H]⁺.

### Preparation of 4-[[2-fluoro-4-(5-methyl-1,2,4-oxadiazol-3-yl)phenyl]methyl]-N-(3-methyl-1,2,4-thiadiazol-5-yl)piperidine-1-carboxamide

The title compound was prepared as a white solid following scheme 1 and in analogy to Example 27 using 3-[3-fluoro-4-(4-piperidylmethyl)phenyl]-5-methyl-1,2,4-oxadiazole and (4-nitrophenyl) N-(3-methyl-1,2,4-thiadiazol-5-yl)carbamate as starting materials and after purification by preparative HPLC.

### Preparation of Example 37: 4-[(4-carbamoyl-2-fluoro-phenyl)methyl]-N-(3-methyl-1,2,4-thiadiazol-5-yl)piperidine-1-carboxamide

### Preparation of 3-fluoro-4-[[1-[(3-methyl-1,2,4-thiadiazol-5-yl)carbamoyl]-4-piperidyl]methyl]benzoic acid

To a mixture of methyl 3-fluoro-4-[[1-[(3-methyl-1,2,4-thiadiazol-5-yl)carbamoyl]-4-piperidyl]methyl]-benzoate (300 mg; 0.76 mmol) in tetrahydrofuran (10 mL) and methanol (10 mL) was added a solution of lithium hydroxide monohydrate (183 mg) in water (2 mL). The reaction mixture was stirred for 12 hours and then placed in an ice-bath. 6N HCl aqueous solution was added dropwise to adjust the pH between 1 and 2. The mixture was then partitioned between water and ethyl acetate. The organic layer was separated, washed with brine, dried over anhydrous Na₂SO₄, filtered and concentrated to afford 3-fluoro-4-[[1-[(3-methyl-1,2,4-thiadiazol-5-yl)carbamoyl]-4-piperidyl]methyl]benzoic acid (292 mg) as a white solid.

MS m/z (+ESI): 379.1 [M+H]⁺.

### Preparation of 4-[(4-carbamoyl-2-fluoro-phenyl)methyl]-N-(3-methyl-1,2,4-thiadiazol-5-yl)piperidine-1-carboxamide

To a mixture of 3-fluoro-4-[[1-[(3-methyl-1,2,4-thiadiazol-5-yl)carbamoyl]-4-piperidyl]methyl]benzoic acid (292 mg; 0.77 mmol) and ammonium chloride (49 mg; 0.92 mmol) in dichloromethane (10 mL) were successively added benzotriazol-1-ol (124 mg; 0.92 mmol), 1-ethyl-3-(3-dimethylaminopropyl)carbodiimide (176 mg; 0.92 mmol) and triethylamine (0.23 mL; 2.30 mmol). After stirring for 12 hours, volatiles were removed and the residue was purified by preparative HPLC to afford 4-[(4-carbamoyl-2-fluoro-phenyl)methyl]-N-(3-methyl-1,2,4-thiadiazol-5-yl)piperidine-1-carboxamide (40 mg) as a white solid.

### Preparation of Example 45: 4-[(2-fluoro-4-pyrrolidin-1-yl-phenyl)methylene]-N-(3-methyl-1,2,4-thiadiazol-5-yl)piperidine-1-carboxamide

### Preparation of tert-butyl 4-[(2-fluoro-4-nitro-phenyl)methylene]piperidine-1-carboxylate

The title compound was prepared as a yellow oil following scheme 1 and in analogy to Example 27 using 1-(diethoxyphosphorylmethyl)-2-fluoro-4-nitro-benzene [127349-57-9] and tert-butyl 4-oxopiperidine-1-carboxylate [79099-07-3] as starting materials.

MS m/z (+ESI): 337.1 [M+H]⁺.

### Preparation of tert-butyl 4-[(4-amino-2-fluoro-phenyl)methylene]piperidine-1-carboxylate

To a solution of tert-butyl 4-[(2-fluoro-4-nitro-phenyl)methylene]piperidine-1-carboxylate (260 mg; 0.73 mmol) in ethanol (27 mL) was added a solution of ammonium chloride (393 mg; 7.34 mmol) in water (3 mL) followed by the addition of iron powder (328 mg; 5.88 mmol). The reaction mixture was heated to 100° C. and stirred for 2 hours. The mixture was cooled to 25° C. and filtered. Volatiles were removed under reduced pressure and the residue was taken up in ethyl acetate. The mixture was washed with water and brine successively. The organic layer was dried over Na₂SO₄, filtered and concentrated to afford tert-butyl 4-[(4-amino-2-fluoro-phenyl)methylene]piperidine-1-carboxylate as a light yellow oil in quantitative yield.

MS m/z (+ESI): 307.2 [M+H]⁺.

### Preparation of tert-butyl 4-[(2-fluoro-4-pyrrolidin-1-yl-phenyl)methylene]piperidine-1-carboxylate

To a solution of tert-butyl 4-[(4-amino-2-fluoro-phenyl)methylene]piperidine-1-carboxylate (140 mg; 0.46 mmol) in N,N-dimethylformamide (8 mL) were added 1,4-dibromobutane (0.082 mL; 0.69 mmol) and potassium carbonate (190 mg; 1.37 mmol). The mixture was heated to 100° C. and stirred for 24 hours. After cooling to room temperature, ethyl acetate was added and the mixture was washed with brine, dried over Na₂SO₄, filtered and concentrated to dryness. The residue was purified by column chromatography (silica gel; petroleum ether:ethyl acetate; 10:1; v/v) to afford tert-butyl 4-[(2-fluoro-4-pyrrolidin-1-yl-phenyl)methylene]piperidine-1-carboxylate (70 mg) as a light yellow solid.

MS m/z (+ESI): 361.2 [M+H]⁺.

### Preparation of 4-[(2-fluoro-4-pyrrolidin-1-yl-phenyl)methylene]piperidine

The title compound was prepared as a white solid following scheme 1 and in analogy to Example 27 using tert-butyl 4-[(2-fluoro-4-pyrrolidin-1-yl-phenyl)methylene]piperidine-1-carboxylate as starting material.

MS m/z (+ESI): 261.2 [M+H]⁺.

### Preparation of 4-[(2-fluoro-4-pyrrolidin-1-yl-phenyl)methylene]-N-(3-methyl-1,2,4-thiadiazol-5-yl)piperidine-1-carboxamide

The title compound was prepared as a white solid following scheme 1 and in analogy to Example 27 using 4-[(2-fluoro-4-pyrrolidin-1-yl-phenyl)methylene]piperidine and (4-nitrophenyl) N-(3-methyl-1,2,4-thiadiazol-5-yl)carbamate as starting materials and after purification by preparative HPLC.

### Preparation of Example 47: 4-[[4-(dimethylamino)-2-fluoro-phenyl]methylene]-N-(3-methyl-1,2,4-thiadiazol-5-yl)piperidine-1-carboxamide

### Preparation of tert-butyl 4-[[4-(dimethylamino)-2-fluoro-phenyl]methylene]piperidine-1-carboxylate

Tert-butyl 4-[(4-amino-2-fluoro-phenyl)methylene]piperidine-1-carboxylate (220 mg; 0.68 mmol) (intermediate of Example 45) was dissolved in 1,2-dichloroethane (10 mL). 37% aqueous formaldehyde solution (0.2 mL; 2.73 mmol) and 1 drop of acetic acid were added. The reaction mixture was stirred for 0.5 hour and treated with sodium triacetoxyborohydride (868 mg; 4.09 mmol). After stirring for 16 hours, the mixture was concentrated. The residue was purified by column chromatography (silica gel; petroleum ether:ethyl acetate; 10:1 to 6:1; v/v) to afford tert-butyl 4-[[4-(dimethylamino)-2-fluoro-phenyl]methylene]piperidine-1-carboxylate (150 mg) as a light yellow oil.

MS m/z (+ESI): 335.2 [M+H]⁺.

¹H-NMR (400 MHz, CDCl₃) δ ppm: 7.04 (dd, J1=J2=8.4 Hz, 1H), 6.38-6.49 (m, 2H), 6.22 (s, 1H), 3.51 (t, J=5.6 Hz, 2H), 3.41 (t, J=5.6 Hz, 2H), 2.96 (s, 6H), 2.36 (t, J=5.6 Hz, 4H), 1.49 (s, 9H).

### Preparation of 3-fluoro-N,N-dimethyl-4-(4-piperidylidenemethyl)aniline

The title compound was prepared as a white solid following scheme 1 and in analogy to Example 27 using tert-butyl 4-[[4-(dimethylamino)-2-fluoro-phenyl]methylene]piperidine-1-carboxylate as starting material.

MS m/z (+ESI): 235.2 [M+H]⁺.

### Preparation of 4-[[4-(dimethylamino)-2-fluoro-phenyl]methylene]-N-(3-methyl-1,2,4-thiadiazol-5-yl)piperidine-1-carboxamide

The title compound was prepared as a white solid following scheme 1 and in analogy to Example 27 using 3-fluoro-N,N-dimethyl-4-(4-piperidylidenemethyl)aniline and (4-nitrophenyl) N-(3-methyl-1,2,4-thiadiazol-5-yl)carbamate as starting materials and after purification by preparative HPLC.

### Preparation of Example 50: 4-[(4-chloro-2-fluoro-phenyl)methyl]-N-(3-methyl-1,2,4-thiadiazol-5-yl)piperazine-1-carboxamide

### Preparation of tert-butyl 4-[(4-chloro-2-fluoro-phenyl)methyl]piperazine-1-carboxylate

To a stirred solution of 4-chloro-2-fluorobenzyl bromide (2700 mg; 12.08 mmol) [CAS 71916-82-0] in N,N-dimethylformamide (40 mL) were added 1-Boc-piperazine (1500 mg; 8.05 mmol) [CAS 57260-71-6] and cesium carbonate (7870 mg; 24.16 mmol). The mixture was heated to 80° C. and stirred for 4 hours. The mixture was cooled, diluted with ethyl acetate (100 mL) and was washed with water and brine successively. The organic layer was dried over anhydrous Na₂SO₄, filtered and concentrated to dryness. The residue was purified by column chromatography (silica gel; petroleum ether:ethyl acetate; 2:1; v/v) to afford tert-butyl 4-[(4-chloro-2-fluoro-phenyl)methyl]piperazine-1-carboxylate (2400 mg) as a yellow oil.

MS m/z (+ESI): 329.1 [M+H]⁺.

### Preparation of 1-[(4-chloro-2-fluoro-phenyl)methyl]piperazine

The title compound was prepared as a white solid following scheme 1 and 3 and in analogy to Example 27 using tert-butyl 4-[(4-chloro-2-fluoro-phenyl)methyl]piperazine-1-carboxylate as starting material.

MS m/z (+ESI): 229.1 [M+H]⁺.

### Preparation of 4-[(4-chloro-2-fluoro-phenyl)methyl]-N-(3-methyl-1,2,4-thiadiazol-5-yl)piperazine-1-carboxamide

The title compound was prepared as a white solid following scheme 1 and 3 and in analogy to Example 27 using 1-[(4-chloro-2-fluoro-phenyl)methyl]piperazine and (4-nitrophenyl) N-(3-methyl-1,2,4-thiadiazol-5-yl)carbamate as starting materials and after purification by preparative HPLC.

### Preparation of Example 70: 4-[(4-chloro-2-fluoro-phenyl)methyl]-N-(3-ethyl-1,2,4-thiadiazol-5-yl)piperidine-1-carboxamide

### Preparation of 4-[(4-chloro-2-fluoro-phenyl)methyl]-N-(3-ethyl-1,2,4-thiadiazol-5-yl)piperidine-1-carboxamide

To a stirred solution of diphosgene (0.09 mL; 0.74 mmol) in dichloromethane (1 mL) was added dropwise a solution of 3-ethyl-1,2,4-thiadiazol-5-amine (160 mg; 1.24 mmol) [17467-41-3], N,N-dimethylpyridin-4-amine (16 mg; 0.12 mmol) and triethylamine (0.18 mL; 1.24 mmol) in dichloromethane (2 mL) at −40° C. The reaction mixture was stirred for 0.5 hour at −40° C. before a solution of 4-[(4-chloro-2-fluoro-phenyl)methyl]piperidine hydrochloride (330 mg; 1.24 mmol) (intermediate Example 9) and triethylamine (0.18 mL; 1.24 mmol) in dichloromethane (2 mL) was added dropwise. The resulting solution was further stirred at 20° C. for 18 hours. Volatiles were removed under reduced pressure and the residue was purified by preparative HPLC to afford 4-[(4-chloro-2-fluoro-phenyl)methyl]-N-(3-ethyl-1,2,4-thiadiazol-5-yl)piperidine-1-carboxamide (125 mg) as a white solid.

### Preparation of Example 77: 4-(4-bromobenzoyl)-N-(3-methyl-1,2,4-thiadiazol-5-yl)piperidine-1-carboxamide

### Preparation of (4-bromophenyl)-(4-piperidyl)methanone, trifluoroacetic acid salt

Under nitrogen atmosphere, to a stirred solution of tert-butyl 4-(4-bromobenzoyl)piperidine-1-carboxylate (100 mg; 0.26 mmol) [CAS 439811-37-7] in dichloromethane (3 mL) was added dropwise trifluoroacetic acid (0.29 mL; 3.87 mmol). The reaction solution was stirred for 0.5 hours and then concentrated to dryness to afford (4-bromophenyl)-(4-piperidyl)methanone, trifluoroacetic acid salt (100 mg) as brown solid.

MS m/z (+ESI): 229.1 [M+H]⁺.

### Preparation of 4-(4-bromobenzoyl)-N-(3-methyl-1,2,4-thiadiazol-5-yl)piperidine-1-carboxamide

The title compound was prepared as a white solid following scheme 1 and 4 and in analogy to Example 27 using (4-bromophenyl)-(4-piperidyl)methanone trifluoroacetic acid salt and (4-nitrophenyl) N-(3-methyl-1,2,4-thiadiazol-5-yl)carbamate as starting materials and after purification by column chromatography (silica gel; cyclohexane:ethyl acetate; 3:2 to 0:1; v/v).

### Preparation of Example 78: 4-[(4-chloro-2-fluoro-phenyl)methylene]-N-[3-(2-hydroxyethyl)-1,2,4-thiadiazol-5-yl]piperidine-1-carboxamide

### Preparation of ethyl 2-[5-[[4-[(4-chloro-2-fluoro-phenyl)methylene]piperidine-1-carbonyl]amino]-1,2,4-thiadiazol-3-yl]acetate

To an ice-cold solution of ethyl 2-(5-amino-1,2,4-thiadiazol-3-yl)acetate (170 mg; 0.86 mmol) [CAS 92738-69-7] and triethylamine (0.49 mL; 3.45 mmol) in dichloromethane (10 mL) was added triphosgene (90 g, 0.30 mmol) and the solution was stirred for 0.5 hours at 0° C. A freshly prepared solution of 4-[(4-chloro-2-fluoro-phenyl)methylene]piperidine, trifluoroacetic acid salt (290 mg; 0.86 mmol) (intermediate of Example 39) and triethylamine (0.25 mL) in dichloromethane (3 mL) was added dropwise to the above solution at 0° C. The solution was stirred for 1 hour and then deactivated with methanol (1 mL). Volatiles were removed under reduced pressure and the residue was dissolved in ethyl acetate. The solution was washed with a saturated aqueous solution of ammonium chloride, brine, dried over MgSO₄, filtered and concentrated to dryness. The crude residue was purified by column chromatography (silica gel; cyclohexane:ethyl acetate; 9:1 to 35:65; v/v) to afford 2-[5-[[4-[(4-chloro-2-fluoro-phenyl)methylene]piperidine-1-carbonyl]amino]-1,2,4-thiadiazol-3-yl]acetate (200 mg) as a light yellow foam.

MS m/z (+ESI): 439.0, 441.0 [M+H]⁺.

¹H-NMR (400 MHz, DMSO-d₆) δ ppm: 11.88 (s, 1H), 7.44-7.47 (m, 1H), 7.28-7.6 (m, 2H), 6.30 (s, 1H), 4.12 (q, J=7.1 Hz, 2H), 3.83 (s, 2H), 3.66 (m, 2H), 3.58 (m, 2H), 2.42 (m, 2H), 2.34 (m, 2H), 1.19 (t, J=7.1 Hz, 3H).

### Preparation of 4-[(4-chloro-2-fluoro-phenyl)methylene]-N-[3-(2-hydroxyethyl)-1,2,4-thiadiazol-5-yl]piperidine-1-carboxamide

To a stirred solution of ethyl 2-[5-[[4-[(4-chloro-2-fluoro-phenyl)methylene]piperidine-1-carbonyl]amino]-1,2,4-thiadiazol-3-yl]acetate (670 mg; 1.45 mmol) in ethanol (20 mL) was added sodium borohydride (450 mg; 11.6 mmol). The mixture was stirred for 16 hours. Additional sodium borohydride (450 mg; 11.6 mmol) was added and the reaction mixture was stirred for 30 hours. Ethyl acetate, water and a saturated aqueous solution of ammonium chloride was added. The organic layer was separated, washed with brine, dried over MgSO₄, filtered and concentrated to dryness. The residue was triturated in fresh ethyl acetate, the resulting suspension was filtered and the cake washed with ethyl acetate. The solid was dried to afford 4-[(4-chloro-2-fluoro-phenyl)methylene]-N-[3-(2-hydroxyethyl)-1,2,4-thiadiazol-5-yl]piperidine-1-carboxamide (240 mg) as a white powder.

### Preparation of Example 79: 4-[(4-chloro-2-fluoro-phenyl)methylene]-N-[3-(2-morpholinoethyl)-1,2,4-thiadiazol-5-yl]piperidine-1-carboxamide

### Preparation of 2-[5-[[4-[(4-chloro-2-fluoro-phenyl)methylene]piperidine-1-carbonyl]amino]-1,2,4-thiadiazol-3-yl]ethyl methanesulfonate

To a stirred suspension of 4-[(4-chloro-2-fluoro-phenyl)methylene]-N-[3-(2-hydroxyethyl)-1,2,4-thiadiazol-5-yl]piperidine-1-carboxamide (50 mg; 0.12 mmol) (Example 78) in dichloromethane (2 mL) cooled to 0° C. was added triethylamine (0.04 mL; 0.31 mmol) followed by methane sulfonyl chloride (0.01 mL; 0.14 mmol). The reaction mixture was stirred for 2 hours at 0° C. and for 3 hours at room temperature. The reaction was then deactivated with the addition of methanol (0.2 mL) and the solution was concentrated to dryness to afford crude 2-[5-[[4-[(4-chloro-2-fluoro-phenyl)methylene]piperidine-1-carbonyl]amino]-1,2,4-thiadiazol-3-yl]ethyl methanesulfonate in quantitative yield as a light yellow oil. The product was used in the next step without further purification.

MS m/z (+ESI): 475.0, 477.0 [M+H]⁺.

### Preparation of 4-[(4-chloro-2-fluoro-phenyl)methylene]-N-[3-(2-morpholinoethyl)-1,2,4-thiadiazol-5-yl]piperidine-1-carboxamide

2-[5-[[4-[(4-chloro-2-fluoro-phenyl)methylene]piperidine-1-carbonyl]amino]-1,2,4-thiadiazol-3-yl]ethyl methanesulfonate (59 mg; 0.12 mmol) was dissolved in methanol (2 mL) and morpholine (0.33 mL; 3.74 mmol) was addede. The reaction solution was heated to 65° C. and stirred for 24 hours. After cooling to room temperature, ethyl acetate was added and the solution was washed with brine, dried over MgSO₄, filtered and concentrated to dryness. The crude was purified by column chromatography (silica gel; dichloromethane:methanol; 1:0 to 9:1; v/v). The product obtained was triturated in diisopropylether for 10 minutes and the resulting suspension was filtered. The solid was dried to afford 4-[(4-chloro-2-fluoro-phenyl)methylene]-N-[3-(2-morpholinoethyl)-1,2,4-thiadiazol-5-yl]piperidine-1-carboxamide (24 mg) as a white powder.

### Preparation of Example 80: 4-[(4-chloro-2-fluoro-phenyl)methylene]-N-[3-(2-cyanoethyl)-1,2,4-thiadiazol-5-yl]piperidine-1-carboxamide

### Preparation of N-[3-(2-bromoethyl)-1,2,4-thiadiazol-5-yl]-4-[(4-chloro-2-fluoro-phenyl)methylene]piperidine-1-carboxamide

To a stirred suspension of 4-[(4-chloro-2-fluoro-phenyl)methylene]-N-[3-(2-hydroxyethyl)-1,2,4-thiadiazol-5-yl]piperidine-1-carboxamide (290 mg; 0.69 mmol) (Example 78) in dichloromethane (7 mL) was added triphenylphosphine (280 mg; 1.04 mmol). The reaction mixture was stirred for 15 minutes and then cooled to 0° C. N-bromosuccinimide (140 mg; 0.76 mmol) was added in portions to get clear solution. The reaction solution was warmed to room temperature and stirred for 5 hours with addition of fresh N-bromosuccinimide (15 mg) at reaction time 1, 2, 3 and 4 hours. Ethyl acetate (40 mL) was added and the solution was washed with brine, dried over MgSO₄, filtered and concentrated to dryness. The crude residue was purified by column chromatography (silica gel; cyclohexane:ethyl acetate; 2:1 to 1:4; v/v) to afford N-[3-(2-bromoethyl)-1,2,4-thiadiazol-5-yl]-4-[(4-chloro-2-fluoro-phenyl)methylene]piperidine-1-carboxamide (225 mg) as a white foam.

MS m/z (+ESI): 458.9, 461.0, 462.9 [M+H]⁺.

### Preparation of 4-[(4-chloro-2-fluoro-phenyl)methylene]-N-[3-(2-cyanoethyl)-1,2,4-thiadiazol-5-yl]piperidine-1-carboxamide

To a stirred solution of N-[3-(2-bromoethyl)-1,2,4-thiadiazol-5-yl]-4-[(4-chloro-2-fluoro-phenyl)methylene]piperidine-1-carboxamide (110 mg; 0.23 mmol) in a mixture of ethanol (2 mL) and water (0.50 mL) was added potassium cyanide (300 mg; 4.55 mmol). The reaction mixture was then heated to 85° C. and stirred for 6 hours. After cooling to room temperature, ethyl acetate and brine were added. The organic layer was separated, washed with brine, dried over MgSO₄, filtered and concentrated to dryness. The residue was purified by column chromatography (silica gel; cyclohexane:ethyl acetate; 4:1 to 1:9; v/v). The product obtained was triturated in diisopropylether for 10 minutes and the resulting suspension was filtered. The solid was dried to afford 4-[(4-chloro-2-fluoro-phenyl)methylene]-N-[3-(2-cyanoethyl)-1,2,4-thiadiazol-5-yl]piperidine-1-carboxamide as a white powder.

### Preparation of example 81: N-[3-(2-amino-2-oxo-ethyl)-1,2,4-thiadiazol-5-yl]-4-[(4-chloro-2-fluoro-phenyl)methylene]piperidine-1-carboxamide

### Preparation of 2-[5-[[4-[(4-chloro-2-fluoro-phenyl)methylene]piperidine-1-carbonyl]amino]-1,2,4-thiadiazol-3-yl]acetic acid

To a stirred solution of ethyl 2-[5-[[4-[(4-chloro-2-fluoro-phenyl)methylene]piperidine-1-carbonyl]amino]-1,2,4-thiadiazol-3-yl]acetate (180 mg; 0.38 mmol) (intermediate of Example 78) in tetrahydrofuran (4 mL) was added dropwise a solution of lithium hydroxide monohydrate (60 mg; 1.52 mmol) in water (1 mL). The mixture was stirred for 1 hour. The pH was adjusted to about 3 using 3N HCl aqueous solution. Ethyl acetate and brine were added. The organic layer was separated, washed with brine, dried over MgSO₄, filtered and concentrated to dryness. The residue was triturated in diisopropylether, the suspension was filtered, washed with diisopropylether and the solid was dried to afford 2-[5-[[4-[(4-chloro-2-fluoro-phenyl)methylene]piperidine-1-carbonyl]amino]-1,2,4-thiadiazol-3-yl]acetic acid (128 mg) as a light yellow powder.

MS m/z (+ESI): 411.0, 413.0 [M+H]⁺.

¹H-NMR (400 MHz, DMSO-d₆) δ ppm: 12.58 (br, 1H), 11.87 (s, 1H), 7.44-7.47 (m, 1H), 7.28-7.36 (m, 2H), 6.30 (s, 1H), 4.04 (s, 2H), 3.66 (m, 2H), 3.58 (m, 2H), 2.42 (m, 2H), 2.34 (m, 2H).

### Preparation of N-[3-(2-amino-2-oxo-ethyl)-1,2,4-thiadiazol-5-yl]-4-[(4-chloro-2-fluoro-phenyl)methylene]piperidine-1-carboxamide

To a stirred solution of 2-[5-[[4-[(4-chloro-2-fluoro-phenyl)methylene]piperidine-1-carbonyl]amino]-1,2,4-thiadiazol-3-yl]acetic acid (50 mg; 0.12 mmol) and triethylamine (0.09 mL; 0.64 mmol) in N,N-dimethylformamide (1 mL) were successively added ammonium chloride (20 mg; 0.35 mmol) and HATU (60 mg; 0.15 mmol) at room temperature. The solution was stirred for 0.5 hours. Ethyl acetate was added and the solution was washed with water and brine, dried over MgSO₄, filtered and concentrated to dryness. The residue was then triturated in cold dichloromethane (1 mL) for 15 minutes, the resulting suspension was filtered and washed with cold dichloromethane (2×0.5 mL). The solid was finally dried to afford N-[3-(2-amino-2-oxo-ethyl)-1,2,4-thiadiazol-5-yl]-4-[(4-chloro-2-fluoro-phenyl)methylene]piperidine-1-carboxamide (34 mg) as a white powder.

### Preparation of Example 82 and 83: (4E)-4-[(4-chloro-2-fluoro-phenyl)methylene]-3-methyl-N-(3-methyl-1,2,4-thiadiazol-5-yl)piperidine-1-carboxamide and (4Z)-4-[(4-chloro-2-fluoro-phenyl)methylene]-3-methyl-N-(3-methyl-1,2,4-thiadiazol-5-yl)piperidine-1-carboxamide

### Preparation of tert-butyl (4E/Z)-4-[(4-chloro-2-fluoro-phenyl)methylene]-3-methyl-piperidine-1-carboxylate

The title compound was prepared as a colorless oil following scheme 1 and in analogy to Example 95 and 27 using tert-butyl 3-methyl-4-oxo-piperidine-1-carboxylate and 4-chloro-1-(diethoxyphosphorylmethyl)-2-fluoro-benzene (intermediate of Example 9) as starting materials and after purification by column chromatography (silica gel; petroleum ether:ethyl acetate; 10:1; v/v).

MS m/z (+ESI): 340.1, 342.1 [M+H]⁺.

¹H-NMR (400 MHz, CDCl₃) δ ppm: 7.04-7.16 (m, 3H), 6.16 and 6.12 (2s, 1H), 4.10-4.40 (m, 1H), 3.80-4.10 (m, 1H), 3.46-3.80 (m, 1H), 2.90-3.46 (m, 1H), 2.56-2.86 (m, 1H), 2.40-2.50 (m, 1H), 2.10-2.24 (m, 1H), 1.48 (s, 9H), 1.15-1.18 (m, 3H).

### Preparation of (4E/Z)-4-[(4-chloro-2-fluoro-phenyl)methylene]-3-methyl-piperidine

The title compound was prepared as a colorless oil following scheme 1 and in analogy to Example 95 and 27 using tert-butyl (4E/Z)-4-[(4-chloro-2-fluoro-phenyl)methylene]-3-methyl-piperidine-1-carboxylate as starting material.

MS m/z (+ESI): 240.1, 242.1 [M+H]⁺.

### Preparation of (4E)-4-[(4-chloro-2-fluoro-phenyl)methylene]-3-methyl-N-(3-methyl-1,2,4-thiadiazol-5-yl)piperidine-1-carboxamide and (4Z)-4-[(4-chloro-2-fluoro-phenyl)methylene]-3-methyl-N-(3-methyl-1,2,4-thiadiazol-5-yl)piperidine-1-carboxamide

The title compounds were prepared both as a white solid following scheme 1 and in analogy to Example 27 using (4E/Z)-4-[(4-chloro-2-fluoro-phenyl)methylene]-3-methyl-piperidine and (4-nitrophenyl) N-(3-methyl-1,2,4-thiadiazol-5-yl)carbamate as starting materials. Isomer E and Isomer Z were purified and separated by preparative HPLC.

### Preparation of Example 84a and 84b: 4-[(4-chloro-2-fluoro-phenyl)methyl]-3-methyl-N-(3-methyl-1,2,4-thiadiazol-5-yl)piperidine-1-carboxamide, isomer 1 and 4-[(4-chloro-2-fluoro-phenyl)methyl]-3-methyl-N-(3-methyl-1,2,4-thiadiazol-5-yl)piperidine-1-carboxamide, isomer 2

### Preparation of tert-butyl 4-[(4-chloro-2-fluoro-phenyl)methyl]-3-methyl-piperidine-1-carboxylate

The title compounds was prepared as a colorless oil following scheme 1 and in analogy to Example 27 using tert-butyl (4E/Z)-4-[(4-chloro-2-fluoro-phenyl)methylene]-3-methyl-piperidine-1-carboxylate as starting material.

MS m/z (+ESI): 342.1, 344.1 [M+H]⁺.

### Preparation of 4-[(4-chloro-2-fluoro-phenyl)methyl]-3-methyl-piperidine

The title compound was prepared as a colorless oil following scheme 1 and in analogy to Example 27 using tert-butyl 4-[(4-chloro-2-fluoro-phenyl)methyl]-3-methyl-piperidine-1-carboxylate as starting material.

MS m/z (+ESI): 242.1, 244.1 [M+H]⁺.

### Preparation of 4-[(4-chloro-2-fluoro-phenyl)methyl]-3-methyl-N-(3-methyl-1,2,4-thiadiazol-5-yl)piperidine-1-carboxamide, isomer 1 and 4-[(4-chloro-2-fluoro-phenyl)methyl]-3-methyl-N-(3-methyl-1,2,4-thiadiazol-5-yl)piperidine-1-carboxamide, isomer 2

The title compounds were prepared as a white solid following scheme 1 and in analogy to Example 27 using 4-[(4-chloro-2-fluoro-phenyl)methyl]-3-methyl-piperidine and (4-nitrophenyl) N-(3-methyl-1,2,4-thiadiazol-5-yl)carbamate as starting materials. Purification by preparative HPLC allowed the separation and the isolation of Isomer 1 (Example 84a) and Isomer 2 (Example 84b), both as mixture of enantiomers.

HPLC retention time:

For isomer 1: 20.3 minutes

For isomer 2: 20.7 minutes

### Preparation of Example 88: 4-[(2-fluoro-4-formyl-phenyl)methylene]-N-(3-methyl-1,2,4-thiadiazol-5-yl)piperidine-1-carboxamide

### Preparation of tert-butyl 4-(bromomethylene)piperidine-1-carboxylate

To a stirred suspension of (bromomethyl)triphenylphosphonium bromide (2000 mg; 4.47 mmol) in tetrahydrofuran (45 mL) cooled to −15° C. was added dropwise lithium bis(trimethylsilyl)amide solution, 1M in tetrahydrofuran (5.82 mL; 5.82 mmol) over 5 minutes. The reaction mixture was stirred for 15 minutes at −15° C. and then treated with a solution of tert-butyl 4-oxopiperidine-1-carboxylate (1000 mg; 4.92 mmol) [CAS 79099-07-3] in tetrahydrofuran (5 mL). The mixture was allowed to warm up gradually to room temperature and further stirred for 2 hours. The reaction mixture was deactivated with a saturated aqueous solution of ammonium chloride and then partitioned between ethyl acetate and brine. The layers were separated. The organic layer was washed with brine, dried over MgSO₄, filtered and concentrated to dryness. The crude residue was purified by column chromatography (silica gel; cyclohexane:ethylacetate; 1:0 to 4:1; v/v) to afford tert-butyl 4-(bromomethylene)piperidine-1-carboxylate (960 mg) as a colorless oil.

¹H-NMR (400 MHz, CDCl₃) δ ppm: 6.02 (s, 1H), 3.42-3.48 (m, 4H), 2.42 (m, 2H), 2.27 (m, 2H), 1.49 (s, 9H).

### Preparation of tert-butyl 4-[(4,4,5,5-tetramethyl-1,3,2-dioxaborolan-2-yl)methylene]piperidine-1-carboxylate

To a re-sealable tube was charged tert-butyl 4-(bromomethylene)piperidine-1-carboxylate (700 mg; 2.51 mmol), potassium acetate (620 mg; 6.27 mmol), bis(pinacolato)diboron (1040 mg; 4.01 mmol) and dioxane (20 mL) at room temperature. Argon was bubbled in the reaction mixture for 10 minutes and triphenylphosphine (70 mg; 0.25 mmol) and tris(dibenzylideneacetone)dipalladium-chloroform adduct (160 mg; 0.15 mmol) were added. The tube was flushed with argon and sealed. The reaction mixture was then heated to 100° C. and stirred for 4 hours. After cooling to room temperature, the reaction mixture was filtered and the cake was washed with ethyl acetate. The filtrate was finally concentrated to dryness. The crude residue was then purified by column chromatography (silica gel; cyclohexane:ethylacetate; 1:0 to 4:1; v/v) to afford tert-butyl 4-[(4,4,5,5-tetramethyl-1,3,2-dioxaborolan-2-yl)methylene]piperidine-1-carboxylate (720 mg) as a light yellow solid.

¹H-NMR (400 MHz, CDCl₃) δ ppm: 5.17 (s, 1H), 3.42-3.48 (m, 4H), 2.62 (m, 2H), 2.28 (m, 2H), 1.49 (s, 9H), 1.28 (s, 12H).

### Preparation of tert-butyl 4-[(2-fluoro-4-formyl-phenyl)methylene]piperidine-1-carboxylate

In a re-sealable tube was charged tert-butyl 4-[(4,4,5,5-tetramethyl-1,3,2-dioxaborolan-2-yl)methylene]piperidine-1-carboxylate (720 mg; 2.01 mmol), 4-bromo-3-fluoro-benzaldehyde (500 mg; 2.41 mmol) [133059-46-5], potassium carbonate (800 mg; 5.73 mmol), water (4 mL) and dioxane (20 mL) at room temperature. Argon was bubbled into the mixture for 10 minutes and tetrakis(triphenylphosphine)palladium(0) (140 mg; 0.12 mmol) was added. Argon was bubbled for additional 5 minutes and the tube was sealed. The reaction mixture was then heated to 95° C. and stirred for 3 hours. After cooling to room temperature, ethyl acetate and water were added and the organic layer was separated, washed with brine, dried over MgSO₄, filtered and concentrated to dryness. The crude residue was then purified by column chromatography (silica gel; cyclohexane:ethylacetate; 1:0 to 65:35; v/v) to afford tert-butyl 4-[(2-fluoro-4-formyl-phenyl)methylene]piperidine-1-carboxylate (400 mg) as a light yellow solid.

¹H-NMR (400 MHz, CDCl₃) δ ppm: 9.98 (s, 1H), 7.54-7.56 (m, 2H), 7.36-7.40 (m, 1H), 6.34 (s, 1H), 3.56 (m, 2H), 3.46 (m, 2H), 2.37-2.43 (m, 4H), 1.50 (s, 9H).

### Preparation of 3-fluoro-4-(4-piperidylidenemethyl)benzaldehyde, trifluoroacetic acid salt

To a stirred solution of tert-butyl 4-[(2-fluoro-4-formyl-phenyl)methylene]piperidine-1-carboxylate (400 mg; 1.19 mmol) in dichloromethane (10 mL) was added dropwise trifluoroacetic acid (1.34 mL; 17.85 mmol). The color of the solution changed instantaneously to dark orange. The solution was stirred for 1.5 hours and then concentrated to dryness to afford 3-fluoro-4-(4-piperidylidenemethyl)benzaldehyde, trifluoroacetic acid salt (410 mg) as an orange solid.

MS m/z (+ESI): 220.2 [M+H]⁺.

### Preparation of 4-[(2-fluoro-4-formyl-phenyl)methylene]-N— (3-methyl-1,2,4-thiadiazol-5-yl)piperidine-1-carboxamide

The title compound was prepared as a white solid following scheme 1 and in analogy to Example 27 using 3-fluoro-4-(4-piperidylidenemethyl)benzaldehyde, trifluoroacetic acid salt and (4-nitrophenyl) N-(3-methyl-1,2,4-thiadiazol-5-yl)carbamate as starting materials and after purification by column chromatography (silica gel; cyclohexane:ethylacetate; 1:1 to 0:1; v/v).

### Preparation of Example 89: 4-[[2-fluoro-4-(hydroxymethyl)phenyl]methylene]-N-(3-methyl-1,2,4-thiadiazol-5-yl)piperidine-1-carboxamide

### Preparation of 4-[[2-fluoro-4-(hydroxymethyl)phenyl]methylene]-N-(3-methyl-1,2,4-thiadiazol-5-yl)piperidine-1-carboxamide

To a stirred solution of 4-[(2-fluoro-4-formyl-phenyl)methylene]-N-(3-methyl-1,2,4-thiadiazol-5-yl)piperidine-1-carboxamide (100 mg; 0.27 mmol) (Example 88) in methanol (3 mL) was added sodium borohydride (21 mg; 0.55 mmol). The reaction solution was stirred for 0.5 hours and then deactivated with water (0.5 mL). Ethyl acetate was added and the mixture was washed with brine twice, dried over MgSO₄, filtered and concentrated to dryness to afford 4-[[2-fluoro-4-(hydroxymethyl)phenyl]methylene]-N-(3-methyl-1,2,4-thiadiazol-5-yl)piperidine-1-carboxamide (95 mg) as a light brown powder.

### Preparation of Example 90: 4-[[2-fluoro-4-[[2-hydroxyethyl(methyl)amino]methyl]phenyl]methylene]-N-(3-methyl-1,2,4-thiadiazol-5-yl)piperidine-1-carboxamide, hydrochloride

### Preparation of 4-[[2-fluoro-4-[[2-hydroxyethyl(methyl)amino]methyl]phenyl]methylene]-N-(3-methyl-1,2,4-thiadiazol-5-yl)piperidine-1-carboxamide hydrochloride

To a stirred suspension of 4-[(2-fluoro-4-formyl-phenyl)methylene]-N-(3-methyl-1,2,4-thiadiazol-5-yl)piperidine-1-carboxamide (50 mg; 0.14 mmol) (Example 88) in 1,2-dichloroethane (2 mL) were added triethylamine and 2-(methylamino)ethanol (0.02 mL; 0.21 mmol) [109-83-1] and the mixture was stirred for 1.5 hours. Solid sodium triacetoxyborohydride (240 mg; 1.10 mmol) was added to the resulting clear solution and the mixture was stirred for 1 hour. Ethyl acetate (15 mL) was added and the mixture was washed with 8% sodium hydrogen carbonate aqueous solution, brine, dried over MgSO₄, filtered and concentrated to dryness. The residue was dissolved in acetonitrile (1 mL) and water (10 mL) was added to get a turbid solution. The turbid solution was cooled to 0° C. and then treated with 3N HCl aqueous solution (0.06 mL). After stirring for 0.5 h at 0° C., the resulting clear solution was freeze-dried to afford 4-[[2-fluoro-4-[[2-hydroxyethyl(methyl)amino]methyl]phenyl]methylene]-N-(3-methyl-1,2,4-thiadiazol-5-yl)piperidine-1-carboxamide hydrochloride (57 mg) as an off-white powder.

### Preparation of Example 92: 4-(4-chloro-2-fluoro-benzoyl)-N-(3-methyl-1,2,4-thiadiazol-5-yl)piperidine-1-carboxamide

### Preparation of tert-butyl 2-(4-chloro-2-fluoro-phenyl)-1-oxa-6-azaspiro[2.5]octane-6-carboxylate

To an ice-cold solution of tert-butyl 4-[(4-chloro-2-fluoro-phenyl)methylene]piperidine-1-carboxylate (300 mg; 0.92 mmol) (intermediate of Example 39) in dichloromethane (10 mL) was added 3-chloroperbenzoic acid, technical ˜70% (290 mg; 1.2 mmol) and the reaction solution was stirred for 1 hour at 0-5° C. The reaction solution was further stirred for 24 hours at room temperature. The reaction solution was washed with 5% Na₂S₂O₃ aqueous solution and the organic layer was dried over MgSO₄, filtered and concentrated to dryness. The residue was purified by column chromatography (silica gel; cyclohexane:ethyl acetate; 95:5 to 45:55; v/v) to afford tert-butyl 2-(4-chloro-2-fluoro-phenyl)-1-oxa-6-azaspiro[2.5]octane-6-carboxylate (290 mg) as a colorless oil.

¹H-NMR (400 MHz, CDCl₃) δ ppm: 7.22-7.26 (m, 1H), 7.10-7.18 (m, 2H), 4.00 (s, 1H), 3.59-3.71 (m, 2H), 3.47-3.53 (m, 1H), 3.33-3.41 (m, 1H), 1.84-1.90 (m, 1H), 1.75-1.80 (m, 1H), 1.48 (s, 9h), 1.37-1.41 (m, 2H).

### Preparation of 4-(4-chloro-2-fluoro-benzoyl)-N-(3-methyl-1,2,4-thiadiazol-5-yl)piperidine-1-carboxamide

In a re-sealable tube, tert-butyl 2-(4-chloro-2-fluoro-phenyl)-1-oxa-6-azaspiro[2.5]octane-6-carboxylate (220 mg; 0.61 mmol) was dissolved in neat 96% sulfuric acid (0.68 mL; 12.23 mmol). After 5 minutes, the tube was sealed and the reaction mixture was heated to 100° C. and stirred for 1 hour. After cooling to room temperature, the tube was placed in an ice-bath and the mixture was diluted with cold water (5 mL). 4N NaOH aqueous solution was added to adjust the pH to about 10. Ethyl acetate and brine were added. The organic layer was separated, washed with brine, dried over MgSO₄, filtered and concentrated to dryness. The residue obtained was then directly dissolved in N,N-dimethylformamide (4 mL). The resulting solution was finally treated with triethylamine (0.26 mL; 1.83 mmol) and (4-nitrophenyl) N-(3-methyl-1,2,4-thiadiazol-5-yl)carbamate (180 mg; 0.61 mmol). The mixture was stirred for 0.5 hours and ethyl acetate was added. The solution was washed with water, 8% NaHCO₃ aqueous solution, brine, dried over MgSO₄, filtered and concentrated to dryness. The crude residue was purified by column chromatography (silica gel; cyclohexane:ethyl acetate; 1:1 to 0:1; v/v) to afford 4-(4-chloro-2-fluoro-benzoyl)-N-(3-methyl-1,2,4-thiadiazol-5-yl)piperidine-1-carboxamide (28 mg) as a light yellow powder.

### Preparation of Example 93: 4-[[2-fluoro-4-(methoxymethyl)phenyl]methylene]-N-(3-methyl-1,2,4-thiadiazol-5-yl)piperidine-1-carboxamide

### Preparation of [3-fluoro-4-[[1-[(3-methyl-1,2,4-thiadiazol-5-yl)carbamoyl]-4-piperidylidene]methyl]phenyl]methyl methanesulfonate

To an ice-cold suspension of 4-[[2-fluoro-4-(hydroxymethyl)phenyl]methylene]-N-(3-methyl-1,2,4-thiadiazol-5-yl)piperidine-1-carboxamide (80 mg; 0.21 mmol) (Example 89) in dichloromethane (2 mL) was successively added triethylamine (0.07 mL; 0.52 mmol) and methane sulfonyl chloride (0.02 mL; 0.25 mmol). The mixture was stirred for 1 hour at 0° C. Methanol (1 mL) was added to quench the reaction and volatiles were removed under reduced pressure to afford [3-fluoro-4-[[1-[(3-methyl-1,2,4-thiadiazol-5-yl)carbamoyl]-4-piperidylidene]methyl]phenyl]methyl methanesulfonate as colorless oil in quantitative yield and was used in the next step without further purification.

MS m/z (+ESI): 441.1 [M+H]⁺.

### Preparation of 4-[[2-fluoro-4-(methoxymethyl)phenyl]methylene]-N-(3-methyl-1,2,4-thiadiazol-5-yl)piperidine-1-carboxamide

Crude [3-fluoro-4-[[1-[(3-methyl-1,2,4-thiadiazol-5-yl)carbamoyl]-4-piperidylidene]methyl]phenyl]-methyl methanesulfonate, obtained from previous step, was dissolved in methanol (2 mL) and then treated dropwise with lithium methoxide solution ˜10% in methanol (0.38 mL; 0.84 mmol). The reaction solution was stirred for 16 hours. Further lithium methoxide solution ˜10% in methanol (0.38 mL; 0.84 mmol) was added. The reaction solution was heated to 60° C. and stirred for 3 hours. After cooling to room temperature, the reaction solution was deactivated with a saturated aqueous solution of ammonium chloride. Ethyl acetate was added. The organic layer was separated, washed with brine, dried over MgSO₄, filtered and concentrated to dryness. The residue was purified by column chromatography (silica gel; cyclohexane:ethyl acetate; 1:1 to 1:0; v/v) to afford 4-[[2-fluoro-4-(methoxymethyl)phenyl]methylene]-N-(3-methyl-1,2,4-thiadiazol-5-yl)piperidine-1-carboxamide (33 mg) as a white solid.

### Preparation of Example 95: 4-[(4-cyano-2,6-difluoro-phenyl)methylene]-N-(3-methyl-1,2,4-thiadiazol-5-yl)piperidine-1-carboxamide

### Preparation of 4-(bromomethyl)-3,5-difluoro-benzonitrile

To an ice-cold solution of 3,5-difluoro-4-(hydroxymethyl)benzonitrile (250 mg; 1.48 mmol) [CAS 228421-83-8] in dichlormethane (10 mL) was added phosphorus tribromide (0.07 mL; 0.74 mmol). The mixture was stirred at 0° C. for 3 hours. The mixture was deactivated by water and diluted with dichloromethane (10 mL). The mixture was washed with a saturated aqueous solution of sodium hydrogen carbonate, dried over anhydrous Na₂SO₄, filtered and concentrated to dryness to afford 4-(bromomethyl)-3,5-difluoro-benzonitrile (187 mg) as a white solid.

¹H-NMR (400 MHz, CDCl₃) δ ppm: 7.27 (s, 1H), 7.25 (s, 1H), 4.50 (s, 2H).

### Preparation of tert-butyl 4-[(4-cyano-2,6-difluoro-phenyl)methylene]piperidine-1-carboxylate

The title compound was prepared as a white solid following scheme 1 and in analogy to Example 27 using 4-(bromomethyl)-3,5-difluoro-benzonitrile, triethyl phosphite and tert-butyl 4-oxopiperidine-1-carboxylate [79099-07-3] as starting materials.

MS m/z (+ESI): 335.2 [M+H]⁺.

¹H-NMR (400 MHz, CDCl₃) δ ppm: 7.23 (s, 1H), 7.21 (s, 1H), 6.00 (s, 1H), 3.54-3.56 (m, 2H), 3.43-3.46 (m, 2H), 2.40-2.43 (m, 2H), 2.11-2.15 (m, 2H), 1.49 (s, 9H).

### Preparation of 4-[(4-cyano-2,6-difluoro-phenyl)methylene]piperidine

To a solution of tert-butyl 4-[(4-cyano-2,6-difluoro-phenyl)methylene]piperidine-1-carboxylate (710 mg; 2.11 mmol) in ethyl acetate (10 mL) was added a solution of 2N HCl in ethyl acetate (30 mL). The mixture was stirred for 2 hours. The reaction mixture was then concentrated to dryness to afford 4-[(4-cyano-2,6-difluoro-phenyl)methylene]piperidine (492 mg) as off-white solid, which was used in the next step without purification.

MS m/z (+ESI): 235.2 [M+H]⁺.

### Preparation of 4-[(4-cyano-2,6-difluoro-phenyl)methylene]-N-(3-methyl-1,2,4-thiadiazol-5-yl)piperidine-1-carboxamide

The title compound was prepared as a white solid following scheme 1 and in analogy to Example 27 using 4-[(4-cyano-2,6-difluoro-phenyl)methylene]piperidine and (4-nitrophenyl) N-(3-methyl-1,2,4-thiadiazol-5-yl)carbamate as starting materials and after purification by preparative HPLC.

### Preparation of Example 99: 4-[(2,6-difluoro-4-hydroxy-phenyl)methylene]-N-(3-methyl-1,2,4-thiadiazol-5-yl)piperidine-1-carboxamide

### Preparation of 4-[(2,6-difluoro-4-hydroxy-phenyl)methylene]-N-(3-methyl-1,2,4-thiadiazol-5-yl)piperidine-1-carboxamide

To a stirred solution of 4-[(2,6-difluoro-4-methoxy-phenyl)methylene]-N-(3-methyl-1,2,4-thiadiazol-5-yl)piperidine-1-carboxamide (80 mg; 0.21 mmol) in dichloromethane (10 mL) was added boron tribromide (530 mg; 2.08 mmol) at −78° C. The reaction mixture was slowly warmed up to 20° C. and stirred for 16 hours. The mixture was deactivated by addition of water and the product was extracted with dichloromethane. The organic layer was then washed with a saturated aqueous solution of sodium hydrogen carbonate, dried over anhydrous Na₂SO₄, filtered and concentrated to dryness. The residue was purified by preparative HPLC to afford 4-[(2,6-difluoro-4-hydroxy-phenyl)methylene]-N-(3-methyl-1,2,4-thiadiazol-5-yl)piperidine-1-carboxamide (38 mg) as a white solid.

### Preparation of Example 101: 4-[[4-(2-aminoethylamino)-2-fluoro-phenyl]methylene]-N-(3-methyl-1,2,4-thiadiazol-5-yl)piperidine-1-carboxamide

### Preparation of tert-butyl N-[2-[3-fluoro-4-[[1-[(3-methyl-1,2,4-thiadiazol-5-yl)carbamoyl]-4-piperidylidene]methyl]anilino]ethyl]carbamate

To a re-sealable tube was charged N-Boc-ethylenediamine (2070 mg; 12.95 mmol) [CAS 57260-73-8], 4-[(4-chloro-2-fluoro-phenyl)methylene]-N-(3-methyl-1,2,4-thiadiazol-5-yl)piperidine-1-carboxamide (1000 mg; 2.59 mmol) (Example 39), tris(dibenzylideneacetone)dipalladium chloroform complex (240 mg; 0.26 mmol), sodium tert-butoxide (500 mg; 5.18 mmol), di-tert-butyl-[3,6-dimethoxy-2-(2,4,6-triisopropylphenyl)phenyl]phosphane (250 mg; 0.52 mmol) and tert-butanol (20 mL). The tube was degassed, back filled with argon and sealed. The reaction mixture was heated to 110° C. and stirred for 18 hours. The reaction mixture was concentrated under reduced pressure and the residue was purified by column chromatography (silica gel; petroleum ether:ethyl acetate; 3:1 to 2:1; v/v) to afford tert-butyl N— [2-[3-fluoro-4-[[1-[(3-methyl-1,2,4-thiadiazol-5-yl)carbamoyl]-4-piperidylidene]methyl]anilino]ethyl]carbamate (930 mg) as a white solid.

MS m/z (+ESI): 491.2 [M+H]⁺.

### Preparation of 4-[[4-(2-aminoethylamino)-2-fluoro-phenyl]methylene]-N-(3-methyl-1,2,4-thiadiazol-5-yl)piperidine-1-carboxamide

To a stirred solution of tert-butyl N-[2-[3-fluoro-4-[[1-[(3-methyl-1,2,4-thiadiazol-5-yl)carbamoyl]-4-piperidylidene]methyl]anilino]ethyl]carbamate (500 mg; 0.92 mmol) in ethyl acetate (10 mL) was added dropwise 2N HCl in ethyl acetate (5 mL). The mixture was stirred for 4 hours and then concentrated to dryness. The residue was purified by preparative HPLC to afford 4-[[4-(2-aminoethylamino)-2-fluoro-phenyl]methylene]-N-(3-methyl-1,2,4-thiadiazol-5-yl)piperidine-1-carboxamide (328 mg) as a white solid.

### Preparation of Example 107: 4-[[2-fluoro-4-(3-hydroxypropoxy)phenyl]methylene]-N-(3-methyl-1,2,4-thiadiazol-5-yl)piperidine-1-carboxamide

### Preparation of 4-[(2-fluoro-4-hydroxy-phenyl)methylene]-N-(3-methyl-1,2,4-thiadiazol-5-yl)piperidine-1-carboxamide

The title compound was prepared as an off-white solid following scheme 1 and in analogy to Example 101 using 4-[(4-chloro-2-fluoro-phenyl)methylene]-N-(3-methyl-1,2,4-thiadiazol-5-yl)piperidine-1-carboxamide (Example 39), tris(dibenzylideneacetone)dipalladium chloroform complex, di-tert-butyl-[3,6-dimethoxy-2-(2,4,6-triisopropylphenyl)phenyl]phosphane, sodium tert-butoxide, tert-butanol and replacing N-Boc-ethylenediamine by water (16 equivalents) as starting materials and after purification by column chromatography (silica gel; petroleum ether:ethyl acetate; 2:1; v/v).

MS m/z (+ESI): 349.1 [M+H]⁺.

### Preparation of 4-[[2-fluoro-4-(3-hydroxypropoxy)phenyl]methylene]-N-(3-methyl-1,2,4-thiadiazol-5-yl)piperidine-1-carboxamide

To a stirred solution of 4-[(2-fluoro-4-hydroxy-phenyl)methylene]-N-(3-methyl-1,2,4-thiadiazol-5-yl)piperidine-1-carboxamide (400 mg; 1.09 mmol) in N,N-dimethylformamide (8 mL) were added potassium carbonate (300 mg; 2.18 mmol) and 3-chloro-1-propanol (310 mg; 3.27 mmol). The reaction mixture was stirred for 48 hours. Insoluble were removed by filtration and the filtrate was concentrated to dryness. The residue was purified by preparative HPLC to afford 4-[[2-fluoro-4-(3-hydroxypropoxy)phenyl]methylene]-N-(3-methyl-1,2,4-thiadiazol-5-yl)piperidine-1-carboxamide (37 mg) as a white solid

### Preparation of Example 108: N-(3-amino-1,2,4-thiadiazol-5-yl)-4-[(4-chloro-2-fluoro-phenyl)methyl]piperidine-1-carboxamide

### Preparation of N-(5-amino-1,2,4-thiadiazol-3-yl)-4-methyl-benzenesulfonamide

A solution of carbamimidoylthiourea (2360 mg; 19.77 mmol) [CAS 2114-02-5] in pyridine (40 mL) was treated with p-toluenesulfonyl chloride (7690 mg; 39.5 mmol). The reaction mixture was heated to 100° C. and stirred for 15 minutes. p-toluenesulfonyl chloride (1920 mg; 10 mmol) was added again and the reaction mixture was stirred for additional 15 minutes. The addition ofp-toluenesulfonyl chloride was repeated. After cooling to room temperature, the crude reaction was cautiously deactivated with 300 g of ice. Concentrated HCl aqueous solution (45 mL) was added. The resulting suspension was filtered and the solid was further purified by column chromatography (silica gel; petroleum ether:ethyl acetate; 5:1 to 1:1; v/v) to afford N-(5-amino-1,2,4-thiadiazol-3-yl)-4-methyl-benzenesulfonamide (250 mg) as an off-white solid.

MS m/z (+ESI): 271.0 [M+H]⁺.

¹H-NMR (400 MHz, DMSO-d₆+D₂O) δ ppm: 7.78 (d, J=8.0 Hz, 2H), 7.35 (d, J=8.4 Hz, 2H), 2.35 (s, 3H).

### Preparation of 4-[(4-chloro-2-fluoro-phenyl)methyl]-N-[3-(p-tolylsulfonylamino)-1,2,4-thiadiazol-5-yl]piperidine-1-carboxamide

The title compound was prepared as an off-white solid following scheme 1 and in analogy to Example 70 using N-(5-amino-1,2,4-thiadiazol-3-yl)-4-methyl-benzenesulfonamide and 4-[(4-chloro-2-fluoro-phenyl)methyl]piperidine hydrochloride (intermediate of Example 9) as starting materials.

MS m/z (+ESI): 524.1, 526.1 [M+H]⁺.

### Preparation of N-(3-amino-1,2,4-thiadiazol-5-yl)-4-[(4-chloro-2-fluoro-phenyl)methyl]piperidine-1-carboxamide

4-[(4-chloro-2-fluoro-phenyl)methyl]-N-[3-(p-tolylsulfonylamino)-1,2,4-thiadiazol-5-yl]piperidine-1-carboxamide (170 mg; 0.26 mmol) was slowly added to 96% sulfuric acid (1 mL) at 0° C. The reaction solution was stirred for 2 hours at 0° C. The reaction solution was diluted with ice-cold water and the pH was adjusted to 7 using 2N sodium hydroxide aqueous solution. The product was extracted with a mixture of ethyl acetate and tetrahydrofuran (100 mL; 1:1; v/v). The organic layer was then washed with brine, dried over anhydrous Na₂SO₄, filtered and concentrated to dryness. The residue was purified by preparative HPLC to afford N-(3-amino-1,2,4-thiadiazol-5-yl)-4-[(4-chloro-2-fluoro-phenyl)methyl]piperidine-1-carboxamide (37 mg) as a white solid.

### Preparation of Example 119: 4-[(4-amino-2-fluoro-phenyl)methylene]-N-(3-methyl-1,2,4-thiadiazol-5-yl)piperidine-1-carboxamide

### Preparation of 4-[[2-fluoro-4-[(4-methoxyphenyl)methylamino]phenyl]methylene]-N-(3-methyl-1,2,4-thiadiazol-5-yl)piperidine-1-carboxamide

The title compound was prepared as an off-white solid following scheme 1 and in analogy to Example 101 using 4-[(4-chloro-2-fluoro-phenyl)methylene]-N-(3-methyl-1,2,4-thiadiazol-5-yl)piperidine-1-carboxamide (Example 39) and 4-methoxybenzylamine [2393-23-9] as starting materials.

MS m/z (+ESI): 468.2 [M+H]⁺.

### Preparation of 4-[(4-amino-2-fluoro-phenyl)methylene]-N-(3-methyl-1,2,4-thiadiazol-5-yl)piperidine-1-carboxamide

4-[[2-fluoro-4-[(4-methoxyphenyl)methylamino]phenyl]methylene]-N-(3-methyl-1,2,4-thiadiazol-5-yl)piperidine-1-carboxamide (240 mg; 0.46 mmol) was dissolved in neat trifluoroacetic acid (3 mL) and triethylsilane (0.37 mL; 2.31 mmol) was added. The reaction mixture was stirred for 16 hours and then concentrated to dryness. The residue was purified by preparative HPLC to afford 4-[(4-amino-2-fluoro-phenyl)methylene]-N-(3-methyl-1,2,4-thiadiazol-5-yl)piperidine-1-carboxamide (26 mg) as a light yellow solid.

### Preparation of Example 122: 4-[[4-[2-aminoethyl(methyl)amino]-2-fluoro-phenyl]methylene]-N-(3-methyl-1,2,4-thiadiazol-5-yl)piperidine-1-carboxamide, trifluoroacetic acid

### Preparation of tert-butyl N-[2-[3-fluoro-N-methyl-4-[[1-[(3-methyl-1,2,4-thiadiazol-5-yl)carbamoyl]-4-piperidylidene]methyl]anilino]ethyl]carbamate

To a solution of tert-butyl N-[2-[3-fluoro-4-[[1-[(3-methyl-1,2,4-thiadiazol-5-yl)carbamoyl]-4-piperidylidene]methyl]anilino]ethyl]carbamate (250 mg; 0.48 mmol)(intermediate Example 101) in 1.2-dichloroethane (4 mL) and methanol (2 mL) were added 37% (w/w) formaldehyde in water (43 mg; 0.53 mmol) and acetic acid (0.003 mL). The resulting reaction mixture was stirred for 10 minutes and sodium cyanoborohydride (91.3 mg; 1.45 mmol) was added. After agitation for 1 hour, the reaction mixture was concentrated. The residue was purified by column chromatography (silica gel; petroleum ether:ethyl acetate; 2:1 to 1:1; v/v) to afford tert-butyl N-[2-[3-fluoro-N-methyl-4-[[1-[(3-methyl-1,2,4-thiadiazol-5-yl)carbamoyl]-4-piperidylidene]methyl]anilino]ethyl]carbamate (170 mg) as a light yellow solid.

MS m/z (+ESI): 505.2 [M+H]⁺.

### Preparation of 4-[[4-[2-aminoethyl(methyl)amino]-2-fluoro-phenyl]methylene]-N-(3-methyl-1,2,4-thiadiazol-5-yl)piperidine-1-carboxamide, trifluoroacetic acid

tert-butyl N-[2-[3-fluoro-N-methyl-4-[[1-[(3-methyl-1,2,4-thiadiazol-5-yl)carbamoyl]-4-piperidylidene]methyl]anilino]ethyl]carbamate (210 mg; 0.39 mmol) was dissolved in 4N HCl in dioxane (3 mL). The reaction mixture was stirred for 1 h and then concentrated to dryness. The residue was purified by preparative HPLC to afford 4-[[4-[2-aminoethyl(methyl)amino]-2-fluoro-phenyl]methylene]-N-(3-methyl-1,2,4-thiadiazol-5-yl)piperidine-1-carboxamide, trifluoroacetic acid (130 mg) as a white solid.

### Preparation of Example 123: 4-[[4-[2-acetamidoethyl(methyl)amino]-2-fluoro-phenyl]methylene]-N-(3-methyl-1,2,4-thiadiazol-5-yl)piperidine-1-carboxamide

### Preparation of 4-[[4-[2-acetamidoethyl(methyl)amino]-2-fluoro-phenyl]methylene]-N-(3-methyl-1,2,4-thiadiazol-5-yl)piperidine-1-carboxamide

HATU (240 mg; 0.63 mmol) was added to a stirred mixture of acetic acid (51 mg; 0.85 mmol) and NaHCO₃ (180 mg; 2.11 mmol) in N,N-dimethylformamide (5 mL). The resulting mixture was stirred for 0.5 hours and then treated with solid 4-[[4-[2-aminoethyl(methyl)amino]-2-fluoro-phenyl]methylene]-N— (3-methyl-1,2,4-thiadiazol-5-yl)piperi-dine-1-carboxamide (190 mg; 0.42 mmol)(Example 122). The reaction mixture was stirred for 1 hour. The reaction mixture was then filtered and the filtrate was purified by preparative HPLC to afford 4-[[4-[2-acetamidoethyl(methyl)amino]-2-fluoro-phenyl]methylene]-N-(3-methyl-1,2,4-thiadiazol-5-yl)piperidine-1-carboxamide (88 mg) as a white solid.

### Preparation of Example 129: 4-[(4-cyano-2,6-difluoro-phenyl)methyl]-N-(3-methyl-1,2,4-thiadiazol-5-yl)piperidine-1-carboxamide

### Preparation of tert-butyl 4-[(4-carbamoyl-2,6-difluoro-phenyl)methyl]piperidine-1-carboxylate

The title compound was prepared as an off-white solid following scheme 1 and in analogy to Examples 88, 37 and 27 using ethyl 4-bromo-3,5-difluoro-benzoate [1562995-70-3] as starting material.

MS m/z (+ESI): 299.1 [M-tBu+H]⁺.

### Preparation of tert-butyl 4-[(4-cyano-2,6-difluoro-phenyl)methyl]piperidine-1-carboxylate

To a stirred solution of tert-butyl 4-[(4-carbamoyl-2,6-difluoro-phenyl)methyl]piperidine-1-carboxylate (114 mg; 0.31 mmol) and triethylamine (0.11 mL; 0.76 mmol) in dichloromethane (5 mL) was gradually added trifluoroacetic anhydride (0.11 mL; 0.76 mmol) at 0° C. The reaction mixture was allowed to warm up to room temperature upon the end of addition. After agitation for 2 hours, the reaction mixture was partitioned between dichloromethane and saturated aqueous potassium carbonate solution. The organic layer was separated, washed with brine, dried over dried over anhydrous Na₂SO₄, filtered and concentrated to dryness. The residue was purified by column chromatography (silica gel; petroleum ether:ethyl acetate; 5:1; v/v) to afford tert-butyl 4-[(4-cyano-2,6-difluoro-phenyl)methyl]piperidine-1-carboxylate (80 mg) as an off-white solid.

MS m/z (+ESI): 281.1 [M-tBu+H]⁺.

### Preparation of 4-[(4-cyano-2,6-difluoro-phenyl)methyl]-N-(3-methyl-1,2,4-thiadiazol-5-yl)piperidine-1-carboxamide

The title compound was then prepared as a white solid following scheme 1 and in analogy to Example 27 using tert-butyl 4-[(4-cyano-2,6-difluoro-phenyl)methyl]piperidine-1-carboxylate as starting material.

### Preparation of Example 130: (4Z)-4-[(4-chloro-2-fluoro-phenyl)methylene]-3-(methoxymethyl)-N-(3-methyl-1,2,4-thiadiazol-5-yl)piperidine-1-carboxamide

### Preparation of tert-butyl 4-hydroxy-3-(hydroxymethyl)piperidine-1-carboxylate

To a stirred solution of 1-tert-butyl 3-methyl 4-oxopiperidine-1,3-dicarboxylate (2350 mg; 8.86 mmol)[161491-24-3] in methanol (50 mL) was added sodium borohydride (1030 mg; 26.6 mmol) at 0° C. The reaction mixture was then heated to 60° C. and stirred for 2 hours. After cooling to room temperature, the reaction mixture was treated with 1N HCl aqueous solution (10 mL) and the product was extracted with diethyl ether (4×40 mL). The combined organic layers were dried over Na₂SO₄, filtered and concentrated to dryness. The residue was purified by column chromatography (silica gel; petroleum ether:acetone; 3:2; v/v) to afford tert-butyl 4-hydroxy-3-(hydroxymethyl)piperidine-1-carboxylate (1900 mg) as a yellow oil.

### Preparation of tert-butyl 3-[[tert-butyl(dimethyl)silyl]oxymethyl]-4-hydroxy-piperidine-1-carboxylate

To a stirred solution of tert-butyl 4-hydroxy-3-(hydroxymethyl)piperidine-1-carboxylate (1900 mg; 6.50 mmol) in dichloromethane (30 mL) was added triethylamine (2.76 mL; 19.7 mmol) and catalytic amount of 4-(dimethylamino)pyridinee (160 mg; 1.30 mmol) at 0° C. The reaction solution was treated with tert-butyldimethylchlorosilane (2970 mg; 19.7 mmol) at 0° C. and then allowed to warm up to room temperature. After stirring for 20 hours, volatiles were evaporated under reduced pressure. The residue was purified by column chromatography (silica gel; petroleum ether:ethyl acetate; 5:1; v/v) to afford tert-butyl 3-[[tert-butyl(dimethyl)silyl]oxymethyl]-4-hydroxy-piperidine-1-carboxylate (2000 mg) as a yellow oil.

¹H-NMR (400 MHz, CDCl₃) δ ppm: 3.25-4.20 (m, 7H), 1.61-1.76 (m, 3H), 1.46 (s, 9H), 0.90 (s, 9H), 0.08 (s, 6H).

### Preparation of tert-butyl 3-[[tert-butyl(dimethyl)silyl]oxymethyl]-4-oxo-piperidine-1-carboxylate

To a stirred solution of tert-butyl 3-[[tert-butyl(dimethyl)silyl]oxymethyl]-4-hydroxy-piperidine-1-carboxylate (1800 mg; 4.40 mmol) in dichloromethane (15 mL) was added Dess-Martin periodinane (3800 mg; 8.80 mmol) at 0° C. After stirring for 2 hours at 15° C., the reaction mixture was concentrated under reduced pressure. The residue was purified by column chromatography (silica gel; petroleum ether:ethyl acetate; 4:1; v/v) to afford tert-butyl 3-[[tert-butyl(dimethyl)silyl]oxymethyl]-4-oxo-piperidine-1-carboxylate (1100 mg) as a colorless oil.

MS m/z (+ESI): 344.1 [M+H]⁺.

### Preparation of tert-butyl 3-[[tert-butyl(dimethyl)silyl]oxymethyl]-4-[(4-chloro-2-fluoro-phenyl)methylene]piperidine-1-carboxylate

To a stirred solution of 4-chloro-1-(diethoxyphosphorylmethyl)-2-fluoro-benzene (52 mg; 0.17 mmol)(intermediate of Example 9) in dry tetrahydrofuran (10 mL) was added dropwise a solution of 1.6 N n-butyl lithium in n-hexane (0.09 mL; 0.15 mmol) at −78° C. The reaction mixture was stirred for 0.5 hours at this temperature and tert-butyl 3-[[tert-butyl(dimethyl)silyl]oxymethyl]-4-oxo-piperidine-1-carboxylate (50 mg; 0.14 mmol) was added. The reaction mixture was allowed to warm up to room temperature and was further stirred for 2 hours. The reaction was deactivated by addition of saturated aqueous ammonium chloride solution (5 mL) and the mixture was extracted with ethyl acetate (3×20 mL). The combined organic layers were washed with brine, dried over Na₂SO₄, filtered and concentrated to dryness. The residue was purified by column chromatography (silica gel; petroleum ether:ethyl acetate; 5:1; v/v) to afford tert-butyl 3-[[tert-butyl(dimethyl)silyl]oxymethyl]-4-[(4-chloro-2-fluoro-phenyl)methylene]piperidine-1-carboxylate (95 mg) as a yellow oil and as mixture of E and Z isomers.

MS m/z (+ESI): 470.2, 472.2 [M+H]⁺.

### Preparation of tert-butyl (4Z)-4-[(4-chloro-2-fluoro-phenyl)methylene]-3-(hydroxymethyl)piperidine-1-carboxylate

To a stirred solution of tert-butyl 3-[[tert-butyl(dimethyl)silyl]oxymethyl]-4-[(4-chloro-2-fluoro-phenyl)methylene]piperidine-1-carboxylate (800 mg; 1.53 mmol) in tetrahydrofuran (5 mL) was added a solution of 1M tetrabutylammonium fluoride in tetrahydrofuran (3.1 mL; 3.1 mmol). The reaction solution was stirred for 3 hours and then concentrated to dryness. The residue was purified by column chromatography (silica gel; petroleum ether:ethyl acetate; 4:1; v/v) to afford tert-butyl (4Z)-4-[(4-chloro-2-fluoro-phenyl)methylene]-3-(hydroxymethyl)piperidine-1-carboxylate (280 mg) as a colorless oil.

MS m/z (+ESI): 356.1, 358.1 [M+H]⁺.

¹H-NMR (400 MHz, CDCl₃) δ ppm: 7.08-7.19 (m, 3H), 6.30 (s, 1H), 4.02-4.30 (m, 2H), 3.62 (m, 2H), 2.64-3.20 (m, 3H), 2.22-2.44 (m, 2H), 1.50 (s, 9H).

### Preparation of tert-butyl (4Z)-4-[(4-chloro-2-fluoro-phenyl)methylene]-3-(methoxymethyl)piperidine-1-carboxylate

To a stirred solution of tert-butyl (4Z)-4-[(4-chloro-2-fluoro-phenyl)methylene]-3-(hydroxymethyl)-piperidine-1-carboxylate (240 mg; 0.64 mmol) in tetrahydrofuran (5 mL) was added 60% sodium hydride in mineral oil (51 mg; 1.28 mmol) at 0° C. The reaction mixture was stirred for 1 hour and then treated with iodomethane (182 mg; 1.28 mmol) at 0° C. The reaction mixture was allowed to warm up naturally to room temperature and then stirred for 2 hours. The reaction was deactivated with methanol (1 mL) and then concentrated to dryness. The residue was purified by column chromatography (silica gel; petroleum ether:ethyl acetate; 4:1; v/v) to afford tert-butyl (4Z)-4-[(4-chloro-2-fluoro-phenyl)methylene]-3-(methoxymethyl)piperidine-1-carboxylate (220 mg) as a colorless oil.

MS m/z (+ESI): 370.1, 372.1 [M+H]⁺.

¹H-NMR (400 MHz, CDCl₃) δ ppm: 7.38 (m, 1H), 7.07-7.12 (m, 2H), 6.33 (s, 1H), 4.19-4.38 (m, 2H), 3.50-3.55 (m, 2H), 3.35 (s, 3H), 2.20-2.91 (m, 5H), 1.50 (s, 9H).

### Preparation of (4Z)-4-[(4-chloro-2-fluoro-phenyl)methylene]-3-(methoxymethyl)-N-(3-methyl-1,2,4-thiadiazol-5-yl)piperidine-1-carboxamide

The title compound was prepared as a white solid following scheme 1 and in analogy to Example 95 using tert-butyl (4Z)-4-[(4-chloro-2-fluoro-phenyl)methylene]-3-(methoxymethyl)piperidine-1-carboxylate as starting material and after purification by preparative HPLC.

### Preparation of Examples 134 and 135: (4Z)-4-[(4-chloro-2-fluoro-phenyl)methylene]-3-(2-methoxyethyl)-N-(3-methyl-1,2,4-thiadiazol-5-yl)piperidine-1-carboxamide and (4E)-4-[(4-chloro-2-fluoro-phenyl)methylene]-3-(2-methoxyethyl)-N-(3-methyl-1,2,4-thiadiazol-5-yl)piperidine-1-carboxamide

### Preparation of tert-butyl 4-cyclohexyliminopiperidine-1-carboxylate

In a 350 mL flask, equipped with a Dean Stark condenser, was charged tert-butyl 4-oxopiperidine-1-carboxylate (5290 mg; 26.00 mmol)[CAS 79099-07-3] and cyclohexane (5 mL). Cyclohexanamine (3160 mg; 32.20 mmol) and trifluoroacetic acid (0.5 mL) were added and the reaction mixture was heated to 95° C. and stirred for 16 hours. After cooling down to room temperature, the reaction mixture was diluted with diethyl ether (150 mL) and the mixture was washed with a saturated aqueous solution of NaHCO_{3 (}50 mL), brine (50 mL), dried over Na₂SO₄, filtered and concentrated to dryness to afford tert-butyl 4-cyclohexyliminopiperidine-1-carboxylate (7290 mg) as a yellow solid, which was used in the next step without further purification.

MS m/z (+ESI): 281.1 [M+H]⁺.

### Preparation of tert-butyl 3-(2-methoxyethyl)-4-oxo-piperidine-1-carboxylate

To a stirred solution of tert-butyl 4-cyclohexyliminopiperidine-1-carboxylate (1030 mg; 3.63 mmol) in tetrahydrofuran (40 mL) was added dropwise a solution of 2N lithium diisopropylamide in tetrahydrofuran (2.2 mL; 4.4 mmol) at −78° C. After stirring for 1 hour at −78° C., the reaction was mixture was treated with 1-bromo-2-methoxyethane (520 mg; 3.63 mmol). The reaction mixture was allowed to warm up to room temperature and further stirred for 16 hours. Saturated aqueous ammonium chloride solution (10 mL) was added and the resulting mixture was extracted with ethyl acetate (3×20 mL). The combined organic layers were washed with brine, dried over Na₂SO₄, filtered and concentrated to dryness. The residue was purified by column chromatography (silica gel; petroleum ether:ethyl acetate; 10:1 to 5:1; v/v) to afford tert-butyl 3-(2-methoxyethyl)-4-oxo-piperidine-1-carboxylate (240 mg) as a light yellow oil.

MS m/z (+ESI): 258.2 [M+H]⁺.

### Preparation of tert-butyl (4Z)-4-[(4-chloro-2-fluoro-phenyl)methylene]-3-(2-methoxyethyl)piperidine-1-carboxylate and tert-butyl (4E)-4-[(4-chloro-2-fluoro-phenyl)methylene]-3-(2-methoxyethyl)piperidine-1-carboxylate

To a stirred solution of 4-chloro-1-(diethoxyphosphorylmethyl)-2-fluoro-benzene (427 mg; 1.51 mmol) (intermediate of Example 9) in dry tetrahydrofuran (20 mL) was added dropwise a solution of 1N lithium bis(trimethylsilyl)amide in tetrahydrofuran (1.81 mL; 1.81 mmol) at −78° C. The reaction mixture was stirred for 0.5 hours at −78° C. and then treated with a solution of tert-butyl 3-(2-methoxyethyl)-4-oxo-piperidine-1-carboxylate (392 mg; 1.51 mmol) in tetrahydrofuran (10 mL). The reaction mixture was allowed to warm up to room temperature and was further stirred for 3 hours. Saturated aqueous ammonium chloride solution (5 mL) was added and the resulting mixture was extracted with ethyl acetate (3×20 mL). The combined organic layers were washed with brine, dried over Na₂SO₄, filtered and concentrated to dryness. The residue was purified by column chromatography (silica gel; petroleum ether:ethyl acetate; 15:1; v/v) to afford tert-butyl (4Z)-4-[(4-chloro-2-fluoro-phenyl)methylene]-3-(2-methoxyethyl)piperidine-1-carboxylate (108 mg) as a yellow oil and tert-butyl (4E)-4-[(4-chloro-2-fluoro-phenyl)methylene]-3-(2-methoxyethyl)piperidine-1-carboxylate (121 mg) as a yellow oil.

For Z isomer:

MS m/z (+ESI): 384.0, 386.0 [M+H]⁺.

¹H-NMR (400 MHz, CDCl₃) δ ppm: 7.23 (m, 1H), 7.07-7.12 (m, 2H), 6.23 (s, 1H), 4.04-4.39 (m, 2H), 3.29-3.38 (m, 2H), 3.22 (s, 3H), 2.14-2.85 (m, 5H), 1.76 (m, 2H), 1.49 (s, 9H).

For E isomer:

MS m/z (+ESI): 384.0, 386.0 [M+H]⁺.

¹H-NMR (400 MHz, CDCl₃) δ ppm: 7.09-7.11 (m, 3H), 6.22 (s, 1H), 3.85-4.06 (m, 2H), 3.46 (m, 1H), 3.35 (s, 3H), 2.83-3.24 (m, 2H), 1.78-2.55 (m, 5H), 1.48 (s, 9H).

### Preparation of (4Z)-4-[(4-chloro-2-fluoro-phenyl)methylene]-3-(2-methoxyethyl)-N-(3-methyl-1,2,4-thiadiazol-5-yl)piperidine-1-carboxamide

The title compound was prepared as a white solid following scheme 1 and in analogy to Example 95 using tert-butyl (4Z)-4-[(4-chloro-2-fluoro-phenyl)methylene]-3-(2-methoxyethyl)piperidine-1-carboxylate as starting material and after purification by preparative HPLC.

### Preparation of (4E)-4-[(4-chloro-2-fluoro-phenyl)methylene]-3-(2-methoxyethyl)-N-(3-methyl-1,2,4-thiadiazol-5-yl)piperidine-1-carboxamide

The title compound was prepared as a white solid following scheme 1 and in analogy to Example 95 using tert-butyl (4E)-4-[(4-chloro-2-fluoro-phenyl)methylene]-3-(2-methoxyethyl)piperidine-1-carboxylate as starting material and after purification by preparative HPLC.

### Preparation of Example 138: 4-[[2,6-difluoro-4-(trifluoromethyl)phenyl]methylene]-N-(2-methyl-4-pyridyl)piperidine-1-carboxamide, trifluoroacetic salt

### Preparation of 4-[[2,6-difluoro-4-(trifluoromethyl)phenyl]methylene]-N-(2-methyl-4-pyridyl)piperidine-1-carboxamide, trifluoroacetic acid

Under nitrogen atmosphere, to a stirred solution of 2-methylpyridin-4-amine (140 mg; 1.30 mmol)[CAS 18437-58-6] and triethylamine (0.26 mL; 2.60 mmol) in dichloromethane (10 mL) was added triphosgene (135 mg; 0.45 mmol) at 0° C. After agitation for 2 hours at 0° C., the reaction mixture was treated with a solution of 4-[[2,6-difluoro-4-(trifluoromethyl)phenyl]methylene]piperidine (200 mg; 0.65 mmol) (intermediate of Example 41) in dichloromethane (2 mL). The reaction mixture was allowed to warm up to room temperature and stirred for 16 hours. After concentration, the residue was purified by preparative HPLC to afford 4-[[2,6-difluoro-4-(trifluoromethyl)phenyl]methylene]-N-(2-methyl-4-pyridyl)piperidine-1-carboxamide, trifluoroacetic acid salt (110 mg) as a white solid.

### Preparation of Example 141: 4-(4-bromo-2-fluoro-benzoyl)-N-(3-methyl-1,2,4-thiadiazol-5-yl)-piperidine-1-carboxamide

### Preparation of 1-[4-(4-bromo-2-fluoro-benzoyl)-1-piperidyl]ethanone

A mixture of 1-bromo-3-fluoro-benzene (923 mg; 5.27 mmol)[CAS 1073-06-9], AlCl₃ (84 mg; 0.63 mmol) and 1-acetylisonipecotoyl chloride (100 mg; 0.53 mmol)[CAS 59084-16-1] was stirred at 100° C. for 3 hours. The hot reaction mixture was poured into ice-water (10 mL) and extracted with dichloromethane (20 mL). The organic layer was separated, dried over Na₂SO₄, filtered and concentrated. The residue was purified by column chromatography (silica gel; petroleum ether:ethyl acetate; 1:2; v/v) to afford 1-[4-(4-bromo-2-fluoro-benzoyl)-1-piperidyl]ethanone (30 mg) as a colorless oil.

MS m/z (+ESI): 328.0, 330.0 [M+H]⁺.

### Preparation of (4-bromo-2-fluoro-phenyl)-(4-piperidyl)methanone

A solution of 1-[4-(4-bromo-2-fluoro-benzoyl)-1-piperidyl]ethanone (150 mg; 0.43 mmol) in 6N HCl aqueous solution (8 mL) was heated to 100° C. and stirred for 4 hours. Then the solution was cooled in an ice bath and basified with 25% aqueous sodium hydroxide to pH about 9. The resulting solution was extracted with dichloromethane (3×20 mL) and the combined organic layers were washed with brine (20 mL), dried over Na₂SO₄, filtered and concentrated to dryness to afford (4-bromo-2-fluoro-phenyl)-(4-piperidyl)methanone (110 mg) as a viscous oil.

MS m/z (+ESI): 286.0, 288.0 [M+H]⁺.

### Preparation of 4-(4-bromo-2-fluoro-benzoyl)-N-(3-methyl-1,2,4-thiadiazol-5-yl)piperidine-1-carboxamide

The title compound was prepared as a white solid following scheme 1 and 4 and in analogy to Example 27 using (4-bromo-2-fluoro-phenyl)-(4-piperidyl)methanone and (4-nitrophenyl) N-(3-methyl-1,2,4-thiadiazol-5-yl)carbamate as starting materials and after purification by preparative HPLC.

### Preparation of Example 142: 4-[2-fluoro-4-(trifluoromethyl)benzoyl]-N-(3-methyl-1,2,4-thiadiazol-5-yl)piperidine-1-carboxamide

### Preparation of tert-butyl 4-[2-fluoro-4-(trifluoromethyl)benzoyl]piperidine-1-carboxylate

To a solution of 1-bromo-2-fluoro-4-(trifluoromethyl)benzene (200 mg; 0.82 mmol)[CAS 40161-54-4] in tetrahydrofuran (15 mL) cooled to −70° C. was added dropwise a solution of 1.6M n-butyllithium in n-hexane (0.7 mL; 1.1 mmol). After agitation at −70° C. for 1 hour, the reaction mixture was treated dropwise with a solution of tert-butyl 4-[methoxy(methyl)carbamoyl]piperidine-1-carboxylate (247 mg; 0.91 mmol) [CAS 139290-70-3] in tetrahydrofuran (5 mL). The reaction mixture was stirred for 0.5 hours at −70° C. and then for 2 hours at room temperature. The reaction mixture was deactivated with a saturated aqueous ammonium chloride solution (20 mL), then ethyl acetate (30 mL) was added. The organic layer was separated, dried over Na₂SO₄, filtered and concentrated to dryness. The residue was purified by column chromatography (silica gel; petroleum ether:ethyl acetate; 30:1; v/v) to afford tert-butyl 4-[2-fluoro-4-(trifluoromethyl)benzoyl]piperidine-1-carboxylate (180 mg) as a white solid.

MS m/z (+ESI): 320.1 [M-tBu+H]⁺.

### Preparation of [2-fluoro-4-(trifluoromethyl)phenyl]-(4-piperidyl)methanone, trifluoroacetic acid salt

The title compound was prepared as a light yellow gum following scheme 4 and in analogy to Example 77 using tert-butyl 4-[2-fluoro-4-(trifluoromethyl)benzoyl]piperidine-1-carboxylate as starting material.

MS m/z (+ESI): 276.1 [M+H]⁺.

### Preparation of 4-[2-fluoro-4-(trifluoromethyl)benzoyl]-N-(3-methyl-1,2,4-thiadiazol-5-yl)piperidine-1-carboxamide

The title compound was prepared as a white solid following schemes 1 and 4 and in analogy to Examples 27 and 77 using 2-fluoro-4-(trifluoromethyl)phenyl]-(4-piperidyl)methanone, trifluoroacetic acid salt and (4-nitrophenyl) N-(3-methyl-1,2,4-thiadiazol-5-yl)carbamate as starting materials and after purification by preparative HPLC.

### Preparation of Example 144: 4-[(4-chloro-2-fluoro-phenyl)methylene]-N-(2-ethyl-4-pyridyl)piperidine-1-carboxamide

### Preparation of phenyl N-(2-ethyl-4-pyridyl)carbamate

To a stirred solution of 2-ethylpyridin-4-amine (200 mg; 1.10 mmol)[CAS 50826-64-7] and triethylamine (0.32 mL; 2.19 mmol) in acetonitrile (10 mL) was gradually added phenyl chloroformate (0.16 mL; 1.21 mmol) at 0° C. The reaction mixture was stirred for 3 hours at 0° C. and then concentratede. The residue was partitioned between ethyl acetate (20 mL) and water (10 mL). The organic layer was separated, dried over anhydrous Na₂SO₄, filtered and concentrated to dryness to afford phenyl N-(2-ethyl-4-pyridyl)carbamate (210 mg) as a light brown solid, used in the next step without further purification.

MS m/z (+ESI): 243.1 [M+H]⁺.

### Preparation of 4-[(4-chloro-2-fluoro-phenyl)methylene]-N-(2-ethyl-4-pyridyl)piperidine-1-carboxamide

To a stirred solution of 4-[(4-chloro-2-fluoro-phenyl)methylene]piperidine (100 mg; 0.42 mmol)(intermediate Example 39) in N,N-dimethylformamide (10 mL) were added phenyl N-(2-ethyl-4-pyridyl)carbamate (136 mg; 0.51 mmol) and triethylamine (0.12 mL; 0.84 mmol). The reaction solution was stirred for 3 hours and then concentrated to dryness. The residue was purified by preparative HPLC to afford 4-[(4-chloro-2-fluoro-phenyl)methylene]-N-(2-ethyl-4-pyridyl)piperidine-1-carboxamide (78 mg) as a white solid.

### Preparation of Example 155: 4-[4-(dimethylamino)-2-fluoro-benzoyl]-N-(3-methyl-1,2,4-thiadiazol-5-yl)piperidine-1-carboxamide

### Preparation of 1-[4-[4-(dimethylamino)-2-fluoro-benzoyl]-1-piperidyl]ethanone

A mixture containing 1-[4-(4-bromo-2-fluoro-benzoyl)-1-piperidyl]ethanone (100 mg; 0.29 mmol) (intermediate of Example 141), tris(dibenzylideneacetone)dipalladium chloroform complex (27 mg; 0.03 mmol), 2,2′-bis(diphenylphosphino)-1,1′-binaphthyl (36 mg; 0.06 mmol), cesium carbonate (142 mg; 0.43 mmol), 2N N-ethylethanamine in tetrahydrofuran (0.7 mL; 1.4 mmol) and toluene (5 mL) was stirred at 80° C. for 16 hours. Then, after cooling down to room temperature, the reaction mixture was filtered and the filtrate was concentrated. The residue was purified by preparative HPLC to afford 1-[4-[4-(dimethylamino)-2-fluoro-benzoyl]-1-piperidyl]ethanone (30 mg) as a light yellow solid.

MS m/z (+ESI): 293.2 [M+H]⁺.

### Preparation of 4-[4-(dimethylamino)-2-fluoro-benzoyl]-N-(3-methyl-1,2,4-thiadiazol-5-yl)piperidine-1-carboxamide

The title compound was prepared as a white solid following scheme 1 and 4 and in analogy to Examples 141 and 27 using 1-[4-[4-(dimethylamino)-2-fluoro-benzoyl]-1-piperidyl]ethanone and (4-nitrophenyl) N-(3-methyl-1,2,4-thiadiazol-5-yl)carbamate as starting materials and after purification by preparative HPLC.

### Preparation of Example 164: 4-[[4-[cyanomethyl(methyl)amino]-2,6-difluoro-phenyl]methylene]-N-(3-methyl-1,2,4-thiadiazol-5-yl)piperidine-1-carboxamide

### Preparation of 2-(4-bromo-3,5-difluoro-anilino)acetonitrile

In a re-sealable tube, to a solution of 4-bromo-3,5-difluoroaniline (500 mg; 2.40 mmol)[CAS 203302-95-8] and N,N-disopropylethylamine (0.50 mL; 2.88 mmol) in tetrahydrofuran (5 mL) was added bromoacetonitrile (0.19 mL; 2.76 mmol). The tube was sealed and the solution was stirred at 80° C. for 16 hours. The solvent was evaporated and the residue was purified by column chromatography (silica gel; petroleum ether:ethyl acetate; 2:1; v/v) to afford 2-(4-bromo-3,5-difluoro-anilino)acetonitrile (160 mg) as a light yellow solid.

MS m/z (+ESI): 244.9, 246.9 [M+H]⁺.

¹H-NMR (400 MHz, CDCl₃) δ ppm: 6.32-6.37 (m, 2H), 4.26-4.31 (m, 1H), 4.10 (d, J=6.8 Hz, 2H).

### Preparation of 2-(4-bromo-3,5-difluoro-N-methyl-anilino)acetonitrile

To a solution of 2-(4-bromo-3,5-difluoro-anilino)acetonitrile (1000 mg; 3.85 mmol) in tetrahydrofuran (10 mL) were added cesium carbonate (2556 mg; 7.69 mmol) and methyliodide (1.22 mL; 19.23 mmol) at room temperature. The resulting mixture was stirred at 40° C. for 60 hours. The solvent was evaporated and the residue was purified by column chromatography (silica gel; petroleum ether:ethyl acetate; 4:1; v/v) to afford of 2-(4-bromo-3,5-difluoro-N-methyl-anilino)acetonitrile (900 mg) as a white solid.

MS m/z (+ESI): 260.9, 262.9 [M+H]⁺.

¹H-NMR (400 MHz, DMSO-d₆) δ ppm: 6.82-6.88 (m, 2H), 4.60 (s, 2H), 2.96 (s, 3H).

### Preparation of 4-[[4-[cyanomethyl(methyl)amino]-2,6-difluoro-phenyl]methylene]-N-(3-methyl-1,2,4-thiadiazol-5-yl)piperidine-1-carboxamide

The title compound was prepared as a white solid following scheme 1 and in analogy to Example 88 using 2-(4-bromo-3,5-difluoro-N-methyl-anilino)acetonitrile as starting material and after purification by preparative HPLC.

### Preparation of Example 166: 4-[(4-chloro-2-fluoro-phenyl)methyl]-N-[3-(methylamino)-1,2,4-thiadiazol-5-yl]piperidine-1-carboxamide

### Preparation of N-(5-amino-1,2,4-thiadiazol-3-yl)-4-methyl-benzenesulfonamide

To a stirred suspension of carbamimidoylthiourea (4720 mg; 39.15 mmol)[CAS 2114-02-5] in pyridine (80 mL) was gradually and cautiously added tosyl chloride (15230 mg; 78.29 mmol). The reaction mixture was heated to 100° C. and stirred for 15 minutes. Additional tosyl chloride (7620 mg; 39.5 mmol) was added in 2 portions at 15 minutes interval. After further agitation for 15 minutes, the reaction mixture was concentrated to dryness. The resulting light yellow oil was poured into a mixture of ice-water (200 mL) containing 37% HCl aqueous solution (22 mL). The resulting precipitate was collected by filtration and was washed with water (2×30 mL). The product was recrystallized in ethanol to afford N-(5-amino-1,2,4-thiadiazol-3-yl)-4-methyl-benzenesulfonamide (1457 mg) as a light yellow powder.

MS m/z (+ESI): 271.0 [M+H]⁺.

### Preparation of N-(5-amino-1,2,4-thiadiazol-3-yl)-N,4-dimethyl-benzenesulfonamide

To a stirred solution of N-(5-amino-1,2,4-thiadiazol-3-yl)-4-methyl-benzenesulfonamide (135 mg; 0.49 mmol) in N,N-dimethylformamide (5 mL) was added sodium hydride 60% in mineral oil (20 mg; 0.49 mmol). The reaction mixture was stirred for 10 minutes and then treated with methyl iodide (71 mg; 0.49 mmol). After agitation for 1 hour, the reaction mixture was concentrated to dryness. The residue was purified by column chromatography (silica gel; petroleum ether:ethyl acetate; 2:1; v/v) to afford N-(5-amino-1,2,4-thiadiazol-3-yl)-N,4-dimethyl-benzenesulfonamide (68 mg) as a light yellow solid.

MS m/z (+ESI): 285.0 [M+H]⁺.

### Preparation of 4-[(4-chloro-2-fluoro-phenyl)methyl]-N-[3-[methyl(p-tolylsulfonyl)amino]-1,2,4-thiadiazol-5-yl]piperidine-1-carboxamide

The title compound was prepared as white solid following scheme 1 and in analogy to Example 22 using 4-[(4-chloro-2-fluoro-phenyl)methyl]piperidine (intermediate of Example 9) and N-(5-amino-1,2,4-thiadiazol-3-yl)-N,4-dimethyl-benzenesulfonamide as starting material and after purification by column chromatography (silica gel; petroleum ether:ethyl acetate; 8:1 to 3:1; v/v).

MS m/z (+ESI): 538.1, 540.1 [M+H]⁺.

### Preparation of 4-[(4-chloro-2-fluoro-phenyl)methyl]-N-[3-(methylamino)-1,2,4-thiadiazol-5-yl]piperidine-1-carboxamide

4-[(4-chloro-2-fluoro-phenyl)methyl]-N-[3-[methyl(p-tolylsulfonyl)amino]-1,2,4-thiadiazol-5-yl]piperidine-1-carboxamide (120 mg; 0.22 mmol) was dissolved in 96% sulfuric acid (3 mL) at 0° C. The reaction mixture was stirred for 0.5 hours at 0° C. and then poured into ice-water (30 mL). The product was extracted from the aqueous solution using ethyl acetate (4×25 mL) and the combined organic layers were washed with brine, dried over Na₂SO₄, filtered and concentrated to dryness. The residue was purified by preparative HPLC to afford 4-[(4-chloro-2-fluoro-phenyl)methyl]-N-[3-(methylamino)-1,2,4-thiadiazol-5-yl]piperidine-1-carboxamide (51 mg) as a white solid.

### Preparation of Example 170: 4-[[2-fluoro-4-(3-hydroxyprop-1-ynyl)phenyl]methylene]-N-(3-methyl-1,2,4-thiadiazol-5-yl)piperidine-1-carboxamide

### Preparation of tert-butyl 4-[[2-fluoro-4-(3-hydroxyprop-1-ynyl)phenyl]methylene]piperidine-1-carboxylate

In a re-sealable tube was charged tert-butyl 4-[(4-chloro-2-fluoro-phenyl)methylene]piperidine-1-carboxylate (200 mg; 0.61 mmol)(intermediate Example 39), copper(I) iodide (10 mg; 0.06 mmol), chloro(2-dicyclohexylphosphino-2′,4′,6′-triisopropylbiphenyl)(2′-aminobiphenyl-2-yl)palladium (II) (120 mg; 0.15 mmol), 2-Dicyclohexylphosphino-2,4,6-triisopropylbiphenyl (10 mg; 0.03 mmol), triethylamine (4.27 mL; 30.39 mmol) and prop-2-yn-1-ol (0.71 mL; 12.15 mmol)[CAS 107-19-7]. Argon was bubbled into the mixture for 5 minutes and the tube was sealed. The reaction mixture was then heated to 90° C. and stirred for 3 hours. More chloro(2-dicyclohexylphosphino-2′,4′,6′-triisopropylbiphenyl)(2′-aminobiphenyl-2-yl)palladium (II) (80 mg) was added and the reaction mixture was stirred for additional 2 hours at 90° C. After cooling to room temperature, the mixture was partitioned between ethyl acetate and water, filtered and the cake was washed with ethyl acetate. 0.1N HCl aqueous solution (10 mL) was added to the filtrate and the mixture was decanted. The organic layer was separated, washed with brine, dried over MgSO₄, filtered and concentrated to dryness. The residue was purified by column chromatography (silica gel; cyclohexane:ethyl acetate; 1:0 to 3:1; v/v) to afford tert-butyl 4-[[2-fluoro-4-(3-hydroxyprop-1-ynyl)phenyl]methylene]piperidine-1-carboxylate (75 mg) as a light yellow oil.

MS m/z (+ESI): 331.1 [M-tBu+HCOOH]⁺.

### Preparation of 3-[3-fluoro-4-(4-piperidylidenemethyl)phenyl]prop-2-yn-1-ol, trifluoroacetic acid salt

To a stirred solution of tert-butyl 4-[[2-fluoro-4-(3-hydroxyprop-1-ynyl)phenyl]methylene]piperidine-1-carboxylate (80 mg; 0.21 mmol) in dichloromethane (2 mL) was added dropwise trifluoroacetic acid (0.31 mL; 4.13 mmol) at room temperature. The reaction solution was stirred for 1 h and then concentrated to dryness to afford 3-[3-fluoro-4-(4-piperidylidenemethyl)phenyl]prop-2-yn-1-ol, trifluoroacetic acid salt (80 mg) as a yellow viscous oil.

MS m/z (+ESI): 246.2 [M+H]⁺.

### Preparation of 4-[[2-fluoro-4-(3-hydroxyprop-1-ynyl)phenyl]methylene]-N-(3-methyl-1,2,4-thiadiazol-5-yl)piperidine-1-carboxamide

The title compound was prepared as a white solid following scheme 1 and in analogy to Example 27 using 3-[3-fluoro-4-(4-piperidylidenemethyl)phenyl]prop-2-yn-1-ol, trifluoroacetic acid and (4-nitro-phenyl) N-(3-methyl-1,2,4-thiadiazol-5-yl)carbamate as starting materials and after purification by column chromatography (silica gel; cyclohexane:ethyl acetate; 3:7 to 0:1; v/v).

### Preparation of Example 177: 4-[(4-chloro-2-fluoro-phenyl)methylene]-N-[2-(hydroxymethyl)-4-pyridyl]piperidine-1-carboxamide, trifluoroacetic acid

### Preparation of tert-butyl-dimethyl-[(4-nitro-2-pyridyl)methoxy]silane

To a stirred solution of (4-nitro-2-pyridyl)methanol (3530 mg; 22.3 mmol)[CAS 98197-88-7] in N,N-dimethylformamide (60 mL) were added imidazole (3070 mg; 44.7 mmol) and tert-butyl-chloro-dimethyl-silane (4080 mg; 26.8 mmol) at 0° C. The reaction mixture was allowed to warm up naturally to room temperature and stirred for 18 hours. The reaction mixture was quenched with distilled water (30 mL) and the product was extracted with ethyl acetate (3×100 mL). The combined organic layers were washed with brine, dried over anhydrous Na₂SO₄, filtered and concentrated to dryness. The residue was purified by column chromatography (silica gel; petroleum ether:ethyl acetate; 25:1; v/v) to afford tert-butyl-dimethyl-[(4-nitro-2-pyridyl)methoxy]silane (5790 mg) as a light yellow oil.

MS m/z (+ESI): 269.0 [M+H]⁺.

¹H-NMR (400 MHz, DMSO-d₆) δ ppm: 8.89 (d, J=5.2 Hz, 1H), 8.05 (d, J=2.4 Hz, 1H), 8.01 (dd, J1=5.2 Hz, J2=2.4 Hz, 1H), 4.90 (s, 2H), 0.94 (s, 9H), 0.13 (s, 6H).

### Preparation of 2-[[tert-butyl(dimethyl)silyl]oxymethyl]pyridin-4-amine

To a stirred solution of tert-butyl-dimethyl-[(4-nitro-2-pyridyl)methoxy]silane (538 mg; 1.98 mmol) in methanol (20 mL) was added 10% palladium on charcoal (219 mg; 0.21 mmol). The reaction mixture was stirred under hydrogen atmosphere (bar) for 18 hours. The reaction mixture was filtered and the filtrate was concentrated to dryness to afford 2-[[tert-butyl(dimethyl)silyl]oxymethyl]pyridin-4-amine (473 mg) as a colorless oil.

MS m/z (+ESI): 239.1 [M+H]⁺.

¹H-NMR (400 MHz, DMSO-d₆) δ ppm: 7.85 (d, J=5.2 Hz, 1H), 6.57 (d, J=2.0 Hz, 1H), 6.30 (dd, J1=5.2 Hz, J2=2.0 Hz, 1H), 5.97 (s, 2H), 4.51 (s, 2H), 0.91 (s, 9H), 0.08 (s, 6H).

### Preparation of N-[2-[[tert-butyl(dimethyl)silyl]oxymethyl]-4-pyridyl]-4-[(4-chloro-2-fluoro-phenyl)methylene]piperidine-1-carboxamide

The title compound was prepared as a white foam following scheme 1 and in analogy to Example 144 using 4-[(4-chloro-2-fluoro-phenyl)methylene]piperidine (intermediate of Example 39) and 2-[[tert-butyl(dimethyl)silyl]oxymethyl]pyridin-4-amine as starting materials and after purification by column chromatography (silica gel; dichloromethane:acetone; 30:1; v/v).

MS m/z (+ESI): 490.1, 492.1 [M+H]⁺.

### Preparation of 4-[(4-chloro-2-fluoro-phenyl)methylene]-N-[2-(hydroxymethyl)-4-pyridyl]piperidine-1-carboxamide. trifluoroacetic acid salt

To a stirred solution of N-[2-[[tert-butyl(dimethyl)silyl]oxymethyl]-4-pyridyl]-4-[(4-chloro-2-fluoro-phenyl)methylene]piperidine-1-carboxamide (215 mg; 0.43 mmol) in dichloromethane (50 mL) was added dropwise a solution of 2N HCl in ethyl acetate (1 mL) at 15° C. After agitation for 4 hours, the reaction mixture was concentrated to dryness. The residue was purified by preparative HPLC to afford 4-[(4-chloro-2-fluoro-phenyl)methylene]-N-[2-(hydroxymethyl)-4-pyridyl]piperidine-1-carboxamide, trifluoroacetic acid salt (103 mg) as a white solid.

### Preparation of Example 178: 4-[(4-chloro-2-fluoro-phenyl)methylene]-N-[2-(cyanomethyl)-4-pyridyl]piperidine-1-carboxamide, formic acid

### Preparation of [4-[[4-[(4-chloro-2-fluoro-phenyl)methylene]piperidine-1-carbonyl]amino]-2-pyridyl]methyl methanesulfonate

To a stirred solution of 4-[(4-chloro-2-fluoro-phenyl)methylene]-N-[2-(hydroxymethyl)-4-pyridyl]piperidine-1-carboxamide, trifluoroacetic acid salt (203 mg; 0.53 mmol)(Example 177) and diisopropylethylamine (0.14 mL; 1.07 mmol) in dichloromethane (10 mL) was gradually added methanesulfonyl chloride (0.097 mL; 0.80 mmol) at 0° C. The reaction mixture was stirred for 1 hour at 0° C. and then concentrated to dryness to afford crude [4-[[4-[(4-chloro-2-fluoro-phenyl)methylene]piperidine-1-carbonyl]amino]-2-pyridyl]methyl methanesulfonate (243 mg) as a light yellow gum, directly used in the next step.

MS m/z (+ESI): 452.0, 454.0 [M+H]⁺.

### Preparation of 4-[(4-chloro-2-fluoro-phenyl)methylene]-N-[2-(cyanomethyl)-4-pyridyl]piperidine-1-carboxamide, formic acid

To a stirred solution of [4-[[4-[(4-chloro-2-fluoro-phenyl)methylene]piperidine-1-carbonyl]amino]-2-pyridyl]methyl methanesulfonate (400 mg; 0.87 mmol) in N,N-dimethylformamide (10 mL) were added potassium cyanide (592 mg; 8.72 mmol) and tetrabutylammonium cyanide (493 mg; 1.74 mmol). After agitation for 2 hours, the reaction mixture was treated with a saturated NaHCO₃ aqueous solution (2 mL) and then heated to 80° C. After stirring for 1.5 hours, volatiles were removed under reduced pressure. The residue was diluted with ethyl acetate (100 mL) and the mixture was successively washed with a saturated NaHCO₃ aqueous solution (3×30 mL), water (30 mL) and brine (30 mL). The organic solution was finally dried over anhydrous Na₂SO₄, filtered and concentrated to dryness. The residue was purified by preparative HPLC to afford 4-[(4-chloro-2-fluoro-phenyl)methylene]-N-[2-(cyanomethyl)-4-pyridyl]piperidine-1-carboxamide, formic acid (57 mg) as a light brown solid.

### Preparation of Example 180: N-[2-(aminomethyl)-4-pyridyl]-4-[(4-chloro-2,6-difluoro-phenyl)methylene]piperidine-1-carboxamide, formic acid

### Preparation of 2-(bromomethyl)-4-nitro-pyridine

To a stirred solution of (4-nitro-pyridin-2-yl)-methanol (1000 mg; 6.42 mmol) in dichloromethane (30 mL) was gradually added phosphorus tribromide (0.79 mL; 8.35 mmol) at 0° C. After addition, the mixture was heated to reflux for 3 hours. The reaction mixture was then cooled to 0° C. and cautiously deactivated with water. A saturated aqueous solution of potassium carbonate was cautiously added to the mixture until basic pH. The organic layer was separated and washed with brine, dried over Na₂SO₄ and then concentrated to dryness to afford crude 2-(bromomethyl)-4-nitro-pyridine (1400 mg) as a brown oil, directly used in the next step without further purification.

MS m/z (+ESI): 216.9, 219.0 [M+H]⁺.

### Preparation of 2-[(4-nitro-2-pyridyl)methyl]isoindoline-1,3-dione

To a solution of 2-(bromomethyl)-4-nitro-pyridine (1400 mg; 18.7 mmol) in N,N-dimethylformamide (40 mL) was added potassium phthalimide (4900 mg; 26.18 mmol). The reaction mixture was stirred for 24 hours. The mixture was partitioned between ethyl acetate and water and then decanted. The organic layer was separated, washed with brine, dried over Na₂SO₄ and concentrated to dryness. The resulting residue was purified by column chromatography (silica gel; dichloromethane:methanol; 10:1; v/v) to afford 2-[(4-nitro-2-pyridyl)methyl]isoindoline-1,3-dione (3690 mg) as a yellow solid.

MS m/z (+ESI): 284.1 [M+H]⁺.

### Preparation of 4-[(4-chloro-2,6-difluoro-phenyl)methylene]-N-[2-[(1,3-dioxoisoindolin-2-yl)methyl]-4-pyridyl]piperidine-1-carboxamide

The tilted compound was prepared as a pink solid following scheme 1 and in analogy to Examples 177 and 144 using 4-[(4-chloro-2-fluoro-phenyl)methylene]piperidine (intermediate of Example 39) and as startings materials and after purification by column chromatography (silica gel; petroleum ether:ethyl acetate; 1:2; v/v).

MS m/z (+ESI): 522.9, 524.9 [M+H]⁺.

### Preparation of N-[2-(aminomethyl)-4-pyridyl]-4-[(4-chloro-2,6-difluoro-phenyl)methylene]piperidine-1-carboxamide, formic acid salt

To a mixture of 4-[(4-chloro-2,6-difluoro-phenyl)methylene]-N-[2-[(1,3-dioxoisoindolin-2-yl)methyl]-4-pyridyl]piperidine-1-carboxamide (1060 mg; 2.01 mmol) in ethanol (50 mL) was added hydrazine monohydrate 85% (0.39 mL; 8.03 mmol). The reaction solution was refluxed for 2 hours. After cooling to room temperature, insolubles were removed by filtration and the filtrate was concentrated to dryness. The residue was purified by preparative HPLC to afford N-[2-(aminomethyl)-4-pyridyl]-4-[(4-chloro-2,6-difluoro-phenyl)methylene]piperidine-1-carboxamide, formic acid salt (349 mg) as a white solid.

### Biological Examples

Cell Culture

The cervical tumor cell line HeLa (ATCC, CCL-2) was cultivated in DMEM medium (Invitrogen cat. no. 11971, 4.5 g/L high glucose) containing 10% fetal calf serum (Sigma cat. no. F9665) and 1% Penicillin/Streptomycin (Sigma cat. no. P0781) at 37° C. in 5% CO₂. HeLa galactose cells (i.e. HeLa cells that grow in high concentrations of galactose) were generated from HeLa glucose cells (i.e. HeLa cells that grow in high concentrations of glucose) by gradually changing the amount of glucose in the media to zero glucose in the presence of galactose as a sugar source (50% galactose/50% glucose media for one week, then 75% galactose/25% glucose media for one week, to 100% galactose media in the third week). Galactose media (Invitrogen cat. no. 11966) was supplemented with 10 mM galactose (Sigma cat. no. G5388).

Cell Growth and Proliferation Assay of HeLa Galactose and Glucose Cells

HeLa galactose cells and HeLa glucose cells were seeded in 96 well plates (TPP, cat. no 92696) at 2000 and 1500 cells/well, respectively, in 100 μL of complete medium. After overnight incubation the cells were incubated for 72 hours in complete medium containing 0.001% DMSO or compounds (final concentration of DMSO 0.001%). After the medium was removed, cells were fixed and stained by adding 50 μL crystal violet staining (0.2% crystal violet (Sigma-Aldrich cat. no. C0775) in 50% methanol) per well. The plates were incubated for 1 hour at room temperature. Subsequently the stain was decanted and plates were washed 4 times with de-mineralized water. Plates were air-dried for 2 hours. The stain was dissolved by adding 100 μL buffer (0.1 M Tris pH 7.5, 0.2% SDS, 20% ethanol) per well and shaking the plates. Absorbance at 590 nm was measured using a SpectraMax® 250 plate reader (Molecular Devices). Antiproliferative/growth inhibition IC50s were calculated from concentration-response curves using GraphPad Prism software.

Oxygen Consumption Assay

Oxygen consumption is one of the most informative and direct measures of mitochondrial function and can be measured, for example, by using the MitoXpress® assay (Luxcel MX-2001, Luxcel Biosciences). The MitoXpress® probe is one of a family of phosphorescent oxygen sensitive probes. The assay exploits the ability of oxygen to quench the excited state of the MitoXpress® probe. As the test material respires (i.e. the cells), oxygen is depleted in the surrounding solution/environment, which increases the probe phosphorescence signal. Changes in oxygen consumption reflecting changes in mitochondrial activity are seen as changes in the MitoXpress® probe signal over time.

Cells were seeded in 96 well black plates with transparent bottoms (Greiner Bio-One cat. no. 655090) at a density of 50′000 cells/well in a final volume of 100 μL. After 24 hours the incubation media was removed and 150 μL of fresh media containing inhibitors at different concentrations was added to each well. Then, 10 μL of MitoXpress® and 150 μL mineral oil were added per well. Reading from the top of the plate, kinetic analysis was performed at 37° C. for 5 hours using a Synergy 4 plate reader (BioTek) and Time-resolved Fluoresence (TRF) wavelengths of 380/11 nm excitation and 650/20 nm emission or 665/40 emission (30 microsecond delay time, 100 microsecond integration time, gain or sensitivity settings set at either medium or high). IC50s were calculated as the concentration that inhibits 50% of the phosphorescent oxygen sensitive probe signal (MitoXpress®) as compared to untreated cells.

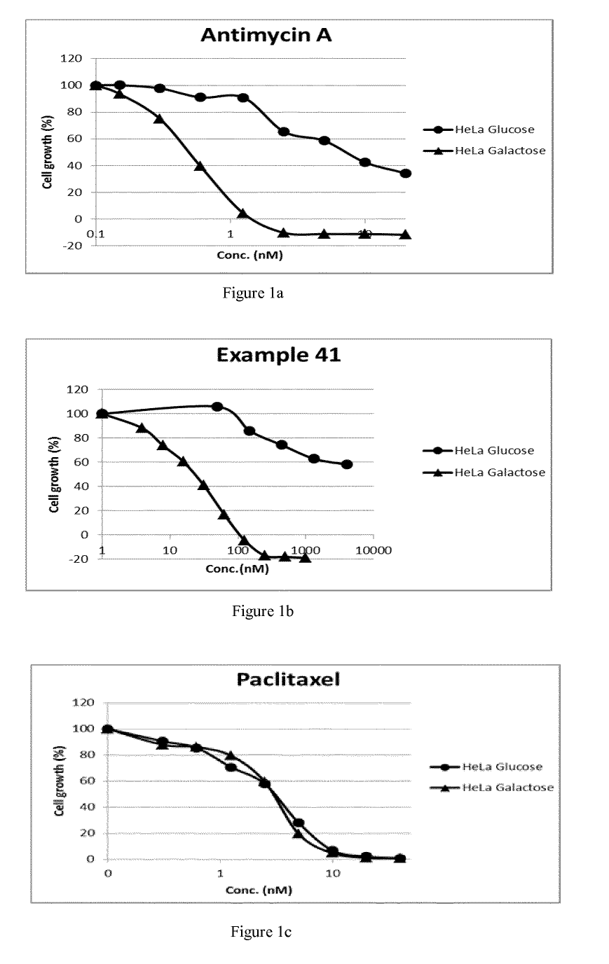

Galactose cells are highly dependent on OXPHOS and more sensitive to mitochondrial inhibitors than glucose cells (Gohil V. M. et al., Nat. Biotechnol., vol. 28, no. 3, pages 249-255, 2010). For example, a differential sensitivity in HeLa glucose versus HeLa galactose cell growth is exhibited by Antimycin A (Sigma-Aldrich cat. no. A8674), an inhibitor of complex III of the electron transport chain of the mitochondria (FIG. 1a), but not by a cytotoxic compound such as Paclitaxel (CAS 33069-62-4) (FIG. 1c). FIG. 1b shows the HeLa glucose versus HeLa galactose cell growth for Example 41, demonstrating that compounds of the invention also exhibit differential sensitivity in HeLa glucose versus HeLa galactose cell growth assays. As such HeLa galactose cells can be used to screen for mitochondrial inhibitors. Moreover, compounds with activity in HeLa galactose cells can be confirmed as true mitochondrial inhibitors by testing Oxygen consumption inhibition as shown in Table 2.

Biological data are given below in Table 2.

TABLE 2

| | IC50 HeLa | IC50 Oxygen |
|---|---|---|
| Example | Galactose (nM) | Consumption (nM) |
| 1 | 161 | 193 |
| 2 | 41 | <100 |
| 3 | 80 | <100 |
| 4 | 460 | 400 |
| 5 | 36 | 100 |
| 6 | 31 | 200 |
| 7 | 142 | 500 |
| 8 | 32 | 200 |
| 9 | 36 | <100 |
| 10 | 36 | 200 |
| 11 | 193 | 1300 |
| 12 | 19 | 500 |
| 13 | 307 | 345 |
| 14 | 146 | 200 |
| 15 | 267 | 412 |
| 16 | 260 | 326 |
| 17 | 162 | 243 |
| 18 | 637 | 515 |
| 19 | 266 | 488 |
| 20 | 122 | 369 |
| 21 | 207 | 208 |
| 22 | 837 | nt |
| 23 | 356 | nt |
| 24 | 138 | 307 |
| 25 | 18 | 55 |
| 26 | 15 | 23 |
| 27 | 59 | 83 |
| 28 | 249 | 334 |
| 29 | 256 | 205 |
| 30 | 59 | 178 |
| 31 | 795 | nt |
| 32 | 43 | 116 |
| 33 | 70 | 102 |
| 34 | 572 | nt |
| 35 | 240 | 473 |
| 36 | 540 | nt |
| 37 | 478 | nt |
| 38 | 61 | 131 |
| 39 | 38 | 69 |
| 40 | 32 | 48 |
| 41 | 23 | 41 |
| 42 | 670 | nt |
| 43 | 439 | nt |
| 44 | 429 | nt |
| 45 | 284 | 440 |
| 46 | 47 | 129 |
| 47 | 25 | 67 |
| 48 | 146 | 278 |
| 49 | 21 | 58 |
| 50 | 197 | 255 |
| 51 | 67 | 182 |
| 52 | 271 | 477 |
| 53 | 329 | 890 |
| 54 | 186 | 235 |
| 55 | 202 | 383 |
| 56 | 736 | 1249 |
| 57 | 775 | nt |
| 58 | 170 | 304 |
| 59 | 41 | 43 |
| 60 | 53 | 68 |
| 61 | 516 | nt |
| 62 | 57 | 122 |
| 63 | 20 | 56 |
| 64 | 89 | 187 |
| 65 | 50 | 126 |
| 66 | 67 | 119 |
| 67 | 125 | 233 |
| 68 | 100 | 179 |
| 69 | 91 | 119 |
| 70 | 30 | 65 |
| 71 | 227 | 338 |
| 72 | 34 | 68 |
| 73 | 465 | nt |
| 74 | 34 | 51 |
| 75 | 54 | 59 |
| 76 | 190 | 332 |
| 77 | 48 | 54 |
| 78 | 163 | 267 |
| 79 | 279 | 378 |
| 80 | 134 | 108 |
| 81 | 888 | nt |
| 82 | 31 | 78 |
| 83 | 115 | 329 |
| 84a | 47 | 57 |
| 84b | 75 | 121 |
| 85 | 38 | 83 |
| 86 | 21 | 61 |
| 87 | 262 | 335 |
| 88 | 418 | nt |
| 89 | 228 | 352 |
| 90 | 693 | nt |
| 91 | 492 | nt |
| 92 | 57 | 62 |
| 93 | 56 | 63 |
| 94 | 97 | 159 |
| 95 | 20 | 30 |
| 96 | 208 | 361 |
| 97 | 28 | 30 |
| 98 | 39 | 76 |
| 99 | 307 | nt |
| 100 | 200 | 402 |
| 101 | 251 | 412 |
| 102 | 189 | 395 |
| 103 | 71 | 156 |
| 104 | 642 | nt |
| 105 | 482 | nt |
| 106 | 467 | nt |
| 107 | 389 | nt |
| 108 | 96 | 132 |
| 109 | 55 | 103 |
| 110 | 66 | 151 |
| 111 | 514 | nt |
| 112 | 12 | 23 |
| 113 | 35 | 52 |
| 114 | 19 | 65 |
| 115 | 29 | 79 |
| 116 | 54 | 150 |
| 117 | 77 | 145 |
| 118 | 43 | 70 |
| 119 | 159 | nt |
| 120 | 32 | 54 |
| 121 | 20 | 54 |
| 122 | 182 | nt |
| 123 | 245 | nt |
| 124 | 70 | nt |
| 125 | 63 | nt |
| 126 | 68 | nt |
| 127 | 15 | 35 |
| 128 | 33 | 168 |
| 129 | 16 | 20 |
| 130 | 355 | nt |
| 131 | 264 | nt |
| 132 | 76 | nt |
| 133 | 100 | nt |
| 134 | 449 | nt |
| 135 | 636 | nt |
| 136 | 81 | nt |
| 137 | 17 | 42 |
| 138 | 21 | 147 |
| 139 | 22 | 152 |
| 140 | 43 | 94 |
| 141 | 29 | 56 |
| 142 | 86 | nt |
| 143 | 43 | 103 |
| 144 | 8 | 53 |
| 145 | 15 | 106 |
| 146 | 13 | 20 |
| 147 | 118 | nt |
| 148 | 9 | 16 |
| 149 | 11 | 13 |
| 150 | 124 | nt |
| 151 | 20 | 42 |
| 152 | 19 | 39 |
| 153 | 34 | 119 |
| 154 | 37 | 192 |
| 155 | 149 | nt |
| 156 | 193 | nt |
| 157 | 51 | 109 |
| 158 | 28 | 40 |
| 159 | 215 | nt |
| 160 | 21 | 65 |
| 161 | 50 | 159 |
| 162 | 162 | nt |
| 163 | 293 | nt |
| 164 | 19 | 16 |
| 165 | 383 | nt |
| 166 | 407 | nt |
| 167 | 500 | nt |
| 168 | 280 | nt |
| 169 | 432 | nt |
| 170 | 415 | nt |
| 171 | 67 | nt |
| 172 | 126 | nt |
| 173 | 54 | 136 |
| 174 | 160 | nt |
| 175 | 58 | nt |
| 176 | 54 | 133 |
| 177 | 96 | nt |
| 178 | 155 | nt |
| 179 | 11 | 41 |
| 180 | 330 | nt |
| 181 | 83 | nt |
| 182 | 13 | 108 |
| 183 | 21 | 42 |

nt = not tested

1. A compound of formula I: 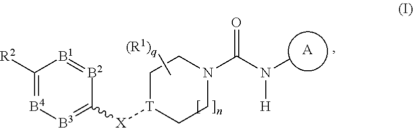
or a pharmaceutically acceptable salt, solvate or hydrate thereof,
wherein:
ring A represents group A-I or A-II 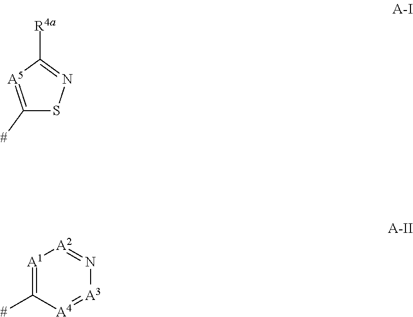
A1, A2, A3, A4 represent independently C(R4aa) or N, wherein no more than one of A1, A2, A3, and A4 represents N;
A5 represents C(R4b) or N;
B1, B2, B3 and B4 represent independently C(R3) or N, wherein no more than two of B1, B2, B3 and B4 represent N;
T represents >N—, >C═ or >CH— when ring A represents group A-I;
T represents >C═ or >CH— when ring A represents group A-II;
X represents —C(R6a)(R6b)-, —C(R6a)=, —O—, —S— or —C(O)—, providing that X is not —C(O)—, —O— or —S— when T is >N—;
R1 represents independently at each occurrence halogen, cyano, hydroxyl, —N(R5a)(R5b), C1-C6alkyl, C1-C6haloalkyl or C1-C6alkyl wherein one or two carbon atoms are independently replaced by —O— or —N(R5a)- and wherein the alkyl moiety is optionally substituted by one or more halogen;
R2 represents halogen, cyano, hydroxyl, mercapto, C1-C6alkyl optionally substituted by one to five R14, C2-C6alkenyl optionally substituted by one to five R14, C2-C6alkynyl optionally substituted by one to five R14, C1-C6alkoxy optionally substituted by one to five R14, —N(R9a)(R9b), —C1-C6alkylene-N(R9a)(R9b), —CHO, —C1-C6alkylene-CHO, —C(O)OR10, —C1-C6alkylene-C(O)OR10, —C(O)N(R11a)(R11b), —C1-C6alkylene-C(O)N(R11a)(R11b), —N(R12)C(O)R13, —C1-C6alkylene-N(R12)C(O)R13, C1-C6alkylthio, C1-C6alkylsulfinyl, C1-C6alkylsulfonyl, Cycle-P, —C1-C6alkylene-Cycle-P, Cycle-Q or —C1-C6alkylene-Cycle-Q;
R3 represents independently at each occurrence hydrogen, halogen, cyano, C1-C4alkyl, C1-C4haloalkyl, C1-C4alkoxy, C1-C4haloalkoxy or —N(R8a)(R8b);
R4a and R4b represent independently hydrogen, amino, —NH(C1-C4alkyl), —N(C1-C4alkyl)₂ or —C1-C4alkylene-R4c;
R4aa represents independently at each occurrence hydrogen, amino, —NH(C1-C4alkyl), —N(C1-C4alkyl)₂, —C1-C4alkylene-R4c or C3-C4cycloalkyl;
R4c represents independently at each occurrence hydrogen, cyano, hydroxyl, amino, C1-C4alkoxy, —CONH₂, —NH(C1-C4alkyl), —N(C1-C4alkyl)₂, Cycle-P or Cycle-Q;
R5a and R5b represent independently at each occurrence hydrogen or C1-C6alkyl;
R6a and R6b represent independently hydrogen or C1-C4alkyl;
each R8a and R8b represents independently at each occurrence hydrogen or C1-C4alkyl;
R9a represents hydrogen, C1-C6alkyl optionally substituted by one to five R14, —C1-C6alkylene-Cycle-P, —C1-C6alkylene-Cycle-Q, Cycle-P or Cycle-Q;
R9b, R11a, R11b and R12 represent independently hydrogen or C1-C6alkyl;
R10 and R13 represent independently at each occurrence C1-C6alkyl;
R14 represents independently at each occurrence halogen, cyano, hydroxyl, C1-C6alkoxy, amino, —NH(C1-C4alkyl), —N(C1-C4alkyl)₂ or —N(R12)C(O)R13;
Cycle-P represents independently at each occurrence a saturated or partially unsaturated 3- to 8-membered carbocyclic ring optionally substituted by 1 to 3 R16, or a saturated or partially unsaturated 3- to 8-membered heterocyclic ring optionally substituted by 1 to 3 R16 containing carbon atoms as ring members and one or two ring members independently selected from N and O, wherein N optionally may bear R15;
Cycle-Q represents independently at each occurrence phenyl optionally substituted by 1 to 3 R17 or a 5- to 6-membered heteroaryl ring containing one to four heteroatoms selected from O, S and N, optionally substituted by 1 to 3 R17;
R15 represents independently at each occurrence hydrogen or C1-C4alkyl;
R16 and R17 represent independently at each occurrence cyano, C1-C4alkyl, C1-C4haloalkyl, C1-C4alkoxy or C1-C4haloalkoxy;
n is 1 or 2; and
q is 0, 1, 2, 3 or 4;
and wherein the compound is not the following compounds:
1-Piperidinecarboxamide, 4-[(4-fluorophenyl)methyl]-N-(3-methyl]-N-1,2,4-thiadiazol-5-yl)-;
1-Piperidinecarboxamide, 4-(4-fluorobenzoyl)-N-(2-methyl-4-pyridinyl)-;
and wherein the compound is optionally not the following compound:
1-Piperidinecarboxamide, 4-(4-chlorobenzoyl)-N-4-pyridinyl-.

2. The compound according to claim 1, or a pharmaceutically acceptable salt, solvate or hydrate thereof, wherein ring A represents group A-I.

3. The compound according to claim 2, or a pharmaceutically acceptable salt, solvate or hydrate thereof, wherein R4a represents hydrogen, amino, C1-C4alkyl, C1-C4alkyl wherein one CH₂ is replaced by —NH— or —N(CH₃)—, —C1-C4alkylene-cyano, —C1-C4alkylene-hydroxyl, —C1-C4alkylene-amino or —C1-C4alkylene-Cycle-P, wherein Cycle-P is a 5- to 6-membered heterocyclic ring.

4. The compound according to claim 3, or a pharmaceutically acceptable salt, solvate or hydrate thereof, wherein R4a represents hydrogen, methyl, ethyl, amino, —CH₂CH₂CN, —CH₂CH₂-morpholinyl or —CH₂CH₂OH; and R4b represents hydrogen.

5. The compound according to claim 4, or a pharmaceutically acceptable salt, solvate or hydrate thereof, wherein R4a represents methyl or ethyl.

6. The compound according to claim 1, or a pharmaceutically acceptable salt, solvate or hydrate thereof, wherein ring A represents group A-II.

7. The compound according to claim 6, or a pharmaceutically acceptable salt, solvate or hydrate thereof, wherein one of A2 and A3 represent C(R4aa) and the other represents CH and A1 and A4 represent CH.

8. The compound according to claim 6, or a pharmaceutically acceptable salt, solvate or hydrate thereof, wherein R4aa represents hydrogen, amino, C1-C4alkyl, C1-C4alkyl wherein one CH₂ is replaced by —NH— or —N(CH₃)—, C3-C4cycloalkyl, —C1-C4alkylene-cyano, —C1-C4alkylene-hydroxyl, —C1-C4alkylene-amino, —C1-C4alkylene-methoxy, —C1-C4alkylene-C3-C4cycloalkyl, or —C1-C4alkylene-Cycle-P, wherein Cycle-P is a 5- to 6-membered heterocyclic ring.

9. The compound according to claim 8, or a pharmaceutically acceptable salt, solvate or hydrate thereof, wherein R4aa represents hydrogen, methyl, ethyl, amino, —CH₂CH₂CN, —CH₂CH₂-morpholinyl, —CH₂OH, —CH₂CH₂OH, —CH₂OCH₃ or cyclopropyl.

10. The compound according to claim 9, or a pharmaceutically acceptable salt, solvate or hydrate thereof, wherein R4aa represents methyl, ethyl or cyclopropyl.

11. The compound according to claim 1, or a pharmaceutically acceptable salt, solvate or hydrate thereof, wherein n is 1.

12. The compound according to claim 1, or a pharmaceutically acceptable salt, solvate or hydrate thereof, wherein n is 2.

13. The compound according to claim 1, or a pharmaceutically acceptable salt, solvate or hydrate thereof, wherein T represents >C═ or >CH—.

14. The compound according to claim 1, or a pharmaceutically acceptable salt, solvate or hydrate thereof, wherein T represents >C═ and X represents ═CH—.

15. The compound according to claim 1, or a pharmaceutically acceptable salt, solvate or hydrate thereof, wherein T represents >CH and X represents —CH₂—.

16. The compound according to claim 1, or a pharmaceutically acceptable salt, solvate or hydrate thereof, wherein T represents >CH and X represents —C(O)—.

17. The compound according to claim 1, or a pharmaceutically acceptable salt, solvate or hydrate thereof, wherein
B1, B2, B3 and B4 represent independently C(R3a), C(R3b) or N;
no more than one of B1, B2, B3 and B4 represents N;
no more than two of B1, B2, B3 and B4 represent C(R3a);
R3a is R3; and
R3b represents hydrogen.

18. The compound according to claim 17, or a pharmaceutically acceptable salt, solvate or hydrate thereof, wherein the ring formed by B1, B2, B3 and B4 is represented by group B-Ia, group B-Ib, group B-IIa or group BIIIa: 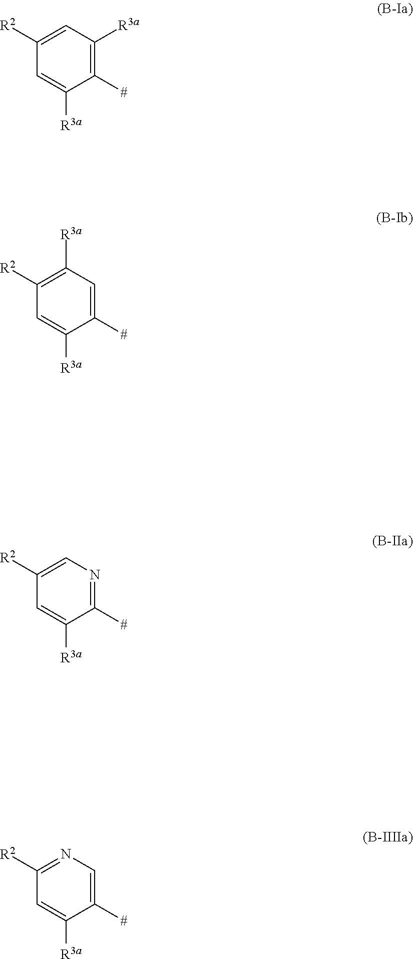
wherein each R3a is independently R3.

19. The compound according to claim 18, or a pharmaceutically acceptable salt, solvate or hydrate thereof, wherein the ring formed by B1, B2, B3 and B4 is represented by group B-Ia.

20. The compound according to claim 1, or a pharmaceutically acceptable salt, solvate or hydrate thereof, wherein R2 represents halogen, cyano, hydroxyl, C1-C6alkyl optionally substituted by one to five R14, C1-C6alkoxy optionally substituted by one to five R14, —N(R9a)(R9b) or —C1-C6alkylene-N(R9a)(R9b).

21. The compound according to claim 20, or a pharmaceutically acceptable salt, solvate or hydrate thereof, wherein R2 represents fluoro, chloro, bromo, cyano, hydroxyl, C1-C6alkyl, C1-C6haloalkyl, C1-C6alkyl wherein one or two non-adjacent carbon atoms in the alkyl other than the connecting carbon atom are replaced independently by —O—, —OH, —NH—, —NH₂, —N(CH₃)—, —NH(CH₃), —N(CH₃)₂, or —CN, or C1-C6haloalkyl wherein one or two non-adjacent carbon atoms in the haloalkyl other than the connecting carbon atom are replaced independently by —O—, —OH, —NH—, —NH₂, —N(CH₃)—, —NH(CH₃), —N(CH₃)₂ or —CN, or C1-C6alkoxy, C1-C6alkoxy wherein one carbon atom in the alkoxy other than the carbon atom connected to the oxygen is replaced by —O—, —OH, —NH—, —NH₂, —N(CH₃)—, —CN, or —N(R9a)(R9b) or —C1-C6alkylene-N(R9a)(R9b) and wherein R9a represents hydrogen, C1-C6alkyl wherein one or two non-adjacent carbon atoms in the alkyl are replaced independently by —O—, —OH, —NH—, —NH₂, —N(CH₃)—, —NH(CH₃), —N(CH₃)₂ or —CN, or R9a represents —C1-C6-alkylene-Cycle-P or Cycle-P, wherein Cycle-P represents a saturated 4- to 6-membered heterocyclic ring containing one or two heteroatoms selected from O and N(R15), wherein the heterocyclic ring is optionally substituted by one to three substituents selected from methyl, R9b represents hydrogen, methyl or ethyl, and R15 represents independently at each occurrence hydrogen or methyl.

22. The compound according to claim 21, or a pharmaceutically acceptable salt, solvate or hydrate thereof, wherein R2 represents fluoro, chloro, bromo, cyano, hydroxyl, C1-C6alkyl, C1-C6haloalkyl, C1-C6alkoxy, C1-C6haloalkoxy, —C1-C4alkylene-methoxy, —N(R9b)-C1-C4alkylene-R18, —N(R9b)-C1-C4alkylene-Cycle-P or —N(R9b)-Cycle-P, wherein Cycle-P represents tetrahydrofuranyl, azetidinyl, pyrrolidinyl, piperidinyl, piperazinyl, dioxanyl or morpholinyl wherein N is substituted by R15 in each case, R9b represents hydrogen, methyl or ethyl, R15 represents independently at each occurrence hydrogen or methyl, and R18 represents —OH, —OCH₃, —CN, —NH₂, —NH(CH₃), or —N(CH₃)₂.

23. The compound according to claim 22, or a pharmaceutically acceptable salt, solvate or hydrate thereof, wherein R2 represents fluoro, chloro, bromo, cyano, methyl, trifluromethyl, N(CH₃)₂, methoxy, methoxymethyl, —N(CH₃)CH₂CH₂OH, —N(CH₃)CH₂CH₂OCH₃ or —N(CH₃)CH₂CN.

24. The compound according to claim 1, or a pharmaceutically acceptable salt, solvate or hydrate thereof, wherein each R3 represents independently at each occurrence hydrogen, fluoro, chloro, bromo, cyano, methyl, halomethyl, methoxy or amino.

25. The compound according to claim 24, or a pharmaceutically acceptable salt, solvate or hydrate thereof, wherein each R3 represents independently at each occurrence hydrogen or fluoro.

26. The compound according to claim 1, or a pharmaceutically acceptable salt, solvate or hydrate thereof, wherein the ring formed by B1, B2, B3 and B4 is represented by one of the following groups:

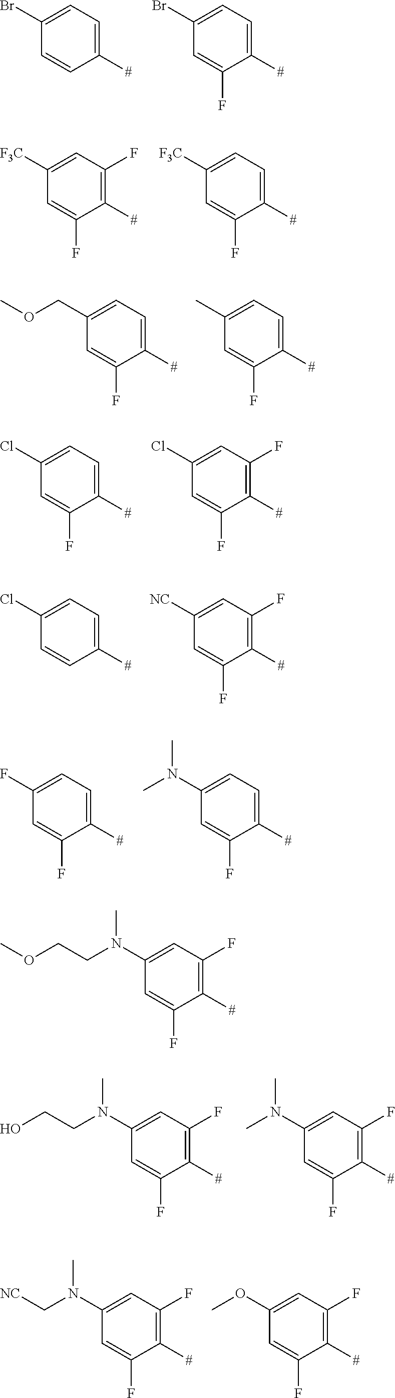

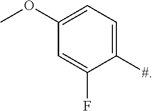

27. The compound according to claim 1, or a pharmaceutically acceptable salt, solvate or hydrate thereof, wherein
A1 and A4 represent CH;
one of A2 and A3 represent C(R4aa) and the other represents CH;
A5 represents CH or N;
B1, B2, B3 and B4 represent independently C(R3a), C(R3b) or N, wherein no more than one of B1, B2, B3 and B4 represents N, no more than two of B1, B2, B3 and B4 represents C(R3a);
T represents >C═ and X represents —CH═, or T represents >CH— and X represents —C(O)— or T represents >CH— and X represents —CH₂—;
R1 represents independently at each occurrence halogen, C1-C4alkyl or C1-C4alkoxy;
R2 represents halogen, cyano, hydroxyl, C1-C6alkyl optionally substituted by one to five R14, C1-C6alkoxy optionally substituted by one to five R14, —N(R9a)(R9b) or —C1-C6alkylene-N(R9a)(R9b);
R3a represents independently at each occurrence hydrogen, halogen, cyano, methyl, halomethyl, methoxy, amino, —NH(CH₃) or —N(CH₃)₂;
R3b represents hydrogen;
R4a represents hydrogen, amino, C1-C4alkyl, C1-C4alkyl wherein one CH₂ is replaced by —NH— or —N(CH₃)—, —C1-C4alkylene-cyano, —C1-C4alkylene-hydroxyl, —C1-C4alkylene-amino or —C1-C4alkylene-Cycle-P, preferably hydrogen, methyl, ethyl, amino, —CH₂CH₂CN, —CH₂CH₂-morpholinyl or —CH₂CH₂OH;
R4aa represents independently at each occurrence hydrogen, amino, C1-C4alkyl, C1-C4alkyl wherein one CH₂ is replaced by —NH— or —N(CH₃)—, C3-C4cycloalkyl, —C1-C4alkylene-cyano, —C1-C4alkylene-hydroxyl, —C1-C4alkylene-amino, —C1-C4alkylene-methoxy, —C1-C4alkylene-C3-C4cycloalkyl or —C1-C4alkylene-Cycle-P, preferably hydrogen, methyl, ethyl, amino, —CH₂CH₂CN, —CH₂CH₂-morpholinyl, —CH₂OH, —CH₂CH₂OH, —CH₂OCH₃ or cyclopropyl;
R9a represents hydrogen, C1-C6alkyl optionally substituted by one to five R14, —C1-C6-alkylene-Cycle-P or Cycle-P;
R9b represents hydrogen or methyl;
R14 represents independently at each occurrence halogen, cyano, hydroxyl, C1-C6alkoxy, amino, —N H(C1-C4alkyl) or —N(C1-C4alkyl)₂;
Cycle-P is a 5- to 6-membered heterocyclic ring;
n is 1 or 2; and
q is 0, 1 or 2.

28. The compound according to claim 1, or a pharmaceutically acceptable salt, solvate or hydrate thereof, wherein
Ring A represents group A-I;
B1, B2, B3 and B4 represent independently C(R3a) or C(R3b);
R3a represents independently at each occurrence hydrogen fluoro, chloro, methyl or methoxy;
R3b represents hydrogen;
T represents >C═ or >CH—;
X represents —CH₂—, —CH═ or —C(O)—;
R4a represents methyl;
n is 1; and
q is 0.

29. The compound according to claim 1, or a pharmaceutically acceptable salt, solvate or hydrate thereof, wherein the compound is:
4-[(4-chloro-2,6-difluoro-phenyl)methyl]-N-(3-methyl-1,2,4-thiadiazol-5-yl)piperidine-1-carboxamide;
4-[(4-chloro-2,6-difluoro-phenyl)methylene]-N-(3-methyl-1,2,4-thiadiazol-5-yl)piperidine-1-carboxamide;
4-[(2-fluoro-4-methyl-phenyl)methyl]-N-(3-methyl-1,2,4-thiadiazol-5-yl)piperidine-1-carboxamide;
4-[(4-chloro-2-fluoro-phenyl)methylene]-N-(3-methyl-1,2,4-thiadiazol-5-yl)piperidine-1-carboxamide;
4-[[2,6-difluoro-4-(trifluoromethyl)phenyl]methyl]-N-(3-methyl-1,2,4-thiadiazol-5-yl)piperidine-1-carboxamide;
4-[[2,6-difluoro-4-(trifluoromethyl)phenyl]methylene]-N-(3-methyl-1,2,4-thiadiazol-5-yl)piperidine-1-carboxamide;
4-[[4-(dimethylamino)-2-fluoro-phenyl]methylene]-N-(3-methyl-1,2,4-thiadiazol-5-yl)piperidine-1-carboxamide;
4-[(4-chloro-2-fluoro-phenyl)methyl]-N-(3-methyl-1,2,4-thiadiazol-5-yl)azepane-1-carboxamide;
4-[(2,4-difluorophenyl)methylene]-N-(3-methyl-1,2,4-thiadiazol-5-yl)piperidine-1-carboxamide;
4-[(2,4-difluorophenyl)methyl]-N-(3-methyl-1,2,4-thiadiazol-5-yl)piperidine-1-carboxamide;
4-[(4-chloro-2-fluoro-phenyl)methyl]-N-(3-methylisothiazol-5-yl)piperidine-1-carboxamide;
4-[(4-chloro-2-fluoro-phenyl)methyl]-N-(3-ethyl-1,2,4-thiadiazol-5-yl)piperidine-1-carboxamide;
4-[(4-chloro-2-fluoro-phenyl)methyl]-N-(2-methyl-4-pyridyl)piperidine-1-carboxamide;
4-(4-chlorobenzoyl)-N-(3-methyl-1,2,4-thiadiazol-5-yl)piperidine-1-carboxamide;
4-(2,4-difluorobenzoyl)-N-(3-methyl-1,2,4-thiadiazol-5-yl)piperidine-1-carboxamide;
4-(4-bromobenzoyl)-N-(3-methyl-1,2,4-thiadiazol-5-yl)piperidine-1-carboxamide;
(4E)-4-[(4-chloro-2-fluoro-phenyl)methylene]-3-methyl-N-(3-methyl-1,2,4-thiadiazol-5-yl)piperidine-1-carboxamide;
4-[(4-chloro-2-fluoro-phenyl)methyl]-3-methyl-N-(3-methyl-1,2,4-thiadiazol-5-yl)piperidine-1-carboxamide;
(4E)-4-[(4-chloro-2-fluoro-phenyl)methylene]-3,3-dimethyl-N-(3-methyl-1,2,4-thiadiazol-5-yl)piperidine-1-carboxamide;
(4E)-4-[(4-chloro-2-fluoro-phenyl)methylene]-2-methyl-N-(3-methyl-1,2,4-thiadiazol-5-yl)piperidine-1-carboxamide;
4-(4-chloro-2-fluoro-benzoyl)-N-(3-methyl-1,2,4-thiadiazol-5-yl)piperidine-1-carboxamide;
4-[[2-fluoro-4-(methoxymethyl)phenyl]methylene]-N-(3-methyl-1,2,4-thiadiazol-5-yl)piperidine-1-carboxamide;
4-[(4-cyano-2,6-difluoro-phenyl)methylene]-N-(3-methyl-1,2,4-thiadiazol-5-yl)piperidine-1-carboxamide;
4-[(2,6-difluoro-4-methoxy-phenyl)methylene]-N-(3-methyl-1,2,4-thiadiazol-5-yl)piperidine-1-carboxamide;
4-[(2,6-difluoro-4-methoxy-phenyl)methyl]-N-(3-methyl-1,2,4-thiadiazol-5-yl)piperidine-1-carboxamide;
4-[[4-(dimethylamino)-2,6-difluoro-phenyl]methylene]-N-(3-methyl-1,2,4-thiadiazol-5-yl)piperidine-1-carboxamide;
4-[[4-(dimethylamino)-2,6-difluoro-phenyl]methyl]-N-(3-methyl-1,2,4-thiadiazol-5-yl)piperidine-1-carboxamide;
4-[(4-chloro-2,6-difluoro-phenyl)methyl]-N-(3-methyl-1,2,4-thiadiazol-5-yl)azepane-1-carboxamide;
(4Z)-4-[[4-(dimethylamino)-2,6-difluoro-phenyl]methylene]-N-(3-methyl-1,2,4-thiadiazol-5-yl)azepane-1-carboxamide;
(4Z)-4-[(4-chloro-2-fluoro-phenyl)methylene]-N-(3-methyl-1,2,4-thiadiazol-5-yl)azepane-1-carboxamide;
4-[[2-fluoro-4-(trifluoromethyl)phenyl]methyl]-N-(3-methyl-1,2,4-thiadiazol-5-yl)azepane-1-carboxamide;
4-[[4-(dimethylamino)-2,6-difluoro-phenyl]methyl]-N-(3-methyl-1,2,4-thiadiazol-5-yl)azepane-1-carboxamide;
4-[(4-chloro-2,6-difluoro-phenyl)methyl]-N-(3-methyl-1,2,4-thiadiazol-5-yl)azepane-1-carboxamide;
4-[(4-cyano-2,6-difluoro-phenyl)methyl]-N-(3-methyl-1,2,4-thiadiazol-5-yl)piperidine-1-carboxamide;
4-[(4-chloro-2,6-difluoro-phenyl)methylene]-N-(2-methyl-4-pyridyl)piperidine-1-carboxamide;
4-(2-fluoro-4-methoxy-benzoyl)-N-(3-methyl-1,2,4-thiadiazol-5-yl)piperidine-1-carboxamide;
4-(4-bromo-2-fluoro-benzoyl)-N-(3-methyl-1,2,4-thiadiazol-5-yl)piperidine-1-carboxamide;
4-[(4-chloro-2-fluoro-phenyl)methylene]-N-(2-ethyl-4-pyridyl)piperidine-1-carboxamide;
4-[[2,6-difluoro-4-[2-methoxyethyl(methyl)amino]phenyl]methylene]-N-(3-methyl-1,2,4-thiadiazol-5-yl)piperidine-1-carboxamide;
4-[[4-(dimethylamino)-2,6-difluoro-phenyl]methylene]-N-(3-methylisothiazol-5-yl)piperidine-1-carboxamide;
4-[[4-(dimethylamino)-2,6-difluoro-phenyl]methylene]-N-(3-ethyl-1,2,4-thiadiazol-5-yl)piperidine-1-carboxamide;
4-[[4-(dimethylamino)-2,6-difluoro-phenyl]methyl]-N-(3-methylisothiazol-5-yl)piperidine-1-carboxamide;
4-[[4-(dimethylamino)-2,6-difluoro-phenyl]methyl]-N-(3-ethyl-1,2,4-thiadiazol-5-yl)piperidine-1-carboxamide;
4-[[2,6-difluoro-4-[2-hydroxyethyl(methyl)amino]phenyl]methylene]-N-(3-methyl-1,2,4-thiadiazol-5-yl)piperidine-1-carboxamide;
4-[(4-chloro-2-fluoro-phenyl)methyl]-6-methoxy-N-(3-methyl-1,2,4-thiadiazol-5-yl)-1,4-diazepane-1-carboxamide;
4-[[4-[cyanomethyl(methyl)amino]-2,6-difluoro-phenyl]methylene]-N-(3-methyl-1,2,4-thiadiazol-5-yl)piperidine-1-carboxamide;
4-[(4-cyano-2,6-difluoro-phenyl)methylene]-N-(2-ethyl-4-pyridyl)piperidine-1-carboxamide;
4-[(4-cyano-2,6-difluoro-phenyl)methylene]-N-(2-cyclopropyl-4-pyridyl)piperidine-1-carboxamide; or
4-[(4-chloro-2-fluoro-phenyl)methylene]-N-(2-cyclopropyl-4-pyridyl)piperidine-1-carboxamide.

30. A method for treatment of a proliferative disease, comprising the step of administering to a subject in need thereof a therapeutically effective amount of a compound of formula I: 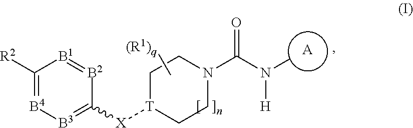
or a pharmaceutically acceptable salt, solvate or hydrate thereof,
wherein:
ring A represents group A-I or A-II 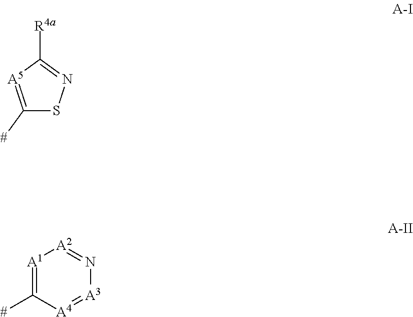
A1, A2, A3, A4 represent independently C(R4aa) or N, wherein no more than one of A1, A2, A3, and A4 represents N;
A5 represents C(R4b) or N;
B1, B2, B3 and B4 represent independently C(R3) or N, wherein no more than two of B1, B2, B3 and B4 represent N;
T represents >N—, >C═ or >CH—;
X represents —C(R6a)(R6b)-, —C(R6a)=, —O—, —S— or —C(O)—, providing that X is not —C(O)—, —O— or —S— when T is >N—;
R1 represents independently at each occurrence halogen, cyano, hydroxyl, —N(R5a)(R5b), C1-C6alkyl, C1-C6haloalkyl or C1-C6alkyl wherein one or two carbon atoms are independently replaced by —O— or —N(R5a)- and wherein the alkyl moiety is optionally substituted by one or more halogen;
R2 represents halogen, cyano, hydroxyl, mercapto, C1-C6alkyl optionally substituted by one to five R14, C2-C6alkenyl optionally substituted by one to five R14, C2-C6alkynyl optionally substituted by one to five R14, C1-C6alkoxy optionally substituted by one to five R14, —N(R9a)(R9b), —C1-C6alkylene-N(R9a)(R9b), —CHO, —C1-C6alkylene-CHO, —C(O)OR10, —C1-C6alkylene-C(O)OR10, —C(O)N(R11a)(R11b), —C1-C6alkylene-C(O)N(R11a)(R11b), —N(R12)C(O)R13, —C1-C6alkylene-N(R12)C(O)R13, C1-C6alkylthio, C1-C6alkylsulfinyl, C1-C6alkylsulfonyl, Cycle-P, —C1-C6alkylene-Cycle-P, Cycle-Q or —C1-C6alkylene-Cycle-Q;
R3 represents independently at each occurrence hydrogen, halogen, cyano, C1-C4alkyl, C1-C4haloalkyl, C1-C4alkoxy, C1-C4haloalkoxy or —N(R8a)(R8b);
R4a, and R4b represent independently hydrogen, amino, —NH(C1-C4alkyl), —N(C1-C4alkyl)₂ or —C1-C4alkylene-R4c;
R4aa represents independently at each occurrence hydrogen, amino, —NH(C1-C4alkyl), —N(C1-C4alkyl)₂, —C1-C4alkylene-R4c or C3-C4cycloalkyl;
R4c represents independently at each occurrence hydrogen, cyano, hydroxyl, amino, C1-C4alkoxy, —CONH₂, —NH(C1-C4alkyl), —N(C1-C4alkyl)₂, Cycle-P or Cycle-Q;
R5a and R5b represent independently at each occurrence hydrogen or C1-C6alkyl;
R6a and R6b represent independently hydrogen or C1-C4alkyl;
each R8a and R8b represents independently at each occurrence hydrogen or C1-C4alkyl;
R9a represents hydrogen, C1-C6alkyl optionally substituted by one to five R14, —C1-C6alkylene-Cycle-P, —C1-C6alkylene-Cycle-Q, Cycle-P or Cycle-Q;
R9b, R11a, R11b and R12 represent independently hydrogen or C1-C6alkyl;
R10 and R13 represent independently at each occurrence C1-C6alkyl;
R14 represents independently at each occurrence halogen, cyano, hydroxyl, C1-C6alkoxy, amino, —NH(C1-C4alkyl), —N(C1-C4alkyl)₂ or —N(R12)C(O)R13;
Cycle-P represents independently at each occurrence a saturated or partially unsaturated 3- to 8-membered carbocyclic ring optionally substituted by 1 to 3 R16, or a saturated or partially unsaturated 3- to 8-membered heterocyclic ring optionally substituted by 1 to 3 R16 containing carbon atoms as ring members and one or two ring members independently selected from N and O, wherein N optionally may bear R15;
Cycle-Q represents independently at each occurrence phenyl optionally substituted by 1 to 3 R17 or a 5- to 6-membered heteroaryl ring containing one to four heteroatoms selected from O, S and N, optionally substituted by 1 to 3 R17;
R15 represents independently at each occurrence hydrogen or C1-C4alkyl;
R16 and R17 represent independently at each occurrence cyano, C1-C4alkyl, C1-C4haloalkyl, C1-C4alkoxy or C1-C4haloalkoxy;
n is 1 or 2; and
q is 0, 1, 2, 3 or 4.

31-35. (canceled)

36. A pharmaceutical composition, comprising a compound according to claim 1, or a pharmaceutically acceptable salt, solvate or hydrate thereof, and a pharmaceutically acceptable excipient.

37. The method according to claim 30, wherein the subject is a mammal.

38. The method according to claim 30, wherein the subject is a human.

39. The method according to claim 30, wherein the proliferative disease is cancer.
